SEQUENCE LISTING (1564 sequences)

SEQ ID NO: 1 - DNA, 10 bp, Homo sapiens
atactccact

SEQ ID NO: 2 - DNA, 10 bp, Homo sapiens
tcagctgcaa

SEQ ID NO: 3 - DNA, 10 bp, Homo sapiens
gtcatcacca

SEQ ID NO: 4 - DNA, 10 bp, Homo sapiens
ccttcaaatc

SEQ ID NO: 5 - DNA, 10 bp, Homo sapiens
acacccatca

SEQ ID NO: 6 - DNA, 10 bp, Homo sapiens
ccaacaccag

SEQ ID NO: 7 - DNA, 10 bp, Homo sapiens
aatagtttcc

SEQ ID NO: 8 - DNA, 10 bp, Homo sapiens
ccaggcgtca

SEQ ID NO: 9 - DNA, 10 bp, Homo sapiens
gaacagctca

SEQ ID NO: 10 - DNA, 10 bp, Homo sapiens
tactcggcca

SEQ ID NO: 11 - DNA, 10 bp, Homo sapiens
gggggagaag

SEQ ID NO: 12 - DNA, 10 bp, Homo sapiens
agtgggctca

SEQ ID NO: 13 - DNA, 10 bp, Homo sapiens
gagcaccgtg

SEQ ID NO: 14 - DNA, 10 bp, Homo sapiens
gatctatcca

SEQ ID NO: 15 - DNA, 10 bp, Homo sapiens
gaacgccaga

SEQ ID NO: 16 - DNA, 10 bp, Homo sapiens
gccctcggag

SEQ ID NO: 17 - DNA, 10 bp, Homo sapiens
acaagcctag

SEQ ID NO: 18 - DNA, 10 bp, Homo sapiens
gtcacaggaa

SEQ ID NO: 19 - DNA, 10 bp, Homo sapiens
gccctcggag

SEQ ID NO: 20 - DNA, 10 bp, Homo sapiens
ctaggatgat

SEQ ID NO: 21 - DNA, 10 bp, Homo sapiens
ccaactatcg

SEQ ID NO: 22 - DNA, 10 bp, Homo sapiens
ctgacgggga

SEQ ID NO: 23 - DNA, 10 bp, Homo sapiens
gagggtttta

SEQ ID NO: 24 - DNA, 10 bp, Homo sapiens
ggggtcccat

SEQ ID NO: 25 - DNA, 10 bp, Homo sapiens
gccaggtcac

SEQ ID NO: 26 - DNA, 10 bp, Homo sapiens
agaacaccaa

SEQ ID NO: 27 - DNA, 10 bp, Homo sapiens
aatcccgccc

SEQ ID NO: 28 - DNA, 10 bp, Homo sapiens
acactgcctc

SEQ ID NO: 29 - DNA, 10 bp, Homo sapiens
agagtccagg

SEQ ID NO: 30 - DNA, 10 bp, Homo sapiens
ccagacgtag

SEQ ID NO: 31 - DNA, 10 bp, Homo sapiens
gaggcccccg

SEQ ID NO: 32 - DNA, 10 bp, Homo sapiens
ctgtgtgccc

SEQ ID NO: 33 - DNA, 10 bp, Homo sapiens
gagaggatgg

SEQ ID NO: 34 - DNA, 10 bp, Homo sapiens
ggctgaacca

SEQ ID NO: 35 - DNA, 10 bp, Homo sapiens
ccaaatcatt

SEQ ID NO: 36 - DNA, 10 bp, Homo sapiens
acggctgggc

SEQ ID NO: 37 - DNA, 10 bp, Homo sapiens
accttcatct

SEQ ID NO: 38 - DNA, 10 bp, Homo sapiens
agggcttgag

SEQ ID NO: 39 - DNA, 10 bp, Homo sapiens
accttcatct

SEQ ID NO: 40 - DNA, 10 bp, Homo sapiens
tcaggccaga

SEQ ID NO: 41 - DNA, 10 bp, Homo sapiens
ctgtgtgccc

SEQ ID NO: 42 - DNA, 10 bp, Homo sapiens
ggatgtcaac

SEQ ID NO: 43 - DNA, 10 bp, Homo sapiens
atctggagca

SEQ ID NO: 44 - DNA, 10 bp, Homo sapiens
gagaggatgg

SEQ ID NO: 45 - DNA, 10 bp, Homo sapiens
atctggagca

SEQ ID NO: 46 - DNA, 10 bp, Homo sapiens
ggatgtcaac

SEQ ID NO: 47 - DNA, 10 bp, Homo sapiens
cacagacaca

SEQ ID NO: 48 - DNA, 10 bp, Homo sapiens
tgctcctaac

SEQ ID NO: 49 - DNA, 10 bp, Homo sapiens
tatacccgga

SEQ ID NO: 50 - DNA, 10 bp, Homo sapiens
tatcctgatg

SEQ ID NO: 51 - DNA, 10 bp, Homo sapiens
ggccctcccg

SEQ ID NO: 52 - DNA, 10 bp, Homo sapiens
gtagcgatgg

SEQ ID NO: 53 - DNA, 10 bp, Homo sapiens
gcaggttgtg

SEQ ID NO: 54 - DNA, 10 bp, Homo sapiens
tgggaaccgg

SEQ ID NO: 55 - DNA, 10 bp, Homo sapiens
acacctctct

SEQ ID NO: 56 - DNA, 10 bp, Homo sapiens
ggaaaacagg

SEQ ID NO: 57 - DNA, 10 bp, Homo sapiens
caggcggcac

SEQ ID NO: 58 - DNA, 10 bp, Homo sapiens
caggttggtc

SEQ ID NO: 59 - DNA, 10 bp, Homo sapiens
gggatataaa

SEQ ID NO: 60 - DNA, 10 bp, Homo sapiens
gtggaaaatc

SEQ ID NO: 61 - DNA, 10 bp, Homo sapiens
gtgtgtgaat

SEQ ID NO: 62 - DNA, 10 bp, Homo sapiens
atgtgacact

SEQ ID NO: 63 - DNA, 10 bp, Homo sapiens
atggtgtaat

SEQ ID NO: 64 - DNA, 10 bp, Homo sapiens
tcacattgat

SEQ ID NO: 65 - DNA, 10 bp, Homo sapiens
taactaaaca

SEQ ID NO: 66 - DNA, 10 bp, Homo sapiens
tgcccgggtc

SEQ ID NO: 67 - DNA, 10 bp, Homo sapiens
tagtcggaaa

SEQ ID NO: 68 - DNA, 10 bp, Homo sapiens
gctatacggg

SEQ ID NO: 69 - DNA, 10 bp, Homo sapiens
tcacacccca

SEQ ID NO: 70 - DNA, 10 bp, Homo sapiens
ctgcccgaac

SEQ ID NO: 71 - DNA, 10 bp, Homo sapiens
agtcacctct

SEQ ID NO: 72 - DNA, 10 bp, Homo sapiens
tcattggttt

SEQ ID NO: 73 - DNA, 10 bp, Homo sapiens
tcctctcctc

SEQ ID NO: 74 - DNA, 10 bp, Homo sapiens
cctctcggcc

SEQ ID NO: 75 - DNA, 10 bp, Homo sapiens
ccactgaagt

SEQ ID NO: 76 - DNA, 10 bp, Homo sapiens
ctggcttgct

SEQ ID NO: 77 - DNA, 10 bp, Homo sapiens
gaaaacagaa

SEQ ID NO: 78 - DNA, 10 bp, Homo sapiens
aaagcacgtc

SEQ ID NO: 79 - DNA, 10 bp, Homo sapiens
gaaaacagaa

SEQ ID NO: 80 - DNA, 10 bp, Homo sapiens
ttgattccat

SEQ ID NO: 81 - DNA, 10 bp, Homo sapiens
aaacaggcac

SEQ ID NO: 82 - DNA, 10 bp, Homo sapiens
cttacagtcc

SEQ ID NO: 83 - DNA, 10 bp, Homo sapiens
gaatggactc

SEQ ID NO: 84 - DNA, 10 bp, Homo sapiens
gaacccaaac

SEQ ID NO: 85 - DNA, 10 bp, Homo sapiens
gaaaacagaa

SEQ ID NO: 86 - DNA, 10 bp, Homo sapiens
actttgtccc

SEQ ID NO: 87 - DNA, 10 bp, Homo sapiens
gtgcgaatcc

SEQ ID NO: 88 - DNA, 10 bp, Homo sapiens
caaaaagtta

SEQ ID NO: 89 - DNA, 10 bp, Homo sapiens
ttaactttat

SEQ ID NO: 90 - DNA, 10 bp, Homo sapiens
cagccaaatg

SEQ ID NO: 91 - DNA, 10 bp, Homo sapiens
gcctgtggtg

SEQ ID NO: 92 - DNA, 10 bp, Homo sapiens
cttagggaca

SEQ ID NO: 93 - DNA, 10 bp, Homo sapiens
ttggaggtga

SEQ ID NO: 94 - DNA, 10 bp, Homo sapiens
attccatttc

SEQ ID NO: 95 - DNA, 10 bp, Homo sapiens
attccatttc

SEQ ID NO: 96 - DNA, 10 bp, Homo sapiens
agagagcgga

SEQ ID NO: 97 - DNA, 10 bp, Homo sapiens
ttctcaatac

SEQ ID NO: 98 - DNA, 10 bp, Homo sapiens
catcctccca

SEQ ID NO: 99 - DNA, 10 bp, Homo sapiens
gtatcgattt

SEQ ID NO: 100 - DNA, 10 bp, Homo sapiens
ttgtaaacag

SEQ ID NO: 101 - DNA, 10 bp, Homo sapiens
gccctgtatt

SEQ ID NO: 102 - DNA, 10 bp, Homo sapiens
ccacattgcc

SEQ ID NO: 103 - DNA, 10 bp, Homo sapiens
cagggcaacg

SEQ ID NO: 104 - DNA, 10 bp, Homo sapiens
aaaagcaaat

SEQ ID NO: 105 - DNA, 10 bp, Homo sapiens
accaatccta

SEQ ID NO: 106 - DNA, 10 bp, Homo sapiens
ctgtgtgtcc

SEQ ID NO: 107 - DNA, 10 bp, Homo sapiens
tcagacaata

SEQ ID NO: 108 - DNA, 10 bp, Homo sapiens
tggtgagatg

SEQ ID NO: 109 - DNA, 10 bp, Homo sapiens
attttttgtt

SEQ ID NO: 110 - DNA, 10 bp, Homo sapiens
acattgagtc

SEQ ID NO: 111 - DNA, 10 bp, Homo sapiens
gtcagtctac

SEQ ID NO: 112 - DNA, 10 bp, Homo sapiens
gtcccacttc

SEQ ID NO: 113 - DNA, 10 bp, Homo sapiens
ggggcccgaa

SEQ ID NO: 114 - DNA, 10 bp, Homo sapiens
tgactcaccc

SEQ ID NO: 115 - DNA, 10 bp, Homo sapiens
gacagcgaca

SEQ ID NO: 116 - DNA, 10 bp, Homo sapiens
ggtgtacata

SEQ ID NO: 117 - DNA, 10 bp, Homo sapiens
tagctataaa

SEQ ID NO: 118 - DNA, 10 bp, Homo sapiens
ggtgtacata

SEQ ID NO: 119 - DNA, 10 bp, Homo sapiens
gtttcatttt

SEQ ID NO: 120 - DNA, 10 bp, Homo sapiens
aataaattgc

SEQ ID NO: 121 - DNA, 10 bp, Homo sapiens
gtttcatttt

SEQ ID NO: 122 - DNA, 10 bp, Homo sapiens
acacattgta

SEQ ID NO: 123 - DNA, 10 bp, Homo sapiens
tacctattgt

SEQ ID NO: 124 - DNA, 10 bp, Homo sapiens
tttagcagaa

SEQ ID NO: 125 - DNA, 10 bp, Homo sapiens
tttagcagaa

SEQ ID NO: 126 - DNA, 10 bp, Homo sapiens
caatttatga

SEQ ID NO: 127 - DNA, 10 bp, Homo sapiens
gtgaaggttt

SEQ ID NO: 128 - DNA, 10 bp, Homo sapiens
tggactttta

SEQ ID NO: 129 - DNA, 10 bp, Homo sapiens
cgatgccacg

SEQ ID NO: 130 - DNA, 10 bp, Homo sapiens
gtgaaggttt

SEQ ID NO: 131 - DNA, 10 bp, Homo sapiens
tggactttta

SEQ ID NO: 132 - DNA, 10 bp, Homo sapiens
ccttcttgtc

SEQ ID NO: 133 - DNA, 10 bp, Homo sapiens
tccattcaag

SEQ ID NO: 134 - DNA, 10 bp, Homo sapiens
cctatgtatc

SEQ ID NO: 135 - DNA, 10 bp, Homo sapiens
acggaccaat

SEQ ID NO: 136 - DNA, 10 bp, Homo sapiens
tattatcttg

SEQ ID NO: 137 - DNA, 10 bp, Homo sapiens
actttatacg

SEQ ID NO: 138 - DNA, 10 bp, Homo sapiens
actttatacg

SEQ ID NO: 139 - DNA, 10 bp, Homo sapiens
cgcagtcccc

SEQ ID NO: 140 - DNA, 10 bp, Homo sapiens
tgtagtgctc

SEQ ID NO: 141 - DNA, 10 bp, Homo sapiens
ctgcttaagt

SEQ ID NO: 142 - DNA, 10 bp, Homo sapiens
acaagtggaa

SEQ ID NO: 143 - DNA, 10 bp, Homo sapiens
aatcccaatg

SEQ ID NO: 144 - DNA, 10 bp, Homo sapiens
actatgcatc

SEQ ID NO: 145 - DNA, 10 bp, Homo sapiens
acgagtcatt

SEQ ID NO: 146 - DNA, 10 bp, Homo sapiens
ttacattgta

SEQ ID NO: 147 - DNA, 10 bp, Homo sapiens
atgccccctc

SEQ ID NO: 148 - DNA, 10 bp, Homo sapiens
ttttattcat

SEQ ID NO: 149 - DNA, 10 bp, Homo sapiens
acagagcatt

SEQ ID NO: 150 - DNA, 10 bp, Homo sapiens
tgaccaatag

SEQ ID NO: 151 - DNA, 10 bp, Homo sapiens
aatcccaatg

SEQ ID NO: 152 - DNA, 10 bp, Homo sapiens
gcgaactggg

SEQ ID NO: 153 - DNA, 10 bp, Homo sapiens
gcaacactaa

SEQ ID NO: 154 - DNA, 10 bp, Homo sapiens
gtaatggatt

SEQ ID NO: 155 - DNA, 10 bp, Homo sapiens
agcagacgtg

SEQ ID NO: 156 - DNA, 10 bp, Homo sapiens
ggattcggtc

SEQ ID NO: 157 - DNA, 10 bp, Homo sapiens
cggaaggcgg

SEQ ID NO: 158 - DNA, 10 bp, Homo sapiens
tgtaagtacg

SEQ ID NO: 159 - DNA, 10 bp, Homo sapiens
gatcagtcat

SEQ ID NO: 160 - DNA, 10 bp, Homo sapiens
gctcagagtt

SEQ ID NO: 161 - DNA, 10 bp, Homo sapiens
taacctcccc

SEQ ID NO: 162 - DNA, 10 bp, Homo sapiens
aggaacaact

SEQ ID NO: 163 - DNA, 10 bp, Homo sapiens
gggtccgtgg

SEQ ID NO: 164 - DNA, 10 bp, Homo sapiens
tagcaaaata

SEQ ID NO: 165 - DNA, 10 bp, Homo sapiens
gctgtgcaca

SEQ ID NO: 166 - DNA, 10 bp, Homo sapiens
cagaaaatca

SEQ ID NO: 167 - DNA, 10 bp, Homo sapiens
gatttgctgg

SEQ ID NO: 168 - DNA, 10 bp, Homo sapiens
gtgccattct

SEQ ID NO: 169 - DNA, 10 bp, Homo sapiens
gatatttgtc

SEQ ID NO: 170 - DNA, 10 bp, Homo sapiens
tatgatttta

SEQ ID NO: 171 - DNA, 10 bp, Homo sapiens
tcactgcaac

SEQ ID NO: 172 - DNA, 10 bp, Homo sapiens
cccagtcaca

SEQ ID NO: 173 - DNA, 10 bp, Homo sapiens
tatgagaacc

SEQ ID NO: 174 - DNA, 10 bp, Homo sapiens
gagtttagtg

SEQ ID NO: 175 - DNA, 10 bp, Homo sapiens
ctccactctg

SEQ ID NO: 176 - DNA, 10 bp, Homo sapiens
atccagtgac

SEQ ID NO: 177 - DNA, 10 bp, Homo sapiens
tgatcttgag

SEQ ID NO: 178 - DNA, 10 bp, Homo sapiens
aatggctgtt

SEQ ID NO: 179 - DNA, 10 bp, Homo sapiens
atactaaaaa

SEQ ID NO: 180 - DNA, 10 bp, Homo sapiens
atactaaaaa

SEQ ID NO: 181 - DNA, 10 bp, Homo sapiens
gtttattaaa

SEQ ID NO: 182 - DNA, 10 bp, Homo sapiens
agaaatcagt

SEQ ID NO: 183 - DNA, 10 bp, Homo sapiens
ttggatatta

SEQ ID NO: 184 - DNA, 10 bp, Homo sapiens
aattgagtag

SEQ ID NO: 185 - DNA, 10 bp, Homo sapiens
tgagtgctgc

SEQ ID NO: 186 - DNA, 10 bp, Homo sapiens
gcagtacagt

SEQ ID NO: 187 - DNA, 10 bp, Homo sapiens
gaattcagga

SEQ ID NO: 188 - DNA, 10 bp, Homo sapiens
gacttcttta

SEQ ID NO: 189 - DNA, 10 bp, Homo sapiens
gaattcagga

SEQ ID NO: 190 - DNA, 10 bp, Homo sapiens
gtttatactg

SEQ ID NO: 191 - DNA, 10 bp, Homo sapiens
gaattcagga

SEQ ID NO: 192 - DNA, 10 bp, Homo sapiens
gcccgtgtag

SEQ ID NO: 193 - DNA, 10 bp, Homo sapiens
tggggtgtgc

SEQ ID NO: 194 - DNA, 10 bp, Homo sapiens
aatttttatg

SEQ ID NO: 195 - DNA, 10 bp, Homo sapiens
tcagtgtctg

SEQ ID NO: 196 - DNA, 10 bp, Homo sapiens
ggaggtcagc

SEQ ID NO: 197 - DNA, 10 bp, Homo sapiens
ttcttctcaa

SEQ ID NO: 198 - DNA, 10 bp, Homo sapiens
ttcttctcaa

SEQ ID NO: 199 - DNA, 10 bp, Homo sapiens
ggttgtctct

SEQ ID NO: 200 - DNA, 10 bp, Homo sapiens
ctttgtttac

SEQ ID NO: 201 - DNA, 10 bp, Homo sapiens
cactatagaa

SEQ ID NO: 202 - DNA, 10 bp, Homo sapiens
tttggttaca

SEQ ID NO: 203 - DNA, 10 bp, Homo sapiens
tcaaaacaat

SEQ ID NO: 204 - DNA, 10 bp, Homo sapiens
tttggttaca

SEQ ID NO: 205 - DNA, 10 bp, Homo sapiens
tatagagcaa

SEQ ID NO: 206 - DNA, 10 bp, Homo sapiens
taataaccag

SEQ ID NO: 207 - DNA, 10 bp, Homo sapiens
ttctatactg

SEQ ID NO: 208 - DNA, 10 bp, Homo sapiens
ggaatacggc

SEQ ID NO: 209 - DNA, 10 bp, Homo sapiens
tgaactggca

SEQ ID NO: 210 - DNA, 10 bp, Homo sapiens
aatgttgggg

SEQ ID NO: 211 - DNA, 10 bp, Homo sapiens
cgacaaacta

SEQ ID NO: 212 - DNA, 10 bp, Homo sapiens
gtagcacaga

SEQ ID NO: 213 - DNA, 10 bp, Homo sapiens
accgtcaatc

SEQ ID NO: 214 - DNA, 10 bp, Homo sapiens
tggatcagtc

SEQ ID NO: 215 - DNA, 10 bp, Homo sapiens
tggctcggtc

SEQ ID NO: 216 - DNA, 10 bp, Homo sapiens
gcgactgcga

SEQ ID NO: 217 - DNA, 10 bp, Homo sapiens
gcactagctg

SEQ ID NO: 218 - DNA, 10 bp, Homo sapiens
gcggccggtt

SEQ ID NO: 219 - DNA, 10 bp, Homo sapiens
cggcagtccc

SEQ ID NO: 220 - DNA, 10 bp, Homo sapiens
gcccacctgt

SEQ ID NO: 221 - DNA, 10 bp, Homo sapiens
cggcggatgg

SEQ ID NO: 222 - DNA, 10 bp, Homo sapiens
ccccaggccg

SEQ ID NO: 223 - DNA, 10 bp, Homo sapiens
cccattccaa

SEQ ID NO: 224 - DNA, 10 bp, Homo sapiens
tcaagaggtg

SEQ ID NO: 225 - DNA, 10 bp, Homo sapiens
ataactgttg

SEQ ID NO: 226 - DNA, 10 bp, Homo sapiens
atgtgtaacg

SEQ ID NO: 227 - DNA, 10 bp, Homo sapiens
ccctgccttg

SEQ ID NO: 228 - DNA, 10 bp, Homo sapiens
gtgcgctgag

SEQ ID NO: 229 - DNA, 10 bp, Homo sapiens
ctggccgctc

SEQ ID NO: 230 - DNA, 10 bp, Homo sapiens
gcccccccgt

SEQ ID NO: 231 - DNA, 10 bp, Homo sapiens
tggccccagg

SEQ ID NO: 232 - DNA, 10 bp, Homo sapiens
ccctggtggg

SEQ ID NO: 233 - DNA, 10 bp, Homo sapiens
agtgaccgaa

SEQ ID NO: 234 - DNA, 10 bp, Homo sapiens
ctgcacttac

SEQ ID NO: 235 - DNA, 10 bp, Homo sapiens
ctggcgagcg

SEQ ID NO: 236 - DNA, 10 bp, Homo sapiens
ttgccgctgc

SEQ ID NO: 237 - DNA, 10 bp, Homo sapiens
tgcgctggcc

SEQ ID NO: 238 - DNA, 10 bp, Homo sapiens
ctcctggaac

SEQ ID NO: 239 - DNA, 10 bp, Homo sapiens
cgcccgtcgt

SEQ ID NO: 240 - DNA, 10 bp, Homo sapiens
ttgcccccgt

SEQ ID NO: 241 - DNA, 10 bp, Homo sapiens
ttgctaaagg

SEQ ID NO: 242 - DNA, 10 bp, Homo sapiens
agccacgttg

SEQ ID NO: 243 - DNA, 10 bp, Homo sapiens
cctgggcact

SEQ ID NO: 244 - DNA, 10 bp, Homo sapiens
gggctcacct

SEQ ID NO: 245 - DNA, 10 bp, Homo sapiens
cttacagcca

SEQ ID NO: 246 - DNA, 10 bp, Homo sapiens
agggccctca

SEQ ID NO: 247 - DNA, 10 bp, Homo sapiens
gggtaatgtg

SEQ ID NO: 248 - DNA, 10 bp, Homo sapiens
ctgacagccc

SEQ ID NO: 249 - DNA, 10 bp, Homo sapiens
tgacctccag

SEQ ID NO: 250 - DNA, 10 bp, Homo sapiens
aaacctcttc

SEQ ID NO: 251 - DNA, 10 bp, Homo sapiens
tcattgcact

SEQ ID NO: 252 - DNA, 10 bp, Homo sapiens
ccccctccgg

SEQ ID NO: 253 - DNA, 10 bp, Homo sapiens
gtaggggcct

SEQ ID NO: 254 - DNA, 10 bp, Homo sapiens
gaacccaaag

SEQ ID NO: 255 - DNA, 10 bp, Homo sapiens
tgtgagcctc

SEQ ID NO: 256 - DNA, 10 bp, Homo sapiens
atctctggag

SEQ ID NO: 257 - DNA, 10 bp, Homo sapiens
aaagtgcatc

SEQ ID NO: 258 - DNA, 10 bp, Homo sapiens
gccttgggtg

SEQ ID NO: 259 - DNA, 10 bp, Homo sapiens
acctcactct

SEQ ID NO: 260 - DNA, 10 bp, Homo sapiens
taaagacttg

SEQ ID NO: 261 - DNA, 10 bp, Homo sapiens
tcggcgccgg

SEQ ID NO: 262 - DNA, 10 bp, Homo sapiens
aacctcgagt

SEQ ID NO: 263 - DNA, 10 bp, Homo sapiens
gtttacccgc

SEQ ID NO: 264 - DNA, 10 bp, Homo sapiens
gcctctgcct

SEQ ID NO: 265 - DNA, 10 bp, Homo sapiens
cctgggtcct

SEQ ID NO: 266 - DNA, 10 bp, Homo sapiens
catctaaact

SEQ ID NO: 267 - DNA, 10 bp, Homo sapiens
gggcaagcca

SEQ ID NO: 268 - DNA, 10 bp, Homo sapiens
attcagcacc

SEQ ID NO: 269 - DNA, 10 bp, Homo sapiens
ttgttattgc

SEQ ID NO: 270 - DNA, 10 bp, Homo sapiens
acagggtgac

SEQ ID NO: 271 - DNA, 10 bp, Homo sapiens
gcttccatct

SEQ ID NO: 272 - DNA, 10 bp, Homo sapiens
gcttccatct

SEQ ID NO: 273 - DNA, 10 bp, Homo sapiens
gagggtggcg

SEQ ID NO: 274 - DNA, 10 bp, Homo sapiens
gcagggtggg

SEQ ID NO: 275 - DNA, 10 bp, Homo sapiens
agccctccct

SEQ ID NO: 276 - DNA, 10 bp, Homo sapiens
atggccatag

SEQ ID NO: 277 - DNA, 10 bp, Homo sapiens
gtgggtgtcc

SEQ ID NO: 278 - DNA, 10 bp, Homo sapiens
tgtagtttga

SEQ ID NO: 279 - DNA, 10 bp, Homo sapiens
ggggctgtgg

SEQ ID NO: 280 - DNA, 10 bp, Homo sapiens
ggggctgtgg

SEQ ID NO: 281 - DNA, 10 bp, Homo sapiens
cacgcaatgc

SEQ ID NO: 282 - DNA, 10 bp, Homo sapiens
ctcacacatt

SEQ ID NO: 283 - DNA, 10 bp, Homo sapiens
caaatgagga

SEQ ID NO: 284 - DNA, 10 bp, Homo sapiens
tgtaagtctg

SEQ ID NO: 285 - DNA, 10 bp, Homo sapiens
accaaggagg

SEQ ID NO: 286 - DNA, 10 bp, Homo sapiens
accaaggagg

SEQ ID NO: 287 - DNA, 10 bp, Homo sapiens
accaaggagg

SEQ ID NO: 288 - DNA, 10 bp, Homo sapiens
tgaggcaggg

SEQ ID NO: 289 - DNA, 10 bp, Homo sapiens
tccacgcacc

SEQ ID NO: 290 - DNA, 10 bp, Homo sapiens
tagggcaatc

SEQ ID NO: 291 - DNA, 10 bp, Homo sapiens
ggtagcctgg

SEQ ID NO: 292 - DNA, 10 bp, Homo sapiens
tcaacagcca

SEQ ID NO: 293 - DNA, 10 bp, Homo sapiens
ctctgtgtgg

SEQ ID NO: 294 - DNA, 10 bp, Homo sapiens
cctatttact

SEQ ID NO: 295 - DNA, 10 bp, Homo sapiens
tgcatctggt

SEQ ID NO: 296 - DNA, 10 bp, Homo sapiens
gctctctatg

SEQ ID NO: 297 - DNA, 10 bp, Homo sapiens
gaaggcatcc

SEQ ID NO: 298 - DNA, 10 bp, Homo sapiens
ccactcctca

SEQ ID NO: 299 - DNA, 10 bp, Homo sapiens
gctgtcatca

SEQ ID NO: 300 - DNA, 10 bp, Homo sapiens
cggctggtga

SEQ ID NO: 301 - DNA, 10 bp, Homo sapiens
aagccaggac

SEQ ID NO: 302 - DNA, 10 bp, Homo sapiens
tgagagggtg

SEQ ID NO: 303 - DNA, 10 bp, Homo sapiens
gcgtgatcct

SEQ ID NO: 304 - DNA, 10 bp, Homo sapiens
ctgccaactt

SEQ ID NO: 305 - DNA, 10 bp, Homo sapiens
ccaaacgtgt

SEQ ID NO: 306 - DNA, 10 bp, Homo sapiens
gcgggagggc

SEQ ID NO: 307 - DNA, 10 bp, Homo sapiens
ggccagccct

SEQ ID NO: 308 - DNA, 10 bp, Homo sapiens
ggccagccct

SEQ ID NO: 309 - DNA, 10 bp, Homo sapiens
tgggcaaagc

SEQ ID NO: 310 - DNA, 10 bp, Homo sapiens
gcaaaaccag

SEQ ID NO: 311 - DNA, 10 bp, Homo sapiens
acttacctgc

SEQ ID NO: 312 - DNA, 10 bp, Homo sapiens
gttggtctgt

SEQ ID NO: 313 - DNA, 10 bp, Homo sapiens
tgctactggt

SEQ ID NO: 314 - DNA, 10 bp, Homo sapiens
gacgacacga

SEQ ID NO: 315 - DNA, 10 bp, Homo sapiens
caagtggcaa

SEQ ID NO: 316 - DNA, 10 bp, Homo sapiens
tactcttggc

SEQ ID NO: 317 - DNA, 10 bp, Homo sapiens
gactgtgcca

SEQ ID NO: 318 - DNA, 10 bp, Homo sapiens
ttgccggtta

SEQ ID NO: 319 - DNA, 10 bp, Homo sapiens
cattgcagga

SEQ ID NO: 320 - DNA, 10 bp, Homo sapiens
caggaacggg

SEQ ID NO: 321 - DNA, 10 bp, Homo sapiens
aataggtcca

SEQ ID NO: 322 - DNA, 10 bp, Homo sapiens
acctcaggaa

SEQ ID NO: 323 - DNA, 10 bp, Homo sapiens
atgactcaag

SEQ ID NO: 324 - DNA, 10 bp, Homo sapiens
atgactcaag

SEQ ID NO: 325 - DNA, 10 bp, Homo sapiens
gcctctgcca

SEQ ID NO: 326 - DNA, 10 bp, Homo sapiens
tgcttgtccc

SEQ ID NO: 327 - DNA, 10 bp, Homo sapiens
ggtggcactc

SEQ ID NO: 328 - DNA, 10 bp, Homo sapiens
gggctggggt

SEQ ID NO: 329 - DNA, 10 bp, Homo sapiens
gggctggggt

SEQ ID NO: 330 - DNA, 10 bp, Homo sapiens
cacaaacggt

SEQ ID NO: 331 - DNA, 10 bp, Homo sapiens
cattgaaggg

SEQ ID NO: 332 - DNA, 10 bp, Homo sapiens
gtgactgcca

SEQ ID NO: 333 - DNA, 10 bp, Homo sapiens
gtgactgcca

SEQ ID NO: 334 - DNA, 10 bp, Homo sapiens
aagacagtgg

SEQ ID NO: 335 - DNA, 10 bp, Homo sapiens
ctggctgcaa

SEQ ID NO: 336 - DNA, 10 bp, Homo sapiens
accgggaggt

SEQ ID NO: 337 - DNA, 10 bp, Homo sapiens
atggagactt

SEQ ID NO: 338 - DNA, 10 bp, Homo sapiens
cagctcatct

SEQ ID NO: 339 - DNA, 10 bp, Homo sapiens
acgtggtgat

SEQ ID NO: 340 - DNA, 10 bp, Homo sapiens
gcggtgaggt

SEQ ID NO: 341 - DNA, 10 bp, Homo sapiens
gtggcacacg

SEQ ID NO: 342 - DNA, 10 bp, Homo sapiens
gtgacaacac

SEQ ID NO: 343 - DNA, 10 bp, Homo sapiens
ctgctatacg

SEQ ID NO: 344 - DNA, 10 bp, Homo sapiens
actggctgct

SEQ ID NO: 345 - DNA, 10 bp, Homo sapiens
ggaagcacgg

SEQ ID NO: 346 - DNA, 10 bp, Homo sapiens
ggaagcacgg

SEQ ID NO: 347 - DNA, 10 bp, Homo sapiens
ctgttggtga

SEQ ID NO: 348 - DNA, 10 bp, Homo sapiens
tcagatcttt

SEQ ID NO: 349 - DNA, 10 bp, Homo sapiens
tggaatgctg

SEQ ID NO: 350 - DNA, 10 bp, Homo sapiens
taaggagctg

SEQ ID NO: 351 - DNA, 10 bp, Homo sapiens
ggctttggag

SEQ ID NO: 352 - DNA, 10 bp, Homo sapiens
cgcaccattg

SEQ ID NO: 353 - DNA, 10 bp, Homo sapiens
cgctggttcc

SEQ ID NO: 354 - DNA, 10 bp, Homo sapiens
gggcctgggg

SEQ ID NO: 355 - DNA, 10 bp, Homo sapiens
ctcgaggagg

SEQ ID NO: 356 - DNA, 10 bp, Homo sapiens
ttggtcctct

SEQ ID NO: 357 - DNA, 10 bp, Homo sapiens
tccctggcat

SEQ ID NO: 358 - DNA, 10 bp, Homo sapiens
gggggctgct

SEQ ID NO: 359 - DNA, 10 bp, Homo sapiens
gggggctgct

SEQ ID NO: 360 - DNA, 10 bp, Homo sapiens
ccaccccgaa

SEQ ID NO: 361 - DNA, 10 bp, Homo sapiens
ctgctaggaa

SEQ ID NO: 362 - DNA, 10 bp, Homo sapiens
aactgcggca

SEQ ID NO: 363 - DNA, 10 bp, Homo sapiens
tggagtggag

SEQ ID NO: 364 - DNA, 10 bp, Homo sapiens
tgaaggagcc

SEQ ID NO: 365 - DNA, 10 bp, Homo sapiens
ggggactgaa

SEQ ID NO: 366 - DNA, 10 bp, Homo sapiens
tgcacgtttt

SEQ ID NO: 367 - DNA, 10 bp, Homo sapiens
ctggatgccg

SEQ ID NO: 368 - DNA, 10 bp, Homo sapiens
ccccctcgtg

SEQ ID NO: 369 - DNA, 10 bp, Homo sapiens
atgatgcggt

SEQ ID NO: 370 - DNA, 10 bp, Homo sapiens
attctccagt

SEQ ID NO: 371 - DNA, 10 bp, Homo sapiens
ccccagttgc

SEQ ID NO: 372 - DNA, 10 bp, Homo sapiens
ccaaggattg

SEQ ID NO: 373 - DNA, 10 bp, Homo sapiens
gaccgaggtg

SEQ ID NO: 374 - DNA, 10 bp, Homo sapiens
gactctctca

SEQ ID NO: 375 - DNA, 10 bp, Homo sapiens
gactctggga

SEQ ID NO: 376 - DNA, 10 bp, Homo sapiens
gactctggga

SEQ ID NO: 377 - DNA, 10 bp, Homo sapiens
cgccgcggtg

SEQ ID NO: 378 - DNA, 10 bp, Homo sapiens
ccagaacaga

SEQ ID NO: 379 - DNA, 10 bp, Homo sapiens
ccagaacaga

SEQ ID NO: 380 - DNA, 10 bp, Homo sapiens
tggtttttgg

SEQ ID NO: 381 - DNA, 10 bp, Homo sapiens
tttttgtaca

SEQ ID NO: 382 - DNA, 10 bp, Homo sapiens
gttctcccac

SEQ ID NO: 383 - DNA, 10 bp, Homo sapiens
gaccctgccc

SEQ ID NO: 384 - DNA, 10 bp, Homo sapiens
gcccgccttg

SEQ ID NO: 385 - DNA, 10 bp, Homo sapiens
ggtgctggag

SEQ ID NO: 386 - DNA, 10 bp, Homo sapiens
ttacctcctt

SEQ ID NO: 387 - DNA, 10 bp, Homo sapiens
aaaccagggc

SEQ ID NO: 388 - DNA, 10 bp, Homo sapiens
ttctggctgc

SEQ ID NO: 389 - DNA, 10 bp, Homo sapiens
ttctggctgc

SEQ ID NO: 390 - DNA, 10 bp, Homo sapiens
cttctcaccg

SEQ ID NO: 391 - DNA, 10 bp, Homo sapiens
gagaaccgta

SEQ ID NO: 392 - DNA, 10 bp, Homo sapiens
gcgaccgtca

SEQ ID NO: 393 - DNA, 10 bp, Homo sapiens
gtcaagacca

SEQ ID NO: 394 - DNA, 10 bp, Homo sapiens
ctgggtctcc

SEQ ID NO: 395 - DNA, 10 bp, Homo sapiens
cgattctgga

SEQ ID NO: 396 - DNA, 10 bp, Homo sapiens
caggaggagt

SEQ ID NO: 397 - DNA, 10 bp, Homo sapiens
caaaatcagg

SEQ ID NO: 398 - DNA, 10 bp, Homo sapiens
ctgggttaat

SEQ ID NO: 399 - DNA, 10 bp, Homo sapiens
ttttctgctg

SEQ ID NO: 400 - DNA, 10 bp, Homo sapiens
ccctggcaat

SEQ ID NO: 401 - DNA, 10 bp, Homo sapiens
aggctacgga

SEQ ID NO: 402 - DNA, 10 bp, Homo sapiens
gaggccatcc

SEQ ID NO: 403 - DNA, 10 bp, Homo sapiens
ctttgatgtt

SEQ ID NO: 404 - DNA, 10 bp, Homo sapiens
ttggacctgg

SEQ ID NO: 405 - DNA, 10 bp, Homo sapiens
ttggacctgg

SEQ ID NO: 406 - DNA, 10 bp, Homo sapiens
gttcgtgcca

SEQ ID NO: 407 - DNA, 10 bp, Homo sapiens
gatgctgcca

SEQ ID NO: 408 - DNA, 10 bp, Homo sapiens
acggctccga

SEQ ID NO: 409 - DNA, 10 bp, Homo sapiens
gagtcaggag

SEQ ID NO: 410 - DNA, 10 bp, Homo sapiens
ggaggctgag

SEQ ID NO: 411 - DNA, 10 bp, Homo sapiens
ggaggctgag

SEQ ID NO: 412 - DNA, 10 bp, Homo sapiens
gtgatggtgt

SEQ ID NO: 413 - DNA, 10 bp, Homo sapiens
tcagatggcg

SEQ ID NO: 414 - DNA, 10 bp, Homo sapiens
atgcgaaagg

SEQ ID NO: 415 - DNA, 10 bp, Homo sapiens
tgctgggtgg

SEQ ID NO: 416 - DNA, 10 bp, Homo sapiens
tgctgggtgg

SEQ ID NO: 417 - DNA, 10 bp, Homo sapiens
tcaaatgcat

SEQ ID NO: 418 - DNA, 10 bp, Homo sapiens
tccaaggaag

SEQ ID NO: 419 - DNA, 10 bp, Homo sapiens
cccagggaga

SEQ ID NO: 420 - DNA, 10 bp, Homo sapiens
tggcctgccc

SEQ ID NO: 421 - DNA, 10 bp, Homo sapiens
tggcctgccc

SEQ ID NO: 422 - DNA, 10 bp, Homo sapiens
ggccaaaggc

SEQ ID NO: 423 - DNA, 10 bp, Homo sapiens
ggcctgctgc

SEQ ID NO: 424 - DNA, 10 bp, Homo sapiens
gtgaagctga

SEQ ID NO: 425 - DNA, 10 bp, Homo sapiens
gtgaagctga

SEQ ID NO: 426 - DNA, 10 bp, Homo sapiens
gaaatgtaag

SEQ ID NO: 427 - DNA, 10 bp, Homo sapiens
gaaatgtaag

SEQ ID NO: 428 - DNA, 10 bp, Homo sapiens
cgtgttaatg

SEQ ID NO: 429 - DNA, 10 bp, Homo sapiens
aggggattcc

SEQ ID NO: 430 - DNA, 10 bp, Homo sapiens
cagctcactg

SEQ ID NO: 431 - DNA, 10 bp, Homo sapiens
gtttggcagt

SEQ ID NO: 432 - DNA, 10 bp, Homo sapiens
ggagctctgt

SEQ ID NO: 433 - DNA, 10 bp, Homo sapiens
tggaactgtg

SEQ ID NO: 434 - DNA, 10 bp, Homo sapiens
tctgcttaca

SEQ ID NO: 435 - DNA, 10 bp, Homo sapiens
agggcttcca

SEQ ID NO: 436 - DNA, 10 bp, Homo sapiens
gagcaaacgg

SEQ ID NO: 437 - DNA, 10 bp, Homo sapiens
tgtgatcaga

SEQ ID NO: 438 - DNA, 10 bp, Homo sapiens
acactacggg

SEQ ID NO: 439 - DNA, 10 bp, Homo sapiens
agccaaaaaa

SEQ ID NO: 440 - DNA, 10 bp, Homo sapiens
gcgggtgtgg

SEQ ID NO: 441 - DNA, 10 bp, Homo sapiens
ttgctagagg

SEQ ID NO: 442 - DNA, 10 bp, Homo sapiens
ggggcttctg

SEQ ID NO: 443 - DNA, 10 bp, Homo sapiens
aactcttgaa

SEQ ID NO: 444 - DNA, 10 bp, Homo sapiens
gtctgacccc

SEQ ID NO: 445 - DNA, 10 bp, Homo sapiens
atgtcatcaa

SEQ ID NO: 446 - DNA, 10 bp, Homo sapiens
tctgtcaaga

SEQ ID NO: 447 - DNA, 10 bp, Homo sapiens
gccccagcga

SEQ ID NO: 448 - DNA, 10 bp, Homo sapiens
ggcaagcccc

SEQ ID NO: 449 - DNA, 10 bp, Homo sapiens
ctcatcagct

SEQ ID NO: 450 - DNA, 10 bp, Homo sapiens
ctgttgattg

SEQ ID NO: 451 - DNA, 10 bp, Homo sapiens
gcttttaagg

SEQ ID NO: 452 - DNA, 10 bp, Homo sapiens
gcctgagcct

SEQ ID NO: 453 - DNA, 10 bp, Homo sapiens
gagcgggatg

SEQ ID NO: 454 - DNA, 10 bp, Homo sapiens
ttcacagtgg

SEQ ID NO: 455 - DNA, 10 bp, Homo sapiens
gcccgtgcca

SEQ ID NO: 456 - DNA, 10 bp, Homo sapiens
ccctaggttg

SEQ ID NO: 457 - DNA, 10 bp, Homo sapiens
ccctgatttt

SEQ ID NO: 458 - DNA, 10 bp, Homo sapiens
gtgttaacca

SEQ ID NO: 459 - DNA, 10 bp, Homo sapiens
aggaaagctg

SEQ ID NO: 460 - DNA, 10 bp, Homo sapiens
ttctctctgt

SEQ ID NO: 461 - DNA, 10 bp, Homo sapiens
ttactaaatg

SEQ ID NO: 462 - DNA, 10 bp, Homo sapiens
gggtgtggtg

SEQ ID NO: 463 - DNA, 10 bp, Homo sapiens
ccactgcagt

SEQ ID NO: 464 - DNA, 10 bp, Homo sapiens
agcctggact

SEQ ID NO: 465 - DNA, 10 bp, Homo sapiens
gtggggtgac

SEQ ID NO: 466 - DNA, 10 bp, Homo sapiens
cactacacgg

SEQ ID NO: 467 - DNA, 10 bp, Homo sapiens
ctcatagcag

SEQ ID NO: 468 - DNA, 10 bp, Homo sapiens
ggaatgtacg

SEQ ID NO: 469 - DNA, 10 bp, Homo sapiens
ctgagggtgg

SEQ ID NO: 470 - DNA, 10 bp, Homo sapiens
aaggtcgagc

SEQ ID NO: 471 - DNA, 10 bp, Homo sapiens
gaatcactgc

SEQ ID NO: 472 - DNA, 10 bp, Homo sapiens
acatcatcga

SEQ ID NO: 473 - DNA, 10 bp, Homo sapiens
gaatgaggac

SEQ ID NO: 474 - DNA, 10 bp, Homo sapiens
cctcgctcag

SEQ ID NO: 475 - DNA, 10 bp, Homo sapiens
tcctagcctg

SEQ ID NO: 476 - DNA, 10 bp, Homo sapiens
aggtgcgggg

SEQ ID NO: 477 - DNA, 10 bp, Homo sapiens
ctccaataaa

SEQ ID NO: 478 - DNA, 10 bp, Homo sapiens
gcgctggagt

SEQ ID NO: 479 - DNA, 10 bp, Homo sapiens
aatttgcaac

SEQ ID NO: 480 - DNA, 10 bp, Homo sapiens
aacgcggcca

SEQ ID NO: 481 - DNA, 10 bp, Homo sapiens
ggtgtatatg

SEQ ID NO: 482 - DNA, 10 bp, Homo sapiens
ggcaacaaaa

SEQ ID NO: 483 - DNA, 10 bp, Homo sapiens
ggcaacaaaa

SEQ ID NO: 484 - DNA, 10 bp, Homo sapiens
tttgtgactg

SEQ ID NO: 485 - DNA, 10 bp, Homo sapiens
atgaggccgg

SEQ ID NO: 486 - DNA, 10 bp, Homo sapiens
tcagtttgtc

SEQ ID NO: 487 - DNA, 10 bp, Homo sapiens
ccctattaag

SEQ ID NO: 488 - DNA, 10 bp, Homo sapiens
tttctagttt

SEQ ID NO: 489 - DNA, 10 bp, Homo sapiens
gggcccttcc

SEQ ID NO: 490 - DNA, 10 bp, Homo sapiens
gggcccttcc

SEQ ID NO: 491 - DNA, 10 bp, Homo sapiens
ccttggtttt

SEQ ID NO: 492 - DNA, 10 bp, Homo sapiens
gctaaggaga

SEQ ID NO: 493 - DNA, 10 bp, Homo sapiens
tgaggggtga

SEQ ID NO: 494 - DNA, 10 bp, Homo sapiens
ccagctgcca

SEQ ID NO: 495 - DNA, 10 bp, Homo sapiens
gggctgtttg

SEQ ID NO: 496 - DNA, 10 bp, Homo sapiens
tggacacaag

SEQ ID NO: 497 - DNA, 10 bp, Homo sapiens
tctccaggaa

SEQ ID NO: 498 - DNA, 10 bp, Homo sapiens
tgatgtttga

SEQ ID NO: 499 - DNA, 10 bp, Homo sapiens
gtggtgcacg

SEQ ID NO: 500 - DNA, 10 bp, Homo sapiens
gtctgcacct

SEQ ID NO: 501 - DNA, 10 bp, Homo sapiens
gatgaccccg

SEQ ID NO: 502 - DNA, 10 bp, Homo sapiens
atcaagggtg

SEQ ID NO: 503 - DNA, 10 bp, Homo sapiens
tctggtctgg

SEQ ID NO: 504 - DNA, 10 bp, Homo sapiens
aggatgaccc

SEQ ID NO: 505 - DNA, 10 bp, Homo sapiens
aaagggggca

SEQ ID NO: 506 - DNA, 10 bp, Homo sapiens
ggctttaccc

SEQ ID NO: 507 - DNA, 10 bp, Homo sapiens
gctttttaga

SEQ ID NO: 508 - DNA, 10 bp, Homo sapiens
ctctgctcgg

SEQ ID NO: 509 - DNA, 10 bp, Homo sapiens
gcctgggact

SEQ ID NO: 510 - DNA, 10 bp, Homo sapiens
ggtagcaggg

SEQ ID NO: 511 - DNA, 10 bp, Homo sapiens
gccgatcctc

SEQ ID NO: 512 - DNA, 10 bp, Homo sapiens
gcagctcagg

SEQ ID NO: 513 - DNA, 10 bp, Homo sapiens
cgcagtgtcc

SEQ ID NO: 514 - DNA, 10 bp, Homo sapiens
cccctattaa

SEQ ID NO: 515 - DNA, 10 bp, Homo sapiens
ttgtaaaagg

SEQ ID NO: 516 - DNA, 10 bp, Homo sapiens
ccacaccggt

SEQ ID NO: 517 - DNA, 10 bp, Homo sapiens
cctggaagag

SEQ ID NO: 518 - DNA, 10 bp, Homo sapiens
tagccgctga

SEQ ID NO: 519 - DNA, 10 bp, Homo sapiens
cctaggacct

SEQ ID NO: 520 - DNA, 10 bp, Homo sapiens
gtggaccctg

SEQ ID NO: 521 - DNA, 10 bp, Homo sapiens
gtggaccctg

SEQ ID NO: 522 - DNA, 10 bp, Homo sapiens
ttgggagcag

SEQ ID NO: 523 - DNA, 10 bp, Homo sapiens
gtctcacgtg

SEQ ID NO: 524 - DNA, 10 bp, Homo sapiens
gtactgtggc

SEQ ID NO: 525 - DNA, 10 bp, Homo sapiens
aagataatgc

SEQ ID NO: 526 - DNA, 10 bp, Homo sapiens
aatacctcgt

SEQ ID NO: 527 - DNA, 10 bp, Homo sapiens
accttgtgcc

SEQ ID NO: 528 - DNA, 10 bp, Homo sapiens
accttgtgcc

SEQ ID NO: 529 - DNA, 10 bp, Homo sapiens
ggagggggct

SEQ ID NO: 530 - DNA, 10 bp, Homo sapiens
gcctatggtc

SEQ ID NO: 531 - DNA, 10 bp, Homo sapiens
gtgctgaatg

SEQ ID NO: 532 - DNA, 10 bp, Homo sapiens
tcgtcgcaga

SEQ ID NO: 533 - DNA, 10 bp, Homo sapiens
gtgacagaag

SEQ ID NO: 534 - DNA, 10 bp, Homo sapiens
tcaacggtgt

SEQ ID NO: 535 - DNA, 10 bp, Homo sapiens
gagccttggt

SEQ ID NO: 536 - DNA, 10 bp, Homo sapiens
tacatccgaa

SEQ ID NO: 537 - DNA, 10 bp, Homo sapiens
gtctgtgaga

SEQ ID NO: 538 - DNA, 10 bp, Homo sapiens
gttaacgtcc

SEQ ID NO: 539 - DNA, 10 bp, Homo sapiens
gtgcgctagg

SEQ ID NO: 540 - DNA, 10 bp, Homo sapiens
cggataaggc

SEQ ID NO: 541 - DNA, 10 bp, Homo sapiens
gtctggggct

SEQ ID NO: 542 - DNA, 10 bp, Homo sapiens
catcctgctg

SEQ ID NO: 543 - DNA, 10 bp, Homo sapiens
tcacaagcaa

SEQ ID NO: 544 - DNA, 10 bp, Homo sapiens
ggctgatgtg

SEQ ID NO: 545 - DNA, 10 bp, Homo sapiens
cccgtccgga

SEQ ID NO: 546 - DNA, 10 bp, Homo sapiens
tccgcgagaa

SEQ ID NO: 547 - DNA, 10 bp, Homo sapiens
gtgctggaga

SEQ ID NO: 548 - DNA, 10 bp, Homo sapiens
tcctcaagat

SEQ ID NO: 549 - DNA, 10 bp, Homo sapiens
caacttagtt

SEQ ID NO: 550 - DNA, 10 bp, Homo sapiens
gggcagctgg

SEQ ID NO: 551 - DNA, 10 bp, Homo sapiens
tttcagagag

SEQ ID NO: 552 - DNA, 10 bp, Homo sapiens
tttcagagag

SEQ ID NO: 553 - DNA, 10 bp, Homo sapiens
gacgcagaag

SEQ ID NO: 554 - DNA, 10 bp, Homo sapiens
ggaagtttcg

SEQ ID NO: 555 - DNA, 10 bp, Homo sapiens
gttgctgccc

SEQ ID NO: 556 - DNA, 10 bp, Homo sapiens
gctggggtgg

SEQ ID NO: 557 - DNA, 10 bp, Homo sapiens
ctcaacatct

SEQ ID NO: 558 - DNA, 10 bp, Homo sapiens
caagcaggac

SEQ ID NO: 559 - DNA, 10 bp, Homo sapiens
ttggcttttc

SEQ ID NO: 560 - DNA, 10 bp, Homo sapiens
tggcaacctt

SEQ ID NO: 561 - DNA, 10 bp, Homo sapiens
gcataatagg

SEQ ID NO: 562 - DNA, 10 bp, Homo sapiens
gggggtaact

SEQ ID NO: 563 - DNA, 10 bp, Homo sapiens
ccttcgagat

SEQ ID NO: 564 - DNA, 10 bp, Homo sapiens
cgggccgtgc

SEQ ID NO: 565 - DNA, 10 bp, Homo sapiens
gtgttgcaca

SEQ ID NO: 566 - DNA, 10 bp, Homo sapiens
cctcggaaaa

SEQ ID NO: 567 - DNA, 10 bp, Homo sapiens
aataaaggct

SEQ ID NO: 568 - DNA, 10 bp, Homo sapiens
aataaaggct

SEQ ID NO: 569 - DNA, 10 bp, Homo sapiens
cttctgtgta

SEQ ID NO: 570 - DNA, 10 bp, Homo sapiens
cttctgtgta

SEQ ID NO: 571 - DNA, 10 bp, Homo sapiens
ggtccagtgt

SEQ ID NO: 572 - DNA, 10 bp, Homo sapiens
agcacctcca

SEQ ID NO: 573 - DNA, 10 bp, Homo sapiens
aagctgagtg

SEQ ID NO: 574 - DNA, 10 bp, Homo sapiens
gtttcttccc

SEQ ID NO: 575 - DNA, 10 bp, Homo sapiens
tgagggaata

SEQ ID NO: 576 - DNA, 10 bp, Homo sapiens
agctctccct

SEQ ID NO: 577 - DNA, 10 bp, Homo sapiens
tacgttgcag

SEQ ID NO: 578 - DNA, 10 bp, Homo sapiens
gggtgtgtat

SEQ ID NO: 579 - DNA, 10 bp, Homo sapiens
ggagggatca

SEQ ID NO: 580 - DNA, 10 bp, Homo sapiens
atcagtggct

SEQ ID NO: 581 - DNA, 10 bp, Homo sapiens
ccccctgccc

SEQ ID NO: 582 - DNA, 10 bp, Homo sapiens
ccccctgccc

SEQ ID NO: 583 - DNA, 10 bp, Homo sapiens
caaaaaaaaa

SEQ ID NO: 584 - DNA, 10 bp, Homo sapiens
acctgccgac

SEQ ID NO: 585 - DNA, 10 bp, Homo sapiens
gaccagaaaa

SEQ ID NO: 586 - DNA, 10 bp, Homo sapiens
agccactgcg

SEQ ID NO: 587 - DNA, 10 bp, Homo sapiens
ttgagccagc

SEQ ID NO: 588 - DNA, 10 bp, Homo sapiens
tttcagggga

SEQ ID NO: 589 - DNA, 10 bp, Homo sapiens
tccggccgcg

SEQ ID NO: 590 - DNA, 10 bp, Homo sapiens
gtgatctccg

SEQ ID NO: 591 - DNA, 10 bp, Homo sapiens
ctgctgagtg

SEQ ID NO: 592 - DNA, 10 bp, Homo sapiens
ctgcttaagg

SEQ ID NO: 593 - DNA, 10 bp, Homo sapiens
tgtggcctcc

SEQ ID NO: 594 - DNA, 10 bp, Homo sapiens
cgttttctga

SEQ ID NO: 595 - DNA, 10 bp, Homo sapiens
ggaaaaaaaa

SEQ ID NO: 596 - DNA, 10 bp, Homo sapiens
ggaaaaaaaa

SEQ ID NO: 597 - DNA, 10 bp, Homo sapiens
gagggagttt

SEQ ID NO: 598 - DNA, 10 bp, Homo sapiens
gactcacttt

SEQ ID NO: 599 - DNA, 10 bp, Homo sapiens
gagaacgggg

SEQ ID NO: 600 - DNA, 10 bp, Homo sapiens
tggctagtgt

SEQ ID NO: 601 - DNA, 10 bp, Homo sapiens
ctgtcatttg

SEQ ID NO: 602 - DNA, 10 bp, Homo sapiens
gttccctggc

SEQ ID NO: 603 - DNA, 10 bp, Homo sapiens
gcatttaaat

SEQ ID NO: 604 - DNA, 10 bp, Homo sapiens
atccacatcg

SEQ ID NO: 605 - DNA, 10 bp, Homo sapiens
ctgctgtgat

SEQ ID NO: 606 - DNA, 10 bp, Homo sapiens
gtgacctcct

SEQ ID NO: 607 - DNA, 10 bp, Homo sapiens
gtggacccca

SEQ ID NO: 608 - DNA, 10 bp, Homo sapiens
gactagtgcg

SEQ ID NO: 609 - DNA, 10 bp, Homo sapiens
ttatgggatc

SEQ ID NO: 610 - DNA, 10 bp, Homo sapiens
tttcagattg

SEQ ID NO: 611 - DNA, 10 bp, Homo sapiens
gtctgagctc

SEQ ID NO: 612 - DNA, 10 bp, Homo sapiens
cacacaatgt

SEQ ID NO: 613 - DNA, 10 bp, Homo sapiens
cacacaatgt

SEQ ID NO: 614 - DNA, 10 bp, Homo sapiens
accccaccca

SEQ ID NO: 615 - DNA, 10 bp, Homo sapiens
ggaggcaggt

SEQ ID NO: 616 - DNA, 10 bp, Homo sapiens
tctcaattct

SEQ ID NO: 617 - DNA, 10 bp, Homo sapiens
ctcttcagga

SEQ ID NO: 618 - DNA, 10 bp, Homo sapiens
ctgggactgc

SEQ ID NO: 619 - DNA, 10 bp, Homo sapiens
gcccagcagg

SEQ ID NO: 620 - DNA, 10 bp, Homo sapiens
gcccagcagg

SEQ ID NO: 621 - DNA, 10 bp, Homo sapiens
gggccagggg

SEQ ID NO: 622 - DNA, 10 bp, Homo sapiens
gggggacggc

SEQ ID NO: 623 - DNA, 10 bp, Homo sapiens
actgggtcta

SEQ ID NO: 624 - DNA, 10 bp, Homo sapiens
gccgaggaag

SEQ ID NO: 625 - DNA, 10 bp, Homo sapiens
cagatctttg

SEQ ID NO: 626 - DNA, 10 bp, Homo sapiens
aggtttcctc

SEQ ID NO: 627 - DNA, 10 bp, Homo sapiens
ccgtccaagg

SEQ ID NO: 628 - DNA, 10 bp, Homo sapiens
gtggcgggcg

SEQ ID NO: 629 - DNA, 10 bp, Homo sapiens
gtggcgggcg

SEQ ID NO: 630 - DNA, 10 bp, Homo sapiens
gtggcgggcg

SEQ ID NO: 631 - DNA, 10 bp, Homo sapiens
ggcaagaaga

SEQ ID NO: 632 - DNA, 10 bp, Homo sapiens
tctttacttg

SEQ ID NO: 633 - DNA, 10 bp, Homo sapiens
ctcctcacct

SEQ ID NO: 634 - DNA, 10 bp, Homo sapiens
ctcctcacct

SEQ ID NO: 635 - DNA, 10 bp, Homo sapiens
gcctgtatga

SEQ ID NO: 636 - DNA, 10 bp, Homo sapiens
gctttatttg

SEQ ID NO: 637 - DNA, 10 bp, Homo sapiens
cttaaggatt

SEQ ID NO: 638 - DNA, 10 bp, Homo sapiens
ggatttggcc

SEQ ID NO: 639 - DNA, 10 bp, Homo sapiens
ggatttggcc

SEQ ID NO: 640 - DNA, 10 bp, Homo sapiens
ggatttggcc

SEQ ID NO: 641 - DNA, 10 bp, Homo sapiens
tcctccctcc

SEQ ID NO: 642 - DNA, 10 bp, Homo sapiens
ggccctctga

SEQ ID NO: 643 - DNA, 10 bp, Homo sapiens
tggctgtgtg

SEQ ID NO: 644 - DNA, 10 bp, Homo sapiens
agaccaaagt

SEQ ID NO: 645 - DNA, 10 bp, Homo sapiens
atggccaact

SEQ ID NO: 646 - DNA, 10 bp, Homo sapiens
aggagctgct

SEQ ID NO: 647 - DNA, 10 bp, Homo sapiens
aggagctgct

SEQ ID NO: 648 - DNA, 10 bp, Homo sapiens
tgtacctgta

SEQ ID NO: 649 - DNA, 10 bp, Homo sapiens
gatcccaaca

SEQ ID NO: 650 - DNA, 10 bp, Homo sapiens
ggccatctct

SEQ ID NO: 651 - DNA, 10 bp, Homo sapiens
aggtgcagag

SEQ ID NO: 652 - DNA, 10 bp, Homo sapiens
gtggcatcac

SEQ ID NO: 653 - DNA, 10 bp, Homo sapiens
tgtgttgaga

SEQ ID NO: 654 - DNA, 10 bp, Homo sapiens
ctgagacaaa

SEQ ID NO: 655 - DNA, 10 bp, Homo sapiens
gcaacgggcc

SEQ ID NO: 656 - DNA, 10 bp, Homo sapiens
gctggctggc

SEQ ID NO: 657 - DNA, 10 bp, Homo sapiens
gccaagatgc

SEQ ID NO: 658 - DNA, 10 bp, Homo sapiens
gccaaggggc

SEQ ID NO: 659 - DNA, 10 bp, Homo sapiens
acggtgatgt

SEQ ID NO: 660 - DNA, 10 bp, Homo sapiens
cccatccgaa

SEQ ID NO: 661 - DNA, 10 bp, Homo sapiens
acaaacttag

SEQ ID NO: 662 - DNA, 10 bp, Homo sapiens
gcctcctccc

SEQ ID NO: 663 - DNA, 10 bp, Homo sapiens
gtgcctgaga

SEQ ID NO: 664 - DNA, 10 bp, Homo sapiens
tccaatactg

SEQ ID NO: 665 - DNA, 10 bp, Homo sapiens
gtggtgcgtg

SEQ ID NO: 666 - DNA, 10 bp, Homo sapiens
aagaagcagg

SEQ ID NO: 667 - DNA, 10 bp, Homo sapiens
acttggagcc

SEQ ID NO: 668 - DNA, 10 bp, Homo sapiens
ccgtggtcac

SEQ ID NO: 669 - DNA, 10 bp, Homo sapiens
acagtgggga

SEQ ID NO: 670 - DNA, 10 bp, Homo sapiens
acaaactgtg

SEQ ID NO: 671 - DNA, 10 bp, Homo sapiens
gtcttaactc

SEQ ID NO: 672 - DNA, 10 bp, Homo sapiens
ctgtgctcgg

SEQ ID NO: 673 - DNA, 10 bp, Homo sapiens
gtggcctgca

SEQ ID NO: 674 - DNA, 10 bp, Homo sapiens
tggtacacgt

SEQ ID NO: 675 - DNA, 10 bp, Homo sapiens
gtactgtatg

SEQ ID NO: 676 - DNA, 10 bp, Homo sapiens
gtactgtatg

SEQ ID NO: 677 - DNA, 10 bp, Homo sapiens
ggccaggtgg

SEQ ID NO: 678 - DNA, 10 bp, Homo sapiens
ggccaggtgg

SEQ ID NO: 679 - DNA, 10 bp, Homo sapiens
agggagaggg

SEQ ID NO: 680 - DNA, 10 bp, Homo sapiens
agggagaggg

SEQ ID NO: 681 - DNA, 10 bp, Homo sapiens
agggagaggg

SEQ ID NO: 682 - DNA, 10 bp, Homo sapiens
gtggcaggtg

SEQ ID NO: 683 - DNA, 10 bp, Homo sapiens
tcttgtgcat

SEQ ID NO: 684 - DNA, 10 bp, Homo sapiens
ccacacaccg

SEQ ID NO: 685 - DNA, 10 bp, Homo sapiens
acaaatcctt

SEQ ID NO: 686 - DNA, 10 bp, Homo sapiens
gtgagacccc

SEQ ID NO: 687 - DNA, 10 bp, Homo sapiens
aaagccaaga

SEQ ID NO: 688 - DNA, 10 bp, Homo sapiens
caaggatcta

SEQ ID NO: 689 - DNA, 10 bp, Homo sapiens
tgaggccagg

SEQ ID NO: 690 - DNA, 10 bp, Homo sapiens
ttttgtgtga

SEQ ID NO: 691 - DNA, 10 bp, Homo sapiens
acagtcttgc

SEQ ID NO: 692 - DNA, 10 bp, Homo sapiens
acagtcttgc

SEQ ID NO: 693 - DNA, 10 bp, Homo sapiens
ccaggcacgc

SEQ ID NO: 694 - DNA, 10 bp, Homo sapiens
agtttcccaa

SEQ ID NO: 695 - DNA, 10 bp, Homo sapiens
ccagtggccc

SEQ ID NO: 696 - DNA, 10 bp, Homo sapiens
gccccgccct

SEQ ID NO: 697 - DNA, 10 bp, Homo sapiens
tctctactaa

SEQ ID NO: 698 - DNA, 10 bp, Homo sapiens
cggcttttct

SEQ ID NO: 699 - DNA, 10 bp, Homo sapiens
tggcccccgc

SEQ ID NO: 700 - DNA, 10 bp, Homo sapiens
tggcccccgc

SEQ ID NO: 701 - DNA, 10 bp, Homo sapiens
ctcctggggc

SEQ ID NO: 702 - DNA, 10 bp, Homo sapiens
aaggagctgg

SEQ ID NO: 703 - DNA, 10 bp, Homo sapiens
aaggagctgg

SEQ ID NO: 704 - DNA, 10 bp, Homo sapiens
aaggagctgg

SEQ ID NO: 705 - DNA, 10 bp, Homo sapiens
ggctttgatt

SEQ ID NO: 706 - DNA, 10 bp, Homo sapiens
actaccttca

SEQ ID NO: 707 - DNA, 10 bp, Homo sapiens
ctgtgcattt

SEQ ID NO: 708 - DNA, 10 bp, Homo sapiens
actccaaaaa

SEQ ID NO: 709 - DNA, 10 bp, Homo sapiens
actccaaaaa

SEQ ID NO: 710 - DNA, 10 bp, Homo sapiens
tcctgcccca

SEQ ID NO: 711 - DNA, 10 bp, Homo sapiens
tcctgcccca

SEQ ID NO: 712 - DNA, 10 bp, Homo sapiens
aagctggagg

SEQ ID NO: 713 - DNA, 10 bp, Homo sapiens
gcacaagaag

SEQ ID NO: 714 - DNA, 10 bp, Homo sapiens
gaaaccgagg

SEQ ID NO: 715 - DNA, 10 bp, Homo sapiens
gaaaccgagg

SEQ ID NO: 716 - DNA, 10 bp, Homo sapiens
gcccgcaagc

SEQ ID NO: 717 - DNA, 10 bp, Homo sapiens
ctttcagatg

SEQ ID NO: 718 - DNA, 10 bp, Homo sapiens
gggcgctgtg

SEQ ID NO: 719 - DNA, 10 bp, Homo sapiens
gtattcccct

SEQ ID NO: 720 - DNA, 10 bp, Homo sapiens
gtattcccct

SEQ ID NO: 721 - DNA, 10 bp, Homo sapiens
ctggccatcg

SEQ ID NO: 722 - DNA, 10 bp, Homo sapiens
gtggtggaca

SEQ ID NO: 723 - DNA, 10 bp, Homo sapiens
gtggtggaca

SEQ ID NO: 724 - DNA, 10 bp, Homo sapiens
gtggtggaca

SEQ ID NO: 725 - DNA, 10 bp, Homo sapiens
cacctaattg

SEQ ID NO: 726 - DNA, 10 bp, Homo sapiens
gacccctgtc

SEQ ID NO: 727 - DNA, 10 bp, Homo sapiens
cccttagctt

SEQ ID NO: 728 - DNA, 10 bp, Homo sapiens
cagagacgtg

SEQ ID NO: 729 - DNA, 10 bp, Homo sapiens
atggctggta

SEQ ID NO: 730 - DNA, 10 bp, Homo sapiens
tcagccttct

SEQ ID NO: 731 - DNA, 10 bp, Homo sapiens
tcgtaacgag

SEQ ID NO: 732 - DNA, 10 bp, Homo sapiens
gcgacgaggc

SEQ ID NO: 733 - DNA, 10 bp, Homo sapiens
gcggggtacc

SEQ ID NO: 734 - DNA, 10 bp, Homo sapiens
tccttctcca

SEQ ID NO: 735 - DNA, 10 bp, Homo sapiens
cagtctctca

SEQ ID NO: 736 - DNA, 10 bp, Homo sapiens
acccttccct

SEQ ID NO: 737 - DNA, 10 bp, Homo sapiens
acccttccct

SEQ ID NO: 738 - DNA, 10 bp, Homo sapiens
tgagtggtca

SEQ ID NO: 739 - DNA, 10 bp, Homo sapiens
gacaatgcca

SEQ ID NO: 740 - DNA, 10 bp, Homo sapiens
atctttctgg

SEQ ID NO: 741 - DNA, 10 bp, Homo sapiens
agctgtcccc

SEQ ID NO: 742 - DNA, 10 bp, Homo sapiens
tcttccagga

SEQ ID NO: 743 - DNA, 10 bp, Homo sapiens
gtgcctagga

SEQ ID NO: 744 - DNA, 10 bp, Homo sapiens
tggacccccc

SEQ ID NO: 745 - DNA, 10 bp, Homo sapiens
acctgtatcc

SEQ ID NO: 746 - DNA, 10 bp, Homo sapiens
acctgctggt

SEQ ID NO: 747 - DNA, 10 bp, Homo sapiens
agtctgatgt

SEQ ID NO: 748 - DNA, 10 bp, Homo sapiens
tctctaccca

SEQ ID NO: 749 - DNA, 10 bp, Homo sapiens
tgattaaggt

SEQ ID NO: 750 - DNA, 10 bp, Homo sapiens
cagcagaagc

SEQ ID NO: 751 - DNA, 10 bp, Homo sapiens
tccctattaa

SEQ ID NO: 752 - DNA, 10 bp, Homo sapiens
gtggaggtgc

SEQ ID NO: 753 - DNA, 10 bp, Homo sapiens
aagatccccg

SEQ ID NO: 754 - DNA, 10 bp, Homo sapiens
gagcggcctc

SEQ ID NO: 755 - DNA, 10 bp, Homo sapiens
aactacatag

SEQ ID NO: 756 - DNA, 10 bp, Homo sapiens
gtaagatttg

SEQ ID NO: 757 - DNA, 10 bp, Homo sapiens
agcctgcaga

SEQ ID NO: 758 - DNA, 10 bp, Homo sapiens
ggaccactga

SEQ ID NO: 759 - DNA, 10 bp, Homo sapiens
ttcaataaaa

SEQ ID NO: 760 - DNA, 10 bp, Homo sapiens
ttcaataaaa

SEQ ID NO: 761 - DNA, 10 bp, Homo sapiens
cgatggtccc

SEQ ID NO: 762 - DNA, 10 bp, Homo sapiens
catttgtaat

SEQ ID NO: 763 - DNA, 10 bp, Homo sapiens
cctgagcccg

SEQ ID NO: 764 - DNA, 10 bp, Homo sapiens
tgaggcctct

SEQ ID NO: 765 - DNA, 10 bp, Homo sapiens
aagagttacg

SEQ ID NO: 766 - DNA, 10 bp, Homo sapiens
gaatccaact

SEQ ID NO: 767 - DNA, 10 bp, Homo sapiens
aggggcgcag

SEQ ID NO: 768 - DNA, 10 bp, Homo sapiens
gcttagaagt

SEQ ID NO: 769 - DNA, 10 bp, Homo sapiens
aagtcattca

SEQ ID NO: 770 - DNA, 10 bp, Homo sapiens
aagtcattca

SEQ ID NO: 771 - DNA, 10 bp, Homo sapiens
taccccaccc

SEQ ID NO: 772 - DNA, 10 bp, Homo sapiens
taccccaccc

SEQ ID NO: 773 - DNA, 10 bp, Homo sapiens
cctagctgga

SEQ ID NO: 774 - DNA, 10 bp, Homo sapiens
tcgtctttat

SEQ ID NO: 775 - DNA, 10 bp, Homo sapiens
ggtttggctt

SEQ ID NO: 776 - DNA, 10 bp, Homo sapiens
taggatgggg

SEQ ID NO: 777 - DNA, 10 bp, Homo sapiens
gtgcatcccg

SEQ ID NO: 778 - DNA, 10 bp, Homo sapiens
cagcgctgca

SEQ ID NO: 779 - DNA, 10 bp, Homo sapiens
gggagcccct

SEQ ID NO: 780 - DNA, 10 bp, Homo sapiens
gggagcccct

SEQ ID NO: 781 - DNA, 10 bp, Homo sapiens
gaagatgtgg

SEQ ID NO: 782 - DNA, 10 bp, Homo sapiens
cctaccacag

SEQ ID NO: 783 - DNA, 10 bp, Homo sapiens
tgctaaaaaa

SEQ ID NO: 784 - DNA, 10 bp, Homo sapiens
cacagagtcc

SEQ ID NO: 785 - DNA, 10 bp, Homo sapiens
gggccaataa

SEQ ID NO: 786 - DNA, 10 bp, Homo sapiens
gcctgctggg

SEQ ID NO: 787 - DNA, 10 bp, Homo sapiens
actgcttgcc

SEQ ID NO: 788 - DNA, 10 bp, Homo sapiens
actgcttgcc

SEQ ID NO: 789 - DNA, 10 bp, Homo sapiens
cggttactgt

SEQ ID NO: 790 - DNA, 10 bp, Homo sapiens
aacccgggag

SEQ ID NO: 791 - DNA, 10 bp, Homo sapiens
aacccgggag

SEQ ID NO: 792 - DNA, 10 bp, Homo sapiens
aacccgggag

SEQ ID NO: 793 - DNA, 10 bp, Homo sapiens
attaacaaag

SEQ ID NO: 794 - DNA, 10 bp, Homo sapiens
ttcagtgccc

SEQ ID NO: 795 - DNA, 10 bp, Homo sapiens
ccgtgctcat

SEQ ID NO: 796 - DNA, 10 bp, Homo sapiens
atccctcagt

SEQ ID NO: 797 - DNA, 10 bp, Homo sapiens
taccatcaat

SEQ ID NO: 798 - DNA, 10 bp, Homo sapiens
tgcaccacag

SEQ ID NO: 799 - DNA, 10 bp, Homo sapiens
gaaccctggg

SEQ ID NO: 800 - DNA, 10 bp, Homo sapiens
gccgtgtccg

SEQ ID NO: 801 - DNA, 10 bp, Homo sapiens
atagaggcaa

SEQ ID NO: 802 - DNA, 10 bp, Homo sapiens
attgtttatg

SEQ ID NO: 803 - DNA, 10 bp, Homo sapiens
taataaaggt

SEQ ID NO: 804 - DNA, 10 bp, Homo sapiens
gggatcaagg

SEQ ID NO: 805 - DNA, 10 bp, Homo sapiens
caagggcttg

SEQ ID NO: 806 - DNA, 10 bp, Homo sapiens
tggtgttgag

SEQ ID NO: 807 - DNA, 10 bp, Homo sapiens
gagtgagtga

SEQ ID NO: 808 - DNA, 10 bp, Homo sapiens
gtggcgcaca

SEQ ID NO: 809 - DNA, 10 bp, Homo sapiens
atgatccgga

SEQ ID NO: 810 - DNA, 10 bp, Homo sapiens
aacctgggag

SEQ ID NO: 811 - DNA, 10 bp, Homo sapiens
aacctgggag

SEQ ID NO: 812 - DNA, 10 bp, Homo sapiens
tgcttcatct

SEQ ID NO: 813 - DNA, 10 bp, Homo sapiens
ataattcttt

SEQ ID NO: 814 - DNA, 10 bp, Homo sapiens
gttcagctgt

SEQ ID NO: 815 - DNA, 10 bp, Homo sapiens
gggaagtcac

SEQ ID NO: 816 - DNA, 10 bp, Homo sapiens
gggtgcttgg

SEQ ID NO: 817 - DNA, 10 bp, Homo sapiens
cagttactta

SEQ ID NO: 818 - DNA, 10 bp, Homo sapiens
gcgaaacccc

SEQ ID NO: 819 - DNA, 10 bp, Homo sapiens
gccttccaat

SEQ ID NO: 820 - DNA, 10 bp, Homo sapiens
ccccctggat

SEQ ID NO: 821 - DNA, 10 bp, Homo sapiens
gacctcctgc

SEQ ID NO: 822 - DNA, 10 bp, Homo sapiens
gacctcctgc

SEQ ID NO: 823 - DNA, 10 bp, Homo sapiens
cagcagtagc

SEQ ID NO: 824 - DNA, 10 bp, Homo sapiens
ttcattataa

SEQ ID NO: 825 - DNA, 10 bp, Homo sapiens
cccccaccta

SEQ ID NO: 826 - DNA, 10 bp, Homo sapiens
ggtggatgtg

SEQ ID NO: 827 - DNA, 10 bp, Homo sapiens
tctggtttgt

SEQ ID NO: 828 - DNA, 10 bp, Homo sapiens
tctggtttgt

SEQ ID NO: 829 - DNA, 10 bp, Homo sapiens
cgcctgtaat

SEQ ID NO: 830 - DNA, 10 bp, Homo sapiens
tcctgctgcc

SEQ ID NO: 831 - DNA, 10 bp, Homo sapiens
tcctgctgcc

SEQ ID NO: 832 - DNA, 10 bp, Homo sapiens
gtgtggtggt

SEQ ID NO: 833 - DNA, 10 bp, Homo sapiens
tgatgtccac

SEQ ID NO: 834 - DNA, 10 bp, Homo sapiens
ccaggaggaa

SEQ ID NO: 835 - DNA, 10 bp, Homo sapiens
gtgaagcccc

SEQ ID NO: 836 - DNA, 10 bp, Homo sapiens
gggagcccgg

SEQ ID NO: 837 - DNA, 10 bp, Homo sapiens
gccatcccct

SEQ ID NO: 838 - DNA, 10 bp, Homo sapiens
cagttggttg

SEQ ID NO: 839 - DNA, 10 bp, Homo sapiens
atccatctgt

SEQ ID NO: 840 - DNA, 10 bp, Homo sapiens
gccaggaagc

SEQ ID NO: 841 - DNA, 10 bp, Homo sapiens
tccagcccct

SEQ ID NO: 842 - DNA, 10 bp, Homo sapiens
gccccccact

SEQ ID NO: 843 - DNA, 10 bp, Homo sapiens
tgtctgtggt

SEQ ID NO: 844 - DNA, 10 bp, Homo sapiens
tcccgtacat

SEQ ID NO: 845 - DNA, 10 bp, Homo sapiens
gtggtgggca

SEQ ID NO: 846 - DNA, 10 bp, Homo sapiens
gtggtgggca

SEQ ID NO: 847 - DNA, 10 bp, Homo sapiens
gtggtgggca

SEQ ID NO: 848 - DNA, 10 bp, Homo sapiens
ctgttagtgt

SEQ ID NO: 849 - DNA, 10 bp, Homo sapiens
ctctcaccct

SEQ ID NO: 850 - DNA, 10 bp, Homo sapiens
tgctggtgtg

SEQ ID NO: 851 - DNA, 10 bp, Homo sapiens
ctaagacttc

SEQ ID NO: 852 - DNA, 10 bp, Homo sapiens
ggaaggacag

SEQ ID NO: 853 - DNA, 10 bp, Homo sapiens
gaagtgtgtc

SEQ ID NO: 854 - DNA, 10 bp, Homo sapiens
gtacccggac

SEQ ID NO: 855 - DNA, 10 bp, Homo sapiens
cctccctgat

SEQ ID NO: 856 - DNA, 10 bp, Homo sapiens
tcatcttcaa

SEQ ID NO: 857 - DNA, 10 bp, Homo sapiens
tcatcttcaa

SEQ ID NO: 858 - DNA, 10 bp, Homo sapiens
tcatcttcaa

SEQ ID NO: 859 - DNA, 10 bp, Homo sapiens
atgtactctg

SEQ ID NO: 860 - DNA, 10 bp, Homo sapiens
cgccggaaca

SEQ ID NO: 861 - DNA, 10 bp, Homo sapiens
aagggagggt

SEQ ID NO: 862 - DNA, 10 bp, Homo sapiens
gaaaaaaaaa

SEQ ID NO: 863 - DNA, 10 bp, Homo sapiens
aaactctgtg

SEQ ID NO: 864 - DNA, 10 bp, Homo sapiens
acacacgcaa

SEQ ID NO: 865 - DNA, 10 bp, Homo sapiens
ccgccgaagt

SEQ ID NO: 866 - DNA, 10 bp, Homo sapiens
tgtgctaaat

SEQ ID NO: 867 - DNA, 10 bp, Homo sapiens
cgaccgtggc

SEQ ID NO: 868 - DNA, 10 bp, Homo sapiens
gcctgggctg

SEQ ID NO: 869 - DNA, 10 bp, Homo sapiens
gcctgggctg

SEQ ID NO: 870 - DNA, 10 bp, Homo sapiens
aaagtcagaa

SEQ ID NO: 871 - DNA, 10 bp, Homo sapiens
tggagcgcta

SEQ ID NO: 872 - DNA, 10 bp, Homo sapiens
gaaatgatga

SEQ ID NO: 873 - DNA, 10 bp, Homo sapiens
tgtcgctggg

SEQ ID NO: 874 - DNA, 10 bp, Homo sapiens
gcccctgcct

SEQ ID NO: 875 - DNA, 10 bp, Homo sapiens
gcccctgcct

SEQ ID NO: 876 - DNA, 10 bp, Homo sapiens
caggcctggc

SEQ ID NO: 877 - DNA, 10 bp, Homo sapiens
caggcctggc

SEQ ID NO: 878 - DNA, 10 bp, Homo sapiens
gcaaaaaaaa

SEQ ID NO: 879 - DNA, 10 bp, Homo sapiens
agccaccacg

SEQ ID NO: 880 - DNA, 10 bp, Homo sapiens
gaggaagaag

SEQ ID NO: 881 - DNA, 10 bp, Homo sapiens
cagctgtagt

SEQ ID NO: 882 - DNA, 10 bp, Homo sapiens
tcttctccct

SEQ ID NO: 883 - DNA, 10 bp, Homo sapiens
tacattctgt

SEQ ID NO: 884 - DNA, 10 bp, Homo sapiens
gggaaacccc

SEQ ID NO: 885 - DNA, 10 bp, Homo sapiens
agccactgca

SEQ ID NO: 886 - DNA, 10 bp, Homo sapiens
tagttgaagt

SEQ ID NO: 887 - DNA, 10 bp, Homo sapiens
gccaagtttg

SEQ ID NO: 888 - DNA, 10 bp, Homo sapiens
ggcggctgca

SEQ ID NO: 889 - DNA, 10 bp, Homo sapiens
aaaaaaaaaa

SEQ ID NO: 890 - DNA, 10 bp, Homo sapiens
aaaaaaaaaa

SEQ ID NO: 891 - DNA, 10 bp, Homo sapiens
aaaaaaaaaa

SEQ ID NO: 892 - DNA, 10 bp, Homo sapiens
aaaaaaaaaa

SEQ ID NO: 893 - DNA, 10 bp, Homo sapiens
tgttccactc

SEQ ID NO: 894 - DNA, 10 bp, Homo sapiens
ctcggtgatg

SEQ ID NO: 895 - DNA, 10 bp, Homo sapiens
cttctcaggg

SEQ ID NO: 896 - DNA, 10 bp, Homo sapiens
ggtagcccac

SEQ ID NO: 897 - DNA, 10 bp, Homo sapiens
gggtttttat

SEQ ID NO: 898 - DNA, 10 bp, Homo sapiens
cctgtaaccc

SEQ ID NO: 899 - DNA, 10 bp, Homo sapiens
gaaacaagat

SEQ ID NO: 900 - DNA, 10 bp, Homo sapiens
gatgagtctc

SEQ ID NO: 901 - DNA, 10 bp, Homo sapiens
ggccctaggc

SEQ ID NO: 902 - DNA, 10 bp, Homo sapiens
tggccccacc

SEQ ID NO: 903 - DNA, 10 bp, Homo sapiens
cagcgcgccc

SEQ ID NO: 904 - DNA, 10 bp, Homo sapiens
aggcgagatc

SEQ ID NO: 905 - DNA, 10 bp, Homo sapiens
gcggggtgga

SEQ ID NO: 906 - DNA, 10 bp, Homo sapiens
ggggccccct

SEQ ID NO: 907 - DNA, 10 bp, Homo sapiens
aaggaacttg

SEQ ID NO: 908 - DNA, 10 bp, Homo sapiens
aaggaacttg

SEQ ID NO: 909 - DNA, 10 bp, Homo sapiens
aattgcaagc

SEQ ID NO: 910 - DNA, 10 bp, Homo sapiens
cctgtgatcc

SEQ ID NO: 911 - DNA, 10 bp, Homo sapiens
ccccgccaag

SEQ ID NO: 912 - DNA, 10 bp, Homo sapiens
ctcaacagca

SEQ ID NO: 913 - DNA, 10 bp, Homo sapiens
aaggtagcag

SEQ ID NO: 914 - DNA, 10 bp, Homo sapiens
aagccagccc

SEQ ID NO: 915 - DNA, 10 bp, Homo sapiens
cagccttgga

SEQ ID NO: 916 - DNA, 10 bp, Homo sapiens
tttgctctcc

SEQ ID NO: 917 - DNA, 10 bp, Homo sapiens
caacattcct

SEQ ID NO: 918 - DNA, 10 bp, Homo sapiens
tactagtcct

SEQ ID NO: 919 - DNA, 10 bp, Homo sapiens
gactctggtg

SEQ ID NO: 920 - DNA, 10 bp, Homo sapiens
gactctggtg

SEQ ID NO: 921 - DNA, 10 bp, Homo sapiens
gtggctcacg

SEQ ID NO: 922 - DNA, 10 bp, Homo sapiens
gtggctcacg

SEQ ID NO: 923 - DNA, 10 bp, Homo sapiens
gtggcgggca

SEQ ID NO: 924 - DNA, 10 bp, Homo sapiens
gtggcgggca

SEQ ID NO: 925 - DNA, 10 bp, Homo sapiens
cctgtggtcc

SEQ ID NO: 926 - DNA, 10 bp, Homo sapiens
tacagcacgg

SEQ ID NO: 927 - DNA, 10 bp, Homo sapiens
gtggcacctg

SEQ ID NO: 928 - DNA, 10 bp, Homo sapiens
tacacgtgag

SEQ ID NO: 929 - DNA, 10 bp, Homo sapiens
tcaggcattt

SEQ ID NO: 930 - DNA, 10 bp, Homo sapiens
ttcacaaagg

SEQ ID NO: 931 - DNA, 10 bp, Homo sapiens
ttcttgtggc

SEQ ID NO: 932 - DNA, 10 bp, Homo sapiens
tccctattag

SEQ ID NO: 933 - DNA, 10 bp, Homo sapiens
tacaagagga

SEQ ID NO: 934 - DNA, 10 bp, Homo sapiens
tcagacgcag

SEQ ID NO: 935 - DNA, 10 bp, Homo sapiens
caggatccag

SEQ ID NO: 936 - DNA, 10 bp, Homo sapiens
tctgtacacc

SEQ ID NO: 937 - DNA, 10 bp, Homo sapiens
gaagcaggac

SEQ ID NO: 938 - DNA, 10 bp, Homo sapiens
gcgccgcccc

SEQ ID NO: 939 - DNA, 10 bp, Homo sapiens
ccctcctggg

SEQ ID NO: 940 - DNA, 10 bp, Homo sapiens
tgggcgcctt

SEQ ID NO: 941 - DNA, 10 bp, Homo sapiens
gtggtacagg

SEQ ID NO: 942 - DNA, 10 bp, Homo sapiens
gtggtacagg

SEQ ID NO: 943 - DNA, 10 bp, Homo sapiens
ggtgagacct

SEQ ID NO: 944 - DNA, 10 bp, Homo sapiens
gagatccgca

SEQ ID NO: 945 - DNA, 10 bp, Homo sapiens
ttggcagccc

SEQ ID NO: 946 - DNA, 10 bp, Homo sapiens
gcctttccct

SEQ ID NO: 947 - DNA, 10 bp, Homo sapiens
ggagtggaca

SEQ ID NO: 948 - DNA, 10 bp, Homo sapiens
ttatggggag

SEQ ID NO: 949 - DNA, 10 bp, Homo sapiens
ttatggggag

SEQ ID NO: 950 - DNA, 10 bp, Homo sapiens
gagtgggggc

SEQ ID NO: 951 - DNA, 10 bp, Homo sapiens
gtggcacgtg

SEQ ID NO: 952 - DNA, 10 bp, Homo sapiens
ctgggcgtgt

SEQ ID NO: 953 - DNA, 10 bp, Homo sapiens
ttggggtttc

SEQ ID NO: 954 - DNA, 10 bp, Homo sapiens
ggctgggcct

SEQ ID NO: 955 - DNA, 10 bp, Homo sapiens
ggctgggcct

SEQ ID NO: 956 - DNA, 10 bp, Homo sapiens
cctgttctcc

SEQ ID NO: 957 - DNA, 10 bp, Homo sapiens
gtgtctcatc

SEQ ID NO: 958 - DNA, 10 bp, Homo sapiens
gtgtctcatc

SEQ ID NO: 959 - DNA, 10 bp, Homo sapiens
acgattgatg

SEQ ID NO: 960 - DNA, 10 bp, Homo sapiens
ttgttgttga

SEQ ID NO: 961 - DNA, 10 bp, Homo sapiens
tggcctcccc

SEQ ID NO: 962 - DNA, 10 bp, Homo sapiens
atcgggcccg

SEQ ID NO: 963 - DNA, 10 bp, Homo sapiens
gccgccatca

SEQ ID NO: 964 - DNA, 10 bp, Homo sapiens
gtgctggacc

SEQ ID NO: 965 - DNA, 10 bp, Homo sapiens
ttgtaatcgt

SEQ ID NO: 966 - DNA, 10 bp, Homo sapiens
taatggtaac

SEQ ID NO: 967 - DNA, 10 bp, Homo sapiens
aacgacctcg

SEQ ID NO: 968 - DNA, 10 bp, Homo sapiens
gcctgcaccc

SEQ ID NO: 969 - DNA, 10 bp, Homo sapiens
gcctgcaccc

SEQ ID NO: 970 - DNA, 10 bp, Homo sapiens
aaggtggagg

SEQ ID NO: 971 - DNA, 10 bp, Homo sapiens
aaggagatgg

SEQ ID NO: 972 - DNA, 10 bp, Homo sapiens
cagttctctg

SEQ ID NO: 973 - DNA, 10 bp, Homo sapiens
gtgaaacctc

SEQ ID NO: 974 - DNA, 10 bp, Homo sapiens
taggttgtct

SEQ ID NO: 975 - DNA, 10 bp, Homo sapiens
cctgtgacag

SEQ ID NO: 976 - DNA, 10 bp, Homo sapiens
ctcataagga

SEQ ID NO: 977 - DNA, 10 bp, Homo sapiens
ggtggctttg

SEQ ID NO: 978 - DNA, 10 bp, Homo sapiens
gctcagctgg

SEQ ID NO: 979 - DNA, 10 bp, Homo sapiens
ggccctgagc

SEQ ID NO: 980 - DNA, 10 bp, Homo sapiens
tctgctaaag

SEQ ID NO: 981 - DNA, 10 bp, Homo sapiens
tctgctaaag

SEQ ID NO: 982 - DNA, 10 bp, Homo sapiens
agccccacaa

SEQ ID NO: 983 - DNA, 10 bp, Homo sapiens
ctgagtctcc

SEQ ID NO: 984 - DNA, 10 bp, Homo sapiens
tgctttggga

SEQ ID NO: 985 - DNA, 10 bp, Homo sapiens
cctgtcctgc

SEQ ID NO: 986 - DNA, 10 bp, Homo sapiens
ggggaaatcg

SEQ ID NO: 987 - DNA, 10 bp, Homo sapiens
tctgcctggg

SEQ ID NO: 988 - DNA, 10 bp, Homo sapiens
caataaactg

SEQ ID NO: 989 - DNA, 10 bp, Homo sapiens
gagtctgagg

SEQ ID NO: 990 - DNA, 10 bp, Homo sapiens
gtggcaggcg

SEQ ID NO: 991 - DNA, 10 bp, Homo sapiens
gtggcaggcg

SEQ ID NO: 992 - DNA, 10 bp, Homo sapiens
cgaggggcca

SEQ ID NO: 993 - DNA, 10 bp, Homo sapiens
gtggggggag

SEQ ID NO: 994 - DNA, 10 bp, Homo sapiens
gagtggctat

SEQ ID NO: 995 - DNA, 10 bp, Homo sapiens
gagtggctat

SEQ ID NO: 996 - DNA, 10 bp, Homo sapiens
gtagactcac

SEQ ID NO: 997 - DNA, 10 bp, Homo sapiens
agggaaagag

SEQ ID NO: 998 - DNA, 10 bp, Homo sapiens
agggaaagag

SEQ ID NO: 999 - DNA, 10 bp, Homo sapiens
cccatcgtcc

SEQ ID NO: 1000 - DNA, 10 bp, Homo sapiens
tcgccgcgac

SEQ ID NO: 1001 - DNA, 10 bp, Homo sapiens
tgtcctggtt

SEQ ID NO: 1002 - DNA, 10 bp, Homo sapiens
ctttttgtgc

SEQ ID NO: 1003 - DNA, 10 bp, Homo sapiens
ataaattggg

SEQ ID NO: 1004 - DNA, 10 bp, Homo sapiens
tatcactctg

SEQ ID NO: 1005 - DNA, 10 bp, Homo sapiens
gtggtgggcg

SEQ ID NO: 1006 - DNA, 10 bp, Homo sapiens
ccactacact

SEQ ID NO: 1007 - DNA, 10 bp, Homo sapiens
tgaccccaca

SEQ ID NO: 1008 - DNA, 10 bp, Homo sapiens
tgatttcact

SEQ ID NO: 1009 - DNA, 10 bp, Homo sapiens
tgatttcact

SEQ ID NO: 1010 - DNA, 10 bp, Homo sapiens
ggctcccact

SEQ ID NO: 1011 - DNA, 10 bp, Homo sapiens
cctgtgtgtg

SEQ ID NO: 1012 - DNA, 10 bp, Homo sapiens
aatcctgtgg

SEQ ID NO: 1013 - DNA, 10 bp, Homo sapiens
aggagcaaag

SEQ ID NO: 1014 - DNA, 10 bp, Homo sapiens
cctttgaaca

SEQ ID NO: 1015 - DNA, 10 bp, Homo sapiens
gtggggctag

SEQ ID NO: 1016 - DNA, 10 bp, Homo sapiens
agggtgaaac

SEQ ID NO: 1017 - DNA, 10 bp, Homo sapiens
cctcaggata

SEQ ID NO: 1018 - DNA, 10 bp, Homo sapiens
cctcaggata

SEQ ID NO: 1019 - DNA, 10 bp, Homo sapiens
ttccactaac

SEQ ID NO: 1020 - DNA, 10 bp, Homo sapiens
cccccgtgaa

SEQ ID NO: 1021 - DNA, 10 bp, Homo sapiens
tgtgctcggg

SEQ ID NO: 1022 - DNA, 10 bp, Homo sapiens
aagccttgct

SEQ ID NO: 1023 - DNA, 10 bp, Homo sapiens
tgttcatcat

SEQ ID NO: 1024 - DNA, 10 bp, Homo sapiens
aactaacaaa

SEQ ID NO: 1025 - DNA, 10 bp, Homo sapiens
gctgttgcgc

SEQ ID NO: 1026 - DNA, 10 bp, Homo sapiens
ggatgtgaaa

SEQ ID NO: 1027 - DNA, 10 bp, Homo sapiens
actggtacgt

SEQ ID NO: 1028 - DNA, 10 bp, Homo sapiens
ttgtattcca

SEQ ID NO: 1029 - DNA, 10 bp, Homo sapiens
ggctgggggc

SEQ ID NO: 1030 - DNA, 10 bp, Homo sapiens
ccactgcact

SEQ ID NO: 1031 - DNA, 10 bp, Homo sapiens
ccactgcact

SEQ ID NO: 1032 - DNA, 10 bp, Homo sapiens
ccactgcact

SEQ ID NO: 1033 - DNA, 10 bp, Homo sapiens
ccactgcact

SEQ ID NO: 1034 - DNA, 10 bp, Homo sapiens
ccactgcact

SEQ ID NO: 1035 - DNA, 10 bp, Homo sapiens
ccactgcact

SEQ ID NO: 1036 - DNA, 10 bp, Homo sapiens
ccactgcact

SEQ ID NO: 1037 - DNA, 10 bp, Homo sapiens
ccactgcact

SEQ ID NO: 1038 - DNA, 10 bp, Homo sapiens
ccactgcact

SEQ ID NO: 1039 - DNA, 10 bp, Homo sapiens
ccactgcact

SEQ ID NO: 1040 - DNA, 10 bp, Homo sapiens
ccactgcact

SEQ ID NO: 1041 - DNA, 10 bp, Homo sapiens
ccactgcact

SEQ ID NO: 1042 - DNA, 10 bp, Homo sapiens
ccactgcact

SEQ ID NO: 1043 - DNA, 10 bp, Homo sapiens
ccactgcact

SEQ ID NO: 1044 - DNA, 10 bp, Homo sapiens
cacttgccct

SEQ ID NO: 1045 - DNA, 10 bp, Homo sapiens
cacttgccct

SEQ ID NO: 1046 - DNA, 10 bp, Homo sapiens
gcaagccaac

SEQ ID NO: 1047 - DNA, 10 bp, Homo sapiens
tagataatgg

SEQ ID NO: 1048 - DNA, 10 bp, Homo sapiens
tcgaagcccc

SEQ ID NO: 1049 - DNA, 10 bp, Homo sapiens
agaaaaaaaa

SEQ ID NO: 1050 - DNA, 10 bp, Homo sapiens
agaaaaaaaa

SEQ ID NO: 1051 - DNA, 10 bp, Homo sapiens
ggcgcctcct

SEQ ID NO: 1052 - DNA, 10 bp, Homo sapiens
ggcgcctcct

SEQ ID NO: 1053 - DNA, 10 bp, Homo sapiens
taaactgttt

SEQ ID NO: 1054 - DNA, 10 bp, Homo sapiens
taaactgttt

SEQ ID NO: 1055 - DNA, 10 bp, Homo sapiens
ggcctttttt

SEQ ID NO: 1056 - DNA, 10 bp, Homo sapiens
ggcctttttt

SEQ ID NO: 1057 - DNA, 10 bp, Homo sapiens
gcgacagctc

SEQ ID NO: 1058 - DNA, 10 bp, Homo sapiens
cccacactac

SEQ ID NO: 1059 - DNA, 10 bp, Homo sapiens
agcagatcag

SEQ ID NO: 1060 - DNA, 10 bp, Homo sapiens
gcataggctg

SEQ ID NO: 1061 - DNA, 10 bp, Homo sapiens
gaggccgacc

SEQ ID NO: 1062 - DNA, 10 bp, Homo sapiens
aaatgccaca

SEQ ID NO: 1063 - DNA, 10 bp, Homo sapiens
agccctacaa

SEQ ID NO: 1064 - DNA, 10 bp, Homo sapiens
ttggtgaagg

SEQ ID NO: 1065 - DNA, 10 bp, Homo sapiens
ccgggcccag

SEQ ID NO: 1066 - DNA, 10 bp, Homo sapiens
ttcatacacc

SEQ ID NO: 1067 - DNA, 10 bp, Homo sapiens
gcagccatcc

SEQ ID NO: 1068 - DNA, 10 bp, Homo sapiens
gccgggtggg

SEQ ID NO: 1069 - DNA, 10 bp, Homo sapiens
gctcccagac

SEQ ID NO: 1070 - DNA, 10 bp, Homo sapiens
agccaccgtg

SEQ ID NO: 1071 - DNA, 10 bp, Homo sapiens
tcagctggcc

SEQ ID NO: 1072 - DNA, 10 bp, Homo sapiens
gggggcgcct

SEQ ID NO: 1073 - DNA, 10 bp, Homo sapiens
cggcccaacg

SEQ ID NO: 1074 - DNA, 10 bp, Homo sapiens
tggccatctg

SEQ ID NO: 1075 - DNA, 10 bp, Homo sapiens
cctcccccgt

SEQ ID NO: 1076 - DNA, 10 bp, Homo sapiens
acttgttcgc

SEQ ID NO: 1077 - DNA, 10 bp, Homo sapiens
aagactggct

SEQ ID NO: 1078 - DNA, 10 bp, Homo sapiens
agcacatttg

SEQ ID NO: 1079 - DNA, 10 bp, Homo sapiens
gtgaaggcag

SEQ ID NO: 1080 - DNA, 10 bp, Homo sapiens
caataaatgt

SEQ ID NO: 1081 - DNA, 10 bp, Homo sapiens
gccagggcgg

SEQ ID NO: 1082 - DNA, 10 bp, Homo sapiens
gtgtaataag

SEQ ID NO: 1083 - DNA, 10 bp, Homo sapiens
ttctgcactg

SEQ ID NO: 1084 - DNA, 10 bp, Homo sapiens
ttctgcactg

SEQ ID NO: 1085 - DNA, 10 bp, Homo sapiens
gtgaaacccc

SEQ ID NO: 1086 - DNA, 10 bp, Homo sapiens
gtgaaacccc

SEQ ID NO: 1087 - DNA, 10 bp, Homo sapiens
gtgaaacccc

SEQ ID NO: 1088 - DNA, 10 bp, Homo sapiens
gtgaaacccc

SEQ ID NO: 1089 - DNA, 10 bp, Homo sapiens
gtgaaacccc

SEQ ID NO: 1090 - DNA, 10 bp, Homo sapiens
gtgaaacccc

SEQ ID NO: 1091 - DNA, 10 bp, Homo sapiens
gtgaaacccc

SEQ ID NO: 1092 - DNA, 10 bp, Homo sapiens
gtgaaacccc

SEQ ID NO: 1093 - DNA, 10 bp, Homo sapiens
gtgaaacccc

SEQ ID NO: 1094 - DNA, 10 bp, Homo sapiens
gtgaaacccc

SEQ ID NO: 1095 - DNA, 10 bp, Homo sapiens
gtgaaacccc

SEQ ID NO: 1096 - DNA, 10 bp, Homo sapiens
gtgaaacccc

SEQ ID NO: 1097 - DNA, 10 bp, Homo sapiens
gtgaaacccc

SEQ ID NO: 1098 - DNA, 10 bp, Homo sapiens
gtgaaacccc

SEQ ID NO: 1099 - DNA, 10 bp, Homo sapiens
gtgaaacccc

SEQ ID NO: 1100 - DNA, 10 bp, Homo sapiens
gacacctcct

SEQ ID NO: 1101 - DNA, 10 bp, Homo sapiens
gacgtgtggg

SEQ ID NO: 1102 - DNA, 10 bp, Homo sapiens
gcaaaacccc

SEQ ID NO: 1103 - DNA, 10 bp, Homo sapiens
taccagtgta

SEQ ID NO: 1104 - DNA, 10 bp, Homo sapiens
cccctcccca

SEQ ID NO: 1105 - DNA, 10 bp, Homo sapiens
ggtgatgagg

SEQ ID NO: 1106 - DNA, 10 bp, Homo sapiens
gtgtgtaaaa

SEQ ID NO: 1107 - DNA, 10 bp, Homo sapiens
ggctcctcga

SEQ ID NO: 1108 - DNA, 10 bp, Homo sapiens
aaaagaaact

SEQ ID NO: 1109 - DNA, 10 bp, Homo sapiens
cagcgcacag

SEQ ID NO: 1110 - DNA, 10 bp, Homo sapiens
ctgggagagg

SEQ ID NO: 1111 - DNA, 10 bp, Homo sapiens
gaaaaatggt

SEQ ID NO: 1112 - DNA, 10 bp, Homo sapiens
atcacgccct

SEQ ID NO: 1113 - DNA, 10 bp, Homo sapiens
tagctctatg

SEQ ID NO: 1114 - DNA, 10 bp, Homo sapiens
gtattggcct

SEQ ID NO: 1115 - DNA, 10 bp, Homo sapiens
cccgacgtgc

SEQ ID NO: 1116 - DNA, 10 bp, Homo sapiens
gaagttatga

SEQ ID NO: 1117 - DNA, 10 bp, Homo sapiens
taaaaaaaaa

SEQ ID NO: 1118 - DNA, 10 bp, Homo sapiens
taaaaaaaaa

SEQ ID NO: 1119 - DNA, 10 bp, Homo sapiens
taaaaaaaaa

SEQ ID NO: 1120 - DNA, 10 bp, Homo sapiens
gccgccctgc

SEQ ID NO: 1121 - DNA, 10 bp, Homo sapiens
tttggggctg

SEQ ID NO: 1122 - DNA, 10 bp, Homo sapiens
gtggcaggca

SEQ ID NO: 1123 - DNA, 10 bp, Homo sapiens
ggctgtaccc

SEQ ID NO: 1124 - DNA, 10 bp, Homo sapiens
agcagggctc

SEQ ID NO: 1125 - DNA, 10 bp, Homo sapiens
aagaagatag

SEQ ID NO: 1126 - DNA, 10 bp, Homo sapiens
tctggggacg

SEQ ID NO: 1127 - DNA, 10 bp, Homo sapiens
gctaggttta

SEQ ID NO: 1128 - DNA, 10 bp, Homo sapiens
tggtgacagt

SEQ ID NO: 1129 - DNA, 10 bp, Homo sapiens
ttaccatatc

SEQ ID NO: 1130 - DNA, 10 bp, Homo sapiens
gtggcgggtg

SEQ ID NO: 1131 - DNA, 10 bp, Homo sapiens
tggatcctag

SEQ ID NO: 1132 - DNA, 10 bp, Homo sapiens
gggtttgaac

SEQ ID NO: 1133 - DNA, 10 bp, Homo sapiens
aatgcaggca

SEQ ID NO: 1134 - DNA, 10 bp, Homo sapiens
acatcgtagg

SEQ ID NO: 1135 - DNA, 10 bp, Homo sapiens
aacgctgcct

SEQ ID NO: 1136 - DNA, 10 bp, Homo sapiens
tggaggtggg

SEQ ID NO: 1137 - DNA, 10 bp, Homo sapiens
tgcctgctcc

SEQ ID NO: 1138 - DNA, 10 bp, Homo sapiens
cttccagcta

SEQ ID NO: 1139 - DNA, 10 bp, Homo sapiens
gtaagtgtac

SEQ ID NO: 1140 - DNA, 10 bp, Homo sapiens
gtaagtgtac

SEQ ID NO: 1141 - DNA, 10 bp, Homo sapiens
gtgtctcgca

SEQ ID NO: 1142 - DNA, 10 bp, Homo sapiens
atccggcgcc

SEQ ID NO: 1143 - DNA, 10 bp, Homo sapiens
tgcctgcacc

SEQ ID NO: 1144 - DNA, 10 bp, Homo sapiens
ttcctattaa

SEQ ID NO: 1145 - DNA, 10 bp, Homo sapiens
caggagttca

SEQ ID NO: 1146 - DNA, 10 bp, Homo sapiens
gtctgcgtgc

SEQ ID NO: 1147 - DNA, 10 bp, Homo sapiens
gaaatacagt

SEQ ID NO: 1148 - DNA, 10 bp, Homo sapiens
gaaatacagt

SEQ ID NO: 1149 - DNA, 10 bp, Homo sapiens
tgagcccggc

SEQ ID NO: 1150 - DNA, 10 bp, Homo sapiens
gtggtgtgtg

SEQ ID NO: 1151 - DNA, 10 bp, Homo sapiens
gtggtgtgtg

SEQ ID NO: 1152 - DNA, 10 bp, Homo sapiens
tcacccacac

SEQ ID NO: 1153 - DNA, 10 bp, Homo sapiens
tcacccacac

SEQ ID NO: 1154 - DNA, 10 bp, Homo sapiens
ctggatctgg

SEQ ID NO: 1155 - DNA, 10 bp, Homo sapiens
gaagatgtgt

SEQ ID NO: 1156 - DNA, 10 bp, Homo sapiens
cggataacca

SEQ ID NO: 1157 - DNA, 10 bp, Homo sapiens
tcagaaggtg

SEQ ID NO: 1158 - DNA, 10 bp, Homo sapiens
gagaaacccc

SEQ ID NO: 1159 - DNA, 10 bp, Homo sapiens
gagaaacccc

SEQ ID NO: 1160 - DNA, 10 bp, Homo sapiens
gagaaacccc

SEQ ID NO: 1161 - DNA, 10 bp, Homo sapiens
ctcgttaaga

SEQ ID NO: 1162 - DNA, 10 bp, Homo sapiens
ttggagatct

SEQ ID NO: 1163 - DNA, 10 bp, Homo sapiens
gaggtccctg

SEQ ID NO: 1164 - DNA, 10 bp, Homo sapiens
ttccgcgtgc

SEQ ID NO: 1165 - DNA, 10 bp, Homo sapiens
cagcccaacc

SEQ ID NO: 1166 - DNA, 10 bp, Homo sapiens
gtggctcaca

SEQ ID NO: 1167 - DNA, 10 bp, Homo sapiens
tagaaaggca

SEQ ID NO: 1168 - DNA, 10 bp, Homo sapiens
taagtagcaa

SEQ ID NO: 1169 - DNA, 10 bp, Homo sapiens
ggtgagacac

SEQ ID NO: 1170 - DNA, 10 bp, Homo sapiens
cccatcgtct

SEQ ID NO: 1171 - DNA, 10 bp, Homo sapiens
ccgatcaccg

SEQ ID NO: 1172 - DNA, 10 bp, Homo sapiens
gaatcggtta

SEQ ID NO: 1173 - DNA, 10 bp, Homo sapiens
aacccaggag

SEQ ID NO: 1174 - DNA, 10 bp, Homo sapiens
ttttgaagca

SEQ ID NO: 1175 - DNA, 10 bp, Homo sapiens
cacaggcaaa

SEQ ID NO: 1176 - DNA, 10 bp, Homo sapiens
tcagcttcac

SEQ ID NO: 1177 - DNA, 10 bp, Homo sapiens
tcagcttcac

SEQ ID NO: 1178 - DNA, 10 bp, Homo sapiens
gagggccggt

SEQ ID NO: 1179 - DNA, 10 bp, Homo sapiens
ccccagccag

SEQ ID NO: 1180 - DNA, 10 bp, Homo sapiens
gtggtgggtg

SEQ ID NO: 1181 - DNA, 10 bp, Homo sapiens
ctgccaagtt

SEQ ID NO: 1182 - DNA, 10 bp, Homo sapiens
gagaaaccct

SEQ ID NO: 1183 - DNA, 10 bp, Homo sapiens
gagaaaccct

SEQ ID NO: 1184 - DNA, 10 bp, Homo sapiens
actaacaccc

SEQ ID NO: 1185 - DNA, 10 bp, Homo sapiens
ttttgggggc

SEQ ID NO: 1186 - DNA, 10 bp, Homo sapiens
ttttgggggc

SEQ ID NO: 1187 - DNA, 10 bp, Homo sapiens
gtgaaaccca

SEQ ID NO: 1188 - DNA, 10 bp, Homo sapiens
gctttcattg

SEQ ID NO: 1189 - DNA, 10 bp, Homo sapiens
gtggcacgca

SEQ ID NO: 1190 - DNA, 10 bp, Homo sapiens
gggtcaaaag

SEQ ID NO: 1191 - DNA, 10 bp, Homo sapiens
gggggtcacc

SEQ ID NO: 1192 - DNA, 10 bp, Homo sapiens
gtgaaaccct

SEQ ID NO: 1193 - DNA, 10 bp, Homo sapiens
gtgaaaccct

SEQ ID NO: 1194 - DNA, 10 bp, Homo sapiens
gtgaaaccct

SEQ ID NO: 1195 - DNA, 10 bp, Homo sapiens
gtgaaaccct

SEQ ID NO: 1196 - DNA, 10 bp, Homo sapiens
gtgaaaccct

SEQ ID NO: 1197 - DNA, 10 bp, Homo sapiens
gtgaaaccct

SEQ ID NO: 1198 - DNA, 10 bp, Homo sapiens
agttgaaatt

SEQ ID NO: 1199 - DNA, 10 bp, Homo sapiens
agaatcgctt

SEQ ID NO: 1200 - DNA, 10 bp, Homo sapiens
aggtcaagag

SEQ ID NO: 1201 - DNA, 10 bp, Homo sapiens
ctaaccagac

SEQ ID NO: 1202 - DNA, 10 bp, Homo sapiens
gggatggcag

SEQ ID NO: 1203 - DNA, 10 bp, Homo sapiens
agacccacaa

SEQ ID NO: 1204 - DNA, 10 bp, Homo sapiens
tcgaagaacc

SEQ ID NO: 1205 - DNA, 10 bp, Homo sapiens
tgaaataaaa

SEQ ID NO: 1206 - DNA, 10 bp, Homo sapiens
actgaggtgc

SEQ ID NO: 1207 - DNA, 10 bp, Homo sapiens
actcagaaga

SEQ ID NO: 1208 - DNA, 10 bp, Homo sapiens
gaacacatcc

SEQ ID NO: 1209 - DNA, 10 bp, Homo sapiens
aactaatact

SEQ ID NO: 1210 - DNA, 10 bp, Homo sapiens
agatgtgtgg

SEQ ID NO: 1211 - DNA, 10 bp, Homo sapiens
gtggtgtgca

SEQ ID NO: 1212 - DNA, 10 bp, Homo sapiens
ggcgtcctgg

SEQ ID NO: 1213 - DNA, 10 bp, Homo sapiens
cctgcaatcc

SEQ ID NO: 1214 - DNA, 10 bp, Homo sapiens
gcctggccat

SEQ ID NO: 1215 - DNA, 10 bp, Homo sapiens
gcctggccat

SEQ ID NO: 1216 - DNA, 10 bp, Homo sapiens
gctgcccttg

SEQ ID NO: 1217 - DNA, 10 bp, Homo sapiens
gctgcccttg

SEQ ID NO: 1218 - DNA, 10 bp, Homo sapiens
gccagcccag

SEQ ID NO: 1219 - DNA, 10 bp, Homo sapiens
tcctattaag

SEQ ID NO: 1220 - DNA, 10 bp, Homo sapiens
attgtgccac

SEQ ID NO: 1221 - DNA, 10 bp, Homo sapiens
ccattgcact

SEQ ID NO: 1222 - DNA, 10 bp, Homo sapiens
gcacctcagc

SEQ ID NO: 1223 - DNA, 10 bp, Homo sapiens
ttggtcaggc

SEQ ID NO: 1224 - DNA, 10 bp, Homo sapiens
ttggtcaggc

SEQ ID NO: 1225 - DNA, 10 bp, Homo sapiens
gggccccgca

SEQ ID NO: 1226 - DNA, 10 bp, Homo sapiens
gtggcacaca

SEQ ID NO: 1227 - DNA, 10 bp, Homo sapiens
gtggcacaca

SEQ ID NO: 1228 - DNA, 10 bp, Homo sapiens
ttggccaggc

SEQ ID NO: 1229 - DNA, 10 bp, Homo sapiens
ttggccaggc

SEQ ID NO: 1230 - DNA, 10 bp, Homo sapiens
ttggccaggc

SEQ ID NO: 1231 - DNA, 10 bp, Homo sapiens
ttggccaggc

SEQ ID NO: 1232 - DNA, 10 bp, Homo sapiens
ttggccaggc

SEQ ID NO: 1233 - DNA, 10 bp, Homo sapiens
ttggccaggc

SEQ ID NO: 1234 - DNA, 10 bp, Homo sapiens
ttggccaggc

SEQ ID NO: 1235 - DNA, 10 bp, Homo sapiens
gtcactgcct

SEQ ID NO: 1236 - DNA, 10 bp, Homo sapiens
gccaccccgt

SEQ ID NO: 1237 - DNA, 10 bp, Homo sapiens
tccctataag

SEQ ID NO: 1238 - DNA, 10 bp, Homo sapiens
cctgtaatcc

SEQ ID NO: 1239 - DNA, 10 bp, Homo sapiens
cctgtaatcc

SEQ ID NO: 1240 - DNA, 10 bp, Homo sapiens
cctgtaatcc

SEQ ID NO: 1241 - DNA, 10 bp, Homo sapiens
cctgtaatcc

SEQ ID NO: 1242 - DNA, 10 bp, Homo sapiens
cctgtaatcc

SEQ ID NO: 1243 - DNA, 10 bp, Homo sapiens
cctgtaatcc

SEQ ID NO: 1244 - DNA, 10 bp, Homo sapiens
cctgtaatcc

SEQ ID NO: 1245 - DNA, 10 bp, Homo sapiens
cctgtaatcc

SEQ ID NO: 1246 - DNA, 10 bp, Homo sapiens
cctgtaatcc

SEQ ID NO: 1247 - DNA, 10 bp, Homo sapiens
cctgtaatcc

SEQ ID NO: 1248 - DNA, 10 bp, Homo sapiens
cctgtaatcc

SEQ ID NO: 1249 - DNA, 10 bp, Homo sapiens
cctgtaatcc

SEQ ID NO: 1250 - DNA, 10 bp, Homo sapiens
cctgtaatcc

SEQ ID NO: 1251 - DNA, 10 bp, Homo sapiens
cctgtaatcc

SEQ ID NO: 1252 - DNA, 10 bp, Homo sapiens
cctgtaatcc

SEQ ID NO: 1253 - DNA, 10 bp, Homo sapiens
cctgtaatcc

SEQ ID NO: 1254 - DNA, 10 bp, Homo sapiens
cctgtaatcc

SEQ ID NO: 1255 - DNA, 10 bp, Homo sapiens
cctgtaatcc

SEQ ID NO: 1256 - DNA, 10 bp, Homo sapiens
cctgtaatcc

SEQ ID NO: 1257 - DNA, 10 bp, Homo sapiens
tccccgtaca

SEQ ID NO: 1258 - DNA, 10 bp, Homo sapiens
gtcacaccac

SEQ ID NO: 1259 - DNA, 10 bp, Homo sapiens
gtcacaccac

SEQ ID NO: 1260 - DNA, 10 bp, Homo sapiens
atggcaaggg

SEQ ID NO: 1261 - DNA, 10 bp, Homo sapiens
ctgttggcat

SEQ ID NO: 1262 - DNA, 10 bp, Homo sapiens
ctagcctcac

SEQ ID NO: 1263 - DNA, 10 bp, Homo sapiens
agtgcaagac

SEQ ID NO: 1264 - DNA, 10 bp, Homo sapiens
cctgtagtcc

SEQ ID NO: 1265 - DNA, 10 bp, Homo sapiens
ttttctgaaa

SEQ ID NO: 1266 - DNA, 10 bp, Homo sapiens
ctcccctgcc

SEQ ID NO: 1267 - DNA, 10 bp, Homo sapiens
tctctttttc

SEQ ID NO: 1268 - DNA, 10 bp, Homo sapiens
gcggacgagg

SEQ ID NO: 1269 - DNA, 10 bp, Homo sapiens
gcggacgagg

SEQ ID NO: 1270 - DNA, 10 bp, Homo sapiens
ggagtcattg

SEQ ID NO: 1271 - DNA, 10 bp, Homo sapiens
gtagcaggtg

SEQ ID NO: 1272 - DNA, 10 bp, Homo sapiens
cgcaagctgg

SEQ ID NO: 1273 - DNA, 10 bp, Homo sapiens
gtgaaacccg

SEQ ID NO: 1274 - DNA, 10 bp, Homo sapiens
aggtcaggag

SEQ ID NO: 1275 - DNA, 10 bp, Homo sapiens
aggtcaggag

SEQ ID NO: 1276 - DNA, 10 bp, Homo sapiens
aggtcaggag

SEQ ID NO: 1277 - DNA, 10 bp, Homo sapiens
gaatgcagtt

SEQ ID NO: 1278 - DNA, 10 bp, Homo sapiens
gaatgcagtt

SEQ ID NO: 1279 - DNA, 10 bp, Homo sapiens
gaatgcagtt

SEQ ID NO: 1280 - DNA, 10 bp, Homo sapiens
gtgagcccat

SEQ ID NO: 1281 - DNA, 10 bp, Homo sapiens
gtaatcctgc

SEQ ID NO: 1282 - DNA, 10 bp, Homo sapiens
tgaagtaaca

SEQ ID NO: 1283 - DNA, 10 bp, Homo sapiens
tgcctgtaat

SEQ ID NO: 1284 - DNA, 10 bp, Homo sapiens
gtagcataaa

SEQ ID NO: 1285 - DNA, 10 bp, Homo sapiens
ccgtggtcgt

SEQ ID NO: 1286 - DNA, 10 bp, Homo sapiens
atgaaacccc

SEQ ID NO: 1287 - DNA, 10 bp, Homo sapiens
aagattggtg

SEQ ID NO: 1288 - DNA, 10 bp, Homo sapiens
atccgtgccc

SEQ ID NO: 1289 - DNA, 10 bp, Homo sapiens
cccttcactg

SEQ ID NO: 1290 - DNA, 10 bp, Homo sapiens
cccttcactg

SEQ ID NO: 1291 - DNA, 10 bp, Homo sapiens
cagctggggc

SEQ ID NO: 1292 - DNA, 10 bp, Homo sapiens
caggccccac

SEQ ID NO: 1293 - DNA, 10 bp, Homo sapiens
tgtttatcct

SEQ ID NO: 1294 - DNA, 10 bp, Homo sapiens
taaccaatca

SEQ ID NO: 1295 - DNA, 10 bp, Homo sapiens
cacctgtagt

SEQ ID NO: 1296 - DNA, 10 bp, Homo sapiens
taccctaaaa

SEQ ID NO: 1297 - DNA, 10 bp, Homo sapiens
taccctaaaa

SEQ ID NO: 1298 - DNA, 10 bp, Homo sapiens
taccctaaaa

SEQ ID NO: 1299 - DNA, 10 bp, Homo sapiens
tgcctctgcg

SEQ ID NO: 1300 - DNA, 10 bp, Homo sapiens
gcaaaaccct

SEQ ID NO: 1301 - DNA, 10 bp, Homo sapiens
aaggaccttt

SEQ ID NO: 1302 - DNA, 10 bp, Homo sapiens
ctggcgccga

SEQ ID NO: 1303 - DNA, 10 bp, Homo sapiens
gaagctttgc

SEQ ID NO: 1304 - DNA, 10 bp, Homo sapiens
gctccgagcg

SEQ ID NO: 1305 - DNA, 10 bp, Homo sapiens
ttgcccaggc

SEQ ID NO: 1306 - DNA, 10 bp, Homo sapiens
ttgcccaggc

SEQ ID NO: 1307 - DNA, 10 bp, Homo sapiens
acccacgtca

SEQ ID NO: 1308 - DNA, 10 bp, Homo sapiens
gctccactgg

SEQ ID NO: 1309 - DNA, 10 bp, Homo sapiens
tttaacggcc

SEQ ID NO: 1310 - DNA, 10 bp, Homo sapiens
cttgtaatcc

SEQ ID NO: 1311 - DNA, 10 bp, Homo sapiens
cacttttggg

SEQ ID NO: 1312 - DNA, 10 bp, Homo sapiens
ccgggtgatg

SEQ ID NO: 1313 - DNA, 10 bp, Homo sapiens
ggggtaagaa

SEQ ID NO: 1314 - DNA, 10 bp, Homo sapiens
tgactggcag

SEQ ID NO: 1315 - DNA, 10 bp, Homo sapiens
caatgtgtta

SEQ ID NO: 1316 - DNA, 10 bp, Homo sapiens
ggctcgggat

SEQ ID NO: 1317 - DNA, 10 bp, Homo sapiens
tgcctgtagt

SEQ ID NO: 1318 - DNA, 10 bp, Homo sapiens
cgccgccggc

SEQ ID NO: 1319 - DNA, 10 bp, Homo sapiens
ggtggggaga

SEQ ID NO: 1320 - DNA, 10 bp, Homo sapiens
gtaaaaccct

SEQ ID NO: 1321 - DNA, 10 bp, Homo sapiens
ggctcctggc

SEQ ID NO: 1322 - DNA, 10 bp, Homo sapiens
agtaggtggc

SEQ ID NO: 1323 - DNA, 10 bp, Homo sapiens
ggaggtgggg

SEQ ID NO: 1324 - DNA, 10 bp, Homo sapiens
cctttggcta

SEQ ID NO: 1325 - DNA, 10 bp, Homo sapiens
agaaagatgt

SEQ ID NO: 1326 - DNA, 10 bp, Homo sapiens
agaacaaaac

SEQ ID NO: 1327 - DNA, 10 bp, Homo sapiens
aactaaaaaa

SEQ ID NO: 1328 - DNA, 10 bp, Homo sapiens
attgcaccac

SEQ ID NO: 1329 - DNA, 10 bp, Homo sapiens
gatcccaact

SEQ ID NO: 1330 - DNA, 10 bp, Homo sapiens
gatcccaact

SEQ ID NO: 1331 - DNA, 10 bp, Homo sapiens
cactactcac

SEQ ID NO: 1332 - DNA, 10 bp, Homo sapiens
ctgtacagac

SEQ ID NO: 1333 - DNA, 10 bp, Homo sapiens
taccctagaa

SEQ ID NO: 1334 - DNA, 10 bp, Homo sapiens
gtaaaacccc

SEQ ID NO: 1335 - DNA, 10 bp, Homo sapiens
gtaaaacccc

SEQ ID NO: 1336 - DNA, 10 bp, Homo sapiens
gtaaaacccc

SEQ ID NO: 1337 - DNA, 10 bp, Homo sapiens
ctgagagctg

SEQ ID NO: 1338 - DNA, 10 bp, Homo sapiens
ggctggtctg

SEQ ID NO: 1339 - DNA, 10 bp, Homo sapiens
acgcagggag

SEQ ID NO: 1340 - DNA, 10 bp, Homo sapiens
gccctcggcc

SEQ ID NO: 1341 - DNA, 10 bp, Homo sapiens
ctcccttgcc

SEQ ID NO: 1342 - DNA, 10 bp, Homo sapiens
cctgtaatct

SEQ ID NO: 1343 - DNA, 10 bp, Homo sapiens
aggtcctagc

SEQ ID NO: 1344 - DNA, 10 bp, Homo sapiens
actgaaggcg

SEQ ID NO: 1345 - DNA, 10 bp, Homo sapiens
aaggaagatg

SEQ ID NO: 1346 - DNA, 10 bp, Homo sapiens
ccgacgggcg

SEQ ID NO: 1347 - DNA, 10 bp, Homo sapiens
gcccccaata

SEQ ID NO: 1348 - DNA, 10 bp, Homo sapiens
aggatgtggg

SEQ ID NO: 1349 - DNA, 10 bp, Homo sapiens
ggaggccgag

SEQ ID NO: 1350 - DNA, 10 bp, Homo sapiens
acccccccgc

SEQ ID NO: 1351 - DNA, 10 bp, Homo sapiens
ctggcctgtg

SEQ ID NO: 1352 - DNA, 10 bp, Homo sapiens
ctggcctgtg

SEQ ID NO: 1353 - DNA, 10 bp, Homo sapiens
ctggcctgtg

SEQ ID NO: 1354 - DNA, 10 bp, Homo sapiens
cacccccagg

SEQ ID NO: 1355 - DNA, 10 bp, Homo sapiens
cacccccagg

SEQ ID NO: 1356 - DNA, 10 bp, Homo sapiens
gtgaaactcc

SEQ ID NO: 1357 - DNA, 10 bp, Homo sapiens
gtgaaactcc

SEQ ID NO: 1358 - DNA, 10 bp, Homo sapiens
agaattgctt

SEQ ID NO: 1359 - DNA, 10 bp, Homo sapiens
agaattgctt

SEQ ID NO: 1360 - DNA, 10 bp, Homo sapiens
atggcctcct

SEQ ID NO: 1361 - DNA, 10 bp, Homo sapiens
aactgtcctt

SEQ ID NO: 1362 - DNA, 10 bp, Homo sapiens
aaggaatcgg

SEQ ID NO: 1363 - DNA, 10 bp, Homo sapiens
tctgtttatc

SEQ ID NO: 1364 - DNA, 10 bp, Homo sapiens
actttttcaa

SEQ ID NO: 1365 - DNA, 10 bp, Homo sapiens
tctgtaatcc

SEQ ID NO: 1366 - DNA, 10 bp, Homo sapiens
tctgtaatcc

SEQ ID NO: 1367 - DNA, 10 bp, Homo sapiens
gtgaaaaccc

SEQ ID NO: 1368 - DNA, 10 bp, Homo sapiens
ggcaggcaca

SEQ ID NO: 1369 - DNA, 10 bp, Homo sapiens
ggggcagggc

SEQ ID NO: 1370 - DNA, 10 bp, Homo sapiens
ggggcagggc

SEQ ID NO: 1371 - DNA, 10 bp, Homo sapiens
gtgaaactct

SEQ ID NO: 1372 - DNA, 10 bp, Homo sapiens
tggaccaggc

SEQ ID NO: 1373 - DNA, 10 bp, Homo sapiens
cctataatcc

SEQ ID NO: 1374 - DNA, 10 bp, Homo sapiens
cctataatcc

SEQ ID NO: 1375 - DNA, 10 bp, Homo sapiens
cctataatcc

SEQ ID NO: 1376 - DNA, 10 bp, Homo sapiens
aactgcttca

SEQ ID NO: 1377 - DNA, 10 bp, Homo sapiens
ggattgtctg

SEQ ID NO: 1378 - DNA, 10 bp, Homo sapiens
cctgtaattc

SEQ ID NO: 1379 - DNA, 10 bp, Homo sapiens
ctgggcctgg

SEQ ID NO: 1380 - DNA, 10 bp, Homo sapiens
acccttggcc

SEQ ID NO: 1381 - DNA, 10 bp, Homo sapiens
atggcgatct

SEQ ID NO: 1382 - DNA, 10 bp, Homo sapiens
ttgtctgcct

SEQ ID NO: 1383 - DNA, 10 bp, Homo sapiens
tgaatctggg

SEQ ID NO: 1384 - DNA, 10 bp, Homo sapiens
agcctttgtt

SEQ ID NO: 1385 - DNA, 10 bp, Homo sapiens
cttttcagca

SEQ ID NO: 1386 - DNA, 10 bp, Homo sapiens
cctggagtgg

SEQ ID NO: 1387 - DNA, 10 bp, Homo sapiens
cggagaccct

SEQ ID NO: 1388 - DNA, 10 bp, Homo sapiens
ccctgggttc

SEQ ID NO: 1389 - DNA, 10 bp, Homo sapiens
atttgagaag

SEQ ID NO: 1390 - DNA, 10 bp, Homo sapiens
acaactcaat

SEQ ID NO: 1391 - DNA, 10 bp, Homo sapiens
cttgattccc

SEQ ID NO: 1392 - DNA, 10 bp, Homo sapiens
ggctggtctc

SEQ ID NO: 1393 - DNA, 10 bp, Homo sapiens
aggtggcaag

SEQ ID NO: 1394 - DNA, 10 bp, Homo sapiens
ctagctttta

SEQ ID NO: 1395 - DNA, 10 bp, Homo sapiens
tcaccggtca

SEQ ID NO: 1396 - DNA, 10 bp, Homo sapiens
ggccgcgttc

SEQ ID NO: 1397 - DNA, 10 bp, Homo sapiens
gagagctccc

SEQ ID NO: 1398 - DNA, 10 bp, Homo sapiens
gagagctccc

SEQ ID NO: 1399 - DNA, 10 bp, Homo sapiens
gagagctccc

SEQ ID NO: 1400 - DNA, 10 bp, Homo sapiens
gagagctccc

SEQ ID NO: 1401 - DNA, 10 bp, Homo sapiens
ccccgtacat

SEQ ID NO: 1402 - DNA, 10 bp, Homo sapiens
tggcgtacgg

SEQ ID NO: 1403 - DNA, 10 bp, Homo sapiens
tccccgacat

SEQ ID NO: 1404 - DNA, 10 bp, Homo sapiens
cctggctaat

SEQ ID NO: 1405 - DNA, 10 bp, Homo sapiens
tcacagctgt

SEQ ID NO: 1406 - DNA, 10 bp, Homo sapiens
tcccattaag

SEQ ID NO: 1407 - DNA, 10 bp, Homo sapiens
gtgcactgag

SEQ ID NO: 1408 - DNA, 10 bp, Homo sapiens
gtgcactgag

SEQ ID NO: 1409 - DNA, 10 bp, Homo sapiens
gcttaccttt

SEQ ID NO: 1410 - DNA, 10 bp, Homo sapiens
ctggcccgga

SEQ ID NO: 1411 - DNA, 10 bp, Homo sapiens
ctggcccgga

SEQ ID NO: 1412 - DNA, 10 bp, Homo sapiens
gggcctgtgc

SEQ ID NO: 1413 - DNA, 10 bp, Homo sapiens
gggcctgtgc

SEQ ID NO: 1414 - DNA, 10 bp, Homo sapiens
gcccctccgg

SEQ ID NO: 1415 - DNA, 10 bp, Homo sapiens
ttgtgatgta

SEQ ID NO: 1416 - DNA, 10 bp, Homo sapiens
ttgtgatgta

SEQ ID NO: 1417 - DNA, 10 bp, Homo sapiens
catcttcacc

SEQ ID NO: 1418 - DNA, 10 bp, Homo sapiens
ttggccagga

SEQ ID NO: 1419 - DNA, 10 bp, Homo sapiens
agaatcactt

SEQ ID NO: 1420 - DNA, 10 bp, Homo sapiens
ttagccagga

SEQ ID NO: 1421 - DNA, 10 bp, Homo sapiens
gttgtggtta

SEQ ID NO: 1422 - DNA, 10 bp, Homo sapiens
caagcatccc

SEQ ID NO: 1423 - DNA, 10 bp, Homo sapiens
gacatatgta

SEQ ID NO: 1424 - DNA, 10 bp, Homo sapiens
agtatctggg

SEQ ID NO: 1425 - DNA, 10 bp, Homo sapiens
accgcctgtg

SEQ ID NO: 1426 - DNA, 10 bp, Homo sapiens
ctcttcgaga

SEQ ID NO: 1427 - DNA, 10 bp, Homo sapiens
atgagctgac

SEQ ID NO: 1428 - DNA, 10 bp, Homo sapiens
gcctctgtct

SEQ ID NO: 1429 - DNA, 10 bp, Homo sapiens
aaggaagatc

SEQ ID NO: 1430 - DNA, 10 bp, Homo sapiens
aaaacattct

SEQ ID NO: 1431 - DNA, 10 bp, Homo sapiens
ctcagacagt

SEQ ID NO: 1432 - DNA, 10 bp, Homo sapiens
cccaagctag

SEQ ID NO: 1433 - DNA, 10 bp, Homo sapiens
cccaagctag

SEQ ID NO: 1434 - DNA, 10 bp, Homo sapiens
tcaatcaaga

SEQ ID NO: 1435 - DNA, 10 bp, Homo sapiens
tgcagcgcct

SEQ ID NO: 1436 - DNA, 10 bp, Homo sapiens
ttcactgtga

SEQ ID NO: 1437 - DNA, 10 bp, Homo sapiens
ctgacctgtg

SEQ ID NO: 1438 - DNA, 10 bp, Homo sapiens
ggggtcaggg

SEQ ID NO: 1439 - DNA, 10 bp, Homo sapiens
ggctttaggg

SEQ ID NO: 1440 - DNA, 10 bp, Homo sapiens
tgggtgagcc

SEQ ID NO: 1441 - DNA, 10 bp, Homo sapiens
agggtgtttt

SEQ ID NO: 1442 - DNA, 10 bp, Homo sapiens
agggtgtttt

SEQ ID NO: 1443 - DNA, 10 bp, Homo sapiens
tggtgtatgc

SEQ ID NO: 1444 - DNA, 10 bp, Homo sapiens
gagtagagaa

SEQ ID NO: 1445 - DNA, 10 bp, Homo sapiens
tgcaggcctg

SEQ ID NO: 1446 - DNA, 10 bp, Homo sapiens
gcgaaaccct

SEQ ID NO: 1447 - DNA, 10 bp, Homo sapiens
gtgaccacgg

SEQ ID NO: 1448 - DNA, 10 bp, Homo sapiens
gtgaccacgg

SEQ ID NO: 1449 - DNA, 10 bp, Homo sapiens
cccatcgtcc

SEQ ID NO: 1450 - DNA, 10 bp, Homo sapiens
tgtgttgaga

SEQ ID NO: 1451 - DNA, 10 bp, Homo sapiens
ggatttggcc

SEQ ID NO: 1452 - DNA, 10 bp, Homo sapiens
cccgtccgga

SEQ ID NO: 1453 - DNA, 10 bp, Homo sapiens
atggctggta

SEQ ID NO: 1454 - DNA, 10 bp, Homo sapiens
gtgaaacccc

SEQ ID NO: 1455 - DNA, 10 bp, Homo sapiens
cctccagcta

SEQ ID NO: 1456 - DNA, 10 bp, Homo sapiens
ttggtcctct

SEQ ID NO: 1457 - DNA, 10 bp, Homo sapiens
tgatttcact

SEQ ID NO: 1458 - DNA, 10 bp, Homo sapiens
cctgtaatcc

SEQ ID NO: 1459 - DNA, 10 bp, Homo sapiens
actttttcaa

SEQ ID NO: 1460 - DNA, 10 bp, Homo sapiens
aaaaaaaaaa

SEQ ID NO: 1461 - DNA, 10 bp, Homo sapiens
gagggagttt

SEQ ID NO: 1462 - DNA, 10 bp, Homo sapiens
gccgaggaag

SEQ ID NO: 1463 - DNA, 10 bp, Homo sapiens
cacctaattg

SEQ ID NO: 1464 - DNA, 10 bp, Homo sapiens
cgccgccggc

SEQ ID NO: 1465 - DNA, 10 bp, Homo sapiens
ggggaaatcg

SEQ ID NO: 1466 - DNA, 10 bp, Homo sapiens
gaaaaatggt

SEQ ID NO: 1467 - DNA, 10 bp, Homo sapiens
gggctggggt

SEQ ID NO: 1468 - DNA, 10 bp, Homo sapiens
gccgggtggg

SEQ ID NO: 1469 - DNA, 10 bp, Homo sapiens
agccctacaa

SEQ ID NO: 1470 - DNA, 10 bp, Homo sapiens
ctgggttaat

SEQ ID NO: 1471 - DNA, 10 bp, Homo sapiens
caaaccatcc

SEQ ID NO: 1472 - DNA, 10 bp, Homo sapiens
tgcacgtttt

SEQ ID NO: 1473 - DNA, 10 bp, Homo sapiens
aggctacgga

SEQ ID NO: 1474 - DNA, 10 bp, Homo sapiens
gcagccatcc

SEQ ID NO: 1475 - DNA, 10 bp, Homo sapiens
ttcaataaaa

SEQ ID NO: 1476 - DNA, 10 bp, Homo sapiens
ctaagacttc

SEQ ID NO: 1477 - DNA, 10 bp, Homo sapiens
tggtgttgag

SEQ ID NO: 1478 - DNA, 10 bp, Homo sapiens
taccatcaat

SEQ ID NO: 1479 - DNA, 10 bp, Homo sapiens
ttcatacacc

SEQ ID NO: 1480 - DNA, 10 bp, Homo sapiens
ccactgcact

SEQ ID NO: 1481 - DNA, 10 bp, Homo sapiens
actaacaccc

SEQ ID NO: 1482 - DNA, 10 bp, Homo sapiens
aaggtggagg

SEQ ID NO: 1483 - DNA, 10 bp, Homo sapiens
agcacctcca

SEQ ID NO: 1484 - DNA, 10 bp, Homo sapiens
cacaaacggt

SEQ ID NO: 1485 - DNA, 10 bp, Homo sapiens
aggaaagctg

SEQ ID NO: 1486 - DNA, 10 bp, Homo sapiens
gtgaaaccct

SEQ ID NO: 1487 - DNA, 10 bp, Homo sapiens
aatcctgtgg

SEQ ID NO: 1488 - DNA, 10 bp, Homo sapiens
ttggggtttc

SEQ ID NO: 1489 - DNA, 10 bp, Homo sapiens
aagacagtgg

SEQ ID NO: 1490 - DNA, 10 bp, Homo sapiens
atttgagaag

SEQ ID NO: 1491 - DNA, 10 bp, Homo sapiens
gccgtgtccg

SEQ ID NO: 1492 - DNA, 10 bp, Homo sapiens
cgccggaaca

SEQ ID NO: 1493 - DNA, 10 bp, Homo sapiens
tctccatacc

SEQ ID NO: 1494 - DNA, 10 bp, Homo sapiens
acatcatcga

SEQ ID NO: 1495 - DNA, 10 bp, Homo sapiens
aacgcggcca

SEQ ID NO: 1496 - DNA, 10 bp, Homo sapiens
agggcttcca

SEQ ID NO: 1497 - DNA, 10 bp, Homo sapiens
ccgtccaagg

SEQ ID NO: 1498 - DNA, 10 bp, Homo sapiens
cgctggttcc

SEQ ID NO: 1499 - DNA, 10 bp, Homo sapiens
ctcaacatct

SEQ ID NO: 1500 - DNA, 10 bp, Homo sapiens
actccaaaaa

SEQ ID NO: 1501 - DNA, 10 bp, Homo sapiens
cctagctgga

SEQ ID NO: 1502 - DNA, 10 bp, Homo sapiens
gtgaaggcag

SEQ ID NO: 1503 - DNA, 10 bp, Homo sapiens
agctctccct

SEQ ID NO: 1504 - DNA, 10 bp, Homo sapiens
taggttgtct

SEQ ID NO: 1505 - DNA, 10 bp, Homo sapiens
ggaccactga

SEQ ID NO: 1506 - DNA, 10 bp, Homo sapiens
aaggagatgg

SEQ ID NO: 1507 - DNA, 10 bp, Homo sapiens
aactaaaaaa

SEQ ID NO: 1508 - DNA, 10 bp, Homo sapiens
ggctgggggc

SEQ ID NO: 1509 - DNA, 10 bp, Homo sapiens
ccagaacaga

SEQ ID NO: 1510 - DNA, 10 bp, Homo sapiens
cccatcgtcc

SEQ ID NO: 1511 - DNA, 10 bp, Homo sapiens
gtgaccacgg

SEQ ID NO: 1512 - DNA, 10 bp, Homo sapiens
tgtgttgaga

SEQ ID NO: 1513 - DNA, 10 bp, Homo sapiens
gtgaaacccc

SEQ ID NO: 1514 - DNA, 10 bp, Homo sapiens
cctgtaatcc

SEQ ID NO: 1515 - DNA, 10 bp, Homo sapiens
ctaagacttc

SEQ ID NO: 1516 - DNA, 10 bp, Homo sapiens
cacctaattg

SEQ ID NO: 1517 - DNA, 10 bp, Homo sapiens
cccgtccgga

SEQ ID NO: 1518 - DNA, 10 bp, Homo sapiens
ttggtcctct

SEQ ID NO: 1519 - DNA, 10 bp, Homo sapiens
atggctggta

SEQ ID NO: 1520 - DNA, 10 bp, Homo sapiens
ttggggtttc

SEQ ID NO: 1521 - DNA, 10 bp, Homo sapiens
ccactgcact

SEQ ID NO: 1522 - DNA, 10 bp, Homo sapiens
tgatttcact

SEQ ID NO: 1523 - DNA, 10 bp, Homo sapiens
actttttcaa

SEQ ID NO: 1524 - DNA, 10 bp, Homo sapiens
gcagccatcc

SEQ ID NO: 1525 - DNA, 10 bp, Homo sapiens
taccatcaat

SEQ ID NO: 1526 - DNA, 10 bp, Homo sapiens
ggatttggcc

SEQ ID NO: 1527 - DNA, 10 bp, Homo sapiens
ccctgggttc

SEQ ID NO: 1528 - DNA, 10 bp, Homo sapiens
gccgaggaag

SEQ ID NO: 1529 - DNA, 10 bp, Homo sapiens
aggctacgga

SEQ ID NO: 1530 - DNA, 10 bp, Homo sapiens
cgccgccggc

SEQ ID NO: 1531 - DNA, 10 bp, Homo sapiens
ttcatacacc

SEQ ID NO: 1532 - DNA, 10 bp, Homo sapiens
agccctacaa

SEQ ID NO: 1533 - DNA, 10 bp, Homo sapiens
cacaaacggt

SEQ ID NO: 1534 - DNA, 10 bp, Homo sapiens
aaggtggagg

SEQ ID NO: 1535 - DNA, 10 bp, Homo sapiens
cttccttgcc

SEQ ID NO: 1536 - DNA, 10 bp, Homo sapiens
tggtgttgag

SEQ ID NO: 1537 - DNA, 10 bp, Homo sapiens
gtgaaaccct

SEQ ID NO: 1538 - DNA, 10 bp, Homo sapiens
ggggaaatcg

SEQ ID NO: 1539 - DNA, 10 bp, Homo sapiens
agcacctcca

SEQ ID NO: 1540 - DNA, 10 bp, Homo sapiens
cctccagcta

SEQ ID NO: 1541 - DNA, 10 bp, Homo sapiens
aagacagtgg

SEQ ID NO: 1542 - DNA, 10 bp, Homo sapiens
ctgggttaat

SEQ ID NO: 1543 - DNA, 10 bp, Homo sapiens
atttgagaag

SEQ ID NO: 1544 - DNA, 10 bp, Homo sapiens
gccgggtggg

SEQ ID NO: 1545 - DNA, 10 bp, Homo sapiens
gggctggggt

SEQ ID NO: 1546 - DNA, 10 bp, Homo sapiens
agggcttcca

SEQ ID NO: 1547 - DNA, 10 bp, Homo sapiens
aaaaaaaaaa

SEQ ID NO: 1548 - DNA, 10 bp, Homo sapiens
gagggagttt

SEQ ID NO: 1549 - DNA, 10 bp, Homo sapiens
gcgaccgtca

SEQ ID NO: 1550 - DNA, 10 bp, Homo sapiens
actaacaccc

SEQ ID NO: 1551 - DNA, 10 bp, Homo sapiens
cgccggaaca

SEQ ID NO: 1552 - DNA, 10 bp, Homo sapiens
tgggcaaagc

SEQ ID NO: 1553 - DNA, 10 bp, Homo sapiens
tgcacgtttt

SEQ ID NO: 1554 - DNA, 10 bp, Homo sapiens
aatcctgtgg

SEQ ID NO: 1555 - DNA, 10 bp, Homo sapiens
caagcatccc

SEQ ID NO: 1556 - DNA, 10 bp, Homo sapiens
ccgtccaagg

SEQ ID NO: 1557 - DNA, 10 bp, Homo sapiens
taggttgtct

SEQ ID NO: 1558 - DNA, 10 bp, Homo sapiens
gccgtgtccg

SEQ ID NO: 1559 - DNA, 10 bp, Homo sapiens
gctttatttg

SEQ ID NO: 1560 - DNA, 10 bp, Homo sapiens
ctagcctcac

SEQ ID NO: 1561 - DNA, 10 bp, Homo sapiens
cctagctgga

SEQ ID NO: 1562 - DNA, 10 bp, Homo sapiens
gcccctgctg

SEQ ID NO: 1563 - DNA, 10 bp, Homo sapiens
acccttggcc

SEQ ID NO: 1564 - DNA, 10 bp, Homo sapiens
aggaaagctg

(19) United States
(12) Patent Application Publication
(10) Pub. No.: US 2011/0033466 A1
(43) Pub. Date: Feb. 10, 2011

(54) HUMAN TRANSCRIPTOMES

(75) Inventors: Victor E. Velculescu, Baltimore, MD (US); Bert Vogelstein, Baltimore, MD (US); Kenneth W. Kinzler, Bel Air, MD (US)

Correspondence Address: BANNER & WITCOFF, LTD., 1100 13th STREET, N.W., SUITE 1200, WASHINGTON, DC, 20005-4051, US

(73) Assignee: The Johns Hopkins University, Baltimore, MD (US)

(21) Appl. No.: 12/858,717

(22) Filed: Aug. 18, 2010

Related U.S. Application Data
(63) Continuation of application No. 11/057,194, filed on Feb. 15, 2005, now abandoned.
Application No. 11/057,194 is a continuation of application No. 10/330,627, filed on Dec. 30, 2002, now abandoned.
Application No. 10/330,627 is a continuation of application No. 09/448,480, filed on Nov. 24, 1999, now abandoned.

(51) Int. Cl. C12Q 1/68; C07H 21/00; C12N 5/071; C12N 15/00; A61K 31/7088; A61K 39/395; A61P 35/00; A61P 43/00

(52) U.S. Cl. 424/138.1; 435/6; 536/23.5; 435/366; 435/455; 514/44 R; 514/44 A

(57) ABSTRACT

Global gene expression patterns have been characterized in normal and cancerous human cells using serial analysis of gene expression (SAGE). Cancer cell-specific, cell-type specific, and ubiquitously expressed genes have been identified. This information can be used to provide combinations of cell type- and cancer-specific gene probes, as well as methods of using these probes to identify particular cell types, screen for useful drugs, reduce cancer-specific gene expression, standardize gene expression, and restore function to a diseased cell or tissue.

This application is a continuation of application Ser. No. 11/057,194 filed on Feb. 15, 2005, which is a continuation of Ser. No. 10/330,627 filed on Dec. 30, 2002, which is a continuation of Ser. No. 09/448,480 filed Nov. 24, 1999. Each of these applications is incorporated herein in its entirety.

This invention was made with government support under CA57345, CA62924, and CA43460 awarded by the National Institutes of Health. The government has certain rights in the invention.

This application incorporates by reference the contents of a 218 kb text file created on Aug. 16, 2010 and named “sequencelisting.txt,” which is the sequence listing for this application.

### BACKGROUND OF THE INVENTION

The characteristics of an organism are largely determined by the genes expressed within its cells and tissues. These expressed genes can be represented by transcriptomes that convey the identity and expression level of each expressed gene in a defined population of cells (1, 2). Although the entire sequence of the human genome will be elucidated in the near future (3), little is known about the many transcriptomes present in the human organism. Basic questions regarding the set of genes expressed in a given cell type, the distribution of expressed genes, and how these compare to genes expressed in other cell types, have remained largely unanswered.

General properties of gene expression patterns in eukaryotic cells were determined many years ago by RNA-cDNA reassociation kinetics (4), but these studies did not provide much information about the identities of the expressed genes within each expression class. Technological constraints have limited other analyses of gene expression to one or few genes at a time (5-9) or were non-quantitative (10, 11). Serial analysis of gene expression (SAGE) (12), one of several recently developed gene expression methods, has permitted the quantitative analysis of transcriptomes in the yeast Saccharomyces cereviseae (1, 13). This effort identified the expression of known and previously unrecognized genes in S. cereviseae (1, 14) and demonstrated that genome-wide expression analyses were practicable in eukaryotes.

Thus, there is a need in the art for the identification of transcriptomes which represent gene expression in particular cell types or under particular physiological conditions in eukaryotes, particularly in humans.

### SUMMARY OF THE INVENTION

It is an object of the present invention to provide such transcriptomes, individual polynucleotides, and methods of using the polynucleotides to identify particular cell types, screen for useful drugs, reduce cancer-specific gene expression, standardize gene expression, and restore function to a diseased cell or tissue. These and other objects of the invention are provided by one or more of the embodiments described below.

One embodiment of the invention is a method of identifying a cell as either a colon epithelial cell, a brain cell, a keratinocyte, a breast epithelial cell, a lung epithelial cell, a melanocyte, a prostate cell, or a kidney epithelial cell. Expression in a test cell of a gene product of at least one gene is determined. The at least one gene comprises a sequence selected from at least one of the following groups:
(a) the sequences shown in SEQ ID NOS:2, 5-18, 20-84, and 85;
(b) the sequences shown in SEQ ID NOS:87-96, 98, 100-103, 105, 107-110, 112-129, 131-150, and 151;
(c) the sequences shown in SEQ ID NOS:152-154 and 155;
(d) the sequences shown in SEQ ID NOS:156-159 and 160;
(e) the sequences shown in SEQ ID NOS:161-166 and 167;
(f) the sequences shown in SEQ ID NOS:168, 170, 172-177, 179-188, 190-207, and 208;
(g) the sequences shown in SEQ ID NOS:209 and 210; and
(h) the sequences shown in SEQ ID NOS:211-224 and 225. Expression of a gene product of at least one gene comprising a sequence shown in (a) identifies the test cell as a colon epithelial cell. Expression of a gene product of at least one gene comprising a sequence shown in (b) identifies the test cell as a brain cell. Expression of a gene product of at least one gene comprising a sequence shown in (c) identifies the test cell as a keratinocyte. Expression of a gene product of at least one gene comprising a sequence shown in (d) identifies the test cell as a breast epithelial cell. Expression of a gene product of at least one gene comprising a sequence shown in (e) identifies the test cell as a lung epithelial cell. Expression of a gene product of at least one gene comprising a sequence shown in (f) identifies the test cell as a melanocyte. Expression of a gene product of at least one gene comprising a sequence shown in (g) identifies the test cell as a prostate cell. Expression of a gene product of at least one gene comprising a sequence shown in (h) identifies the test cell as a kidney epithelial cell.

Another embodiment of the invention is an isolated polynucleotide comprising a sequence selected from the group consisting of SEQ ID NOS:2, 5, 6, 8, 10, 12, 13, 15, 17, 18, 21, 24-26, 28, 30, 31, 34-36, 38, 40, 47-51, 53-57, 59-62, 65-69, 71-76, 78, 80-84, 98, 103, 113, 115, 122, 129, 132, 134, 135, 140, 144, 149, 150, 153-168, 174-176, 182, 185, 186, 188, 190, 200, 201, 205-213, 216-224, 237, 239, 257, 263, 485, 487, 495, 499, 514, 586, 686, 751, 835, 844, 878, 910, 925, 932, 951, 1000, 1005, 1070, 1122, 1130, 1170, 1173, 1187, 1189, 1200, 1213, 1220, 1237, 1257, 1264, 1273, 1293, 1300, 1320, 1367, 1371, 1401, 1403, 1404, 1406, 1418, and 1419.

Still another embodiment of the invention is a solid support comprising at least one polynucleotide. The polynucleotide comprises a sequence selected from at least one of the following groups:
(a) the sequences shown in SEQ ID NOS:2, 5, 6, 8, 10, 12, 13, 15, 17, 18, 21, 24-26, 28, 30, 31, 34-36, 38, 40, 47-51, 53-57, 59-62, 65-69, 71-76, 78, 80-83, and 84;
(b) the sequences shown in SEQ ID NOS:98, 103, 113, 115, 122, 129, 132, 134, 135, 140, 144, 149, and 150;
(c) the sequences shown in SEQ ID NOS:153-154 and 155;
(d) the sequences shown in SEQ ID NOS:156-157 and 160;
(e) the sequences shown in SEQ ID NOS:161-166 and 167;
(f) the sequences shown in SEQ ID NOS:168, 174-176, 182, 185, 186, 188, 190, 200, 201, 205-207 and 208;
(g) the sequences shown in SEQ ID NOS:209 and 210;
(h) the sequences shown in SEQ ID NOS:211-213, 216-223, and 224;
(i) the sequences shown in SEQ ID NOS:237, 239, 257, and 263; or
(j) the sequences shown in SEQ ID NOS:485, 487, 495, 499, 514, 586, 686, 751, 835, 844, 878, 910, 925, 932, 951, 1000, 1005, 1070, 1122, 1130, 1170, 1173, 1187, 1189, 1200, 1213, 1220, 1237, 1257, 1264, 1273, 1293, 1300, 1320, 1367, 1371, 1401, 1403, 1404, 1406, 1418, and 1419.

Even another embodiment of the invention is a method of identifying a test cell as a cancer cell. Expression in a test cell of a gene product of at least one gene is determined. The at least one gene comprises a sequence selected from the group consisting of SEQ ID NOS:228, 230-257, 259-260, and 262-265. An increase in expression of at least two-fold relative to expression of the at least one gene in a normal cell identifies the test cell as a cancer cell.

Yet another embodiment of the invention is a method of reducing expression of a cancer-specific gene in a human cell. A reagent which specifically binds to an expression product of a cancer-specific gene is administered to the cell. The cancer-specific gene comprises a sequence selected from the group consisting of SEQ ID NOS:228, 230-257, 259-260, and 262-265. Expression of the cancer-specific gene is thereby reduced relative to expression of the cancer-specific gene in the absence of the reagent.

Even another embodiment of the invention is a method for comparing expression of a gene in a test sample to expression of a gene in a standard sample. A first ratio and a second ratio are determined. The first ratio is an amount of an expression product of a test gene in a test sample to an amount of an expression product of at least one gene comprising a sequence selected from the group consisting of SEQ ID NOS:266-375, 377-652, 654-796, and 798-1448 in the test sample. The second ratio is an amount of an expression product of the test gene in a standard sample to an amount of an expression product of the at least one gene in the standard sample. The first and second ratios are compared. A difference between the first and second ratios indicates a difference in the amount of the expression product of the test gene in the test sample.

Still another embodiment of the invention is a method of screening candidate anti-cancer drugs. A cancer cell is contacted with a test compound. Expression of a gene product of at least one gene in the cancer cell is measured. The at least one gene comprises a sequence selected from the group consisting of SEQ ID NOS:228, 230-257, 259, 260, 262-263, and 265. A decrease in expression of the gene product in the presence of a test compound relative to expression of the gene product in the absence of the test compound identifies the test compound as a potential anti-cancer drug.

Still another embodiment of the invention is a method of screening test compounds for the ability to increase an organ or cell function. A selected from the group consisting of a colon epithelial cell, a brain cell, a keratinocyte, a breast epithelial cell, a lung epithelial cell, a melanocyte, a prostate cell, and a kidney cell is contacted with a test compound. Expression in the cell of a gene product of at least one gene is measured. The gene comprises a sequence selected from at least one of the following groups:
(a) the sequences shown in SEQ ID NOS:2, 5-18, 20-84, and 85;
(b) the sequences shown in SEQ ID NOS:87-96, 98, 100-103, 105, 107-110, 112-129, 131-150, and 151;
(c) the sequences shown in SEQ ID NOS:152-154 and 155;
(d) the sequences shown in SEQ ID NOS:156-159 and 160;
(e) the sequences shown in SEQ ID NOS:161-166 and 167;
(f) the sequences shown in SEQ ID NOS:168, 170, 172-177, 179-188, 190-207 and 208;
(g) the sequences shown in SEQ ID NOS:209 and 210; and
(h) the sequences shown in SEQ ID NOS:211-224 and 225. An increase in expression of a gene product of at least one gene comprising a sequence shown in (a) identifies the test compound as a potential drug for increasing a function of a colon cell. An increase in expression of a gene product of at least one gene comprising a sequence shown in (b) identifies the test compound as a potential drug for increasing a function of a brain cell. An increase in expression of a gene product of at least one gene comprising a sequence shown in (c) identifies the test compound as a potential drug for increasing a function of a skin cell. An increase in expression of a gene product of at least one gene comprising a sequence shown in (d) identifies the test compound as a potential drug for increasing a function of a breast cell. An increase in expression of a gene product of at least one gene comprising a sequence shown in (e) identifies the test compound as a potential drug for increasing a function of a lung cell. An increase in expression of a gene product of at least one gene comprising a sequence shown in (f) identifies the test compound as a potential drug for increasing a function of a melanocyte. An increase in expression of a gene product of at least one gene comprising a sequence shown in (g) identifies the test compound as a potential drug for increasing a function of a prostate cell. An increase in expression of a gene product of at least one gene comprising a sequence shown in (h) identifies the test compound as a potential drug for increasing a function of a kidney cell.

Yet another embodiment of the invention is a method to restore function to a diseased tissue. A gene is delivered to a diseased cell selected from the group consisting of a colon epithelial cell, a brain cell, a keratinocyte, a breast epithelial cell, a lung epithelial cell, a melanocyte, a prostate cell, and a kidney cell. The gene comprises a nucleotide sequence selected from at least one of the following groups:
(a) the sequences shown in SEQ ID NOS:2, 5-18, 20-84, and 85;
(b) the sequences shown in SEQ ID NOS:87-96, 98, 100-103, 105, 107-110, 112-129, 131-150, and 151;
(c) the sequences shown in SEQ ID NOS:152-154 and 155;
(d) the sequences shown in SEQ ID NOS:156-159 and 160;
(e) the sequences shown in SEQ ID NOS:161-166 and 167;
(f) the sequences shown in SEQ ID NOS:168, 170, 172-177, 179-188, 190-207, and 208;
(g) the sequences shown in SEQ ID NOS:209 and 210; and
(h) the sequences shown in SEQ ID NOS:211-224 and 225. Expression of the gene in the diseased cell is less than expression of the gene in a corresponding cell which is normal. If the diseased cell is a colon epithelial cell, then the nucleotide sequence is selected from (a). If the diseased cell is a brain cell, then the nucleotide sequence is selected from (b). If the diseased cell is a keratinocyte, then the nucleotide sequence is selected from (c). If the diseased cell is a breast epithelial cell, then the nucleotide sequence is selected from (d). If the diseased cell is a lung epithelial cell, then the nucleotide sequence is selected from (e). If the diseased cell is a melanocyte, then the nucleotide sequence is selected from (f). If the diseased cell is a prostate cell, then the nucleotide sequence is selected from (g). If the diseased cell is a kidney cell, then the nucleotide sequence is selected from (h).

Thus, the invention provides transcriptomes, polynucleotides, and methods of identifying particular cell types, reducing cancer-specific gene expression, identifying cancer cells, standardizing gene expression, screening test compounds for the ability to increase an organ or a cell function, and restoring function to a diseased tissue.

### BRIEF DESCRIPTION OF THE DRAWINGS

FIG. 1. Sampling of gene expression in colon cancer cells. Analysis of transcripts at increasing increments of transcript tags indicates that the fraction of new transcripts identified approaches 0 at approximately 650,000 total tags.

FIG. 2. Colon cancer cell Rot curve.

FIGS. 3A-3C. Gene expression in different tissues. FIG. 3A. Fold reduction or induction of unique transcripts for each of the comparisons analyzed. The source of the transcripts included in each comparison are displayed in FIG. 3C. The relative expression of each transcript was determined by dividing the number of transcript tags in each comparison in the order displayed in FIG. 3C. To avoid division by 0, we used a tag value of 1 for any tag that was not detectable in one of the samples. We then rounded these ratios to the nearest integer; their distribution is plotted on the X axis. The number of transcripts displaying each ratio is plotted on the Y axis. Each comparison is represented by a specific color (see below or FIG. 3C). FIG. 3B. Expression of transcripts for each comparison, where values on X and Y axes represent the observed transcript tag abundances in each of the two compared sets. Light Blue symbols: DLD1 in different physiologic conditions; Yellow symbols: DLD1 cells (X axis) versus HCT116 cells (Y axis); Red symbols: colon cancer cells (X axis) versus normal brain (Y axis); and Dark Blue symbols: colon cancer cells (X axis) versus hemangiopericytoma (Y axis). FIG. 3C. Fraction of transcripts with dramatically altered expression. For each comparison, Expression Change denotes the number of transcripts induced or reduced 10 fold, and (%) denotes the number of altered transcripts divided by the number of unique transcripts in each case. Differences between expression changes were evaluated using the chi squared test, where the expected expression changes were assumed to be the average expression change for any two comparisons.

### TABLE LEGENDS

Table 1. Table of tissues and transcript tags analyzed. “Tissues” represents the source of the RNA analyzed, “Libraries” indicates the number of SAGE libraries analyzed, “Total Transcripts” is the total number of transcripts analyzed from each tissue, and “Unique Transcripts” denotes the number of unique transcripts observed in each tissue.

Table 2. Table of transcript abundance. “Copies/cell” denotes the category of expression level analyzed in transcript copies per cell, “Unique Transcripts” represents the number of unique transcripts observed and those matching GenBank genes or ESTs, and “Mass fraction mRNA” represents the fraction of mRNA molecules contained in each expression category.

Table 3. Table showing tissue-specific transcripts. The number in parentheses adjacent to the tissue type indicates the percent of transcripts exclusively expressed in a given tissue at 10 copies per cell. “Transcript tag” denotes the 10 by tag adjacent to 4 bp NlaIII anchoring enzyme site, “Copies/cell” denotes the transcript copies per cell expressed, and “UniGene Description” provides a functional description of each matching UniGene cluster (from UniGene Build No. 67). As UniGene cluster numbers change over time, the most recent cluster assignment for each tag can be obtained individually at the Uniform Resource Locator (URL) address for the http file type found on the www host server that has a domain name of ncbi.nlm.gov, a path to the SAGE directory, and file name of SAGEtag.cgi (Lal et al., “A public database for gene expression in human cancers,” Cancer Research, in press) or for the entire table at the URL address: http file type, www host server, domain name sagenet.org, transcriptome directory.

Table 4. Table showing ubiquitously expressed genes. “Copies/cell” denotes the average expression level of each transcript from all tissues examined, “Range” represents the range in expression for each transcript tag among all tissues analyzed in copies per cell, and “Range/Avg” is the ratio of the range to the average expression level and provides a measure of uniformity of expression. Other table columns are the same as in Table 5. The entire table of uniformly expressed transcripts also is available at the URL address: http file type, www host server, domain name sagenet.org, transcriptome directory.

Table 5. Table showing transcripts uniformly elevated in human cancers. Transcripts expressed at 3 copies/cell whose expression is at least 2-fold higher in each cancer compared to its corresponding normal tissue. CC, colon cancer; BC, brain cancer; BrC, breast cancer; LC, lung cancer; M, melanoma; NC, normal colon epithelium; NB, normal brain; NBr, normal breast epithelium; NL, normal lung epithelium; NM, normal melanocytes. “Avg T/N” is the average ratio of expression in tumor tissue divided by normal tissue (for the purpose of obtaining this ratio, expression values of 0 are converted to 0.5). Other table columns are the same as in Table 5.

Table 6. Table showing transcripts expressed in colon cancer cells at a level of at least 500 copies per cell.

Table 7. Table showing transcripts expressed at a level of at least 500 copies per cell.

### DETAILED DESCRIPTION OF THE INVENTION

It is a discovery of the present invention that particular sets of expressed genes (“transcriptomes”) are expressed only in cancer cells; expression of these genes can be used, inter alia, to identify a test cell as cancerous and to screen for anti-cancer drugs. These cancer-specific genes can also provide targets for therapeutic intervention.

It is another discovery of the invention that other transcriptomes are differentially associated with distinct cell types; expression of genes of these transcriptomes can therefore be used to identify a test cell as belonging to one of these distinct cell types.

It is yet another discovery of the invention that genes of another transcriptome are expressed ubiquitously; expression of genes of this transcriptome can be used to standardize expression of other genes in a variety of gene expression assays.

To identify the transcriptomes described herein we used the SAGE method, as described in Velculescu et al. (1) and Velculescu et al. (12), to analyze gene expression in a variety of different human cell and tissue types. The SAGE method is also described in U.S. Pat. Nos. 5,866,330 and 5,695,937. A total of 84 SAGE libraries were generated from 19 tissues (Table 1). Diseased tissues included cancers of the colon, pancreas, breast, lung, and brain, as well as melanoma, hemangiopericytoma, and polycystic kidney disease. Normal tissues included epithelia of the colon, breast, lung, and kidney, melanocytes, chondrocytes, monocytes, cardiomyocytes, keratinocytes, and cells of prostate and brain white matter and astrocytes.

A total of 3,496,829 transcript tags were analyzed and found to represent 134,135 unique transcripts after correcting for sequencing errors (transcript data available at the URL address: http file type, www host server, domain name sagenet.org, transcriptome directory). Expression levels for these transcripts ranged from 0.3 to a high of 9,417 transcript copies per cell in lung epithelium. Comparison against the GenBank and UniGene collections of characterized genes and expressed sequence tags (ESTs) revealed that 6,900 transcript tags matched known genes, while 65,735 matched ESTs. The remaining 61,500 transcript tags (46%) had no matches to existing databases and corresponded to previously uncharacterized or partially sequenced transcripts.

Each of the genes or transcripts whose expression can be measured in the methods of the invention comprises a unique sequence of at least 10 contiguous nucleotides (the “SAGE tag”). Genes which are differentially expressed in colon, lung, kidney, and breast epithelial cells, brain cells, prostate cells, keratinocytes, or melanocytes are shown in Table 3. Ubiquitously expressed genes are shown in Table 4. Transcripts which are expressed only in cancer tissues, e.g., colon cancer, breast cancer, brain cancer, liver cancer, and melanoma, are shown in Table 5.

This information provides heretofore unavailable picture of human transcriptomes. These results, like the human genome sequence, provide basic information integral to future experimentation in normal and disease states. Because SAGE analyses provide absolute expression levels, future SAGE data can be directly integrated with those described here to provide progressively deeper insights into gene expression patterns. Eventually, a relatively complete description of the transcripts expressed in diverse cell types and in various physiologic states can be obtained.

### Isolated Polynucleotides

The invention provides isolated polynucleotides comprising either deoxyribonucleotides or ribonucleotides. Isolated DNA polynucleotides according to the invention contain less than a whole chromosome and can be either genomic DNA or DNA which lacks introns, such as cDNA. Isolated DNA polynucleotides can comprise a gene or a coding sequence of a gene comprising a sequence as shown in SEQ ID NOS:1-1563, such as polynucleotides which comprise a sequence selected from the group consisting of SEQ ID NOS:2, 5, 6, 8, 10, 12, 13, 15, 17, 18, 21, 24-26, 28, 30, 31, 34-36, 38, 40, 47-51, 53-57, 59-62, 65-69, 71-76, 78, 80-84, 98, 103, 113, 115, 122, 129, 132, 134, 135, 140, 144, 149, 150, 153-168, 174-176, 182, 185, 186, 188, 190, 200, 201, 205-213, 216-224, 237, 239, 257, 263, 485, 487, 495, 499, 514, 586, 686, 751, 835, 844, 878, 910, 925, 932, 951, 1000, 1005, 1070, 1122, 1130, 1170, 1173, 1187, 1189, 1200, 1213, 1220, 1237, 1257, 1264, 1273, 1293, 1300, 1320, 1367, 1371, 1401, 1403, 1404, 1406, 1418, and 1419.

Any technique for obtaining a polynucleotide can be used to obtain isolated polynucleotides of the invention. Preferably the polynucleotides are isolated free of other cellular components such as membrane components, proteins, and lipids. They can be made by a cell and isolated, or synthesized using an amplification technique, such as PCR, or by using an automatic synthesizer. Methods for purifying and isolating polynucleotides are routine and are known in the art.

Isolated polynucleotides also include oligonucleotide probes, which comprise at least one of the sequences shown in SEQ ID NOS:1-1563. An oligonucleotide probe is preferably at least 10, 11, 12, 13, 14, 15, 20, 30, 40, or 50 or more nucleotides in length. If desired, a single oligonucleotide probe can comprise 2, 3, 4, or 5 or more of the sequences shown in SEQ ID NOS:1-1563. The probes may or may not be labeled. They may be used, for example, as primers for amplification reactions, such as PCR, in Southern or Northern blots, or for in situ hybridization.

Oligonucleotide probes of the invention can be made by expressing cDNA molecules comprising one or more of the sequences shown in SEQ ID NOS:1-1563 in an expression vector in an appropriate host cell. Alternatively, oligonucleotide probes can be synthesized chemically, for example using an automated oligonucleotide synthesizer, as is known in the art.

Solid Supports Comprising Polynucleotides

Polynucleotides, particularly oligonucleotide probes, preferably are immobilized on a solid support. A solid support can be any surface to which a polynucleotide can be attached. Suitable solid supports include, but are not limited to, glass or plastic slides, tissue culture plates, microtiter wells, tubes, probe arrays such as GENECHIPS®, or particles such as beads, including but not limited to latex, polystyrene, or glass beads. Any method known in the art can be used to attach a polynucleotide to a solid support, including use of covalent and non-covalent linkages, passive absorption, or pairs of binding moieties attached respectively to the polynucleotide and the solid support.

Polynucleotides are preferably present on an array so that multiple polynucleotides can be simultaneously tested for hybridization to polynucleotides present in a single biological sample. The polynucleotides can be spotted onto the array or synthesized in situ on the array. Such methods include older technologies, such as “dot blot” and “slot blot” hybridization (53, 54), as well as newer “microarray” technologies (55-58). A single array contains at least one polynucleotide, but can contain more than 100, 500, 1,000, 10,000, or 100,000 or more different probes in discrete locations.

Determining Expression of a Gene Product

Each of the methods of the invention involves measuring expression of a gene product of at least one of the genes identified in Tables 3, 4, and 5 (SEQ ID NOS:1-1448). If desired, expression of gene products of at least 2, 3, 4, 5, 6, 7, 8, 9, 10, 15, 20, 25, 30, 50, 75, 100, 125, 250, 500, 1,000, 1,250, or more genes can be determined.

Either protein or RNA products of the disclosed genes can be determined. Either qualitative or quantitative methods can be used. The presence of protein products of the disclosed genes can be determined, for example, using a variety of techniques known to the art, including immunochemical methods such as radioimmunoassay, Western blotting, and immunohistochemistry. Alternatively, protein synthesis can be determined in vivo, in a cell culture, or in an in vitro translation system by detecting incorporation of labeled amino acids into protein products.

RNA expression can be determined, for example, using at least 1, 2, 3, 4, 5, 10, 15, 20, 25, 30, 50, 75, 100, 125, 250, 500, 1,000, 5,000, 10,000, or 100,000 or more oligonucleotide probes, either in solution or immobilized on a solid support, as described above. Expression of the disclosed genes is preferably determined using an array of oligonucleotide probes immobilized on a solid support. In situ hybridization can also be used to detect RNA expression.

### Identification of Cell Types

Cell-type specific genes are expressed at a level greater than 10 copies per cell in a particular cell type, such as epithelial cells of the colon, breast, lung, and kidney, keratinocytes, melanocytes, and cells from the prostate and brain, but are not expressed in cells of other tissues. Such cell-type specific genes represent “cell-type specific transcriptomes.” The fraction of cell-type-specific transcripts ranges from 0.05% in normal prostate to 1.76% in normal colon epithelium. Approximately 50% of these transcripts tags match known genes or ESTs. The vast majority of these cell-type-specific genes have not been previously reported in the literature to be cell-type specific.

Cell type-specific genes are shown in Table 3. Genes which comprise the sequences shown in SEQ ID NOS:1-85 are uniquely expressed in colon epithelial cells. Genes which comprise the sequences shown in SEQ ID NOS:86-151 are uniquely expressed in brain cells. Genes which comprise the sequences shown in SEQ ID NOS:152-155 are uniquely expressed in keratinocytes. Genes which comprise the sequences shown in SEQ ID NOS:156-160 are uniquely expressed in breast epithelial cells. Genes which comprises the sequences shown in SEQ ID NOS:161-167 are uniquely expressed in lung epithelial cells. Genes which comprises the sequences shown in SEQ ID NOS:168-208 are uniquely expressed in melanocytes. Genes which comprise the sequences shown in SEQ ID NOS:209 and 210 are uniquely expressed in prostate cells. Genes which comprise the sequences shown in SEQ ID NOS:211-225 are uniquely expressed in kidney epithelial cells. Thus, determination of expression of at least one gene from each of these uniquely expressed groups, particularly those not previously known to be uniquely expressed, can be used to identify a test cell as an epithelial cell of the colon, breast, lung, and kidney, a keratinocyte, a melanocyte, or a cell from the prostate or brain.

Test cells can be obtained, for example, from biopsy or surgical samples, forensic samples, cell lines, or primary cell cultures. Test cells include normal as well as cancer cells, such as primary or metastatic cancer cells.

To identify a test cell as an epithelial cell of the colon, breast, lung, and kidney, a keratinocyte, a melanocyte, or a cell from the prostate or brain, expression of a gene product of at least one gene is determined, using methods such as those described above. If a test cell expresses a gene comprising a sequence shown in SEQ ID NOS:2, 5-18, and 20-85, the test cell is identified as a colon epithelial cell. If a test cell expresses a gene comprising a sequence shown in SEQ ID NOS:87-96, 98, 100-103, 105, 107-110, 112-129, and 131-151, the test cell is identified as a brain cell. If a test cell expresses a gene comprising a sequence shown in SEQ ID NOS:152-155, the test cell is identified as a keratinocyte. If a test cell expresses a gene comprising a sequence shown in SEQ ID NOS:156-160, the test cell is identified as a breast epithelial cell. If a test cell expresses a gene comprising a sequence shown in SEQ ID NOS:161-167, the test cell is identified as a lung epithelial cell. Expression of a gene comprising a sequence shown in SEQ ID NOS:168, 170, 172-177, 179-188, and 190-208 identifies the test cell as a melanocyte. Expression of a gene comprising a sequence shown in SEQ ID NOS:209 and 210 identifies the test cell as a prostate cell. Expression of a gene which comprises a sequence shown in SEQ ID NOS:211-225 identifies the test cell as a kidney epithelial cell.

### Identifying a Test Cell as a Cancer Cell

A cancer-specific gene is expressed at a level of at least 3 copies per cancer cell, such as a colon cancer, breast cancer, brain cancer, lung cancer, or melanoma cell, at a level which is at least two-fold higher than expression of the same gene in a corresponding normal cell. Cancer-specific genes which comprise the sequences shown in SEQ ID NOS:226-265 (Table 5) represent a “cancer transcriptome.” SEQ ID NOS:237, 239, 257, and 263 are sequences which are found in transcripts of novel cancer-specific genes of the invention. Oligonucleotide probes corresponding to cancer-specific genes can be used, for example, to detect and/or measure expression of cancer-specific genes for diagnostic purposes, to assess efficacy of various treatment regimens, and to screen for potential anti-cancer drugs.

For example, determination of the expression level of any of these genes in a test cell relative to the expression level of the same gene in a normal cell (a cell which is known not to be a cancer cell) can be used to determine whether the test cell is a cancer cell or a non-cancer cell.

Test cells can be any human cell suspected of being a cancer cell, including but not limited to a colon epithelial cell, a breast epithelial cell, a lung epithelial cell, a kidney epithelial cell, a melanocyte, a prostate cell, and a brain cell. Test cells can be obtained, for example, from biopsy samples, surgically excised tissues, forensic samples, cell lines, or primary cell cultures. Comparison can be made to a non-cancer cell type, including to the corresponding non-cancer cell type, either at the time expression is measured in the test cell or by reference to a previously determined expression standard.

To identify a test cell as a cancer cell, expression of a gene product of at least one gene is determined, using methods such as those described above. The at least one gene comprises a sequence selected from the group consisting of SEQ ID NOS:226-265, particularly from the group consisting of SEQ ID NOS:228, 230-236, 238, 240-256, 258-260, and 262-265. An increase in expression of the at least one gene in the test cell which is at least two-fold more than the expression of the at least one gene in a cell which is not cancerous identifies the test cell as a cancer cell.

### Reducing Cancer-Specific Gene Expression

Cancer-specific genes provide potential therapeutic targets for treating cancer or for use in model systems, for example, to screen for agents which will enhance the effect of a particular compound on a potential therapeutic target. Thus, a reagent can be administered to a human cell, either in vitro or in vivo, to reduce expression of a cancer-specific gene. The reagent specifically binds to an expression product of a gene comprising a sequence selected from the group consisting of SEQ ID NOS:226-265, particularly from the group consisting of SEQ ID NOS:228, 230-236, 238, 240-256, 258-260, and 262-265.

If the expression product is a protein, the reagent is preferably an antibody. Protein products of cancer-specific genes can be used as immunogens to generate antibodies, such as a polyclonal, monoclonal, or single-chain antibodies, as is known in the art. Protein products of cancer-specific genes can be isolated from primary or metastatic tumors, such as primary colon adenocarcinomas, lung cancers, astrocytomas, glioblastomas, breast cancers, and melanomas. Alternatively, protein products can be prepared from cancer cell lines such as SW480, HCT116, DLD1, HT29, RKO, 21-PT, MDA-468, A549, and the like. If desired, cancer-specific gene coding sequences can be expressed in a host cell or in an in vitro translation system. An antibody which specifically binds to a protein product of a cancer-specific gene provides a detection signal at least 5-, 10-, or 2-fold higher than a detection signal provided with other proteins when used in an immunochemical assay. Preferably, the antibody does not detect other proteins in immunochemical assays and can immunoprecipitate the cancer-specific protein product from solution.

For administration in vitro, an antibody can be added to a tissue culture preparation, either as a component of the medium or in addition to the medium. In another embodiment, antibodies are delivered to specific tissues in vivo using receptor-mediated targeted delivery. Receptor-mediated DNA delivery techniques are taught in, for example, Findeis et al. Trends in Biotechnol. 11, 202-05, (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24, 1988; Wu et al., J. Biol. Chem. 269, 542-46, 1994; Zenke et al., Proc. Natl. Acad. Sci. U.S.A. 87, 3655-59, 1990; Wu et al., J. Biol. Chem. 266, 338-42, 1991.

If single-chain antibodies are used, polynucleotides encoding the antibodies can be constructed and introduced into cells using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide. The nucleotide sequence of an antisense oligonucleotide is complementary to at least a portion of the sequence of the cancer-specific gene. Preferably, the antisense oligonucleotide sequence is at least 10 nucleotides in length, but can be at least 11, 12, 15, 20, 25, 30, 35, 40, 45, or 50 or more nucleotides long. Longer sequences also can be used. An antisense oligonucleotide which specifically binds to an mRNA product of a cancer-specific gene preferably hybridizes with no more than 3 or 2 mismatches, preferably with no more than 1 mismatch, even more preferably with no mismatches.

Antisense oligonucleotides can be deoxyribonucleotides, ribonucleotides, or a combination of both. Oligonucleotides, including modified oligonucleotides, can be prepared by methods well known in the art (47-52) and introduced into human cells using techniques such as those described above. The cells can be in a primary culture of human tumor cells, in a human tumor cell line, or can be primary or metastatic tumor cells present in a human body.

Preferably, a reagent reduces expression of a cancer-specific gene by at least 10%, 20%, 30%, 40%, 50%, 60%, 70%, or 80% relative to expression of the gene in the absence of the reagent. Most preferably, the level of gene expression is decreased by at least 90%, 95%, 99%, or 100%. The effectiveness of the mechanism chosen to decrease the level of expression of a cancer-specific gene can be assessed using methods well known in the art, such as hybridization of nucleotide probes to cancer-specific gene mRNA, quantitative RT-PCR, or immunologic detection of a protein product of the cancer-specific gene.

### Screening for Anti-Cancer Drugs

According to the invention, test compounds can be screened for potential use as anti-cancer drugs by assessing their ability to suppress or decrease the expression of at least one cancer-specific gene. The cancer-specific gene comprises a sequence selected from the group consisting of SEQ ID NOS:226-265, particularly from the group consisting of SEQ ID NOS:228, 230-236, 238, 240-256, 258-260, and 262-265. Test compounds can be pharmacologic agents already known in the art or can be compounds previously unknown to have any pharmacological activity, including small molecules from compound libraries. Test substances can be naturally occurring or designed in the laboratory. They can be isolated from microorganisms, animals, or plants, or can be produced recombinantly or synthesized by chemical methods known in the art.

To screen a test compound for use as a possible anti-cancer drug, a cancer cell is contacted with the test compound. The cancer cell can be a cell of a primary or metastatic tumor, such as a tumor of the colon, breast, lung, prostate, brain, or kidney, or a melanoma, which is isolated from a patient. Alternatively, a cancer cell line, such as colon cancer cell lines HCT116, DLD1, HT29, Caco2, SW837, SW480, and RKO, breast cancer cell lines 21-PT, 21-MT, MDA-468, SK-BR3, and BT-474, the A549 lung cancer cell line, and the H392 glioblastoma cell line, can be used.

Expression of a gene product of at least one gene is determined using methods such as those described above. The gene comprises a sequence selected from the group consisting of SEQ ID NOS:226-265, preferably from the group consisting of SEQ ID NOS:228, 230-236, 238, 240-256, 258-260, and 262-265, even more preferably from the group consisting of SEQ ID NOS:237, 239, 257, and 263. A decrease in expression of the gene in the cancer cell identifies the test compound as a potential anti-cancer drug.

### Standardizing Expression of a Test Gene

Genes which comprise the sequences shown in SEQ ID NOS:266-1448 (Table 4) are expressed at a level of at least five transcript copies per cell in every cell type analyzed, including epithelia of the colon, breast, lung, and kidney, melanocytes, chondrocytes, monocytes, cardiomyocytes, keratinocytes, prostate cells, and astrocytes, oligodendrocytes, and other cells present in the white matter of brain. These genes thus represent members of the “minimal transcriptome,” the set of genes expressed in all human cells. The minimal transcriptome includes well known genes which are often used as experimental controls to normalize gene expression, such as glyceraldehyde 3-phosphate dehydrogenase, elongation factor 1 alpha, and gamma actin.

Ubiquitously expressed genes can be used to compare expression of a test gene in a test sample to expression of a gene in a standard sample. A ubiquitously expressed gene preferably comprises a sequence shown in SEQ ID NOS:266-375, 377-652, 654-796, and 798-1448, and more preferably comprises a sequence shown in SEQ ID NOS:282, 288, 300, 302, 308, 320, 323, 363, 368, 379, 381, 444, 453, 518, 531, 535, 538, 542, 579, 580, 594, 600, 604, 617, 626, 641, 650, 717, 728, 776, 777, 794, 818, 822, 842, 885, 887, 899, 900, 902, 904, 914, 930, 960, 964, 1001, 1015, 1020, 1027, 1035, 1090, 1113, 1119, 1146, 1151, 1163, 1233, 1235, 1252, 1255, 1270, 1340, 1345, 1356, 1359, 1360, 1362, 1385, 1415, and 1441.

Two ratios are determined using gene expression assays such as those described above. The first ratio is an amount of an expression product of a test gene in a test sample to an amount of an expression product of at least one ubiquitously expressed gene comprising a sequence selected from the group consisting of SEQ ID NOS:266-375, 377-652, 798-1447, and 1448 in the test sample. The second ratio is an amount of an expression product of the test gene in a standard sample to an amount of an expression product of the ubiquitously expressed gene in the standard sample. Expression of either the test gene or the ubiquitously expressed gene can be used as the denominator. If desired, multiple ratios can be determined, such as (a) an amount of an expression product of more than one test gene to that of a single ubiquitously expressed gene, (b) an amount of an expression product of a single test gene to that of more than one ubiquitously expressed genes, or (c) an amount of an expression product of more than one test gene to that of more than one ubiquitously expressed gene. Optionally, the ratio in the standard sample can be pre-determined.

The ratios determined in the test and standard samples are compared. A different between the ratios indicates a difference in the amount of the expression product of the test gene in the test sample.

The standard and test samples can be matched samples, such as whole cell cultures or homogenates of cells (such as a biopsy sample) and differ only in that the test biological sample has been subjected to a different environmental condition, such as a test compound, a drug whose effect is known or unknown, or altered temperature or other environmental condition. Alternatively, the test and standard samples can be corresponding cell types which differ according to developmental age. In one embodiment, the test sample is a cancer cell, such as a colon cancer, breast cancer, lung cancer, melanoma, or brain cancer cell, and the standard sample is a normal cell.

The test gene can be a gene which encodes a protein whose biological function is known or unknown. Preferably the ratio of expression between the test gene and expression of the ubiquitously expressed gene is consistent in the standard sample. Even more preferably, expression of the ubiquitously expressed gene is not altered in the test sample. A difference between the first ratio of expression in the test sample and a second ratio of expression in the standard sample can therefore be used to indicate a difference in expression of the test gene in the test sample.

### Screening for Compounds for Increasing an Organ or Cell Function

Test compounds can be screened for the ability to increase an organ or cell function by assessing their ability to increase expression of at least one tissue-specific gene. The tissue-specific gene comprises a sequence selected from at least one of the following groups:
(a) the sequences shown in SEQ ID NOS:2, 5-18, 20-84, and 85;
(b) the sequences shown in SEQ ID NOS:87-96, 98, 100-103, 105, 107-110, 112-129, 131-150, and 151;
(c) the sequences shown in SEQ ID NOS:152-154, and 155;
(d) the sequences shown in SEQ ID NOS:156-159 and 160;
(e) the sequences shown in SEQ ID NOS:161-166 and 167;
(f) the sequences shown in SEQ ID NOS:168, 170, 172-177, 179-188, 190-207, and 208;
(g) the sequences shown in SEQ ID NOS:209 and 210; and
(h) the sequences shown in SEQ ID NOS:211-224 and 225.

As with the anti-cancer drug screening method described above, test compounds can be pharmacologic agents already known in the art or can be compounds previously unknown to have any pharmacological activity, including small molecules from compound libraries. Test substances can be naturally occurring or designed in the laboratory. They can be isolated from microorganisms, animals, or plants, or can be produced recombinantly or synthesized by chemical methods known in the art.

To screen a test compound for the ability to increase an organ or cell function, a cell, such as a colon epithelial cell, a brain cell, a keratinocyte, a breast epithelial cell, a lung epithelial cell, a melanocyte, a prostate cell, or a kidney cell, is contacted with the test compound. The cell can be a primary culture, such as an explant culture, of tissue obtained from a human, or can originate from an established cell line.

Expression of a gene product of at least one gene is determined using methods such as those described above. An increase in expression of a gene product of at least one gene comprising a sequence selected from (a) identifies the test compound as a potential drug for increasing a function of a colon cell. An increase in expression of a gene product of at least one gene comprising a sequence selected from (b) identifies the test compound as a potential drug for increasing a function of a brain cell. An increase in expression of a gene product of at least one gene comprising a sequence selected from (c) identifies the test compound as a potential drug for increasing a function of a skin cell. An increase in expression of a gene product of at least one gene comprising a sequence selected from (d) identifies the test compound as a potential drug for increasing a function of a breast cell. An increase in expression of a gene product of at least one gene comprising a sequence selected from (e) identifies the test compound as a potential drug for increasing a function of a lung cell. An increase in expression of a gene product of at least one gene comprising a sequence selected from (f) identifies the test compound as a potential drug for increasing a function of a melanocyte. An increase in expression of a gene product of at least one gene comprising a sequence selected from (g) identifies the test compound as a potential drug for increasing a function of a prostate cell. An increase in expression of a gene product of at least one gene comprising a sequence selected from (h) identifies the test compound as a potential drug for increasing a function of a kidney cell.

### Restoring Function to a Diseased Tissue or Cell

Function can be restored to a diseased tissue or cell, such as a melanocyte or a colon, brain, keratinocyte, breast, lung, prostate, or kidney cell, by delivering an appropriate tissue-specific gene to cells of that tissue. The tissue specific gene comprises a nucleotide sequence selected from at least one of the following groups:
(a) the sequences shown in SEQ ID NOS:2, 5-18, 20-84, and 85 (colon-specific);
(b) the sequences shown in SEQ ID NOS:87-96, 98, 100-103, 105, 107-110, 112-129, 131-150, and 151 (brain-specific);
(c) the sequences shown in SEQ ID NOS:152-154, and 155 (keratinocyte-specific);
(d) the sequences shown in SEQ ID NOS:156-159 and 160 (breast-specific);
(e) the sequences shown in SEQ ID NOS:161-166 and 167 (lung-specific);
(f) the sequences shown in SEQ ID NOS:168, 170, 172-177, 179-188, 190-207, and 208 (melanocyte-specific);
(g) the sequences shown in SEQ ID NOS:209 and 210 (prostate-specific); and
(h) the sequences shown in SEQ ID NOS:211-224 and 225 (kidney-specific).

Expression of the gene in a cell of the diseased tissue preferably is 10, 20, 30, 40, 50, 60, 70, 80, or 90% less than expression of the gene in a cell of the corresponding tissue which is normal. In some cases, the diseased cell fails to express the gene. A tissue-specific gene which is administered to cells for this purpose includes a polynucleotide comprising a coding sequence which is intron-free, such as a cDNA, as well as a polynucleotide which comprises elements in addition to the coding sequence, such as regulatory elements.

Coding sequences of many of the tissue-specific genes disclosed herein are publicly available. For the novel tissue-specific genes identified here, coding sequences can be obtained using a variety of methods, such as restriction-site PCR (Sarkar, PCR Methods Applic. 2:318-322, 1993), inverse PCR (Triglia et al., Nucleic Acids Res. 16:8186, 1988), capture PCR (Lagerstrom, et al., PCR Methods Applic. 1:111-119, 1991). Alternatively, the partial sequences disclosed herein can be nick-translated or end-labeled with ³²P using polynucleotide kinase using labeling methods known to those with skill in the art (BASIC METHODS IN MOLECULAR BIOLOGY, Davis et al., eds., Elsevier Press, N.Y., 1986). A lambda library prepared from the appropriate human tissue can then be directly screened with the labeled sequences of interest.

Many methods for introducing polynucleotides into cells or tissues are available and can be used to deliver a tissue-specific gene to a cell in vitro or in vivo. Introduction of the tissue-specific gene into a cell can be accomplished by any method by which a nucleic acid molecule can be inserted into a cell, such as transfection, electroporation, microinjection, lipofection, adsorption, and protoplast fusion. For in vitro administration, a tissue-specific gene can be added to a tissue culture preparation, either as a component of the medium or in addition to the medium. In vivo administration can be by means of direct injection of a vector comprising a tissue-specific gene to the particular tissue or cells to which the tissue-specific gene is to be delivered. Alternatively, the tissue-specific gene can be included in a vector which is capable of targeting a particular tissue and administered systemically (59-61).

For in vitro administration, suitable concentrations of a tissue-specific gene in the culture medium range from at least about 10 pg to 100 pg/ml, about 100 pg to about 500 pg/ml, about 500 pg to about 1 ng/ml, about 1 ng to about 10 ng/ml, about 10 ng to about 100 ng/ml, or about 100 ng/ml to about 500 ng/ml. For local administration, effective dosages of a tissue-specific gene range from at least about 10 ng to about 100 ng, about 50 ng to 150 ng, about 100 ng to about 250 ng, about 1 μg to about 10 μg, about 5 μg to about 50 μg, about 25 μg to about 100 μg, about 75 μg to about 250 μg, about 100 μg to about 250 μg, about 200 μg to about 500 μg, about 500 μg to about 1 mg, about 1 mg to about 10 mg, about 5 mg to about 50 mg, about 25 mg to about 100 mg, or about 50 mg to about 200 mg of DNA per injection. Suitable concentrations for systemic administration range from at least about 500 ng to about 50 mg, about 1 μg to about 2 mg, about 5 μg to about 500 μg, and about 20 μg to about 100 μg of DNA per kg of body weight.

Recombinant DNA technologies can be used to improve expression of the tissue-specific gene by manipulating, for example, the number of copies of the gene in the cell, the efficiency with which the gene is transcribed, the efficiency with which the resultant transcripts are translated, and the efficiency of post-translational modifications. Recombinant techniques useful for increasing the expression of a tissue-specific gene in a cell include, but are not limited to, providing the tissue-specific gene in a high-copy number plasmid, integrating the tissue-specific gene into one or more host cell chromosomes, adding vector stability sequences to plasmids, substituting or modifying transcription control signals (e.g., promoters, operators, enhancers), substituting or modulating translational control signals (e.g., ribosome binding sites, Shine-Dalgarno sequences), and deleting sequences that destabilize transcripts. (See Dow et al., U.S. Pat. No. 5,935,568).

Preferably, delivery of the tissue-specific gene increases expression of a gene product of the tissue-specific gene in the cell or tissue by at least 10, 20, 30, 40, 50, 60 70, 80, 90, 95, 98, 99, or 100% relative to expression of the tissue-specific gene in a diseased cell or tissue to which the gene has not been delivered. Expression of a protein product of the tissue-specific gene can be determined immunologically, using methods such as radioimmunoassay, Western blotting, and immunohistochemistry. Alternatively, incorporation of labeled amino acids into a protein product can be determined. RNA expression is preferably determined using one or more oligonucleotide probes, either in solution or immobilized on a solid support, as described above.

All documents cited in this disclosure are expressly incorporated herein. The above disclosure generally describes the present invention, and all references cited in this disclosure are incorporated by reference herein. A more complete understanding can be obtained by reference to the following specific examples which are provided for purposes of illustration only and are not intended to limit the scope of the invention.

### Example 1

### Tissue Samples and the Sage Method

RNA for normal tissues was obtained from the following sources: colon epithelial cells isolated from sections of normal colon mucosa from two patients (41); HaCaT keratinocyte cells (42), normal mammary epithelial cells from two individuals (Clonetics); normal bronchial epithelial cell from two individuals (43); normal melanocytes from two individuals (Cascade Biologics); normal cultured monocytes, dendritic cells and TNF activated dendritic cells; two normal kidney epithelial cell lines; cultured chondrocyte cells from two normal individuals and one patient with osteoarthritic disease; normal fetal cardiomyocytes in normoxic and hypoxic conditions; and normal brain white matter from two patients and normal cultured astrocyte cells.

RNA for diseased tissues was obtained from the following sources: primary colon adenocarcinomas from two patients, HCT116, DLD1, HT29, Caco2, SW837, SW480, and RKO colon cancer cell lines cultured in vitro in a variety of different cellular conditions including log phase growth, G1/G2 phase growth arrest, and apoptosis (40, 41, 44, 45); primary pancreatic adenocarcinomas from two patients and ASPC-1 and PL-45 pancreatic cancer cell lines (41); breast cancer cell lines 21-PT, 21-MT, MDA-468, SK-BR3, and BT-474; primary lung squamous cell cancers from two patients (43), primary lung adenocarcinoma from one patient, and the A549 lung cancer cell line (43); primary melanomas from 3 patients; kidney epithelial cells lines from two patients with polycystic kidney disease; hemangiopericytomas from 5 patients; primary glioblastoma tumors from two patients; and the H392 glioblastoma cell line.

Isolation of polyadenylate RNA and the SAGE method for all tissues was performed as previously described (1, 12; see also U.S. Pat. Nos. 5,866,330 and 5,695,937).

### Example 2

### Data Analysis

The SAGE software (12) was used to analyze raw sequence data and to identify a total of 3,668,175 SAGE tags. Of these, 171,346 tags (4.7%) corresponded to linker sequences and were removed from further analysis. The remaining 3,496,829 tags were derived from transcript sequences, but a small fraction of these contained sequencing errors. SAGE analysis of yeast (1), for which the entire genome sequence is known, demonstrated a sequencing error rate of ˜0.7% per bp, translating to a tag error rate of 6.8% (1-0.993; 10), in accord with sequence errors measured in the current data set.

To provide as accurate an estimate of unique genes as possible, we accounted for sequencing errors in two ways. First, we only considered tags that occurred twice in the data set. Although this requirement might have removed legitimate transcript tags expressed at very low levels (less than approximately 0.2 copies per cell, or 2 copies in 3,496,829 transcript tags), it eliminated the majority of sequencing errors (172,276 tags).

Second, because of the size of the data set utilized, it was possible that the same sequencing error in a given tag may be observed multiple times. To account for these, tags with expression levels high enough to give multiple redundant errors were analyzed for single base substitutions, insertions, and deletions. If the observed expression level of a tag did not exceed its expected incidence due to redundant errors by a factor of five, it was assumed to be the result of a repeated sequencing error. This identified and removed an additional 27,051 unique tags (156,174 total tags), a number very similar to estimates of multiple sequencing errors obtained by Monte Carlo simulations.

In total, these corrections amount to a sequencing error rate of approximately 9.4%, suggesting that our analyses more than fully accounted for sequencing errors and that the remaining 134,135 unique transcript tags represented a conservative accounting of legitimate transcripts.

Transcript tags were matched to known genes and ESTs by use of tables containing matching 10 by transcript sequences, UniGene clusters, GenBank accession numbers, and functional descriptions downloaded from the SAGEmap web site (URL address: http file type, www server, domain name ncbi.nlm.nih.gov, SAGE directory) (Lal et al., in press) on Feb. 23, 1999 (UniGene build 70, at the URL address: http file type, www server, domain name ncbi.nlm.nih.gov, UniGene directory) and the Microsoft Access software. As UniGene clusters numbers may change over time, the most recent tag to cluster mapping can be obtained for each transcript tag individually at the URL address: http file type, www host server, domain name ncbi.nlm.nih.gov, SAGE directory, file name SAGEtag.cgi, or for the entire data set at the URL address: http file type, www host server, domain name sagenet.org, transcriptome directory. A total of 37,534 distinct transcripts from the UniGene database contained polyadenylation signals or polyadenylated tails and matched the collection of SAGE transcript tags; these corresponded to 23,534 unique UniGene clusters.

Transcript abundance per cell was determined simply by dividing the observed number of tags for a given transcript by the total number of transcripts obtained. An estimate of about 300,000 transcripts per cell was used to convert the abundances to copies per cell (46). For tissue specific transcripts, only transcript tags expressed at nominally ≧10 transcript copies per cell were considered in order to normalize for tissues with fewer total tags analyzed.

The following transcript data from this analysis are available electronically at the SAGEnet website (that has a URL address: http file type, www host server, domain name sagenet.org, transcriptome directory) with the corresponding expression levels and UniGene descriptions: 134,135 unique transcript tags identified from 3.5 million total transcripts tags; 69,381 transcript tags identified from colon cancer cells; 217 transcripts that are exclusively expressed in colon epithelium, keratinocytes, breast epithelium, lung epithelium, melanocytes, kidney epithelium and cells from prostate and brain; 987 transcripts that were expressed in all tissues. Individual transcript libraries from a total of ˜800,000 transcript tags from colon epithelium, normal brain, colon cancer, and brain cancer are available at the SAGEmap website (at the URL address: http file type, www host server, domain name ncbi.nlm.nih.gov, SAGE directory) (Lal et al., in press).

### Example 3

### Estimation of the Number of Genes Present in the Human Genome

The transcripts detected by SAGE provides an estimate of the number of genes present in the human genome. Historically, estimates of the number of unique genes in the genome have ranged from 60,000 to over 100,000 genes using analyses of EST clustering (15), frequency of genes in characterized genomic regions, frequency of CpG islands (16), and RNA-cDNA reassociation kinetics (4). If one were to assume that each unique transcript tag observed by SAGE corresponded to a unique gene, our data would indicate that there are approximately 134,000 genes in the human genome.

However, such an approach is likely to overestimate the number of unique genes in the genome, as distinct transcripts can be derived from a single gene. Multiple sites for polyadenylation (17), alternative splicing, premature transcriptional termination (18), as well as polymorphisms in the SAGE tag or nearby restriction endonuclease site could lead to multiple transcript tags for any one gene. An analysis of all publicly available 3′ end-derived ESTs revealed that this was the case for many transcripts, and provided an estimate of the multiplicity of transcripts expected for individual genes. 37,534 distinct 3′ transcripts containing polyadenylation signals or polyadenylated tails were observed to correspond to 23,534 unique UniGene clusters, an average 1.6 different transcripts per gene. Applying a similar calculation to our SAGE data would suggest that the 134,135 transcripts observed corresponded to 84,103 unique genes. As our SAGE data is by no means a complete analysis of transcripts from all possible tissues, this estimate would provide a lower boundary for the number of unique genes in the genome. This figure is significantly higher than the 65,538 genes estimated from a clustering of 982,808 ESTs (UniGene Build 70) (15), and suggests that a substantial number of genes expressed at low levels may not be present in current EST databases.

### Example 4

### Assessment of Transcriptome Complexity

Assessment of transcriptome complexity requires a relatively complete sampling of a transcriptome for the cell type under analysis. Human cells are thought to contain close to 300,000 mRNA molecules, and therefore an analysis of at least several hundred thousand transcripts would be needed. Approximately 350,000 and 300,000 transcripts were analyzed from DLD1 and HCT116 colorectal cancer cells, respectively. As these cancer cells are diploid, have similar genetic and phenotypic properties, and have very similar gene expression patterns (see below), transcript tags obtained from these cells were analyzed in combination as well as individually.

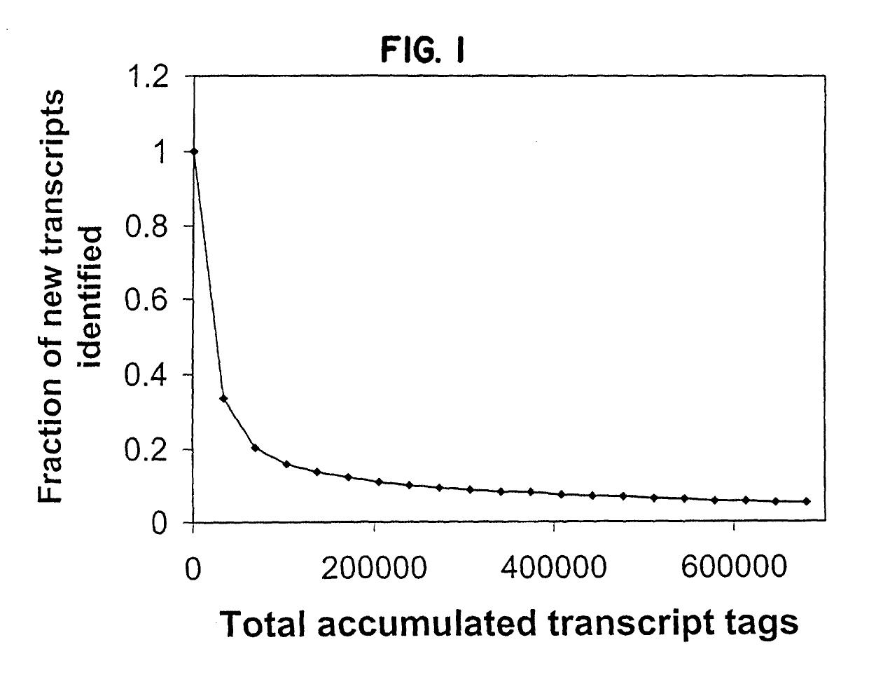

Analysis of either cell line afforded approximately a one fold coverage of the 300,000 mRNA molecules in a cell, while the combined set represented a two fold coverage even for mRNA molecules present at a single copy per cell. Measurement of ascertained new tags at increasing increments of tags indicated that the fraction of new transcripts from analysis of additional tags approached 0 at approximately 650,000 tags in the combined set (FIG. 1). This suggested that generation of further SAGE tags would yield few additional genes, and Monte Carlo simulations indicated that analysis of 643,283 tags would identify at least one tag for a given transcript 96% of the time if its expression level was at least two transcript copies per cell, and 83% of the time if its expression level was at least one transcript copy per cell.

The combined 643,283 transcript tags represented 69,381 unique transcripts, of which 44,174 corresponded to known genes or ESTs in the GenBank or UniGene databases while 25,207 represented previously undescribed transcripts (Table 2). Even when accounting for multiple unique transcripts per gene, these transcripts would represent at least 43,502 unique genes. This is substantially higher than the previous estimate of 15,000-25,000 expressed genes obtained by RNA-DNA reassociation kinetics in a variety of human cell types (4), and suggests that a significant fraction of the genome may be expressed in individual cell types. As the kinetics of reassociation of a particular class of RNA and cDNA may be affected by a number of experimental variables and may underestimate transcripts of low abundance (4), it is not surprising that our studies have detected a higher number of expressed genes than estimated by hybridization analysis in both human cells (Table 2) and yeast.

### Example 5

### Expression Levels of Transcripts in Colon Cancer Cells

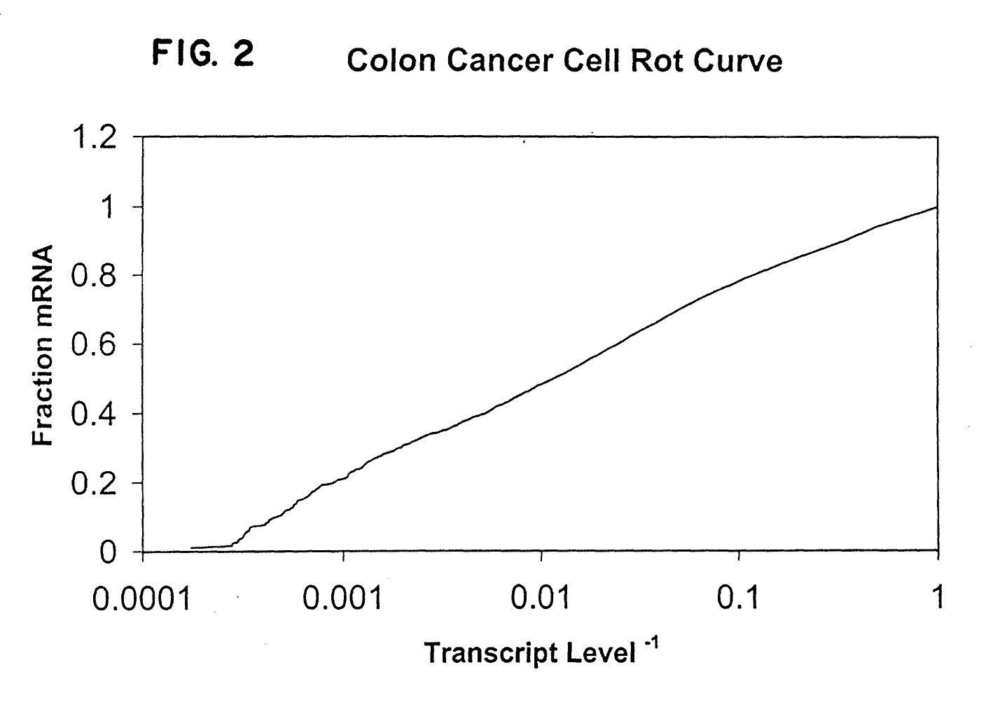

Expression levels of transcripts in the colon cancer cell ranged from 0.5 to 2341 copies per cell. The 61 transcripts expressed at over 500 transcript copies per cell made up nearly ¼ of the mRNA mass of the cell and the most highly expressed 623 genes accounted for ½ of the mRNA content. In contrast, the vast majority of unique transcripts were expressed at low levels, with just under 23% of the mRNA mass of the cell comprising 90% of the unique transcripts expressed (Table 2). A “virtual rot” analysis of the expressed transcripts identified a relatively continuous distribution of gene expression without markedly discrete abundance classes, similar to those observed in previous rot studies of human cancer cells (20) (FIG. 2).

The identities of the expressed genes reveal the diversity of expression of a human transcriptome (data available at the URL address: http file type, www host server, domain name sagenet.org, transcriptome directory). For example, highly expressed genes often encoded proteins important in protein synthesis, energy metabolism, cellular structure and certain tissue specific functions. Moderate and low abundance genes accounted for a multitude of cellular processes including protein modification enzymes, DNA replication machinery, cell surface receptors, components of signal transduction pathways and transcription factors as well as many other transcripts with currently unknown functions.

### Example 6

### Differences in Gene Expression Between Different Tissues

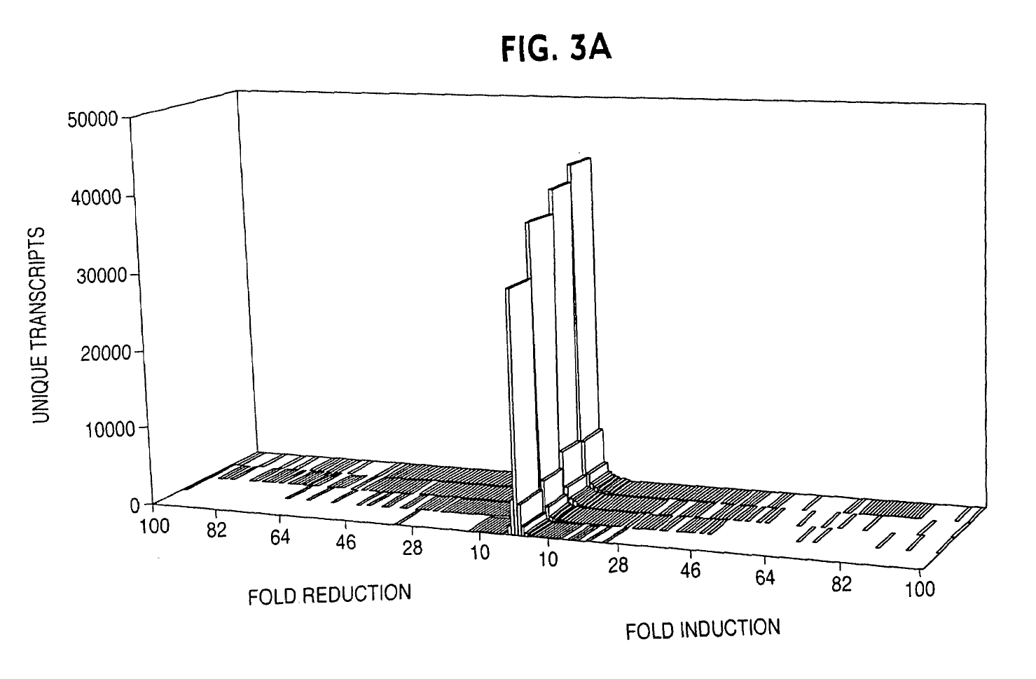

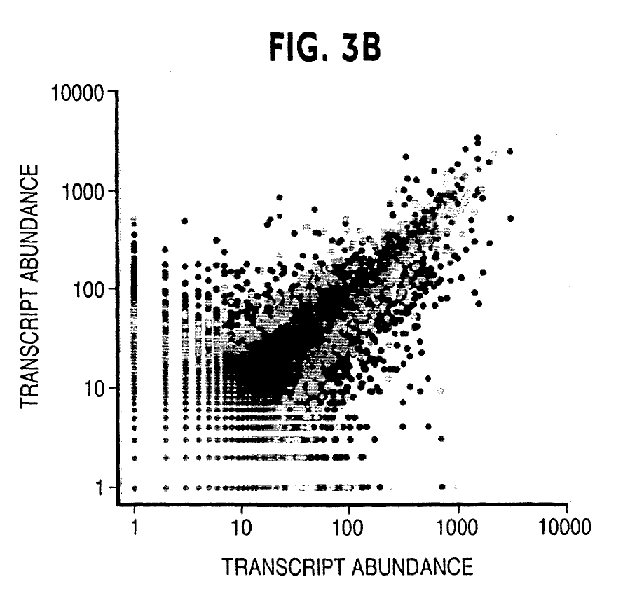

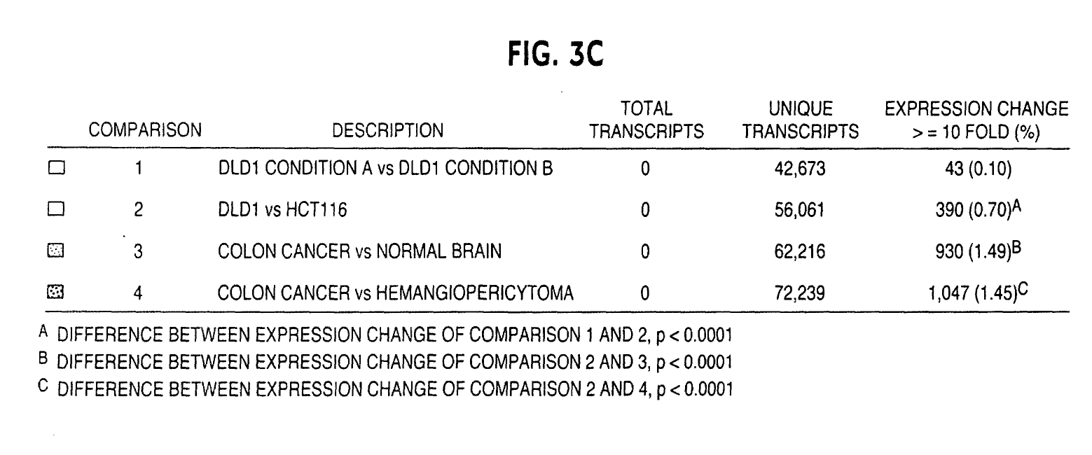

Differences in gene expression between different tissues may provide insights into the specialized processes underlying human physiology in normal and diseased states. In line with previous observations, overall gene expression patterns among the 19 different tissues analyzed were similar (examples in FIGS. 3A-3C). Changes in gene expression between physiologic states of a particular cell type or between patient samples of the same tissue were less than changes between cell types of different origins (FIGS. 3A-3C). Likewise, only a small fraction of transcripts was exclusively expressed in a particular normal or disease tissue. Detailed analysis of transcripts from epithelia of colon, breast, lung, and kidney, melanocytes, and cells from prostate and brain, identified transcripts that were nominally expressed at greater than 10 copies per cell in one tissue but not in any other tissue studied. The fraction of these tissue-specific transcripts ranged from 0.05% in normal prostate to 1.76% in normal colon epithelium (Table 3). Approximately 50% of these transcript tags matched known genes or ESTs (examples in Table 3 and data available at the URL address: http file type, www host server, domain name sagenet.org, transcriptome directory). Some of these transcripts identified genes already reported to be important for tissue specific processes. For example, brain specific transcripts such as GABA receptor, myelin basic protein, and synaptopodin are known to be important for synaptic transmission (21) formation and maintenance of the myelin sheath (22) and dendrite shape and motility (23), respectively. Likewise, guanylin/uroguanylin (24), carbonic anhydrase 1 (25), and CDX2 (26) are known to be expressed in colonic epithelium. 5,6-dihydroxyindole-2-carboxylic acid oxidase has been shown to have an important role for normal melanocyte pigment synthesis (27), while expression of MART-1 and melastatin may have clinical implications for melanoma patients (28, 29). However, the vast majority of the tissue specific transcripts observed have not been previously reported in the literature and their roles in the tissue examined remain to be elucidated.

### Example 7

### Minimal Transcriptome

Nearly 1000 transcripts were detected that were expressed at 5 transcript copies per cell in every cell type analyzed. These expressed genes represent a view into the “minimal transcriptome,” the set of genes expressed in all human cells. Such genes, listed in order of their uniformity of expression in Table 4 (and available at the URL address: http file type, www host server, domain name sagenet.org, transcriptome directory), largely represent well known constitutive or housekeeping genes thought to provide the molecular machinery necessary for basic functions of cellular life (4). Genes involved in DNA, RNA, protein, lipid and oligosaccharide biosynthesis as well as in energy metabolism were among those observed. Additionally, genes from other functional classes including structural proteins (e.g., dystroglycan and myosin light chain), signaling molecules (e.g., 14-3-3 proteins and MAPKK2), proteins with compartmentalized functions (e.g., lysosome-associated membrane glycoprotein and ER lumen retaining protein receptor 1), cell surface receptors (e.g., FGF receptor and STRL22 G protein coupled receptor), proteins involved in intracellular transport (e.g., syntaxin and alpha SNAP), membrane transporters (e.g., Na+/K+ ATPase and mitochondrial F1/F0 ATPase), and enzymes involved in post-translational modification and protein degradation (e.g., kinases, phosphatases and proteasome components) were observed and were not previously known to be ubiquitously expressed. Well known genes often used as experimental controls such as glyceraldehyde 3-phosphate dehydrogenase, elongation factor 1 alpha, and gamma actin were observed but varied in expression as much as 6 fold among different cell types.

### Example 8

### Genes Involved in Tumorigenesis

Genes that are uniformly expressed in cancers but expressed at lower levels in normal tissues may turn out to be important for tumorigenesis, and demonstrate how gene expression patterns might be useful in the analysis of disease states. We detected 40 genes that were expressed in all cancer tissues examined at levels 3 transcript copies per cell and whose expression was at least 2-fold higher in each cancer compared to its corresponding normal tissue (Table 5). Four of these transcripts had no matches to known genes and 15 matched ESTs with no known function. Several of the highly induced transcripts provided tantalizing clues about their roles in tumorigenesis. For example, S100A4 has been thought to play a role in late stage tumorigenesis as it is overexpressed in colorectal adenocarcinomas but not adenomas (30), and its induction can promote (while its inhibition can prevent) metastasis in tumor models. Midkine, a heparin-binding growth factor has been reported to be overexpressed in certain cancers (34), to transform cells in vitro (35), and to promote tumor angiogenesis in vivo. Finally, overexpression of survivin, an IAP apoptosis inhibitor (37) has been recently shown to predict shorter survival rates in colorectal cancer patients and may carry out its antiapoptotic functions as a mitotic spindle checkpoint factor (39). The observed elevated expression of such genes in many tumor types indicates a potentially general role for these genes in tumorigenesis and suggests they may be useful as diagnostic markers or targets for therapeutic intervention.

### Example 9

### Estimate of Gene Number

The 134,135 distinct transcripts identified in this study, corresponding to approximately 84,103 unique genes, provided an estimate of gene number substantially higher than the recent estimate (˜65,000 genes) derived from extant EST clusters. What could account for the difference between these estimates, considering that both are derived from sequencing of transcripts from similar cell types? One explanation is that the clustering estimate is based on the number of observed EST clusters (62,236) divided by a measure of the completeness of the EST database. The latter value is calculated as the fraction of “characterized” genes in GenBank that already have EST matches (˜95%). The characterized genes in GenBank have been assumed to be representative of the rest of the genes in the human genome, but our SAGE data indicated that their average expression was more than 10 fold higher than the mean levels of gene expression. Similarly, the number of ESTs that were present in clusters with characterized genes was approximately 12 fold higher than clusters composed entirely of ESTs. Such highly expressed genes would be more likely to be represented in transcript databases, thereby leading to an overestimation of the completeness of the EST databases, and an underestimation of the number of unique genes. Indeed, the number of UniGene clusters continues to grow as a greater diversity of tissues is analyzed through the Cancer Genome Anatomy Project, and as of the date of submission of this manuscript already exceeds the recent EST derived estimate (71,849 gene clusters in Build 80 versus 65,538 predicted from Build 70).

Like other genome-wide analyses, studies of human transcriptomes using SAGE have several potential limitations. First, a small number of transcripts would be expected to lack the restriction enzyme site required to produce the 14 by tags, and would therefore not be detected by our analyses (12). Second, our study was limited to the 19 tissues analyzed. Genes uniquely expressed in other tissues would not have been detected, and accordingly, genes observed to be tissue specific in our studies may turn out to be expressed in other normal or disease states. Finally, identification of genes corresponding to specific tags is mainly based on large but incomplete databases of ESTs and characterized genes. SAGE tags without matches to existing databases can directly be used to identify previously uncharacterized genes (1, 12, 40), but additional 3′ EST data, as well as that of genomic regions would make gene identification more rapid.

### REFERENCES

1. Velculescu et al., Cell 88, 243-251 (1997).
2. Pietu et al., Genome Res 9 195-209 (1999).
3. Wadman, Nature 398, 177 (1999).
4. Lewin, Gene Expression 2, 694-727 (1980).
5. Adams et al., Nature 377, 3 ff. (1995)
6. Okubo et al., DNA Res 1, 37-45 (1994).
7. Alwine et al. Proc Natl Acad Sci USA 74, 5350-5354 (1977).
8. Zinn et al. Cell 34, 865-879 (1983).
9. Veres et al. Science 237, 415-417 (1987).
10. Hedrick et al. Nature 308, 149-153 (1984).
11. Liang & Pardee, Science 257, 967-971 (1992).
12. Velculescu et al. Science 270, 484-487 (1995).
13. Kal et al., Mol Biol Cell 10, 1859-1872 (1999).
14. Basrai et al., NORF5/HUG1 is a component of the MEC1 mediated checkpoint response to DNA damage and replication arrest in S. cerevisiae. submitted.
15. Fields et al. Nat Genet. 7, 345-346 (1994).
16. Antequera et al. Proc Natl Acad Sci USA 90 11995-11999 (1993).
17. Gautheret et al. Genome Res 8, 524-530 (1998).
18. Bouck et al. Trends Genet. 15, 159-62 (1999).
19. Bentley & Groudine, Cell 53, 245-256 (1988).
20. Bishop et al. Nature 250, 199-204 (1974).
21. Mody et al. Trends Neurosci 17, 517-25 (1994).
22. Staugaitis et al. Bioessays 18, 13-18 (1996).
23. Mundel et al., J Cell Biol 139, 193-204 (1997).
24. Wiegand et al. FEBS Lett 311, 150-154 (1992).
25. Sowden et al. Differentiation 53, 67-74 (1993).
26. Suh & Traber, Mol Cell Biol 16, 619-625 (1996).
27. Blarzino et al., Free Radic Biol Med 26, 446-453 (1999).
28. Busam et al. Adv Anat Pathol 6, 12-18 (1999).
29. Duncan et al., Cancer Res 58, 1515-1520 (1998).
30. Takenage et al., Clin Cancer Res 3, 2309-2316 (1997).
31. Lloyd et al. Oncogene 17, 465-473 (1998).
32. Maelandsmo et al., Cancer Res 56, 5490-5498 (1996).
33. Muramatsu & Muramatsu, Biochem Biophy Res Commun 177, 652-658 (1991).
34. Tsutsui et al., Cancer Res 53, 1281-1285 (1993).
35. Kadomatsu et al., Br J Cancer 75, 354-359 (1997).
36. Choudhuri et al. Cancer Res. 57, 1814-1819 (1997).
37. Ambrosini et al. Nat Med 3, 917-921 (1997).
38. Kawasaki et al., Cancer Res 58, 5071-5074 (1998).
39. Li et al., Nature 396, 580-584 (1998).
40. Polyak et al. Nature 389, 300-304 (1997).
41. Zhang et al., Science 276, 1268-1272 (1997).
42. Boukam et al., J Cell Biol 106, 761-771 (1988).
43. Hibi et al., Cancer Res 58, 5690-5694 (1998).
44. Hermeking et al., Molecular Cell 1, 3-11 (1997).
45. He et al., Science 281, 1509-1512 (1998).
46. Hastie & Bishop, Cell 9, 761-774 (1976).
47. Agrawal et al., Trends Biotechnol. 10, 152-158 (1992)
48. Uhlmann et al., Chem. Rev. 90, 543-584 (1990)
49. Uhlmann et al., Tetrahedron. Lett. 215, 3539-3542 (1987)
50. Brown, Meth. Mol. Biol. 20, 1-8 (1994)
51. Sonveaux, Meth. Mol. Biol. 26, 1-72 (1994)
52. Uhlmann et al., Chem. Rev. 90, 543-583 (1990)
53. White & Bancroft, J. Biol. Chem. 257, 8569 (1982)
54. Sambrook et al., MOLECULAR CLONING. A LABORATORY MANUAL, 2d ed., pages 7.53-7.57 (1989)
55. Chee et al., Science 274, 610-14 (1996)
56. DeRisi et al., Nat. Genet. 14, 457-60 (1996)
57. Schena, Bioessays 18, 427-31 (1996)
58. Lockhart et al., Nature Biotechnology, 14 (1996)
59. Romanczuk et al., Hum. Gene. Ther. 10, 2615-26
60. Lanzov, Mol. Genet. Metab. 68, 276-82 (1999)
61. Lai & Lien, Exp. Nephrol. 7, 11-14 (1999)

TABLE 1
Tissues and transcript tags analyzed

| | Libraries | Total Transcripts | Unique Genes |
|---|---|---|---|
| Normal tissues | | | |
| Colon epithelium^{1,2} | 2 | 98,089 | 12,941 |
| Keratinocytes³ | 2 | 83,835 | 12,598 |
| Breast epithelium³ | 2 | 107,632 | 13,429 |
| Lung epithelium⁴ | 2 | 111,848 | 11,636 |
| Melanocytes³ | 2 | 110,631 | 14,824 |
| Prostate³ | 2 | 98,010 | 9,786 |
| Monocytes³ | 3 | 66,673 | 9,504 |
| Kidney epithelium³ | 2 | 103,836 | 15,094 |
| Chondrocytes³ | 4 | 88,875 | 11,628 |
| Cardiomyocytes³ | 4 | 77,374 | 9,449 |
| Brain² | 3 | 202,448 | 23,580 |
| Diseased Tissues | | | |
| Colon cancer^{1,2,3} | 22 | 1,004,509 | 56,153 |
| Pancreatic cancer¹ | 4 | 126,414 | 17,050 |
| Breast cancer³ | 5 | 226,630 | 18,685 |
| Lung cancer⁴ | 5 | 221,302 | 22,783 |
| Melanoma³ | 10 | 269,332 | 25,600 |
| Polycystic kidney | 2 | 112,839 | 16,280 |
| disease³ | | | |
| Hemangiopericytoma³ | 5 | 199,985 | 31,351 |
| Brain cancer² | 3 | 186,567 | 23,108 |
| Total | 84 | 3,496,829 | 84,103 |

¹Ref. 5, 6, 7, 8
²Ref. 9
³unpublished
⁴Ref. 10

TABLE 2
Expressed transcripts (>500 copies per cell)

| | | |
|---|---|---|
| | Copies/ | |
| Tag Sequence | Cell | Description |

| | | |
|---|---|---|
| CCCATCGTCC | 3022 | Tag matches mitochondrial sequence |
| GTGACCACGG | 2435 | Tag matches ribosomal RNA sequence/Human N-methyl-D-aspartate receptor 2C subunit |
| | | precursor (NMDAR2C) mRNA |
| TGTGTTGAGA | 1557 | Translation elongation factor 1-alpha-1 |
| GTGAAACCCC | 1466 | Multiple matches |
| CCTGTAATCC | 1403 | Multiple matches |
| CTAAGACTTC | 1349 | Tag matches mitochondrial sequence |
| CACCTAATTG | 1333 | Tag matches mitochondrial sequence |
| CCCGTCCGGA | 1282 | 60S RIBOSOMAL PROTEIN L13 |
| TTGGTCCTCT | 1238 | 60S RIBOSOMAL PROTEIN L41 |
| ATGGCTGGTA | 1126 | 40S RIBOSOMAL PROTEIN S2 |
| TTGGGGTTTC | 1099 | Ferritin heavy chain |
| CCACTGCACT | 964 | Multiple matches |
| TGATTTCACT | 942 | Tag matches mitochondrial sequence/EST |
| ACTTTTTCAA | 899 | Tag matches mitochondrial sequence |
| GCAGCCATCC | 886 | Ribosomal protein L28 |
| TACCATCAAT | 874 | Glyceraldehyde-3-phosphate dehydrogenase |
| GGATTTGGCC | 854 | Ribosomal protein, large P2/Ribosomal protein S26/Human mRNA for PIG-B |
| CCCTGGGTTC | 844 | Ferritin, light polypeptide |
| GCCGAGGAAG | 836 | Human mRNA for ribosomal protein S12 |
| AGGCTACGGA | 820 | 60S RIBOSOMAL PROTEIN L13A |
| CGCCGCCGGC | 805 | Human ribosomal protein L35 mRNA, complete cds |
| TTCATACACC | 804 | Tag matches mitochondrial sequence |
| AGCCCTACAA | 801 | Tag matches mitochondrial sequence |
| CACAAACGGT | 799 | 40S RIBOSOMAL PROTEIN S27 |
| AAGGTGGAGG | 786 | 60S RIBOSOMAL PROTEIN L18A |
| CTTCCTTGCC | 777 | Keratin 17 |
| TGGTGTTGAG | 770 | Human DNA sequence from clone 1033B10 on chromosome 6p21.2-21.31 |
| GTGAAACCCT | 728 | Multiple matches |
| GGGGAAATCG | 724 | THYMOSIN BETA-10 |
| AGCACCTCCA | 718 | Eukaryotic translation elongation factor 2 |
| CCTCCAGCTA | 711 | Keratin 8 |
| AAGACAGTGG | 699 | Ribosomal protein L37a |
| CTGGGTTAAT | 699 | 40S RIBOSOMAL PROTEIN S19 |
| ATTTGAGAAG | 689 | Tag matches mitochondrial sequence |
| GCCGGGTGGG | 687 | Basigin |
| GGGCTGGGGT | 683 | H. sapiens mRNA for ribosomal protein L29/Homo sapiens sperm acrosomal |
| | | protein mRNA |
| AGGGCTTCCA | 663 | UBIQUINOL-CYTOCHROME C REDUCTASE COMPLEX SUBUNIT VI REQUIRING PROTEIN |
| AAAAAAAAAA | 650 | Multiple matches |
| GAGGGAGTTT | 648 | Ribosomal protein L27a |
| GCGACCGTCA | 637 | Aldolase A |
| ACTAACACCC | 631 | Tag matches mitochondrial sequence |
| CGCCGGAACA | 616 | Ribosomal protein L4 |
| TGGGCAAAGC | 592 | Translation elongation factor 1 gamma |
| TGCACGTTTT | 586 | Human mRNA for antileukoprotease (ALP) from cervix uterus |
| AATCCTGTGG | 569 | Ribosomal protein L8 |
| CAAGCATCCC | 565 | Tag matches mitochondrial sequence |
| CCGTCCAAGG | 559 | Ribosomal protein S16 |
| TAGGTTGTCT | 551 | TRANSLATIONALLY CONTROLLED TUMOR PROTEIN |
| GCCGTGTCCG | 540 | Human ribosomal protein S6 mRNA, complete cds |
| GCTTTATTTG | 540 | Human mRNA fragment encoding cytoplasmic actin |
| CTAGCCTCAC | 539 | Actin, gamma 1 |
| CCTAGCTGGA | 537 | PEPTIDYL-PROLYL CIS-TRANS ISOMERASE A |
| GCCCCTGCTG | 534 | Keratin 5 (epidermolysis bullosa simplex, Dowling-Meara/Kobner/Weber- |
| | | Cockayne types) |
| ACCCTTGGCC | 526 | Tag matches mitochondrial sequence |
| AGGAAAGCTG | 513 | ESTs, Highly similar to 60S RIBOSOMAL PROTEIN L36 [Rattus norvegicus] |

TABLE 3
Transcripts expressed in Colon Cancer Cells (>500 copies/cell)

| | | |
|---|---|---|
| Tag | Copies/cell | Unigene Description |

| | | |
|---|---|---|
| CCCATCGTCC | 2672 | Tag matches mitochondrial sequence |
| TGTGTTGAGA | 1672 | Translation elongation factor 1-alpha-1 |
| GGATTTGGCC | 1663 | Ribosomal protein, large P2/Ribosomal protein S26/Human mRNA for PIG-B, |
| | | complete cds |
| CCCGTCCGGA | 1559 | 60S RIBOSOMAL PROTEIN L13 |
| ATGGCTGGTA | 1555 | 40S RIBOSOMAL PROTEIN S2 |
| GTGAAACCCC | 1482 | Multiple matches |
| CCTCCAGCTA | 1468 | Keratin 8 |
| TTGGTCCTCT | 1453 | 60S RIBOSOMAL PROTEIN L41 |
| TGATTTCACT | 1434 | EST/Tag matches mitochondrial sequence |
| CCTGTAATCC | 1372 | Multiple matches |
| ACTTTTTCAA | 1367 | Tag matches mitochondrial sequence |
| AAAAAAAAAA | 1357 | Multiple matches |
| GAGGGAGTTT | 1290 | Ribosomal protein L27a |
| GCCGAGGAAG | 1141 | Human mRNA for ribosomal protein S12 |
| CACCTAATTG | 1137 | Tag matches mitochondrial sequence |
| CGCCGCCGGC | 1098 | Human ribosomal protein L35 mRNA, complete cds |
| GGGGAAATCG | 1092 | THYMOSIN BETA-10 |
| GAAAAATGGT | 1056 | Laminin receptor (2H5 epitope) |
| GGGCTGGGGT | 1028 | H. sapiens mRNA for ribosomal protein L29/Homo sapiens sperm acrosomal |
| | | protein mRNA |
| GCCGGGTGGG | 986 | Basigin |
| AGCCCTACAA | 945 | Tag matches mitochondrial sequence |
| CTGGGTTAAT | 943 | 40S RIBOSOMAL PROTEIN S19 |
| CAAACCATCC | 927 | Keratin 18 |
| TGCACGTTTT | 916 | Human mRNA for antileukoprotease (ALP) from cervix uterus |
| AGGCTACGGA | 905 | 60S RIBOSOMAL PROTEIN L13A |
| GCAGCCATCC | 861 | Ribosomal protein L28 |
| TTCAATAAAA | 851 | Ribosomal protein, large, P1/TRANSCOBALAMIN I PRECURSOR |
| CTAAGACTTC | 833 | Tag matches mitochondrial sequence |
| TGGTGTTGAG | 830 | Human DNA sequence from clone 1033B10 on chromosome 6p21.2-21.31 |
| TACCATCAAT | 828 | Glyceraldehyde-3-phosphate dehydrogenase |
| TTCATACACC | 814 | Tag matches mitochondrial sequence |
| CCACTGCACT | 800 | Multiple matches |
| ACTAACACCC | 795 | Tag matches mitochondrial sequence |
| AAGGTGGAGG | 794 | 60S RIBOSOMAL PROTEIN L18A |
| AGCACCTCCA | 787 | Eukaryotic translation elongation factor 2 |
| CACAAACGGT | 761 | 40S RIBOSOMAL PROTEIN S27 |
| AGGAAAGCTG | 732 | ESTs, Highly similar to 60S RIBOSOMAL PROTEIN L36 [Rattus norvegicus] |
| GTGAAACCCT | 729 | Multiple matches |
| AATCCTGTGG | 711 | Ribosomal protein L8 |
| TTGGGGTTTC | 698 | Ferritin heavy chain |
| AAGACAGTGG | 696 | Ribosomal protein L37a |
| ATTTGAGAAG | 680 | Tag matches mitochondrial sequence |
| GCCGTGTCCG | 679 | Human ribosomal protein S6 mRNA, complete cds |
| CGCCGGAACA | 678 | Ribosomal protein L4 |
| TCTCCATACC | 661 | Tag matches mitochondrial sequence |
| ACATCATCGA | 661 | Ribosomal protein L12 |
| AACGCGGCCA | 644 | Macrophage migration inhibitory factor |
| AGGGCTTCCA | 643 | UBIQUINOL-CYTOCHROME C REDUCTASE COMPLEX SUBUNIT VI REQUIRING |
| | | PROTEIN |
| CCGTCCAAGG | 631 | Ribosomal protein S16 |
| CGCTGGTTCC | 626 | Homo sapiens ribosomal protein L11 mRNA, complete cds |
| CTCAACATCT | 615 | Ribosomal protein, large, P0 |
| ACTCCAAAAA | 608 | H. sapiens mRNA for transmembrane protein rnp24/Human insulinoma rig-analog mRNA |
| | | encoding DNA-binding protein |
| CCTAGCTGGA | 606 | PEPTIDYL-PROLYL CIS-TRANS ISOMERASE A |
| GTGAAGGCAG | 596 | Ribosomal protein S3A |
| AGCTCTCCCT | 551 | 60S RIBOSOMAL PROTEIN L23 |
| TAGGTTGTCT | 537 | TRANSLATIONALLY CONTROLLED TUMOR PROTEIN |
| GGACCACTGA | 522 | Ribosomal protein L3 |
| AAGGAGATGG | 521 | Ribosomal protein L31 |
| AACTAAAAAA | 510 | Ubiquitin A-52 residue ribosomal protein fusion product 1 |
| GGCTGGGGGC | 507 | Human profilin mRNA, complete cds |
| CCAGAACAGA | 503 | Deoxythymidylate kinase/60S RIBOSOMAL PROTEIN L30 |

TABLE 4
Transcript abundance

| | Colon Cancer | | | All | | |
|---|---|---|---|---|---|---|
| | Cells | | | Tissues | | |
| | | | Mass | | | Mass |
| | | | fraction | | | fraction |
| | Unique | | mRNA | Unique | | mRNA |
| Copies/Cell | transcripts | | (%) | transcripts | | (%) |
| >500 | 61 | | 20 | 55 | | 18 |
| Match | | | | | | |
| GenBank (%) | 61 | (100) | | 55 | (100) | |
| 50 to 500 | 562 | | 27 | 578 | | 27 |
| Match | | | | | | |
| GenBank (%) | 554 | (99) | | 576 | (100) | |
| 5 to 50 | 6,358 | | 30 | 6,160 | | 30 |
| Match | | | | | | |
| GenBank (%) | 6,023 | (95) | | 5,913 | (96) | |
| <=5 | 62,400 | | 23 | 127,342 | | 25 |
| Match | | | | | | |
| GenBank (%) | 37,536 | (60) | | 66,091 | (52) | |
| Total | 69,381 | | 100 | 134,135 | | 100 |
| Match | | | | | | |
| GenBank (%) | 44,174 | (64) | | 72,635 | (54) | |

TABLE 5
Tissue specific genes

| | | | |
|---|---|---|---|
| | | Copies/ | |
| Tag sequence | Observed | cell | Unigene Description |

| | | | |
|---|---|---|---|
| Colon epithelium | | | |
| (1.76%) | | | |
| ATACTCCACT | 141 | 431 | Guanylate cyclase activator 2 (guanylin, intestinal, heat-stable) |
| TCAGCTGCAA | 72 | 220 | No match |
| GTCATCACCA | 57 | 174 | H. sapiens mRNA for GCAP-II/uroguanylin precursor |
| CCTTCAAATC | 46 | 141 | Carbonic anhydrase I |
| ACACCCATCA | 29 | 89 | No match |
| CCAACACCAG | 28 | 86 | No match |
| AATAGTTTCC | 23 | 70 | Pregnancy-specific beta-1 glycoprotein 6 |
| CCAGGCGTCA | 18 | 55 | No match |
| GAACAGCTCA | 18 | 55 | ESTs |
| TACTCGGCCA | 15 | 46 | No match |
| GGGGGAGAAG | 12 | 37 | ESTs |
| AGTGGGCTCA | 11 | 34 | No match |
| GAGCACCGTG | 11 | 34 | No match |
| GATCTATCCA | 10 | 31 | ESTs |
| GAACGCCAGA | 9 | 28 | No match |
| GCCCTCGGAG | 9 | 28 | ESTs |
| ACAAGCCTAG | 9 | 28 | No match |
| GTCACAGGAA | 9 | 28 | No match |
| GCCCTCGGAG | 9 | 28 | Human homeobox protein Cdx2 mRNA, complete cds |
| CTAGGATGAT | 9 | 28 | ESTs |
| CCAACTATCG | 8 | 24 | No match |
| CTGACGGGGA | 8 | 24 | ESTs |
| GAGGGTTTTA | 8 | 24 | Homo sapiens C19steroid specific UDP-glucuronosyltransferase |
| | | | mRNA, complete cds |
| GGGGTCCCAT | 8 | 24 | No match |
| GCCAGGTCAC | 7 | 21 | No match |
| AGAACACCAA | 7 | 21 | No match |
| AATCCCGCCC | 7 | 21 | Homo sapiens hAQP8 mRNA for aquaporin 8, complete cds |
| ACACTGCCTC | 6 | 18 | No match |
| AGAGTCCAGG | 6 | 18 | Homo sapiens carcinoembryonic antigen (CGM2) mRNA, complete cds |
| CCAGACGTAG | 6 | 18 | No match |
| GAGGCCCCCG | 6 | 18 | No match |
| CTGTGTGCCC | 5 | 15 | ESTs, Weakly similar to tryptase-III [H. sapiens] |
| GAGAGGATGG | 5 | 15 | ESTs |
| GGCTGAACCA | 5 | 15 | No match |
| CCAAATCATT | 5 | 15 | No match |
| ACGGCTGGGC | 5 | 15 | No match |
| ACCTTCATCT | 5 | 15 | EST |
| AGGGCTTGAG | 5 | 15 | No match |
| ACCTTCATCT | 5 | 15 | Human rearranged metabotropic glutamate receptor type II (GLUR2) |
| | | | mRNA, complete cds |
| TCAGGCCAGA | 5 | 15 | No match |
| CTGTGTGCCC | 5 | 15 | ESTs |
| GGATGTCAAC | 5 | 15 | Human RecA-like protein (hREC2) mRNA, complete cds |
| ATCTGGAGCA | 5 | 15 | Alcohol dehydrogenase 1 (class I), alpha polypeptide |
| GAGAGGATGG | 5 | 15 | INTEGRAL MEMBRANE PROTEIN E16 |
| ATCTGGAGCA | 5 | 15 | Alcohol dehydrogenase 3 (class I), gamma polypeptide |
| GGATGTCAAC | 5 | 15 | Polymeric immunoglobulin receptor |
| CACAGACACA | 4 | 12 | No match |
| TGCTCCTAAC | 4 | 12 | No match |
| TATACCCGGA | 4 | 12 | No match |
| TATCCTGATG | 4 | 12 | No match |
| GGCCCTCCCG | 4 | 12 | No match |
| GTAGCGATGG | 4 | 12 | Pim-1 oncogene |
| GCAGGTTGTG | 4 | 12 | No match |
| TGGGAACCGG | 3 | 9 | No match |
| ACACCTCTCT | 3 | 9 | No match |
| GGAAAACAGG | 3 | 9 | No match |
| CAGGCGGCAC | 3 | 9 | No match |
| CAGGTTGGTC | 3 | 9 | Homo sapiens hRVP1 mRNA for RVP1, complete cds |
| GGGATATAAA | 3 | 9 | No match |
| GTGGAAAATC | 3 | 9 | No match |
| GTGTGTGAAT | 3 | 9 | No match |
| ATGTGACACT | 3 | 9 | No match |
| ATGGTGTAAT | 3 | 9 | ESTs |
| TCACATTGAT | 3 | 9 | H. sapiens mRNA for LI-cadherin |
| TAACTAAACA | 3 | 9 | No match |
| TGCCCGGGTC | 3 | 9 | No match |
| TAGTCGGAAA | 3 | 9 | No match |
| GCTATACGGG | 3 | 9 | No match |
| TCACACCCCA | 3 | 9 | No match |
| CTGCCCGAAC | 3 | 9 | ESTs |
| AGTCACCTCT | 3 | 9 | No match |
| TCATTGGTTT | 3 | 9 | No match |
| TCCTCTCCTC | 3 | 9 | No match |
| CCTCTCGGCC | 3 | 9 | No match |
| CCACTGAAGT | 3 | 9 | No match |
| CTGGCTTGCT | 3 | 9 | No match |
| GAAAACAGAA | 3 | 9 | EST |
| AAAGCACGTC | 3 | 9 | No match |
| GAAAACAGAA | 3 | 9 | ESTs, Weakly similar to synapse-associated protein sap47-1 |
| | | | [D. melanogaster] |
| TTGATTCCAT | 3 | 9 | No match |
| AAACAGGCAC | 3 | 9 | No match |
| CTTACAGTCC | 3 | 9 | No match |
| GAATGGACTC | 3 | 9 | No match |
| GAACCCAAAC | 3 | 9 | No match |
| GAAAACAGAA | 3 | 9 | ESTs |
| Normal Brain | | | |
| (1.36) | | | |
| ACTTTGTCCC | 160 | 237 | Glial fibrillary acidic protein |
| GTGCGAATCC | 79 | 117 | ESTs |
| CAAAAAGTTA | 36 | 53 | ESTs |
| TTAACTTTAT | 33 | 49 | Homo sapiens neuroendocrine-specific protein A (NSP) mRNA, |
| | | | complete cds |
| CAGCCAAATG | 29 | 43 | ESTs |
| GCCTGTGGTG | 28 | 41 | Homo sapiens LY6H mRNA, complete cds |
| CTTAGGGACA | 26 | 39 | ESTs |
| TTGGAGGTGA | 22 | 33 | ESTs |
| ATTCCATTTC | 20 | 30 | ESTs |
| ATTCCATTTC | 20 | 30 | ESTs, Highly similar to RAS-RELATED PROTEIN RAB-10 [Canis |
| | | | familiaris] |
| AGAGAGCGGA | 19 | 28 | Human guanine nucleotide-binding regulatory protein (Go-alpha) |
| | | gene | |
| TTCTCAATAC | 19 | 28 | Homo sapiens mRNA for synaptopodin |
| CATCCTCCCA | 19 | 28 | No match |
| GTATCGATTT | 16 | 24 | Homo sapiens GABA-B receptor mRNA, complete cds |
| TTGTAAACAG | 15 | 22 | ESTs, Weakly similar to cyclin I [H. sapiens] |
| GCCCTGTATT | 15 | 22 | ESTs |
| CCACATTGCC | 15 | 22 | Homo sapiens chromosome 7q22 sequence |
| CAGGGCAACG | 15 | 22 | No match |
| AAAAGCAAAT | 15 | 22 | Human mRNA for MOBP (myelin-associated oligodendrocytic basic |
| | | | protein), complete cds, clone hOPRP1 |
| ACCAATCCTA | 14 | 21 | Human guanine nucleotide-binding regulatory protein (Go-alpha) |
| | | gene | |
| CTGTGTGTCC | 13 | 19 | AXONIN-1 PRECURSOR |
| TCAGACAATA | 12 | 18 | ESTs |
| TGGTGAGATG | 12 | 18 | ESTs |
| ATTTTTTGTT | 12 | 18 | ESTs |
| ACATTGAGTC | 12 | 18 | Homo sapiens mRNA for MEGF4, partial cds |
| GTCAGTCTAC | 11 | 16 | Glutamate receptor, metabotropic 3 |
| GTCCCACTTC | 11 | 16 | ESTs |
| GGGGCCCGAA | 11 | 16 | No match |
| TGACTCACCC | 10 | 15 | Homo sapiens calmodulin-stimulated phosphodiesterase PDE1B1 |
| | | | mRNA, complete cds |
| GACAGCGACA | 10 | 15 | No match |
| GGTGTACATA | 10 | 15 | ESTs |
| TAGCTATAAA | 10 | 15 | ESTs |
| GGTGTACATA | 10 | 15 | ESTs |
| GTTTCATTTT | 10 | 15 | ESTs |
| AATAAATTGC | 10 | 15 | ESTs |
| GTTTCATTTT | 10 | 15 | ESTs |
| ACACATTGTA | 10 | 15 | No match |
| TACCTATTGT | 10 | 15 | ESTs |
| TTTAGCAGAA | 10 | 15 | Homo sapiens cyclin E2 mRNA, complete cds |
| TTTAGCAGAA | 10 | 15 | ESTs |
| CAATTTATGA | 9 | 13 | ESTs |
| GTGAAGGTTT | 9 | 13 | Homo sapiens (huc) mRNA, complete cds |
| TGGACTTTTA | 9 | 13 | ESTs |
| CGATGCCACG | 9 | 13 | No match |
| GTGAAGGTTT | 9 | 13 | Neuron-specific RNA recognition motifs (RRMs)-containing protein |
| | | | [human, hippocampus, mRNA, 1992 nt] |
| TGGACTTTTA | 9 | 13 | ESTs |
| CCTTCTTGTC | 9 | 13 | No match |
| TCCATTCAAG | 9 | 13 | Human clone 23586 mRNA sequence |
| CCTATGTATC | 8 | 12 | No match |
| ACGGACCAAT | 8 | 12 | No match |
| TATTATCTTG | 8 | 12 | ESTs |
| ACTTTATACG | 8 | 12 | ESTs |
| ACTTTATACG | 8 | 12 | ESTs, Weakly similar to EPIDERMAL GROWTH FACTOR RECEPTOR |
| | | | KINASE SUBSTRATE EPS8 [H. sapiens] |
| CGCAGTCCCC | 8 | 12 | BETA-NEOENDORPHIN-DYNORPHIN PRECURSOR |
| TGTAGTGCTC | 8 | 12 | No match |
| CTGCTTAAGT | 8 | 12 | ESTs, Weakly similar to unknown [H. sapiens] |
| ACAAGTGGAA | 8 | 12 | Human mRNA for KIAA0027 gene, partial cds |
| AATCCCAATG | 7 | 10 | Homo sapiens mRNA for KIAA0283 gene, partial cds |
| ACTATGCATC | 7 | 10 | No match |
| ACGAGTCATT | 7 | 10 | ESTs |
| TTACATTGTA | 7 | 10 | Homo sapiens clone 24461 mRNA sequence |
| ATGCCCCCTC | 7 | 10 | ESTs, Highly similar to HYPOTHETICAL 52.2 KD PROTEIN ZK512.6 IN |
| | | | CHROMOSOME III [Caenorhabditis elegans] |
| TTTTATTCAT | 7 | 10 | ESTs |
| ACAGAGCATT | 7 | 10 | No match |
| TGACCAATAG | 7 | 10 | No match |
| AATCCCAATG | 7 | 10 | Plastin 1 (I isoform) |
| Keratinocytes | | | |
| (0.087%) | | | |
| GCGAACTGGG | 5 | 18 | ORPHAN RECEPTOR TR4 |
| GCAACACTAA | 3 | 11 | No match |
| GTAATGGATT | 3 | 11 | No match |
| AGCAGACGTG | 3 | 11 | No match |
| Breast Epithelium | | | |
| (0.14%) | | | |
| GGATTCGGTC | 6 | 17 | No match |
| CGGAAGGCGG | 5 | 14 | No match |
| TGTAAGTACG | 5 | 14 | No match |
| GATCAGTCAT | 4 | 11 | No match |
| GCTCAGAGTT | 4 | 11 | No match |
| Lung epithelium | | | |
| (0.17%) | | | |
| TAACCTCCCC | 90 | 241 | No match |
| AGGAACAACT | 6 | 16 | No match |
| GGGTCCGTGG | 6 | 16 | No match |
| TAGCAAAATA | 5 | 13 | No match |
| GCTGTGCACA | 4 | 11 | No match |
| CAGAAAATCA | 4 | 11 | No match |
| GATTTGCTGG | 4 | 11 | No match |
| Melanocyte | | | |
| (0.93%) | | | |
| GTGCCATTCT | 114 | 309 | No match |
| GATATTTGTC | 40 | 108 | 5,6-DIHYDROXYINDOLE-2-CARBOXYLIC ACID OXIDASE |
| | | | PRECURSOR |
| TATGATTTTA | 39 | 106 | ESTs |
| TCACTGCAAC | 27 | 73 | 5,6-DIHYDROXYINDOLE-2-CARBOXYLIC ACID OXIDASE |
| | | | PRECURSOR |
| CCCAGTCACA | 21 | 57 | ESTs, Weakly similar to LACTOSE PERMEASE [Escherichia coli] |
| TATGAGAACC | 17 | 46 | ESTs, Highly similar to HIGH AFFIMMUNOGLOBULIN GAMMA FC |
| | | | RECEPTOR I PRECURSOR [Homo sapiens] |
| GAGTTTAGTG | 16 | 43 | No match |
| CTCCACTCTG | 15 | 41 | No match |
| ATCCAGTGAC | 14 | 38 | No match |
| TGATCTTGAG | 14 | 38 | ESTs, Moderately similar to PAS protein 5 [H. sapiens] |
| AATGGCTGTT | 12 | 33 | Human melanoma antigen recognized by T-cells (MART-1) mRNA |
| ATACTAAAAA | 12 | 33 | Human cysteine protease CPP32 isoform alpha mRNA, complete cds |
| ATACTAAAAA | 12 | 33 | EST |
| GTTTATTAAA | 10 | 27 | PROTEIN-TYROSINE PHOSPHATASE ZETA PRECURSOR |
| AGAAATCAGT | 9 | 24 | No match |
| TTGGATATTA | 9 | 24 | Homo sapiens clone 23785 mRNA sequence |
| AATTGAGTAG | 9 | 24 | Human DNA sequence from PAC 257A7 on chromosome 6p24. Contains |
| | | | two unknown genes and ESTs, STSs and a GSS |
| TGAGTGCTGC | 9 | 24 | No match |
| GCAGTACAGT | 8 | 22 | No match |
| GAATTCAGGA | 7 | 19 | Homo sapiens mRNA for KIAA0679 protein, partial cds |
| GACTTCTTTA | 7 | 19 | No match |
| GAATTCAGGA | 7 | 19 | Homo sapiens melastatin 1 (MLSN1) mRNA, complete cds |
| GTTTATACTG | 7 | 19 | No match |
| GAATTCAGGA | 7 | 19 | Homo sapiens mRNA for synaptosome associated protein of 23 |
| | | | kilodaltons, isoform A |
| GCCCGTGTAG | 6 | 16 | Msh (Drosophila) homeo box homolog 1 (formerly homeo box 7) |
| TGGGGTGTGC | 6 | 16 | Homo sapiens thyroid receptor interactor (TRIP8) mRNA, 3′ end of cds |
| AATTTTTATG | 5 | 14 | Interferon regulatory factor 4 |
| TCAGTGTCTG | 5 | 14 | ESTs |
| GGAGGTCAGC | 5 | 14 | ESTs |
| TTCTTCTCAA | 5 | 14 | ESTs |
| TTCTTCTCAA | 5 | 14 | ESTs |
| GGTTGTCTCT | 5 | 14 | ESTs, Weakly similar to line-1 protein ORF2 [H. sapiens] |
| CTTTGTTTAC | 5 | 14 | No match |
| CACTATAGAA | 5 | 14 | No match |
| TTTGGTTACA | 4 | 11 | EST |
| TCAAAACAAT | 4 | 11 | Human R kappa B mRNA, complete cds |
| TTTGGTTACA | 4 | 11 | Homo sapiens clone 23688 mRNA sequence |
| TATAGAGCAA | 4 | 11 | No match |
| TAATAACCAG | 4 | 11 | No match |
| TTCTATACTG | 4 | 11 | No match |
| GGAATACGGC | 4 | 11 | No match |
| Prostate (0.05%) | | | |
| TGAACTGGCA | 3 | 9 | No match |
| AATGTTGGGG | 3 | 9 | No match |
| Normal Kidney | | | |
| (0.27%) | | | |
| CGACAAACTA | 4 | 12 | No match |
| GTAGCACAGA | 4 | 12 | No match |
| ACCGTCAATC | 4 | 12 | No match |
| TGGATCAGTC | 4 | 12 | Human mRNA for KIAA0259 gene, partial cds |
| TGGCTCGGTC | 4 | 12 | EST |
| GCGACTGCGA | 4 | 12 | No match |
| GCACTAGCTG | 3 | 9 | No match |
| GCGGCCGGTT | 3 | 9 | No match |
| CGGCAGTCCC | 3 | 9 | No match |
| GCCCACCTGT | 3 | 9 | No match |
| CGGCGGATGG | 3 | 9 | No match |
| CCCCAGGCCG | 3 | 9 | No match |
| CCCATTCCAA | 3 | 9 | No match |
| TCAAGAGGTG | 3 | 9 | No match |

TABLE 6
Ubiquitously expressed transcripts

| | | | | |
|---|---|---|---|---|
| | Copies/ | | Range/ | |
| Tag sequence | cell | Range | Avg | Unigene Description |

| | | | | |
|---|---|---|---|---|
| CATCTAAACT | 44 | 22-62 | 0.91 | Human mRNA for KIAA0038 gene, partial cds |
| GGGCAAGCCA | 27 | 14-40 | 1.00 | STEROID HORMONE RECEPTOR ERR1 |
| ATTCAGCACC | 29 | 11-40 | 1.03 | ESTs, Highly similar to signal peptidase:SUBUNIT = 12 kD |
| TTGTTATTGC | 15 | 6-21 | 1.04 | Annexin VII (synexin) |
| ACAGGGTGAC | 115 | 47-165 | 1.04 | Homo sapiens mRNA for EDF-1 protein |
| GCTTCCATCT | 39 | 17-58 | 1.06 | H. sapiens BAT1 mRNA for nuclear RNA helicase (DEAD |
| | | | | family) |
| GCTTCCATCT | 39 | 17-58 | 1.06 | BB1 = malignant cell expression-enhanced gene/tumor |
| | | | | progression-enhanced gene |
| GAGGGTGGCG | 21 | 9-32 | 1.08 | Human DR-nm23 mRNA, complete cds |
| GCAGGGTGGG | 34 | 15-53 | 1.10 | V-akt murine thymoma viral oncogene homolog 2 |
| AGCCCTCCCT | 85 | 42-136 | 1.12 | Homo sapiens autoantigen p542 mRNA, complete cds |
| ATGGCCATAG | 15 | 5-22 | 1.12 | Human mRNA for YSK1, complete cds |
| GTGGGTGTCC | 20 | 9-32 | 1.13 | ESTs |
| TGTAGTTTGA | 41 | 14-62 | 1.14 | Transcription elongation factor B (SIII), polypeptide 1-like |
| GGGGCTGTGG | 14 | 6-21 | 1.15 | Human TFIIIC Box B-binding subunit mRNA, complete cds |
| GGGGCTGTGG | 14 | 6-21 | 1.15 | Homo sapiens mRNA for smallest subunit of ubiquinol- |
| | | | | cytochrome c reductase, complete cds |
| CACGCAATGC | 111 | 53-182 | 1.17 | Human homolog of Drosophila enhancer of split m9/m10 |
| | | | | mRNA, complete cds |
| CTCACACATT | 49 | 20-78 | 1.18 | LYSOSOME-ASSOCIATED MEMBRANE |
| | | | | GLYCOPROTEIN 1 PRECURSOR |
| CAAATGAGGA | 36 | 15-58 | 1.19 | Neuroblastoma RAS viral (v-ras) oncogene homolog |
| TGTAAGTCTG | 21 | 8-33 | 1.19 | Human p62 mRNA, complete cds |
| ACCAAGGAGG | 63 | 25-100 | 1.19 | ESTs |
| ACCAAGGAGG | 63 | 25-100 | 1.19 | DNA-DIRECTED RNA POLYMERASE II 23 KD |
| | | | | POLYPEPTIDE |
| ACCAAGGAGG | 63 | 25-100 | 1.19 | Human mRNA for transcription elongation factor S-II, hS- |
| | | | | II-T1, complete cds |
| TGAGGCAGGG | 17 | 7-27 | 1.20 | Syntaxin 5A |
| TCCACGCACC | 39 | 14-61 | 1.20 | ESTs |
| TAGGGCAATC | 40 | 14-62 | 1.21 | H. sapiens mRNA for SMT3B protein |
| GGTAGCCTGG | 61 | 25-98 | 1.21 | Damage-specific DNA binding protein 1 (127 kD) |
| TCAACAGCCA | 14 | 6-23 | 1.21 | Human translation initiation factor 3 47 kDa subunit |
| | | | | mRNA, complete cds |
| CTCTGTGTGG | 18 | 7-29 | 1.21 | Homo sapiens EB1 mRNA, complete cds |
| CCTATTTACT | 115 | 51-193 | 1.23 | Cytochrome c oxidase subunit IV |
| TGCATCTGGT | 104 | 32-162 | 1.24 | 78 KD GLUCOSE REGULATED PROTEIN PRECURSOR |
| GCTCTCTATG | 72 | 21-111 | 1.25 | H. sapiens mRNA for rat translocon-associated protein |
| | | | | delta homolog |
| GAAGGCATCC | 39 | 16-64 | 1.25 | PROBABLE 26S PROTEASE SUBUNIT TBP-1 |
| CCACTCCTCA | 59 | 19-93 | 1.26 | DEFENDER AGAINST CELL DEATH 1 |
| GCTGTCATCA | 31 | 8-47 | 1.27 | 26S PROTEASE REGULATORY SUBUNIT 4 |
| CGGCTGGTGA | 63 | 24-105 | 1.28 | Proteasome component C5 |
| AAGCCAGGAC | 65 | 26-110 | 1.31 | Homo sapiens chromosome 19, cosmid R32469 |
| TGAGAGGGTG | 32 | 15-57 | 1.32 | 14-3-3 PROTEIN TAU |
| GCGTGATCCT | 33 | 10-54 | 1.32 | ALCOHOL DEHYDROGENASE |
| CTGCCAACTT | 51 | 11-78 | 1.33 | COFILIN, NON-MUSCLE ISOFORM |
| CCAAACGTGT | 148 | 56-254 | 1.33 | HISTONE H3.3 |
| GCGGGAGGGC | 45 | 12-72 | 1.34 | ADP-RIBOSYLATION FACTOR-LIKE PROTEIN 2 |
| GGCCAGCCCT | 70 | 20-114 | 1.34 | ESTs |
| GGCCAGCCCT | 70 | 20-114 | 1.34 | Phosphofructokinase (liver type) |
| TGGGCAAAGC | 608 | 189-1014 | 1.36 | Translation elongation factor 1 gamma |
| GCAAAACCAG | 29 | 12-52 | 1.36 | Human mRNA for KIAA0002 gene, complete cds |
| ACTTACCTGC | 107 | 33-179 | 1.36 | Cytochrome c oxidase subunit VIb |
| GTTGGTCTGT | 32 | 11-54 | 1.36 | ESTs |
| TGCTACTGGT | 18 | 7-32 | 1.36 | Surfeit 1 |
| GACGACACGA | 401 | 71-618 | 1.37 | Ribosomal protein S28 |
| CAAGTGGCAA | 18 | 5-31 | 1.37 | Homo sapiens Grf40 adaptor protein (Grf40) mRNA, |
| | | | | complete cds |
| TACTCTTGGC | 72 | 16-114 | 1.37 | HETEROGENEOUS NUCLEAR RIBONUCLEOPROTEIN L |
| GACTGTGCCA | 75 | 15-118 | 1.37 | Human cytoplasmic dynein light chain 1 (hdlc1) mRNA, |
| | | | | complete cds |
| TTGCCGGTTA | 19 | 9-34 | 1.37 | Homo sapiens clone 24592 mRNA sequence |
| CATTGCAGGA | 14 | 5-25 | 1.38 | Homo sapiens Chromosome 16 BAC clone CIT987SK-A- |
| | | | | 152E5 |
| CAGGAACGGG | 97 | 26-159 | 1.38 | DUAL SPECIFICITY MITOGEN-ACTIVATED PROTEIN |
| | | | | KINASE KINASE 2 |
| AATAGGTCCA | 219 | 64-371 | 1.40 | Ribosomal protein S25 |
| ACCTCAGGAA | 67 | 32-126 | 1.41 | Human high density lipoprotein binding protein (HBP) |
| | | | | mRNA, complete cds |
| ATGACTCAAG | 26 | 12-48 | 1.41 | Human mRNA for protein tyrosine phosphatase (PTP- |
| | | | | BAS, type 2), complete cds |
| ATGACTCAAG | 26 | 12-48 | 1.41 | Homo sapiens mRNA, chromosome 1 specific transcript |
| | | | | KIAA0488 |
| GCCTCTGCCA | 26 | 12-48 | 1.41 | Human mRNA for KIAA0272 gene, partial cds |
| TGCTTGTCCC | 62 | 25-112 | 1.42 | ADP-ribosylation factor 1 |
| GGTGGCACTC | 112 | 41-199 | 1.42 | Aplysia ras-related homolog 12 |
| GGGCTGGGGT | 659 | 168-1102 | 1.42 | H. sapiens mRNA for ribosomal protein L29 |
| GGGCTGGGGT | 659 | 168-1102 | 1.42 | Homo sapiens sperm acrosomal protein mRNA, complete |
| | | | | cds |
| CACAAACGGT | 844 | 252-1449 | 1.42 | 40S RIBOSOMAL PROTEIN S27 |
| CATTGAAGGG | 37 | 13-66 | 1.42 | Homo sapiens clone 24433 myelodysplasia/myeloid |
| | | | | leukemia factor 2 mRNA, complete cds |
| GTGACTGCCA | 38 | 15-69 | 1.42 | DPH2L = candidate tumor suppressor gene {ovarian cancer |
| | | | | critical region of deletion} |
| GTGACTGCCA | 38 | 15-69 | 1.42 | Homo sapiens clone 24722 unknown mRNA, partial cds |
| AAGACAGTGG | 678 | 222-1190 | 1.43 | Ribosomal protein L37a |
| CTGGCTGCAA | 86 | 24-147 | 1.43 | Cytochrome c oxidase subunit Vb |
| ACCGGGAGGT | 18 | 5-30 | 1.43 | Human DNA from chromosome 19-specific cosmid |
| | | | | R27090, genomic sequence |
| ATGGAGACTT | 26 | 8-46 | 1.43 | Homo sapiens citrate synthase mRNA, complete cds |
| CAGCTCATCT | 40 | 17-74 | 1.44 | Homo sapiens hJTB mRNA, complete cds |
| ACGTGGTGAT | 52 | 6-81 | 1.44 | ESTs, Highly similar to LEYDIG CELL TUMOR 10 KD |
| | | | | PROTEIN [Rattus norvegicus] |
| GCGGTGAGGT | 37 | 9-62 | 1.44 | Homo sapiens small glutamine-rich tetratricopeptide |
| | | | | repeat (TPR) containing protein |
| GTGGCACACG | 105 | 24-176 | 1.44 | Eukaryotic translation initiation factor 3 (eIF-3) p36 subunit |
| GTGACAACAC | 42 | 11-71 | 1.45 | Voltage-dependent anion channel 1 |
| CTGCTATACG | 226 | 70-396 | 1.45 | Ribosomal protein L5 |
| ACTGGCTGCT | 27 | 10-50 | 1.46 | ESTs |
| GGAAGCACGG | 53 | 16-93 | 1.46 | Human antisecretory factor-1 mRNA, complete cds |
| GGAAGCACGG | 53 | 16-93 | 1.46 | Tag matches ribosomal RNA sequence |
| CTGTTGGTGA | 295 | 86-516 | 1.46 | 40S RIBOSOMAL PROTEIN S23 |
| TCAGATCTTT | 358 | 141-663 | 1.46 | Ribosomal protein S4, X-linked |
| TGGAATGCTG | 78 | 37-151 | 1.46 | Homo sapiens NADH:ubiquinone dehydrogenase 51 kDa |
| | | | | subunit (NDUFV1) mRNA, nuclear gene encoding |
| | | | | mitochondrial protein, complete cds |
| TAAGGAGCTG | 289 | 71-493 | 1.46 | Ribosomal protein S26 |
| GGCTTTGGAG | 41 | 15-75 | 1.46 | ESTs |
| CGCACCATTG | 41 | 14-74 | 1.46 | GCN5-like 1 = GCN5 homolog/putative regulator of |
| | | | | transcriptional activation {clone GCN5L1} |
| CGCTGGTTCC | 443 | 177-825 | 1.46 | Homo sapiens ribosomal protein L11 mRNA, complete cds |
| GGGCCTGGGG | 62 | 13-105 | 1.46 | ESTs |
| CTCGAGGAGG | 43 | 10-73 | 1.47 | Human ribosomal protein L23-related mRNA, complete |
| | | | | cds |
| TTGGTCCTCT | 1233 | 363-2177 | 1.47 | 60S RIBOSOMAL PROTEIN L41 |
| TCCCTGGCAT | 15 | 5-27 | 1.47 | Heterogeneous nuclear ribonucleoprotein K |
| GGGGGCTGCT | 11 | 6-23 | 1.47 | ESTs |
| GGGGGCTGCT | 11 | 6-23 | 1.47 | Human lysyl oxidase-related protein (WS9-14) mRNA, |
| | | | | complete cds |
| CCACCCCGAA | 109 | 14-174 | 1.48 | Testis enhanced gene transcript |
| CTGCTAGGAA | 21 | 9-40 | 1.48 | H. sapiens mRNA for TRAMP protein |
| AACTGCGGCA | 15 | 7-29 | 1.48 | ESTs |
| TGGAGTGGAG | 134 | 56-254 | 1.48 | Human guanylate kinase (GUK1) mRNA, complete cds |
| TGAAGGAGCC | 107 | 33-191 | 1.48 | ATP SYNTHASE LIPID-BINDING PROTEIN P2 |
| | | | | PRECURSOR |
| GGGGACTGAA | 77 | 24-138 | 1.48 | Homo sapiens mRNA for low molecular mass ubiquinone- |
| | | | | binding protein, complete cds |
| TGCACGTTTT | 526 | 196-979 | 1.49 | Human mRNA for antileukoprotease (ALP) from cervix |
| | | | | uterus |
| CTGGATGCCG | 33 | 11-59 | 1.49 | Radin blood group |
| CCCCCTCGTG | 24 | 8-44 | 1.49 | Adrenergic, beta, receptor kinase 1 |
| ATGATGCGGT | 41 | 13-74 | 1.49 | Cytoplasmic antiproteinase = 38 kda intracellular serine |
| | | | | proteinase inhibitor |
| ATTCTCCAGT | 356 | 86-618 | 1.50 | Ribosomal protein L17 |
| CCCCAGTTGC | 219 | 90-418 | 1.50 | Calpain, small polypeptide |
| CCAAGGATTG | 21 | 6-38 | 1.50 | Solute carrier family 5 (sodium/glucose cotransporter), |
| | | | | member 2 |
| GACCGAGGTG | 25 | 6-43 | 1.50 | Ewing sarcoma breakpoint region 1 |
| GACTCTCTCA | 13 | 5-25 | 1.50 | ESTs |
| GACTCTGGGA | 21 | 6-37 | 1.51 | ESTs, Moderately similar to T13H5.2 [C. elegans] |
| GACTCTGGGA | 21 | 6-37 | 1.51 | Actin, gamma 1 |
| CGCCGCGGTG | 207 | 54-368 | 1.51 | Homo sapiens Chromosome 16 BAC clone CIT987SK-A- |
| | | | | 761H5 |
| CCAGAACAGA | 361 | 119-666 | 1.52 | 60S RIBOSOMAL PROTEIN L30 |
| CCAGAACAGA | 361 | 119-666 | 1.52 | Deoxythymidylate kinase |
| TGGTTTTTGG | 26 | 5-43 | 1.52 | Homo sapiens acyl-protein thioesterase mRNA, complete |
| | | | | cds |
| TTTTTGTACA | 38 | 13-71 | 1.52 | ER LUMEN PROTEIN RETAINING RECEPTOR 1 |
| GTTCTCCCAC | 65 | 24-122 | 1.52 | ESTs, Highly similar to PROTEIN TRANSPORT |
| | | | | PROTEIN SEC61 ALPHA SUBUNIT |
| GACCCTGCCC | 192 | 30-323 | 1.52 | Human FK-506 binding protein homologue (FKBP38) |
| | | | | mRNA, complete cds |
| GCCCGCCTTG | 49 | 16-91 | 1.52 | Homo sapiens (clone mf.18) RNA polymerase II mRNA, |
| | | | | complete cds |
| GGTGCTGGAG | 24 | 8-45 | 1.53 | Homo sapiens mRNA for putative methyltransferase |
| TTACCTCCTT | 78 | 21-141 | 1.53 | Homo sapiens 3-phosphoglycerate dehydrogenase |
| | | | | mRNA, complete cds |
| AAACCAGGGC | 18 | 5-33 | 1.53 | ESTs |
| TTCTGGCTGC | 85 | 11-141 | 1.53 | Ubiquinol-cytochrome c reductase core protein I |
| TTCTGGCTGC | 85 | 11-141 | 1.53 | Human BAC clone RG114A06 from 7q31 |
| CTTCTCACCG | 33 | 8-58 | 1.54 | Ubiquitin-conjugating enzyme E2I (homologous to yeast |
| | | | | UBC9) |
| GAGAACCGTA | 48 | 13-87 | 1.54 | ESTs, Moderately similar to regulatory protein |
| GCGACCGTCA | 658 | 51-1076 | 1.56 | Aldolase A |
| GTCAAGACCA | 28 | 11-54 | 1.56 | Adaptin, beta 1 (beta prime) |
| CTGGGTCTCC | 42 | 12-78 | 1.56 | 60S RIBOSOMAL PROTEIN L13 |
| CGATTCTGGA | 27 | 11-53 | 1.56 | H. sapiens mRNA for ras-related GTP-binding protein |
| CAGGAGGAGT | 73 | 19-132 | 1.56 | PROBABLE PROTEIN DISULFIDE ISOMERASE ER-60 |
| | | | | PRECURSOR |
| CAAAATCAGG | 44 | 12-81 | 1.56 | Human mRNA for cyclin I, complete cds |
| CTGGGTTAAT | 615 | 116-1081 | 1.57 | 40S RIBOSOMAL PROTEIN S19 |
| TTTTCTGCTG | 34 | 6-60 | 1.57 | Hydroxyacyl-Coenzyme A dehydrogenase/3-ketoacyl- |
| | | | | Coenzyme A thiolase/enoyl-Coenzyme A hydratase |
| | | | | (trifunctional protein), beta subunit |
| CCCTGGCAAT | 30 | 14-61 | 1.57 | ESTs |
| AGGCTACGGA | 807 | 199-1472 | 1.58 | 60S RIBOSOMAL PROTEIN L13A |
| GAGGCCATCC | 23 | 8-45 | 1.58 | Homo sapiens chromosome 19, cosmid R30783 |
| CTTTGATGTT | 26 | 11-52 | 1.58 | Homo sapiens mRNA for NORI-1, complete cds |
| TTGGACCTGG | 113 | 29-206 | 1.58 | ESTs, Weakly similar to MALONYL COA-ACYL CARRIER |
| | | | | PROTEIN TRANSACYLASE [E. coli] |
| TTGGACCTGG | 113 | 29-206 | 1.58 | ATP synthase, H+ transporting, mitochondrial F1 complex, |
| | | | | delta subunit |
| GTTCGTGCCA | 213 | 43-379 | 1.58 | Ribosomal protein L35a |
| GATGCTGCCA | 154 | 34-277 | 1.58 | Human mRNA for Epstein-Barr virus small RNAs |
| | | | | (EBERs)associated protein (EAP) |
| ACGGCTCCGA | 27 | 8-50 | 1.58 | ESTs |
| GAGTCAGGAG | 29 | 6-53 | 1.59 | ESTs, Highly similar to COATOMER ZETA SUBUNIT |
| | | | | [Bos taurus] |
| GGAGGCTGAG | 84 | 37-171 | 1.59 | Homo sapiens mRNA for KIAA0792 protein, complete cds |
| GGAGGCTGAG | 84 | 37-171 | 1.59 | Homo sapiens putative fatty acid desaturase MLD mRNA, |
| | | | | complete cds |
| GTGATGGTGT | 75 | 24-143 | 1.59 | Thyroid autoantigen 70 kD (Ku antigen) |
| TCAGATGGCG | 45 | 6-78 | 1.59 | Homo sapiens hD54 + ins2 isoform (hD54) mRNA, |
| | | | | complete cds |
| ATGCGAAAGG | 32 | 9-59 | 1.59 | Dodecenoyl-Coenzyme A delta isomerase (3,2 trans- |
| | | | | enoyl-Coenzyme A isomerase) |
| TGCTGGGTGG | 67 | 26-133 | 1.60 | ESTs, Highly similar to NADH-UBIQUINONE |
| | | | | OXIDOREDUCTASE ASHI SUBUNIT PRECURSOR [Bos |
| | | | | taurus] |
| TGCTGGGTGG | 67 | 26-133 | 1.60 | Homo sapiens folylpolyglutamate synthetase mRNA, |
| | | | | complete cds |
| TCAAATGCAT | 37 | 9-68 | 1.60 | HETEROGENEOUS NUCLEAR |
| | | | | RIBONUCLEOPROTEINS C1/C2 |
| TCCAAGGAAG | 13 | 5-26 | 1.60 | Homo sapiens DBI-related protein mRNA, complete cds |
| CCCAGGGAGA | 49 | 11-90 | 1.60 | Homo sapiens chaperonin containing t-complex |
| | | | | polypeptide 1, delta subunit (Cctd) mRNA, complete cds |
| TGGCCTGCCC | 54 | 15-102 | 1.60 | ESTs |
| TGGCCTGCCC | 54 | 15-102 | 1.60 | ESTs, Moderately similar to PEANUT PROTEIN |
| | | | | [Drosophila melanogaster] |
| GGCCAAAGGC | 39 | 14-77 | 1.60 | Human mRNA for KIAA0064 gene, complete cds |
| GGCCTGCTGC | 69 | 13-125 | 1.60 | ESTs, Highly similar to C10 [H. sapiens] |
| GTGAAGCTGA | 22 | 7-41 | 1.61 | ESTs, Highly similar to HYPOTHETICAL 6.3 KD |
| | | | | PROTEIN ZK652.2 IN CHROMOSOME III [Caenorhabditis |
| | | | | elegans] |
| GTGAAGCTGA | 22 | 7-41 | 1.61 | ESTs, Highly similar to thymic epithelial cell surface |
| | | | | antigen [M. musculus] |
| GAAATGTAAG | 50 | 12-93 | 1.62 | ESTs |
| GAAATGTAAG | 50 | 12-93 | 1.62 | H. sapiens hnRNP-E2 mRNA |
| CGTGTTAATG | 73 | 31-148 | 1.62 | CELLULAR NUCLEIC ACID BINDING PROTEIN |
| AGGGGATTCC | 19 | 9-40 | 1.62 | Human arginine-rich protein (ARP) gene, complete cds |
| CAGCTCACTG | 186 | 23-326 | 1.63 | Homo sapiens CAG-isl 7 mRNA, complete cds |
| GTTTGGCAGT | 35 | 13-70 | 1.63 | Homo sapiens mRNA for EDF-1 protein |
| GGAGCTCTGT | 48 | 13-92 | 1.63 | ESTs, Moderately similar to NADH-UBIQUINONE |
| | | | | OXIDOREDUCTASE B15 SUBUNIT [Bos taurus] |
| TGGAACTGTG | 22 | 5-42 | 1.63 | ESTs, Weakly similar to !!!! ALU SUBFAMILY SQ |
| | | | | WARNING ENTRY !!!! [H. sapiens] |
| TCTGCTTACA | 58 | 18-114 | 1.63 | Human ribosomal protein L10 mRNA, complete cds |
| AGGGCTTCCA | 643 | 205-1257 | 1.64 | UBIQUINOL-CYTOCHROME C REDUCTASE COMPLEX |
| | | | | SUBUNIT VI REQUIRING PROTEIN |
| GAGCAAACGG | 20 | 5-37 | 1.64 | Homo sapiens chromosome 19, cosmid R26445 |
| TGTGATCAGA | 88 | 27-171 | 1.64 | Homo sapiens F1F0-type ATP synthase subunit g mRNA, |
| | | | | complete cds |
| ACACTACGGG | 37 | 6-66 | 1.64 | ESTs, Weakly similar to putative progesterone binding |
| | | | | protein [H. sapiens] |
| AGCCAAAAAA | 41 | 12-79 | 1.64 | H. sapiens hnRNP-E2 mRNA |
| GCGGGTGTGG | 16 | 5-32 | 1.64 | Human methionine aminopeptidase mRNA, complete cds |
| TTGCTAGAGG | 39 | 13-78 | 1.65 | ESTs, Weakly similar to F35H10.6 gene product |
| | | | | [C. elegans] |
| GGGGCTTCTG | 15 | 6-30 | 1.65 | Human mRNA for cysteine protease, complete cds |
| AACTCTTGAA | 45 | 14-87 | 1.65 | Human translation initiation factor eIF3 p40 subunit mRNA, |
| | | | | complete cds |
| GTCTGACCCC | 44 | 8-80 | 1.65 | PROTEIN PHOSPHATASE PP2A, 65 KD REGULATORY |
| | | | | SUBUNIT, ALPHA ISOFORM |
| ATGTCATCAA | 48 | 12-92 | 1.65 | Human clathrin assembly protein 50 (AP50) mRNA, |
| | | | | complete cds |
| TCTGTCAAGA | 40 | 15-81 | 1.66 | ATP synthase, H+ transporting, mitochondrial F1 complex, |
| | | | | O subunit (oligomycin sensitivity conferring protein) |
| GCCCCAGCGA | 23 | 8-46 | 1.66 | ESTs |
| GGCAAGCCCC | 425 | 119-824 | 1.66 | Heat shock 27 kD protein 1 |
| CTCATCAGCT | 48 | 16-95 | 1.66 | ADENYLYL CYCLASE-ASSOCIATED PROTEIN 1 |
| CTGTTGATTG | 137 | 49-276 | 1.66 | Heterogeneous nuclear ribonucleoprotein A1 |
| GCTTTTAAGG | 171 | 27-312 | 1.66 | 40S RIBOSOMAL PROTEIN S20 |
| GCCTGAGCCT | 13 | 6-28 | 1.66 | ESTs |
| GAGCGGGATG | 57 | 21-116 | 1.66 | Proteasome (prosome, macropain) subunit, beta type, 6 |
| TTCACAGTGG | 56 | 13-107 | 1.67 | Calcineurin B |
| GCCCGTGCCA | 23 | 8-46 | 1.67 | ESTs, Highly similar to HYPOTHETICAL 38.2 KD |
| | | | | PROTEIN IN BEM2-SPT2 INTERGENIC REGION |
| | | | | [Saccharomyces cerevisiae] |
| CCCTAGGTTG | 51 | 14-98 | 1.67 | Human mRNA for KIAA0315 gene, partial cds |
| CCCTGATTTT | 33 | 12-66 | 1.67 | Human p97 mRNA, complete cds |
| GTGTTAACCA | 314 | 73-599 | 1.67 | Human ribosomal protein L10 mRNA, complete cds |
| AGGAAAGCTG | 469 | 162-948 | 1.68 | ESTs, Highly similar to 60S RIBOSOMAL PROTEIN L36 |
| | | | | [Rattus norvegicus] |
| TTCTCTCTGT | 31 | 8-60 | 1.68 | ADP-ribosylation factor 5 |
| TTACTAAATG | 26 | 5-48 | 1.68 | Calnexin |
| GGGTGTGGTG | 18 | 5-36 | 1.68 | ESTs |
| CCACTGCAGT | 14 | 5-29 | 1.68 | GLYCOPROTEIN HORMONES ALPHA CHAIN |
| | | | | PRECURSOR |
| AGCCTGGACT | 47 | 17-95 | 1.69 | Human mRNA for Mr 110,000 antigen, complete cds |
| GTGGGGTGAC | 24 | 6-47 | 1.69 | ESTs, Weakly similar to HYPOTHETICAL 21.5 KD |
| | | | | PROTEIN IN SEC15-SAP4 INTERGENIC REGION |
| | | | | [S. cerevisiae] |
| CACTACACGG | 46 | 11-88 | 1.69 | FK506-BINDING PROTEIN PRECURSOR |
| CTCATAGCAG | 92 | 31-187 | 1.69 | TRANSLATIONALLY CONTROLLED TUMOR PROTEIN |
| GGAATGTACG | 94 | 27-187 | 1.70 | Human mitochondrial ATP synthase subunit 9, P3 gene |
| | | | | copy, mRNA, nuclear gene encoding mitochondrial |
| | | | | protein, complete cds |
| CTGAGGGTGG | 17 | 8-36 | 1.70 | ESTs |
| AAGGTCGAGC | 75 | 9-136 | 1.70 | 60S RIBOSOMAL PROTEIN L24 |
| GAATCACTGC | 18 | 5-35 | 1.70 | Homo sapiens ribosomal protein L33-like protein mRNA, |
| | | | | complete cds |
| ACATCATCGA | 374 | 86-722 | 1.70 | Ribosomal protein L12 |
| GAATGAGGAC | 27 | 6-51 | 1.70 | Human mRNA for reticulocalbin, complete cds |
| CCTCGCTCAG | 44 | 14-89 | 1.70 | Hydroxyacyl-Coenzyme A dehydrogenase/3-ketoacyl- |
| | | | | Coenzyme A thiolase/enoyl-Coenzyme A hydratase |
| | | | | (trifunctional protein), alpha subunit |
| TCCTAGCCTG | 16 | 5-33 | 1.70 | Homo sapiens SPF31 (SPF31) mRNA, complete cds |
| AGGTGCGGGG | 35 | 5-64 | 1.71 | Human hASNA-I mRNA, complete cds |
| CTCCAATAAA | 14 | 7-31 | 1.71 | Homo sapiens clone 24775 mRNA sequence |
| GCGCTGGAGT | 73 | 23-147 | 1.71 | ESTs, Weakly similar to HYPOTHETICAL 9.9 KD |
| | | | | PROTEIN B0495.6 IN CHROMOSOME II [C. elegans] |
| AATTTGCAAC | 21 | 5-40 | 1.71 | Homo sapiens histone macroH2A1.2 mRNA, complete cds |
| AACGCGGCCA | 448 | 22-790 | 1.71 | Macrophage migration inhibitory factor |
| GGTGTATATG | 21 | 7-42 | 1.71 | Homo sapiens chromosome 9, P1 clone 11659 |
| GGCAACAAAA | 35 | 6-66 | 1.71 | Human (clone E5.1) RNA-binding protein mRNA, complete |
| | | | | cds |
| GGCAACAAAA | 35 | 6-66 | 1.71 | Homo sapiens importin beta subunit mRNA, complete cds |
| TTTGTGACTG | 28 | 13-62 | 1.71 | Homo sapiens phosphoprotein CtBP mRNA, complete cds |
| ATGAGGCCGG | 23 | 7-47 | 1.72 | No match |
| TCAGTTTGTC | 39 | 15-81 | 1.72 | Human HS1 binding protein HAX-1 mRNA, nuclear gene |
| | | | | encoding mitochondrial protein, complete cds |
| CCCTATTAAG | 69 | 10-129 | 1.72 | No match |
| TTTCTAGTTT | 55 | 28-123 | 1.72 | Human mRNA for KIAA0108 gene, complete cds |
| GGGCCCTTCC | 20 | 5-40 | 1.72 | Homo sapiens clone 24684 mRNA sequence |
| GGGCCCTTCC | 20 | 5-40 | 1.72 | Fibulin 1 |
| CCTTGGTTTT | 24 | 6-47 | 1.72 | Homo sapiens DNA-binding protein (CROC-1B) mRNA, |
| | | | | complete cds |
| GCTAAGGAGA | 81 | 21-161 | 1.72 | Human ras-related C3 botulinum toxin substrate (rac) |
| | | | | mRNA, complete cds |
| TGAGGGGTGA | 27 | 8-56 | 1.72 | Human Gps1 (GPS1) mRNA, complete cds |
| CCAGCTGCCA | 63 | 19-128 | 1.73 | Ubiquitin activating enzyme E1 |
| GGGCTGTTTG | 16 | 5-34 | 1.73 | No match |
| TGGACACAAG | 18 | 5-36 | 1.73 | Arginyl-tRNA synthetase |
| TCTCCAGGAA | 44 | 12-89 | 1.73 | ESTs, Weakly similar to PUTATIVE MITOCHONDRIAL |
| | | | | CARRIER C16C10.1 [C. elegans] |
| TGATGTTTGA | 24 | 8-49 | 1.73 | Human mRNA for KIAA0058 gene, complete cds |
| GTGGTGCACG | 82 | 13-155 | 1.73 | No match |
| GTCTGCACCT | 32 | 8-64 | 1.73 | ESTs, Weakly similar to NUCLEAR PROTEIN SNF7 |
| | | | | [Saccharomyces cerevisiae] |
| GATGACCCCG | 32 | 11-66 | 1.73 | ESTs, Weakly similar to F08G12.1 [C. elegans] |
| ATCAAGGGTG | 269 | 27-494 | 1.73 | Ribosomal protein L9 |
| TCTGGTCTGG | 34 | 12-72 | 1.74 | Human surface antigen mRNA, complete cds |
| AGGATGACCC | 42 | 6-79 | 1.74 | ESTs, Weakly similar to ion channel homolog RIC |
| | | | | [M. musculus] |
| AAAGGGGGCA | 28 | 9-58 | 1.74 | H. sapiens mRNA for activin beta-C chain |
| GGCTTTACCC | 178 | 56-365 | 1.74 | Eukaryotic translation initiation factor 5A |
| GCTTTTTAGA | 39 | 10-78 | 1.74 | Human non-histone chromosomal protein HMG-14 mRNA, |
| | | | | complete cds |
| CTCTGCTCGG | 18 | 6-37 | 1.74 | Homo sapiens clone 638 unknown mRNA, complete |
| | | | | sequence |
| GCCTGGGACT | 58 | 28-130 | 1.74 | ESTs |
| GGTAGCAGGG | 26 | 5-50 | 1.74 | Homo sapiens clone 23930 mRNA sequence |
| GCCGATCCTC | 31 | 7-61 | 1.74 | Homo sapiens cofactor A protein mRNA, complete cds |
| GCAGCTCAGG | 50 | 13-101 | 1.74 | Cathepsin D (lysosomal aspartyl protease) |
| CGCAGTGTCC | 118 | 20-225 | 1.75 | Vacuolar H+ ATPase proton channel subunit |
| CCCCTATTAA | 62 | 13-121 | 1.75 | No match |
| TTGTAAAAGG | 23 | 8-47 | 1.75 | Homo sapiens chromosome 9, P1 clone 11659 |
| CCACACCGGT | 17 | 6-36 | 1.75 | Heme oxygenase (decycling) 2 |
| CCTGGAAGAG | 192 | 60-396 | 1.75 | Procollagen-proline, 2-oxoglutarate 4-dioxygenase (proline |
| | | | | 4-hydroxylase), beta polypeptide (protein disulfide |
| | | | | isomerase; thyroid hormone binding protein p55) |
| TAGCCGCTGA | 37 | 7-72 | 1.75 | Homo sapiens alpha SNAP mRNA, complete cds |
| CCTAGGACCT | 19 | 5-39 | 1.75 | Homo sapiens Arp2/3 protein complex subunit p20-Arc |
| | | | | (ARC20) mRNA, complete cds |
| GTGGACCCTG | 26 | 9-54 | 1.75 | Surfeit 1 |
| GTGGACCCTG | 26 | 9-54 | 1.75 | ESTs, Weakly similar to R05G6.4 gene product |
| | | | | [C. elegans] |
| TTGGGAGCAG | 32 | 6-63 | 1.76 | Isoleucine-tRNA synthetase |
| GTCTCACGTG | 23 | 9-49 | 1.76 | ESTs |
| GTACTGTGGC | 114 | 24-225 | 1.76 | Homo sapiens nuclear chloride ion channel protein |
| | | | | (NCC27) mRNA, complete cds |
| AAGATAATGC | 12 | 5-27 | 1.76 | ESTs, Weakly similar to Yel007c-ap [S. cerevisiae] |
| AATACCTCGT | 31 | 7-61 | 1.76 | ESTs |
| ACCTTGTGCC | 23 | 6-47 | 1.76 | ESTs, Weakly similar to alpha 2,6-sialyltransferase |
| | | | | [R. norvegicus] |
| ACCTTGTGCC | 23 | 6-47 | 1.76 | Sorbitol dehydrogenase |
| GGAGGGGGCT | 88 | 16-172 | 1.77 | LAMIN A |
| GCCTATGGTC | 39 | 9-78 | 1.77 | ESTs, Highly similar to SEX-REGULATED PROTEIN |
| | | | | JANUS-A [Drosophila melanogaster] |
| GTGCTGAATG | 459 | 219-1031 | 1.77 | MYOSIN LIGHT CHAIN ALKALI, SMOOTH-MUSCLE |
| | | | | ISOFORM |
| TCGTCGCAGA | 37 | 9-75 | 1.77 | ESTs, Highly similar to NADH-UBIQUINONE |
| | | | | OXIDOREDUCTASE SUBUNIT B14.5A [Bos taurus] |
| GTGACAGAAG | 178 | 36-351 | 1.77 | Eukaryotic translation initiation factor 4A (eIF-4A) isoform 1 |
| TCAACGGTGT | 15 | 5-31 | 1.77 | Homo sapiens mRNA for RanBPM, complete cds |
| GAGCCTTGGT | 58 | 11-113 | 1.77 | Protein phosphatase 1, catalytic subunit, alpha isoform |
| TACATCCGAA | 19 | 6-40 | 1.78 | ESTs |
| GTCTGTGAGA | 29 | 12-64 | 1.78 | Homo sapiens mRNA for Hrs, complete cds |
| GTTAACGTCC | 95 | 18-187 | 1.78 | Homo sapiens Bruton's tyrosine kinase (BTK), alpha-D- |
| | | | | galactosidase A (GLA), L44-like ribosomal protein (L44L) |
| | | | | and FTP3 (FTP3) genes, complete cds |
| GTGCGCTAGG | 141 | 27-277 | 1.78 | ESTs, Weakly similar to F49C12.12 [C. elegans] |
| CGGATAAGGC | 17 | 6-36 | 1.78 | ESTs |
| GTCTGGGGCT | 204 | 49-413 | 1.78 | SM22-ALPHA HOMOLOG |
| CATCCTGCTG | 64 | 12-125 | 1.78 | Human mRNA for 26S proteasome subunit p97, complete |
| | | | | cds |
| TCACAAGCAA | 142 | 52-305 | 1.78 | H. sapiens alpha NAC mRNA |
| GGCTGATGTG | 73 | 15-146 | 1.78 | Glycyl-tRNA synthetase |
| CCCGTCCGGA | 1272 | 293-2564 | 1.78 | 60S RIBOSOMAL PROTEIN L13 |
| TCCGCGAGAA | 98 | 33-208 | 1.78 | ESTs, Weakly similar to SEX-DETERMINING |
| | | | | TRANSFORMER PROTEIN 1 [Caenorhabditis elegans] |
| GTGCTGGAGA | 98 | 12-187 | 1.79 | Human SnRNP core protein Sm D2 mRNA, complete cds |
| TCCTCAAGAT | 26 | 8-54 | 1.79 | Human enhancer of rudimentary homolog mRNA, |
| | | | | complete cds |
| CAACTTAGTT | 60 | 20-127 | 1.79 | Human myosin regulatory light chain mRNA, complete cds |
| GGGCAGCTGG | 35 | 12-75 | 1.79 | ESTs |
| TTTCAGAGAG | 43 | 8-84 | 1.79 | Human calmodulin mRNA, complete cds |
| TTTCAGAGAG | 43 | 8-84 | 1.79 | Signal recognition particle 9 kD protein |
| GACGCAGAAG | 17 | 6-36 | 1.79 | ESTs, Highly similar to ALPHA-ADAPTIN [Mus musculus] |
| GGAAGTTTCG | 35 | 9-72 | 1.79 | ESTs, Weakly similar to similar to oxysterol-binding |
| | | | | proteins: partial CDS [C. elegans] |
| GTTGCTGCCC | 34 | 5-65 | 1.79 | Homo sapiens mRNA for putative seven transmembrane |
| | | | | domain protein |
| GCTGGGGTGG | 21 | 6-44 | 1.79 | H. sapiens mRNA for mediator of receptor-induced toxicity |
| CTCAACATCT | 456 | 99-918 | 1.80 | Ribosomal protein, large, P0 |
| CAAGCAGGAC | 42 | 8-84 | 1.80 | ESTs, Weakly similar to transmembrane protein |
| | | | | [H. sapiens] |
| TTGGCTTTTC | 27 | 8-57 | 1.80 | ESTs |
| TGGCAACCTT | 38 | 17-85 | 1.80 | ESTs, Highly similar to GLUTATHIONE S- |
| | | | | TRANSFERASE, MITOCHONDRIAL [Rattus norvegicus] |
| GCATAATAGG | 391 | 83-786 | 1.80 | Ribosomal protein L21 |
| GGGGGTAACT | 43 | 9-86 | 1.80 | RNA-BINDING PROTEIN FUS/TLS |
| CCTTCGAGAT | 274 | 55-549 | 1.80 | Ribosomal protein S5 |
| CGGGCCGTGC | 18 | 6-38 | 1.80 | H. sapiens mRNA for Glyoxalase II |
| GTGTTGCACA | 210 | 42-421 | 1.80 | Ribosomal protein S13 |
| CCTCGGAAAA | 158 | 27-312 | 1.81 | 60S RIBOSOMAL PROTEIN L38 |
| AATAAAGGCT | 56 | 9-110 | 1.81 | Myosin, light polypeptide 3, alkali; ventricular, skeletal, |
| | | | | slow |
| AATAAAGGCT | 56 | 9-110 | 1.81 | Aplysia ras-related homolog 9 |
| CTTCTGTGTA | 21 | 9-47 | 1.81 | Homo sapiens immunophilin homolog ARA9 mRNA, |
| | | | | complete cds |
| CTTCTGTGTA | 21 | 9-47 | 1.81 | Human mRNA for KIAA0190 gene, partial cds |
| GGTCCAGTGT | 144 | 26-286 | 1.81 | Phosphoglycerate mutase 1 (brain) |
| AGCACCTCCA | 701 | 197-1467 | 1.81 | Eukaryotic translation elongation factor 2 |
| AAGCTGAGTG | 39 | 12-82 | 1.81 | Human M4 protein mRNA, complete cds |
| GTTTCTTCCC | 27 | 11-60 | 1.81 | ESTs |
| TGAGGGAATA | 191 | 51-397 | 1.82 | Triosephosphate isomerase 1 |
| AGCTCTCCCT | 447 | 150-962 | 1.82 | 60S RIBOSOMAL PROTEIN L23 |
| TACGTTGCAG | 18 | 8-40 | 1.82 | Homo sapiens GC20 protein mRNA, complete cds |
| GGGTGTGTAT | 16 | 6-35 | 1.82 | Homo sapiens angio-associated migratory cell protein |
| | | | | (AAMP) mRNA, complete cds |
| GGAGGGATCA | 37 | 12-79 | 1.82 | Homo sapiens integrin-linked kinase (ILK) mRNA, |
| | | | | complete cds |
| ATCAGTGGCT | 64 | 25-143 | 1.82 | PROTEASOME BETA CHAIN PRECURSOR |
| CCCCCTGCCC | 57 | 17-121 | 1.83 | ESTs |
| CCCCCTGCCC | 57 | 17-121 | 1.83 | ESTs |
| CAAAAAAAAA | 94 | 8-180 | 1.83 | Cholinergic receptor, nicotinic, alpha polypeptide 3 |
| ACCTGCCGAC | 18 | 5-37 | 1.83 | Homo sapiens growth suppressor related (DOC-1R) |
| | | | | mRNA, complete cds |
| GACCAGAAAA | 81 | 17-165 | 1.83 | CYTOCHROME C OXIDASE POLYPEPTIDE VIA-LIVER |
| | | | | PRECURSOR |
| AGCCACTGCG | 33 | 9-69 | 1.83 | No match |
| TTGAGCCAGC | 43 | 21-101 | 1.83 | Human KH type splicing regulatory protein KSRP mRNA, |
| | | | | complete cds |
| TTTCAGGGGA | 51 | 9-103 | 1.84 | ESTs, Moderately similar to N-methyl-D-aspartate receptor |
| | | | | glutamate-binding chain [R. norvegicus] |
| TCCGGCCGCG | 75 | 32-169 | 1.84 | ESTs |
| GTGATCTCCG | 22 | 6-46 | 1.84 | ESTs |
| CTGCTGAGTG | 46 | 6-90 | 1.84 | ESTs, Highly similar to HYPOTHETICAL 14.1 KD |
| | | | | PROTEIN C31A2.02 IN CHROMOSOME I |
| | | | | [Schizosaccharomyces pombe] |
| CTGCTTAAGG | 16 | 6-36 | 1.84 | ESTs, Highly similar to HYPOTHETICAL 68.7 KD |
| | | | | PROTEIN ZK757.1 IN CHROMOSOME III [Caenorhabditis |
| | | | | elegans] |
| TGTGGCCTCC | 33 | 14-74 | 1.84 | ESTs, Weakly similar to No definition line found |
| | | | | [C. elegans] |
| CGTTTTCTGA | 20 | 6-43 | 1.84 | Human protein-tyrosine phosphatase (HU-PP-1) mRNA, |
| | | | | partial sequence |
| GGAAAAAAAA | 97 | 8-187 | 1.84 | Hepatocyte growth factor (hepapoietin A; scatter factor) |
| GGAAAAAAAA | 97 | 8-187 | 1.84 | ESTs, Highly similar to ATP SYNTHASE EPSILON |
| | | | | CHAIN, MITOCHONDRIAL PRECURSOR [Bos taurus] |
| GAGGGAGTTT | 548 | 162-1172 | 1.84 | Ribosomal protein L27a |
| GACTCACTTT | 156 | 27-315 | 1.84 | Peptidylprolyl isomerase B (cyclophilin B) |
| GAGAACGGGG | 33 | 7-67 | 1.85 | ESTs, Highly similar to CORONIN [Dictyostelium |
| | | | | discoideum] |
| TGGCTAGTGT | 57 | 20-125 | 1.85 | Human mRNA for proteasome subunit z, complete cds |
| CTGTCATTTG | 20 | 5-42 | 1.85 | PRE-MRNA SPLICING FACTOR SRP20 |
| GTTCCCTGGC | 320 | 98-690 | 1.85 | Finkel-Biskis-Reilly murine sarcoma virus (FBR-MuSV) |
| | | | | ubiquitously expressed (fox derived) |
| GCATTTAAAT | 76 | 7-148 | 1.85 | ELONGATION FACTOR 1-BETA |
| ATCCACATCG | 69 | 17-144 | 1.85 | ESTs, Weakly similar to CASEIN KINASE I HOMOLOG |
| | | | | HRR25 [Saccharomyces cerevisiae] |
| CTGCTGTGAT | 29 | 6-59 | 1.85 | Human mRNA for U1 small nuclear RNP-specific C protein |
| GTGACCTCCT | 116 | 38-253 | 1.85 | CYTOCHROME C OXIDASE POLYPEPTIDE VIII- |
| | | | | LIVER/HEART PRECURSOR |
| GTGGACCCCA | 47 | 9-97 | 1.86 | Human siah binding protein 1 (SiahBP1) mRNA, partial |
| | | | | cds |
| GACTAGTGCG | 18 | 6-39 | 1.86 | ESTs |
| TTATGGGATC | 247 | 31-490 | 1.86 | GUANINE NUCLEOTIDE-BINDING PROTEIN BETA |
| | | | | SUBUNIT-LIKE PROTEIN 12.3 |
| TTTCAGATTG | 29 | 5-60 | 1.86 | Human transcriptional coactivator PC4 mRNA, complete |
| | | | | cds |
| GTCTGAGCTC | 58 | 14-122 | 1.86 | ESTs, Weakly similar to HYPOTHETICAL 15.4 KD |
| | | | | PROTEIN C16C10.11 IN CHROMOSOME III [C. elegans] |
| CACACAATGT | 22 | 9-49 | 1.86 | Homo sapiens peroxisomal phytanoyl-CoA alpha- |
| | | | | hydroxylase (PAHX) mRNA, complete cds |
| CACACAATGT | 22 | 9-49 | 1.86 | Cytochrome c oxidase subunit IV |
| ACCCCACCCA | 26 | 6-55 | 1.86 | H. sapiens mRNA for 1-acylglycerol-3-phosphate O- |
| | | | | acyltransferase |
| GGAGGCAGGT | 31 | 9-67 | 1.86 | Homo sapiens chromosome 1p33-p34 beta-1,4- |
| | | | | galactosyltransferase mRNA, complete cds |
| TCTCAATTCT | 27 | 8-58 | 1.87 | Cell division cycle 42 (GTP-binding protein, 25 kD) |
| CTCTTCAGGA | 19 | 6-40 | 1.87 | Homo sapiens phosphomevalonate kinase mRNA, |
| | | | | complete cds |
| CTGGGACTGC | 18 | 7-40 | 1.87 | Homo sapiens mRNA for follistain-related protein (FRP), |
| | | | | complete cds |
| GCCCAGCAGG | 26 | 8-57 | 1.87 | ESTs |
| GCCCAGCAGG | 26 | 8-57 | 1.87 | ESTs |
| GGGCCAGGGG | 44 | 16-98 | 1.87 | ESTs |
| GGGGGACGGC | 42 | 12-89 | 1.87 | ESTs, Weakly similar to Y48E1B.1 [C. elegans] |
| ACTGGGTCTA | 154 | 29-317 | 1.87 | Non-metastatic cells 2, protein (NM23B) expressed in |
| GCCGAGGAAG | 778 | 113-1570 | 1.87 | Human mRNA for ribosomal protein S12 |
| CAGATCTTTG | 90 | 14-182 | 1.88 | Ubiquitin A-52 residue ribosomal protein fusion product 1 |
| AGGTTTCCTC | 21 | 6-45 | 1.88 | Homo sapiens mRNA for proteasome subunit p58, |
| | | | | complete cds |
| CCGTCCAAGG | 532 | 59-1058 | 1.88 | Ribosomal protein S16 |
| GTGGCGGGCG | 81 | 21-174 | 1.88 | Biliary glycoprotein |
| GTGGCGGGCG | 81 | 21-174 | 1.88 | Homo sapiens malignancy-associated protein mRNA, |
| | | | | partial cds |
| GTGGCGGGCG | 81 | 21-174 | 1.88 | Homo sapiens mRNA for KIAA0565 protein, complete cds |
| GGCAAGAAGA | 252 | 34-507 | 1.88 | Ribosomal protein L27 |
| TCTTTACTTG | 23 | 6-49 | 1.88 | Homo sapiens Arp2/3 protein complex subunit p21-Arc |
| | | | | (ARC21) mRNA, complete cds |
| CTCCTCACCT | 255 | 56-536 | 1.88 | 60S RIBOSOMAL PROTEIN L13A |
| CTCCTCACCT | 255 | 56-536 | 1.88 | Human Bak mRNA, complete cds |
| GCCTGTATGA | 392 | 116-853 | 1.88 | Ribosomal protein S24 |
| GCTTTATTTG | 560 | 147-1203 | 1.88 | Human mRNA fragment encoding cytoplasmic actin. |
| | | | | (isolated from cultured epidermal cells grown from human |
| | | | | foreskin) |
| CTTAAGGATT | 27 | 9-60 | 1.88 | ESTs, Highly similar to transcription factor ARF6 chain B |
| | | | | [M. musculus] |
| GGATTTGGCC | 656 | 165-1401 | 1.88 | Ribosomal protein, large P2 |
| GGATTTGGCC | 656 | 165-1401 | 1.88 | Ribosomal protein S26 |
| GGATTTGGCC | 656 | 165-1401 | 1.88 | Human mRNA for PIG-B, complete cds |
| TCCTCCCTCC | 31 | 5-62 | 1.89 | Human mRNA for proteasome subunit HsC7-I, complete |
| | | | | cds |
| GGCCCTCTGA | 46 | 9-96 | 1.89 | Human peptidyl-prolyl isomerase and essential mitotic |
| | | | | regulator (PIN1) mRNA, complete cds |
| TGGCTGTGTG | 47 | 8-97 | 1.89 | ESTs |
| AGACCAAAGT | 38 | 6-79 | 1.89 | DNAJ PROTEIN HOMOLOG 1 |
| ATGGCCAACT | 28 | 12-64 | 1.89 | ESTs |
| AGGAGCTGCT | 81 | 12-165 | 1.89 | ESTs |
| AGGAGCTGCT | 81 | 12-165 | 1.89 | Human mitochondrial NADH dehydrogenase-ubiquinone |
| | | | | Fe—S protein 8, 23 kDa subunit precursor (NDUFS8) |
| | | | | nuclear mRNA encoding mitochondrial protein, complete |
| | | | | cds |
| TGTACCTGTA | 245 | 8-473 | 1.90 | Human alpha-tubulin mRNA, complete cds |
| GATCCCAACA | 70 | 11-143 | 1.90 | ATP synthase, H+ transporting, mitochondrial F1 complex, |
| | | | | beta polypeptide |
| GGCCATCTCT | 38 | 8-80 | 1.90 | 14-3-3 PROTEIN TAU |
| AGGTGCAGAG | 26 | 9-58 | 1.90 | Homo sapiens pescadillo mRNA, complete cds |
| GTGGCATCAC | 32 | 7-68 | 1.90 | ESTs, Weakly similar to C25A1.6 [C. elegans] |
| TGTGTTGAGA | 1663 | 321-3487 | 1.90 | Translation elongation factor 1-alpha-1 |
| CTGAGACAAA | 98 | 14-199 | 1.91 | Basic transcription factor 3 |
| GCAACGGGCC | 54 | 6-108 | 1.91 | Homo sapiens mRNA for brain acyl-CoA hydrolase, |
| | | | | complete cds |
| GCTGGCTGGC | 113 | 27-243 | 1.91 | Homo sapiens chaperonin containing t-complex |
| | | | | polypeptide 1, eta subunit (Ccth) mRNA, complete cds |
| GCCAAGATGC | 55 | 11-116 | 1.91 | ESTs |
| GCCAAGGGGC | 28 | 8-61 | 1.91 | Oxoglutarate dehydrogenase (lipoamide) |
| ACGGTGATGT | 37 | 11-81 | 1.91 | ESTs |
| CCCATCCGAA | 353 | 77-753 | 1.91 | Ribosomal protein L26 |
| ACAAACTTAG | 60 | 24-139 | 1.91 | Human calmodulin mRNA, complete cds |
| GCCTCCTCCC | 94 | 23-203 | 1.92 | ESTs |
| GTGCCTGAGA | 72 | 10-149 | 1.92 | LAMIN A |
| TCCAATACTG | 22 | 5-47 | 1.92 | Human dynamitin mRNA, complete cds |
| GTGGTGCGTG | 39 | 11-86 | 1.92 | Homo sapiens X-ray repair cross-complementing protein 2 |
| | | | | (XRCC2) mRNA, complete cds |
| AAGAAGCAGG | 38 | 15-88 | 1.92 | Homo sapiens unknown mRNA, complete cds |
| ACTTGGAGCC | 42 | 13-95 | 1.92 | Human calmodulin mRNA, complete cds |
| CCGTGGTCAC | 88 | 15-185 | 1.92 | H. sapiens mRNS for clathrin-associated protein |
| ACAGTGGGGA | 65 | 21-146 | 1.92 | Human (p23) mRNA, complete cds |
| ACAAACTGTG | 69 | 22-154 | 1.92 | H. sapiens mRNA for Sop2p-like protein |
| GTCTTAACTC | 23 | 6-50 | 1.93 | Homo sapiens Dim1p homolog (hdim1+) mRNA, complete |
| | | | | cds |
| CTGTGCTCGG | 34 | 11-77 | 1.93 | ENOYL-COA HYDRATASE, MITOCHONDRIAL |
| | | | | PRECURSOR |
| GTGGCCTGCA | 22 | 5-46 | 1.93 | ESTs, Weakly similar to K01G5.8 [C. elegans] |
| TGGTACACGT | 100 | 43-236 | 1.93 | Human calmodulin mRNA, complete cds |
| GTACTGTATG | 23 | 9-54 | 1.93 | ESTs |
| GTACTGTATG | 23 | 9-54 | 1.93 | Homo sapiens importin beta subunit mRNA, complete cds |
| GGCCAGGTGG | 25 | 5-53 | 1.93 | Homo sapiens calmodulin-stimulated phosphodiesterase |
| | | | | PDE1B1 mRNA, complete cds |
| GGCCAGGTGG | 25 | 5-53 | 1.93 | Metallopeptidase 1 (33 kD) |
| AGGGAGAGGG | 20 | 5-43 | 1.93 | Homo sapiens forkhead protein FREAC-2 mRNA, |
| | | | | complete cds |
| AGGGAGAGGG | 20 | 5-43 | 1.93 | Ferritin heavy chain |
| AGGGAGAGGG | 20 | 5-43 | 1.93 | UBIQUITIN CARBOXYL-TERMINAL HYDROLASE T |
| GTGGCAGGTG | 100 | 19-213 | 1.93 | Human mRNA for KIAA0340 gene, partial cds |
| TCTTGTGCAT | 143 | 26-302 | 1.93 | L-LACTATE DEHYDROGENASE M CHAIN |
| CCACACACCG | 21 | 8-49 | 1.94 | ESTs, Highly similar to HYPOTHETICAL 43.2 KD |
| | | | | PROTEIN C34E10.1 IN CHROMOSOME III |
| | | | | [Caenorhabditis elegans] |
| ACAAATCCTT | 45 | 7-95 | 1.94 | FK506-binding protein 1 (12 kD) |
| GTGAGACCCC | 45 | 11-98 | 1.94 | No match |
| AAAGCCAAGA | 29 | 10-67 | 1.94 | Electron-transfer-flavoprotein, beta polypeptide |
| CAAGGATCTA | 27 | 12-65 | 1.94 | Fibroblast growth factor receptor 2 |
| TGAGGCCAGG | 47 | 15-107 | 1.94 | High mobility group box |
| TTTTGTGTGA | 16 | 5-37 | 1.94 | ESTs, Weakly similar to 50S RIBOSOMAL PROTEIN L20 |
| | | | | [E. coli] |
| ACAGTCTTGC | 17 | 6-38 | 1.94 | CYTOCHROME P450 IVF3 |
| ACAGTCTTGC | 17 | 6-38 | 1.94 | Human mRNA for KIAA0102 gene, complete cds |
| CCAGGCACGC | 40 | 9-87 | 1.95 | Human HXC-26 mRNA, complete cds |
| AGTTTCCCAA | 40 | 21-100 | 1.95 | Homo sapiens SULT1C sulfotransferase (SULT1C) |
| | | | | mRNA, complete cds |
| CCAGTGGCCC | 274 | 48-582 | 1.95 | Ribosomal protein S9 |
| GCCCCGCCCT | 30 | 11-69 | 1.95 | Homo sapiens chromosome 19, cosmid R32184 |
| TCTCTACTAA | 41 | 6-85 | 1.95 | Tropomyosin 4 (fibroblast) |
| CGGCTTTTCT | 32 | 9-71 | 1.95 | Spectrin, beta, non-erythrocytic 1 |
| TGGCCCCCGC | 26 | 6-56 | 1.95 | ESTs |
| TGGCCCCCGC | 26 | 6-56 | 1.95 | Human helix-loop-helix zipper protein mRNA |
| CTCCTGGGGC | 48 | 6-101 | 1.95 | ESTs |
| AAGGAGCTGG | 16 | 5-37 | 1.96 | ESTs, Highly similar to YME1 PROTEIN [Saccharomyces |
| | | | | cerevisiae] |
| AAGGAGCTGG | 16 | 5-37 | 1.96 | ESTs |
| AAGGAGCTGG | 16 | 5-37 | 1.96 | Homo sapiens clone lambda MEN1 region unknown |
| | | | | protein mRNA, complete cds |
| GGCTTTGATT | 18 | 5-40 | 1.96 | COATOMER BETA′ SUBUNIT |
| ACTACCTTCA | 27 | 8-61 | 1.96 | ESTs, Weakly similar to B0334.4 [C. elegans] |
| CTGTGCATTT | 33 | 11-75 | 1.96 | Human 54 kDa protein mRNA, complete cds |
| ACTCCAAAAA | 210 | 40-452 | 1.96 | Human insulinoma rig-analog mRNA encoding DNA- |
| | | | | binding protein, complete cds |
| ACTCCAAAAA | 210 | 40-452 | 1.96 | H. sapiens mRNA for transmembrane protein rnp24 |
| TCCTGCCCCA | 72 | 14-155 | 1.96 | Parathymosin |
| TCCTGCCCCA | 72 | 14-155 | 1.96 | Homo sapiens mRNA for KIAA0511 protein, partial cds |
| AAGCTGGAGG | 56 | 15-125 | 1.96 | Human translation initiation factor elF3 p66 subunit mRNA, |
| | | | | complete cds |
| GCACAAGAAG | 90 | 19-195 | 1.96 | ESTs |
| GAAACCGAGG | 47 | 11-104 | 1.97 | ESTs, Weakly similar to HYPOTHETICAL 16.8 KD |
| | | | | PROTEIN IN SMY2-RPS101 INTERGENIC REGION |
| | | | | [S. cerevisiae] |
| GAAACCGAGG | 47 | 11-104 | 1.97 | Human mRNA for KIAA0029 gene, partial cds |
| GCCCGCAAGC | 16 | 5-36 | 1.97 | H. sapiens HUNKI mRNA |
| CTTTCAGATG | 44 | 12-98 | 1.97 | Phosphofructokinase, platelet |
| GGGCGCTGTG | 117 | 30-260 | 1.97 | Homo sapiens mRNA for smallest subunit of ubiquinol- |
| | | | | cytochrome c reductase, complete cds |
| GTATTCCCCT | 36 | 8-79 | 1.97 | Homo sapiens poly(A) binding protein II (PABP2) gene, |
| | | | | complete cds |
| GTATTCCCCT | 36 | 8-79 | 1.97 | ESTs, Highly similar to elastin like protein |
| | | | | [D. melanogaster] |
| CTGGCCATCG | 19 | 6-43 | 1.98 | ESTs |
| GTGGTGGACA | 33 | 6-72 | 1.98 | Human nicotinic acetylcholine receptor alpha6 subunit |
| | | | | precursor, mRNA, complete cds |
| GTGGTGGACA | 33 | 6-72 | 1.98 | Homo sapiens mRNA for PBK1 protein |
| GTGGTGGACA | 33 | 6-72 | 1.98 | Breast cancer 1, early onset |
| CACCTAATTG | 1247 | 410-2884 | 1.98 | Tag matches mitochondrial sequence |
| GACCCCTGTC | 18 | 6-41 | 1.98 | Homo sapiens (clone s153) mRNA fragment |
| CCCTTAGCTT | 47 | 21-114 | 1.98 | Human mRNA for myosin regulatory light chain |
| CAGAGACGTG | 30 | 9-68 | 1.98 | Human dystroglycan (DAG1) mRNA, complete cds |
| ATGGCTGGTA | 1064 | 174-2287 | 1.98 | 40S RIBOSOMAL PROTEIN S2 |
| TCAGCCTTCT | 46 | 14-106 | 1.99 | Homo sapiens flotillin-1 mRNA, complete cds |
| TCGTAACGAG | 23 | 9-54 | 1.99 | ESTs |
| GCGACGAGGC | 178 | 17-371 | 1.99 | 60S RIBOSOMAL PROTEIN L38 |
| GCGGGGTACC | 59 | 17-133 | 1.99 | Human mRNA for pM5 protein |
| TCCTTCTCCA | 58 | 12-128 | 1.99 | ALPHA-ACTININ 1, CYTOSKELETAL ISOFORM |
| CAGTCTCTCA | 107 | 16-229 | 1.99 | Ribosomal protein S10 |
| ACCCTTCCCT | 56 | 12-124 | 1.99 | ESTs, Weakly similar to VON EBNER'S GLAND PROTEIN |
| | | | | PRECURSOR [H. sapiens] |
| ACCCTTCCCT | 56 | 12-124 | 1.99 | Signal sequence receptor, beta |
| TGAGTGGTCA | 20 | 7-47 | 1.99 | ESTs, Highly similar to HYPOTHETICAL 13.6 KD |
| | | | | PROTEIN IN NUP170-ILS1 INTERGENIC REGION |
| | | | | [Saccharomyces cerevisiae] |
| GACAATGCCA | 48 | 11-107 | 1.99 | Human mRNA for ATP synthase gamma-subunit (L-type), |
| | | | | complete cds |
| ATCTTTCTGG | 80 | 15-176 | 2.00 | Tyrosine 3-monooxygenase/tryptophan 5-monooxygenase |
| | | | | activation protein, zeta polypeptide |
| AGCTGTCCCC | 23 | 5-50 | 2.00 | Tag matches mitochondrial sequence |
| TCTTCCAGGA | 52 | 11-114 | 2.00 | Human ribosomal protein L10 mRNA, complete cds |
| GTGCCTAGGA | 29 | 9-67 | 2.00 | ESTs |
| TGGACCCCCC | 26 | 6-57 | 2.00 | ESTs, Weakly similar to K04G2.2 [C. elegans] |
| ACCTGTATCC | 158 | 24-341 | 2.00 | INTERFERON-INDUCIBLE PROTEIN 1-8U |
| ACCTGCTGGT | 17 | 6-40 | 2.00 | Homo sapiens clone 23675 mRNA sequence |
| AGTCTGATGT | 39 | 5-84 | 2.00 | ESTs, Weakly similar to weak similarity to rat TEGT |
| | | | | protein [C. elegans] |
| TCTCTACCCA | 71 | 27-169 | 2.00 | Amyloid beta (A4) precursor-like protein 2 |
| TGATTAAGGT | 26 | 6-58 | 2.00 | HEAT SHOCK FACTOR PROTEIN 1 |
| CAGCAGAAGC | 191 | 75-459 | 2.01 | Homo sapiens 4F5rel mRNA, complete cds |
| TCCCTATTAA | 5970 | 987-12977 | 2.01 | No match |
| GTGGAGGTGC | 42 | 6-91 | 2.01 | Human 100 kDa coactivator mRNA, complete cds |
| AAGATCCCCG | 63 | 15-142 | 2.01 | Homo sapiens DNA sequence from cosmid ICK0721Q on |
| | | | | chromosome 6. |
| GAGCGGCCTC | 29 | 9-68 | 2.01 | Human ORF mRNA, complete cds |
| AACTACATAG | 21 | 9-50 | 2.02 | ESTs |
| GTAAGATTTG | 33 | 9-76 | 2.02 | Human 150 kDa oxygen-regulated protein ORP150 |
| | | | | mRNA, complete cds |
| AGCCTGCAGA | 65 | 17-147 | 2.02 | Homo sapiens chromosome 19, cosmid R33729 |
| GGACCACTGA | 498 | 174-1182 | 2.02 | Ribosomal protein L3 |
| TTCAATAAAA | 377 | 51-813 | 2.02 | TRANSCOBALAMIN I PRECURSOR |
| TTCAATAAAA | 377 | 51-813 | 2.02 | Ribosomal protein, large, P1 |
| CGATGGTCCC | 55 | 9-120 | 2.02 | Human B-cell receptor associated protein (hBAP) mRNA, |
| | | | | partial cds |
| CATTTGTAAT | 142 | 23-309 | 2.02 | Tag matches mitochondrial sequence |
| CCTGAGCCCG | 60 | 14-135 | 2.03 | ESTs, Weakly similar to ALBUMIN B-32 PROTEIN [Zea |
| | | | | mays] |
| TGAGGCCTCT | 29 | 6-65 | 2.03 | ESTs |
| AAGAGTTACG | 17 | 8-43 | 2.03 | ESTs, Highly similar to 50S RIBOSOMAL PROTEIN L2 |
| | | | | [Bacillus stearothermophilus] |
| GAATCCAACT | 46 | 6-100 | 2.03 | ESTs |
| AGGGGCGCAG | 29 | 8-67 | 2.03 | Human SH3-containing protein EEN mRNA, complete cds |
| GCTTAGAAGT | 31 | 6-69 | 2.03 | HEAT SHOCK PROTEIN HSP 90-ALPHA |
| AAGTCATTCA | 31 | 10-74 | 2.03 | Homo sapiens NADH-ubiquinone oxidoreductase subunit |
| | | | | CI-B14 mRNA, complete cds |
| AAGTCATTCA | 31 | 10-74 | 2.03 | H. sapiens mRNA for prcc protein |
| TACCCCACCC | 57 | 17-132 | 2.03 | ESTs |
| TACCCCACCC | 57 | 17-132 | 2.03 | Human zinc finger protein (MAZ) mRNA |
| CCTAGCTGGA | 511 | 132-1172 | 2.03 | PEPTIDYL-PROLYL CIS-TRANS ISOMERASE A |
| TCGTCTTTAT | 126 | 18-275 | 2.04 | 40S RIBOSOMAL PROTEIN S7 |
| GGTTTGGCTT | 70 | 14-156 | 2.04 | UBIQUINOL-CYTOCHROME C REDUCTASE COMPLEX |
| | | | | 11 KD PROTEIN PRECURSOR |
| TAGGATGGGG | 88 | 28-207 | 2.04 | Sodium/potassium-transporting ATPase beta-3 subunit |
| GTGCATCCCG | 43 | 16-105 | 2.04 | Casein kinase 2, beta polypeptide |
| CAGCGCTGCA | 37 | 11-87 | 2.04 | Human CDC37 homolog mRNA, complete cds |
| GGGAGCCCCT | 55 | 12-125 | 2.04 | ESTs, Highly similar to BETA-ARRESTIN 2 [Homo |
| | | | | sapiens] |
| GGGAGCCCCT | 55 | 12-125 | 2.04 | ESTs |
| GAAGATGTGG | 58 | 6-125 | 2.04 | Homo sapiens clone 23967 unknown mRNA, partial cds |
| CCTACCACAG | 21 | 9-52 | 2.05 | ESTs, Highly similar to GOLIATH PROTEIN [Drosophila |
| | | | | melanogaster] |
| TGCTAAAAAA | 26 | 9-61 | 2.06 | Myosin, heavy polypeptide 9, non-muscle |
| CACAGAGTCC | 28 | 7-64 | 2.06 | Low density lipoprotein-related protein-associated protein |
| | | | | 1 (alpha-2-macroglobulin receptor-associated protein 1 |
| GGGCCAATAA | 30 | 8-70 | 2.06 | Untitled |
| GCCTGCTGGG | 220 | 49-503 | 2.07 | Phospholipid hydroperoxide glutathione peroxidase |
| ACTGCTTGCC | 52 | 12-118 | 2.07 | S-ADENOSYLMETHIONINE SYNTHETASE GAMMA |
| | | | | FORM |
| ACTGCTTGCC | 52 | 12-118 | 2.07 | H. sapiens mRNA for Sop2p-like protein |
| CGGTTACTGT | 81 | 20-187 | 2.07 | Homo sapiens NADH:ubiquinone oxidoreductase NDUFS6 |
| | | | | subunit mRNA, nuclear gene encoding mitochondrial |
| | | | | protein, complete cds |
| AACCCGGGAG | 179 | 50-420 | 2.07 | Homo sapiens KIAA0408 mRNA, complete cds |
| AACCCGGGAG | 179 | 50-420 | 2.07 | Cytokine receptor family II, member 4 |
| AACCCGGGAG | 179 | 50-420 | 2.07 | H. sapiens mRNA for delta 4-3-oxosteroid 5 beta-reductase |
| ATTAACAAAG | 98 | 18-220 | 2.07 | Guanine nucleotide binding protein (G protein), alpha |
| | | | | stimulating activity polypeptide 1 |
| TTCAGTGCCC | 18 | 6-43 | 2.07 | ESTs, Weakly similar to GLUCOSE-6-PHOSPHATASE |
| | | | | [Rattus norvegicus] |
| CCGTGCTCAT | 51 | 18-123 | 2.07 | ESTs, Highly similar to ADIPOCYTE P27 PROTEIN [Mus |
| | | | | musculus] |
| ATCCCTCAGT | 78 | 24-184 | 2.07 | Activating transcription factor 4 (tax-responsive enhancer |
| | | | | element B67) |
| TACCATCAAT | 864 | 194-1985 | 2.07 | Glyceraldehyde-3-phosphate dehydrogenase |
| TGCACCACAG | 34 | 14-84 | 2.08 | Homo sapiens signal peptidase complex 18 kDa subunit |
| | | | | mRNA, partial cds |
| GAACCCTGGG | 46 | 9-104 | 2.08 | ESTs |
| GCCGTGTCCG | 542 | 60-1185 | 2.08 | Human ribosomal protein S6 mRNA, complete cds |
| ATAGAGGCAA | 28 | 7-65 | 2.08 | Human mRNA for KIAA0026 gene, complete cds |
| ATTGTTTATG | 83 | 11-184 | 2.08 | Human non-histone chromosomal protein HMG-17 mRNA, |
| | | | | complete cds |
| TAATAAAGGT | 229 | 46-523 | 2.09 | 40S RIBOSOMAL PROTEIN S8 |
| GGGATCAAGG | 26 | 7-61 | 2.09 | ESTs, Weakly similar to coded for by C. elegans cDNA |
| | | | | yk157f8.5 [C. elegans] |
| CAAGGGCTTG | 28 | 8-68 | 2.09 | ESTs, Highly similar to RAS-RELATED PROTEIN RAP- |
| | | | | 1B [Homo sapiens; Bos taurus] |
| TGGTGTTGAG | 828 | 147-1876 | 2.09 | Human DNA sequence from clone 1033B10 on |
| | | | | chromosome 6p21.2-21.31. |
| GAGTGAGTGA | 19 | 8-48 | 2.09 | ESTs, Weakly similar to C44C1.2 gene product |
| | | | | [C. elegans] |
| GTGGCGCACA | 42 | 9-98 | 2.09 | Human mRNA for KIAA0072 gene, partial cds |
| ATGATCCGGA | 22 | 5-52 | 2.10 | ATPase, Ca++ transporting, cardiac muscle, slow twitch 2 |
| AACCTGGGAG | 108 | 37-263 | 2.10 | Human DNA fragmentation factor-45 mRNA, complete cds |
| AACCTGGGAG | 108 | 37-263 | 2.10 | Homo sapiens mRNA for KIAA0563 protein, complete cds |
| TGCTTCATCT | 53 | 9-120 | 2.10 | Homo sapiens androgen receptor associated protein 24 |
| | | | | (ARA24) mRNA, complete cds |
| ATAATTCTTT | 205 | 37-467 | 2.10 | Ribosomal protein S29 |
| GTTCAGCTGT | 41 | 9-95 | 2.10 | Voltage-dependent anion channel 2 |
| GGGAAGTCAC | 22 | 5-50 | 2.10 | Human FX protein mRNA, complete cds |
| GGGTGCTTGG | 26 | 8-63 | 2.10 | Human mRNA for ORF, Xq terminal portion |
| CAGTTACTTA | 52 | 11-120 | 2.10 | Tyrosine 3-monooxygenase/tryptophan 5-monooxygenase |
| | | | | activation protein, beta polypeptide |
| GCGAAACCCC | 207 | 70-506 | 2.10 | Human G protein-coupled receptor (STRL22) mRNA, |
| | | | | complete cds |
| GCCTTCCAAT | 85 | 11-191 | 2.11 | P68 PROTEIN |
| CCCCCTGGAT | 485 | 33-1056 | 2.11 | Cell division cycle 2-like 1 (PITSLRE proteins) |
| GACCTCCTGC | 21 | 5-49 | 2.12 | Homo sapiens mRNA for kinesin-like DNA binding protein, |
| | | | | complete cds |
| GACCTCCTGC | 21 | 5-49 | 2.12 | Human SH3 domain-containing proline-rich kinase (sprk) |
| | | | | mRNA, complete cds |
| CAGCAGTAGC | 23 | 6-55 | 2.12 | H. sapiens mRNA for 218 kD Mi-2 protein |
| TTCATTATAA | 47 | 8-108 | 2.12 | Prothymosin alpha |
| CCCCCACCTA | 64 | 15-150 | 2.12 | INTESTINAL MEMBRANE A4 PROTEIN |
| GGTGGATGTG | 30 | 6-69 | 2.12 | Homo sapiens methyl-CpG binding protein MBD3 (MBD3) |
| | | | | mRNA, complete cds |
| TCTGGTTTGT | 41 | 5-91 | 2.12 | Homo sapiens mRNA for integral membrane protein |
| | | | | Tmp21-I (p23) |
| TCTGGTTTGT | 41 | 5-91 | 2.12 | THYMOSIN BETA-10 |
| CGCCTGTAAT | 48 | 8-111 | 2.13 | CDC21 HOMOLOG |
| TCCTGCTGCC | 45 | 6-101 | 2.13 | ESTs |
| TCCTGCTGCC | 45 | 6-101 | 2.13 | ESTs, Weakly similar to F46F6.1 [C. elegans] |
| GTGTGGTGGT | 27 | 6-64 | 2.13 | Homo sapiens mRNA for GDP dissociation inhibitor beta |
| TGATGTCCAC | 10 | 5-27 | 2.14 | ESTs |
| CCAGGAGGAA | 222 | 77-551 | 2.14 | HEAT SHOCK COGNATE 71 KD PROTEIN |
| GTGAAGCCCC | 42 | 9-99 | 2.14 | No match |
| GGGAGCCCGG | 32 | 7-75 | 2.15 | Homo sapiens herpesvirus entry protein B (HVEB) mRNA, |
| | | | | complete cds |
| GCCATCCCCT | 64 | 14-150 | 2.15 | Tag matches mitochondrial sequence |
| CAGTTGGTTG | 28 | 8-69 | 2.15 | Homo sapiens mRNA for E1B-55 kDa-associated protein |
| ATCCATCTGT | 21 | 9-54 | 2.15 | H. sapiens hnRNP-E2 mRNA |
| GCCAGGAAGC | 32 | 6-75 | 2.15 | ESTs, Weakly similar to C01A2.5 [C. elegans] |
| TCCAGCCCCT | 32 | 9-78 | 2.15 | ESTs, Weakly similar to T08G11.1 [C. elegans] |
| GCCCCCCACT | 24 | 6-58 | 2.15 | Human MAP kinase activated protein kinase 2 mRNA, |
| | | | | complete cds |
| TGTCTGTGGT | 18 | 5-45 | 2.15 | H. sapiens BAT1 mRNA for nuclear RNA helicase (DEAD |
| | | | | family) |
| TCCCGTACAT | 258 | 37-592 | 2.15 | No match |
| GTGGTGGGCA | 61 | 12-144 | 2.15 | Cholinergic receptor, nicotinic, delta polypeptide |
| GTGGTGGGCA | 61 | 12-144 | 2.15 | Isovaleryl Coenzyme A dehydrogenase |
| GTGGTGGGCA | 61 | 12-144 | 2.15 | Homo sapiens josephin MJD1 mRNA, complete cds |
| CTGTTAGTGT | 54 | 13-130 | 2.16 | MALATE DEHYDROGENASE, CYTOPLASMIC |
| CTCTCACCCT | 68 | 28-175 | 2.16 | Ribonuclease/angiogenin inhibitor |
| TGCTGGTGTG | 30 | 8-74 | 2.16 | Human mRNA, clone HH109 (screened by the monoclonal |
| | | | | antibody of insulin receptor substrate-1 (IRS-1)) |
| CTAAGACTTC | 1455 | 317-3462 | 2.16 | Tag matches mitochondrial sequence |
| GGAAGGACAG | 39 | 5-90 | 2.16 | ATPase, H+ transporting, lysosomal (vacuolar proton |
| | | | | pump) 31 kD |
| GAAGTGTGTC | 23 | 9-60 | 2.16 | ESTs, Highly similar to HYPOTHETICAL 37.2 KD |
| | | | | PROTEIN C12C2.09C IN CHROMOSOME I |
| | | | | [Schizosaccharomyces pombe] |
| GTACCCGGAC | 33 | 9-81 | 2.17 | ESTs, Weakly similar to W08E3.1 [C. elegans] |
| CCTCCCTGAT | 35 | 10-86 | 2.17 | Homo sapiens dynamin (DNM) mRNA, complete cds |
| TCATCTTCAA | 19 | 5-46 | 2.17 | CALRETICULIN PRECURSOR |
| TCATCTTCAA | 19 | 5-46 | 2.17 | ESTs |
| TCATCTTCAA | 19 | 5-46 | 2.17 | RAB6, member RAS oncogene family |
| ATGTACTCTG | 38 | 6-89 | 2.17 | IMP (inosine monophosphate) dehydrogenase 2 |
| CGCCGGAACA | 648 | 123-1530 | 2.17 | Ribosomal protein L4 |
| AAGGGAGGGT | 78 | 14-184 | 2.17 | Human phosphotyrosine independent ligand p62 for the |
| | | | | Lck SH2 domain mRNA, complete cds |
| GAAAAAAAAA | 112 | 12-255 | 2.17 | Cell division cycle 10 (homologous to CDC10 of S. cerevisiae |
| AAACTCTGTG | 27 | 6-64 | 2.18 | Homo sapiens p120 catenin isoform 1A (CTNND1) mRNA, |
| | | | | alternatively spliced, complete cds |
| ACACACGCAA | 22 | 8-56 | 2.18 | ESTs |
| CCGCCGAAGT | 50 | 7-116 | 2.18 | Ribosomal protein L12 |
| TGTGCTAAAT | 169 | 46-415 | 2.18 | 60S RIBOSOMAL PROTEIN L34 |
| CGACCGTGGC | 24 | 6-57 | 2.18 | ESTs |
| GCCTGGGCTG | 44 | 18-114 | 2.18 | ESTs |
| GCCTGGGCTG | 44 | 18-114 | 2.18 | Homo sapiens molybdopterin synthase sulfurylase |
| | | | | (MOCS3) mRNA, complete cds |
| AAAGTCAGAA | 24 | 12-65 | 2.19 | Ubiquinol-cytochrome c reductase core protein II |
| TGGAGCGCTA | 31 | 5-71 | 2.19 | ESTs, Weakly similar to PUTATIVE MITOCHONDRIAL |
| | | | | CARRIER C16C10.1 [C. elegans] |
| GAAATGATGA | 70 | 14-167 | 2.19 | Homo sapiens mRNA for c-myc binding protein, complete |
| | | | | cds |
| TGTCGCTGGG | 73 | 14-173 | 2.19 | C4/C2 activating component of Ra-reactive factor |
| GCCCCTGCCT | 39 | 6-91 | 2.19 | Homo sapiens DNA-binding protein (CROC-1B) mRNA, |
| | | | | complete cds |
| GCCCCTGCCT | 39 | 6-91 | 2.19 | Glutathione S-transferase M4 |
| CAGGCCTGGC | 20 | 7-50 | 2.19 | ESTs |
| CAGGCCTGGC | 20 | 7-50 | 2.19 | ESTs |
| GCAAAAAAAA | 153 | 35-371 | 2.20 | No match |
| AGCCACCACG | 33 | 8-81 | 2.20 | Human mRNA for KIAA0149 gene, complete cds |
| GAGGAAGAAG | 52 | 16-130 | 2.20 | Homologue of mouse tumor rejection antigen gp96 |
| CAGCTGTAGT | 20 | 9-54 | 2.20 | Human mRNA for KIAA0174 gene, complete cds |
| TCTTCTCCCT | 40 | 10-99 | 2.20 | Human mRNA for hepatoma-derived growth factor, |
| | | | | complete cds |
| TACATTCTGT | 30 | 7-74 | 2.20 | Myeloid cell leukemia sequence 1 (BCL2-related) |
| GGGAAACCCC | 39 | 11-98 | 2.21 | ESTs, Weakly similar to HYPOTHETICAL 68.7 KD |
| | | | | PROTEIN ZK757.1 IN CHROMOSOME III [C. elegans] |
| AGCCACTGCA | 67 | 8-155 | 2.21 | Homo sapiens mRNA for 26S proteasome subunit p55, |
| | | | | complete cds |
| TAGTTGAAGT | 55 | 13-136 | 2.21 | UBIQUINOL-CYTOCHROME C REDUCTASE COMPLEX |
| | | | | 14 KD PROTEIN |
| GCCAAGTTTG | 17 | 5-43 | 2.21 | Human mRNA for proteasome subunit p112, complete cds |
| GGCGGCTGCA | 36 | 9-89 | 2.21 | Excision repair cross-complementing rodent repair |
| | | | | deficiency, complementation group 1 (includes overlapping |
| | | | | antisense sequence) |
| AAAAAAAAAA | 469 | 38-1076 | 2.21 | H. sapiens mRNA for sodium-phophate transport system 1 |
| AAAAAAAAAA | 469 | 38-1076 | 2.21 | Homo sapiens GPI-linked anchor protein (GFRA1) mRNA, |
| | | | | complete cds |
| AAAAAAAAAA | 469 | 38-1076 | 2.21 | Enolase 1, (alpha) |
| AAAAAAAAAA | 469 | 38-1076 | 2.21 | Calcium channel, voltage-dependent, P/Q type, alpha 1A |
| | | | | subunit |
| TGTTCCACTC | 18 | 5-46 | 2.21 | Homo sapiens CD39L2 (CD39L2) mRNA, complete cds |
| CTCGGTGATG | 30 | 10-76 | 2.22 | H. sapiens mRNA for ras-related GTP-binding protein |
| CTTCTCAGGG | 17 | 5-43 | 2.22 | ESTs, Highly similar to PUTATIVE CYSTEINYL-TRNA |
| | | | | SYNTHETASE C29E6.06C [Schizosaccharomyces |
| | | | | pombe] |
| GGTAGCCCAC | 16 | 5-40 | 2.22 | ESTs |
| GGGTTTTTAT | 65 | 7-150 | 2.22 | Homo sapiens dbpB-like protein mRNA, complete cds |
| CCTGTAACCC | 39 | 12-99 | 2.23 | Human translation initiation factor elF-2alpha mRNA, |
| | | | | 3′UTR |
| GAAACAAGAT | 58 | 5-133 | 2.23 | Phosphoglycerate kinase 1 |
| GATGAGTCTC | 71 | 18-175 | 2.23 | Homo sapiens proteasome subunit XAPC7 mRNA, |
| | | | | complete cds |
| GGCCCTAGGC | 43 | 6-101 | 2.23 | H. sapiens ERF-2 mRNA |
| TGGCCCCACC | 440 | 59-1041 | 2.23 | Pyruvate kinase, muscle |
| CAGCGCGCCC | 66 | 5-152 | 2.23 | ESTs |
| AGGCGAGATC | 91 | 27-231 | 2.24 | Homo sapiens proteasome subunit XAPC7 mRNA, |
| | | | | complete cds |
| GCGGGGTGGA | 64 | 12-155 | 2.24 | H. sapiens ERF-1 mRNA 3′ end |
| GGGGCCCCCT | 21 | 6-54 | 2.24 | Homo sapiens mRNA for NA14 protein |
| AAGGAACTTG | 24 | 8-61 | 2.24 | ESTs |
| AAGGAACTTG | 24 | 8-61 | 2.24 | Homo sapiens clone 24655 mRNA sequence |
| AATTGCAAGC | 18 | 5-47 | 2.24 | COFILIN, NON-MUSCLE ISOFORM |
| CCTGTGATCC | 66 | 22-171 | 2.25 | No match |
| CCCCGCCAAG | 66 | 11-159 | 2.25 | Human adult heart mRNA for neutral calponin, complete |
| | | | | cds |
| CTCAACAGCA | 60 | 12-147 | 2.25 | Human translation initiation factor 3 47 kDa subunit |
| | | | | mRNA, complete cds |
| AAGGTAGCAG | 56 | 17-143 | 2.25 | ADENYLYL CYCLASE-ASSOCIATED PROTEIN 1 |
| AAGCCAGCCC | 78 | 5-180 | 2.25 | Protein kinase C substrate 80K-H |
| CAGCCTTGGA | 21 | 5-52 | 2.25 | ESTs, Weakly similar to siah binding protein 1 [H. sapiens] |
| TTTGCTCTCC | 24 | 8-61 | 2.25 | Vinculin |
| CAACATTCCT | 41 | 14-106 | 2.26 | Dopachrome tautomerase (dopachrome delta-isomerase, |
| | | | | tyrosine-related protein 2) |
| TACTAGTCCT | 77 | 13-187 | 2.26 | HEAT SHOCK PROTEIN HSP 90-ALPHA |
| GACTCTGGTG | 59 | 6-139 | 2.26 | Homo sapiens chromosome 19, cosmid R29381 |
| GACTCTGGTG | 59 | 6-139 | 2.26 | 40S RIBOSOMAL PROTEIN S15A |
| GTGGCTCACG | 102 | 16-248 | 2.26 | Homo sapiens KIAA0414 mRNA, partial cds |
| GTGGCTCACG | 102 | 16-248 | 2.26 | Human Tax1 binding protein mRNA, partial cds |
| GTGGCGGGCA | 71 | 16-177 | 2.27 | H. sapiens mRNA for urea transporter |
| GTGGCGGGCA | 71 | 16-177 | 2.27 | Homo sapiens mRNA for KIAA0472 protein, partial cds |
| CCTGTGGTCC | 86 | 18-215 | 2.27 | No match |
| TACAGCACGG | 27 | 6-68 | 2.27 | Homo sapiens microsomal glutathione S-transferase 3 |
| | | | | (MGST3) mRNA, complete cds |
| GTGGCACCTG | 20 | 5-51 | 2.27 | ESTs, Highly similar to NEUROGENIC LOCUS NOTCH |
| | | | | PROTEIN HOMOLOG PRECURSOR [Xenopus laevis] |
| TACACGTGAG | 40 | 14-103 | 2.27 | ESTs, Weakly similar to GOLIATH PROTEIN [Drosophila |
| | | | | melanogaster] |
| TCAGGCATTT | 69 | 24-180 | 2.27 | ESTs, Highly similar to RAS-RELATED PROTEIN RAB-1A |
| | | | | [H. sapiens] |
| TTCACAAAGG | 25 | 7-63 | 2.27 | PROTEASOME ZETA CHAIN |
| TTCTTGTGGC | 245 | 54-610 | 2.27 | Ribosomal protein S11 |
| TCCCTATTAG | 91 | 14-220 | 2.27 | No match |
| TACAAGAGGA | 208 | 49-521 | 2.27 | Ribosomal protein L6 |
| TCAGACGCAG | 344 | 78-862 | 2.28 | Prothymosin alpha |
| CAGGATCCAG | 35 | 6-86 | 2.28 | Human putative tumor suppressor (SNC6) mRNA, |
| | | | | complete cds |
| TCTGTACACC | 55 | 11-135 | 2.28 | Ribosomal protein S11 |
| GAAGCAGGAC | 352 | 54-856 | 2.28 | COFILIN, NON-MUSCLE ISOFORM |
| GCGCCGCCCC | 27 | 5-68 | 2.28 | ESTs, Moderately similar to nuclear autoantigen |
| | | | | [H. sapiens] |
| CCCTCCTGGG | 69 | 23-181 | 2.29 | ESTs |
| TGGGCGCCTT | 35 | 6-85 | 2.29 | Uroporphyrinogen decarboxylase |
| GTGGTACAGG | 121 | 35-312 | 2.29 | Homo sapiens microtubule-based motor (HsKIFC3) |
| | | | | mRNA, complete cds |
| GTGGTACAGG | 121 | 35-312 | 2.29 | ESTs |
| GGTGAGACCT | 93 | 43-255 | 2.29 | Prostatic binding protein |
| GAGATCCGCA | 59 | 16-153 | 2.30 | INTERFERON GAMMA UP-REGULATED I-5111 |
| | | | | PROTEIN PRECURSOR |
| TTGGCAGCCC | 48 | 5-115 | 2.30 | Ribosomal protein L27a |
| GCCTTTCCCT | 22 | 8-59 | 2.30 | APOPTOSIS REGULATOR BCL-X |
| GGAGTGGACA | 190 | 29-465 | 2.30 | 60S RIBOSOMAL PROTEIN L18 |
| TTATGGGGAG | 29 | 6-74 | 2.30 | H factor (complement)-like 1 |
| TTATGGGGAG | 29 | 6-74 | 2.30 | TRANSFORMATION-SENSITIVE PROTEIN IEF SSP |
| | | | | 3521 |
| GAGTGGGGGC | 43 | 9-108 | 2.30 | ESTs, Highly similar to LYSOSOMAL PRO-X |
| | | | | CARBOXYPEPTIDASE PRECURSOR [Homo sapiens] |
| GTGGCACGTG | 192 | 36-479 | 2.30 | No match |
| CTGGGCGTGT | 126 | 41-331 | 2.31 | ESTs |
| TTGGGGTTTC | 1243 | 255-3123 | 2.31 | Ferritin heavy chain |
| GGCTGGGCCT | 93 | 14-229 | 2.31 | Clathrin, light polypeptide (Lcb) |
| GGCTGGGCCT | 93 | 14-229 | 2.31 | EST |
| CCTGTTCTCC | 28 | 8-73 | 2.31 | ESTs |
| GTGTCTCATC | 26 | 6-67 | 2.31 | ESTs |
| GTGTCTCATC | 26 | 6-67 | 2.31 | Enolase 1, (alpha) |
| ACGATTGATG | 23 | 6-60 | 2.31 | ESTs, Highly similar to HYPOTHETICAL 27.5 KD |
| | | | | PROTEIN IN SPX19-GCR2 INTERGENIC REGION |
| | | | | [Saccharomyces cerevisiae] |
| TTGTTGTTGA | 75 | 20-194 | 2.31 | Calmodulin 1 (phosphorylase kinase, delta) |
| TGGCCTCCCC | 49 | 9-122 | 2.32 | H. sapiens mRNA for rho GDP-dissociation Inhibitor 1 |
| ATCGGGCCCG | 51 | 19-136 | 2.32 | ESTs, Weakly similar to zinc finger protein [H. sapiens] |
| GCCGCCATCA | 45 | 8-111 | 2.33 | Human protein disulfide isomerase-related protein P5 |
| | | | | mRNA, partial cds |
| GTGCTGGACC | 63 | 15-162 | 2.33 | Human mRNA for proteasome activator hPA28 subunit |
| | | | | beta, complete cds |
| TTGTAATCGT | 206 | 59-540 | 2.33 | Human mRNA for ornithine decarboxylase antizyme, ORF |
| | | | | 1 and ORF 2 |
| TAATGGTAAC | 30 | 5-75 | 2.33 | Homo sapiens nuclear-encoded mitochondrial cytochrome |
| | | | | c oxidase Va subunit mRNA, complete cds |
| AACGACCTCG | 156 | 6-369 | 2.33 | Homo sapiens clone 24703 beta-tubulin mRNA, complete |
| | | | | cds |
| GCCTGCACCC | 18 | 7-49 | 2.34 | Human neuronal olfactomedin-related ER localized protein |
| | | | | mRNA, partial cds |
| GCCTGCACCC | 18 | 7-49 | 2.34 | ESTs |
| AAGGTGGAGG | 809 | 156-2051 | 2.34 | 60S RIBOSOMAL PROTEIN L18A |
| AAGGAGATGG | 467 | 132-1226 | 2.34 | Ribosomal protein L31 |
| CAGTTCTCTG | 41 | 9-105 | 2.34 | Human BTK region clone ftp-3 mRNA |
| GTGAAACCTC | 111 | 38-297 | 2.35 | Homo sapiens intrinsic factor-B12 receptor precursor, |
| | | | | mRNA, complete cds |
| TAGGTTGTCT | 546 | 104-1386 | 2.35 | TRANSLATIONALLY CONTROLLED TUMOR PROTEIN |
| CCTGTGACAG | 61 | 8-150 | 2.35 | Human mRNA for KIAA0106 gene, complete cds |
| CTCATAAGGA | 572 | 118-1463 | 2.35 | Tag matches mitochondrial sequence |
| GGTGGCTTTG | 23 | 8-61 | 2.35 | Homo sapiens NADH:ubiquinone oxidoreductase B12 |
| | | | | subunit mRNA, nuclear gene encoding mitochondrial |
| | | | | protein, complete cds |
| GCTCAGCTGG | 171 | 29-432 | 2.36 | Eukaryotic translation elongation factor 1 delta (guanine |
| | | | | nucleotide exchange protein) |
| GGCCCTGAGC | 141 | 14-348 | 2.36 | Human RNA polymerase II subunit (hsRPB10) mRNA, |
| | | | | complete cds |
| TCTGCTAAAG | 53 | 5-130 | 2.36 | High-mobility group (nonhistone chromosomal) protein 1 |
| TCTGCTAAAG | 53 | 5-130 | 2.36 | ESTs |
| AGCCCCACAA | 18 | 5-46 | 2.37 | ESTs |
| CTGAGTCTCC | 80 | 9-198 | 2.37 | Guanine nucleotide binding protein (G protein), alpha |
| | | | | inhibiting activity polypeptide 2 |
| TGCTTTGGGA | 53 | 14-139 | 2.37 | ESTs, Weakly similar to No definition line found |
| | | | | [C. elegans] |
| CCTGTCCTGC | 60 | 7-149 | 2.37 | ESTs, Moderately similar to GTP-binding protein- |
| | | | | associated protein [M. musculus] |
| GGGGAAATCG | 708 | 96-1772 | 2.37 | THYMOSIN BETA-10 |
| TCTGCCTGGG | 48 | 15-130 | 2.37 | ESTs, Weakly similar to orf, len: 159, CAI: 0.12 |
| | | | | [S. cerevisiae] |
| CAATAAACTG | 97 | 12-242 | 2.37 | PROTEIN TRANSLATION FACTOR SUI1 HOMOLOG |
| GAGTCTGAGG | 24 | 9-66 | 2.37 | U1 snRNP 70K protein |
| GTGGCAGGCG | 87 | 16-223 | 2.37 | Human pancreatic zymogen granule membrane protein |
| | | | | GP-2 mRNA, complete cds |
| GTGGCAGGCG | 87 | 16-223 | 2.37 | Nuclear factor of kappa light polypeptide gene enhancer in |
| | | | | B-cells 2 (p49/p100) |
| CGAGGGGCCA | 188 | 33-480 | 2.38 | Human non-muscle alpha-actinin mRNA, complete cds |
| GTGGGGGGAG | 19 | 5-49 | 2.38 | Human DNA sequence from cosmid F0811 on |
| | | | | chromosome 6. Contains Daxx, BING1, Tapasin, RGL2, |
| | | | | KE2, BING4, BING5, ESTs and CpG islands |
| GAGTGGCTAT | 28 | 8-75 | 2.38 | Homo sapiens KIAA0419 mRNA, complete cds |
| GAGTGGCTAT | 28 | 8-75 | 2.38 | Homo sapiens mRNA for GDP dissociation inhibitor beta |
| GTAGACTCAC | 17 | 5-46 | 2.38 | LARGE PROLINE-RICH PROTEIN BAT2 |
| AGGGAAAGAG | 27 | 7-72 | 2.39 | Human G10 homolog (edg-2) mRNA, complete cds |
| AGGGAAAGAG | 27 | 7-72 | 2.39 | Homo sapiens mRNA for KIAA0632 protein, partial cds |
| CCCATCGTCC | 3108 | 714-8145 | 2.39 | Tag matches mitochondrial sequence |
| TCGCCGCGAC | 34 | 8-90 | 2.40 | No match |
| TGTCCTGGTT | 150 | 39-398 | 2.40 | CYCLIN-DEPENDENT KINASE INHIBITOR 1 |
| CTTTTTGTGC | 42 | 6-107 | 2.40 | Tyrosine 3-monooxygenase/tryptophan 5-monooxygenase |
| | | | | activation protein, beta polypeptide |
| ATAAATTGGG | 23 | 8-62 | 2.40 | ATP synthase, H+ transporting, mitochondrial F0 complex, |
| | | | | subunit b, isoform 1 |
| TATCACTCTG | 21 | 6-57 | 2.40 | Human male-enhanced antigen mRNA (Mea), complete |
| | | | | cds |
| GTGGTGGGCG | 61 | 9-156 | 2.40 | No match |
| CCACTACACT | 38 | 6-98 | 2.41 | Human TNF-related apoptosis inducing ligand TRAIL |
| | | | | mRNA, complete cds |
| TGACCCCACA | 29 | 11-81 | 2.41 | ESTs, Weakly similar to F25H5.h [C. elegans] |
| TGATTTCACT | 803 | 132-2064 | 2.41 | EST |
| TGATTTCACT | 803 | 132-2064 | 2.41 | Tag matches mitochondrial sequence |
| GGCTCCCACT | 142 | 36-379 | 2.41 | HEAT SHOCK PROTEIN HSP 90-BETA |
| CCTGTGTGTG | 32 | 6-82 | 2.41 | ESTs |
| AATCCTGTGG | 514 | 135-1377 | 2.42 | Ribosomal protein L8 |
| AGGAGCAAAG | 43 | 9-112 | 2.42 | Human mRNA for NADPH-flavin reductase, complete cds |
| CCTTTGAACA | 43 | 7-111 | 2.42 | Human Chromosome 16 BAC clone CIT987SK-A-61E3 |
| GTGGGGCTAG | 30 | 8-81 | 2.42 | H. sapiens mRNA for protein phosphatase 5 |
| AGGGTGAAAC | 29 | 5-75 | 2.43 | Human splicing factor SRp30c mRNA, complete cds |
| CCTCAGGATA | 270 | 72-728 | 2.43 | ESTs |
| CCTCAGGATA | 270 | 72-728 | 2.43 | Tag matches mitochondrial sequence |
| TTCCACTAAC | 55 | 12-147 | 2.44 | Human plectin (PLEC1) mRNA, complete cds |
| CCCCCGTGAA | 86 | 18-228 | 2.44 | Homo sapiens interleukin-1 receptor-associated kinase |
| | | | | (IRAK) mRNA, complete cds |
| TGTGCTCGGG | 107 | 35-295 | 2.44 | Human mRNA for KIAA0088 gene, partial cds |
| AAGCCTTGCT | 20 | 6-54 | 2.44 | ESTs |
| TGTTCATCAT | 40 | 15-114 | 2.45 | ESTs, Weakly similar to neuroendocrine-specific protein C |
| | | | | [H. sapiens] |
| AACTAACAAA | 86 | 24-234 | 2.45 | Ubiquitin A-52 residue ribosomal protein fusion product 1 |
| GCTGTTGCGC | 158 | 33-419 | 2.45 | 40S RIBOSOMAL PROTEIN S20 |
| GGATGTGAAA | 45 | 7-118 | 2.45 | Antigen identified by monoclonal antibodies 12E7, F21 and |
| | | | | O13 |
| ACTGGTACGT | 34 | 8-90 | 2.45 | Homo sapiens F1Fo-ATPase synthase f subunit mRNA, |
| | | | | complete cds |
| TTGTATTCCA | 16 | 5-45 | 2.45 | H. sapiens mRNA for alpha 4 protein |
| GGCTGGGGGC | 437 | 48-1124 | 2.46 | Human profilin mRNA, complete cds |
| CCACTGCACT | 925 | 181-2460 | 2.47 | Thyroid autoantigen 70 kD (Ku antigen) |
| CCACTGCACT | 925 | 181-2460 | 2.47 | Enhancer of zeste (Drosophila) homolog 1 |
| CCACTGCACT | 925 | 181-2460 | 2.47 | CD19 antigen |
| CCACTGCACT | 925 | 181-2460 | 2.47 | Human clone 23732 mRNA, partial cds |
| CCACTGCACT | 925 | 181-2460 | 2.47 | Annexin II (lipocortin II) |
| CCACTGCACT | 925 | 181-2460 | 2.47 | Alkaline phosphatase, placental (Regan isozyme) |
| CCACTGCACT | 925 | 181-2460 | 2.47 | Homo sapiens clone 24760 mRNA sequence |
| CCACTGCACT | 925 | 181-2460 | 2.47 | Homo sapiens carbonic anhydrase precursor (CA 12) |
| | | | | mRNA, complete cds |
| CCACTGCACT | 925 | 181-2460 | 2.47 | Homo sapiens methyl-CpG binding protein MBD4 (MBD4) |
| | | | | mRNA, complete cds |
| CCACTGCACT | 925 | 181-2460 | 2.47 | Phosphodiesterase 4C, cAMP-specific (dunce |
| | | | | (Drosophila)-homolog phosphodiesterase E1) |
| CCACTGCACT | 925 | 181-2460 | 2.47 | Human SNRPN mRNA, 3′ UTR, partial sequence |
| CCACTGCACT | 925 | 181-2460 | 2.47 | Homo sapiens brachyury variant A (TBX1) mRNA, |
| | | | | complete cds |
| CCACTGCACT | 925 | 181-2460 | 2.47 | H. sapiens beta glucuronidase pseudogene |
| CCACTGCACT | 925 | 181-2460 | 2.47 | G PROTEIN-ACTIVATED INWARD RECTIFIER |
| | | | | POTASSIUM CHANNEL 4 |
| CACTTGCCCT | 109 | 21-290 | 2.47 | ESTs, Highly similar to ACETYL-COENZYME A |
| | | | | SYNTHETASE [Escherichia coli] |
| CACTTGCCCT | 109 | 21-290 | 2.47 | ESTs, Highly similar to NADH-UBIQUINONE |
| | | | | OXIDOREDUCTASE B22 SUBUNIT [Bos taurus] |
| GCAAGCCAAC | 100 | 17-264 | 2.47 | Tag matches mitochondrial sequence |
| TAGATAATGG | 49 | 5-126 | 2.47 | Homo sapiens clone 24703 beta-tubulin mRNA, complete |
| | | | | cds |
| TCGAAGCCCC | 251 | 60-682 | 2.47 | Tag matches mitochondrial sequence |
| AGAAAAAAAA | 115 | 9-294 | 2.48 | Enolase 1, (alpha) |
| AGAAAAAAAA | 115 | 9-294 | 2.48 | Human mRNA for KIAA0099 gene, complete cds |
| GGCGCCTCCT | 66 | 9-172 | 2.48 | Eukaryotic translation initiation factor 4A (eIF-4A) isoform 1 |
| GGCGCCTCCT | 66 | 9-172 | 2.48 | TRANSALDOLASE |
| TAAACTGTTT | 29 | 7-79 | 2.48 | ESTs |
| TAAACTGTTT | 29 | 7-79 | 2.48 | 40S RIBOSOMAL PROTEIN S14 |
| GGCCTTTTTT | 36 | 6-95 | 2.48 | Human mRNA for histone H1x, complete cds |
| GGCCTTTTTT | 36 | 6-95 | 2.48 | Homo sapiens mRNA for KIAA0529 protein, partial cds |
| GCGACAGCTC | 44 | 5-115 | 2.48 | 60S RIBOSOMAL PROTEIN L24 |
| CCCACACTAC | 57 | 17-159 | 2.49 | Human signal-transducing guanine nucleotide-binding |
| | | | | regulatory (G) protein beta subunit mRNA, complete cds |
| AGCAGATCAG | 390 | 65-1034 | 2.49 | S100 calcium-binding protein A10 (annexin II ligand, |
| | | | | calpactin I, light polypeptide (p11)) |
| GCATAGGCTG | 90 | 15-240 | 2.49 | ELONGATION FACTOR TU, MITOCHONDRIAL |
| | | | | PRECURSOR |
| GAGGCCGACC | 25 | 9-72 | 2.49 | Basigin |
| AAATGCCACA | 42 | 6-110 | 2.49 | ESTs, Weakly similar to neuroendocrine-specific protein C |
| | | | | [H. sapiens] |
| AGCCCTACAA | 754 | 208-2089 | 2.49 | Tag matches mitochondrial sequence |
| TTGGTGAAGG | 399 | 57-1053 | 2.50 | Human thymosin beta-4 mRNA, complete cds |
| CCGGGCCCAG | 46 | 9-125 | 2.50 | Homo sapiens mRNA for TRIP6 (thyroid receptor |
| | | | | interacting protein) |
| TTCATACACC | 772 | 125-2055 | 2.50 | Tag matches mitochondrial sequence |
| GCAGCCATCC | 790 | 96-2072 | 2.50 | Ribosomal protein L28 |
| GCCGGGTGGG | 668 | 126-1796 | 2.50 | Basigin |
| GCTCCCAGAC | 53 | 9-142 | 2.50 | Homo sapiens mRNA for synaptogyrin 2 |
| AGCCACCGTG | 39 | 8-105 | 2.51 | No match |
| TCAGCTGGCC | 16 | 6-47 | 2.51 | Human nuclear factor NF90 mRNA, complete cds |
| GGGGGCGCCT | 22 | 6-62 | 2.52 | Adenine nucleotide translocator 3 (liver) |
| CGGCCCAACG | 59 | 14-161 | 2.52 | H. sapiens mRNA for arginine methyltransferase, splice |
| | | | | variant, 1262 bp |
| TGGCCATCTG | 65 | 14-177 | 2.52 | ESTs, Weakly similar to N-methyl-D-aspartate receptor |
| | | | | glutamate-binding chain [R. norvegicus] |
| CCTCCCCCGT | 59 | 11-159 | 2.52 | Homo sapiens breakpoint cluster region protein 1 |
| | | | | (BCRG1) mRNA, complete cds |
| ACTTGTTCGC | 27 | 6-73 | 2.52 | ESTs |
| AAGACTGGCT | 30 | 6-81 | 2.52 | ESTs, Highly similar to Surf-4 protein [M. musculus] |
| AGCACATTTG | 42 | 5-112 | 2.53 | ESTs, Highly similar to deduced protein product shows |
| | | | | significant homology to coactosin from Dictyostelium |
| | | | | discoideum [H. sapiens] |
| GTGAAGGCAG | 467 | 83-1265 | 2.53 | Ribosomal protein S3A |
| CAATAAATGT | 227 | 43-620 | 2.54 | Ribosomal protein L37 |
| GCCAGGGCGG | 46 | 5-121 | 2.54 | ESTs, Highly similar to HYPOTHETICAL 52.8 KD |
| | | | | PROTEIN T05E11.5 IN CHROMOSOME IV |
| | | | | [Caenorhabditis elegans] |
| GTGTAATAAG | 57 | 9-154 | 2.54 | Heterogeneous nuclear ribonucleoprotein A2/B1 |
| TTCTGCACTG | 25 | 6-70 | 2.54 | Collagen, type I, alpha-2 |
| TTCTGCACTG | 25 | 6-70 | 2.54 | ESTs |
| GTGAAACCCC | 1352 | 514-3963 | 2.55 | Myelin oligodendrocyte glycoprotein {alternative products} |
| GTGAAACCCC | 1352 | 514-3963 | 2.55 | Dihydrolipoamide branched chain transacylase (E2 |
| | | | | component of branched chain keto acid dehydrogenase |
| | | | | complex) |
| GTGAAACCCC | 1352 | 514-3963 | 2.55 | Human mRNA for platelet-activating factor acetylhydrolase |
| | | | | 2, complete cds |
| GTGAAACCCC | 1352 | 514-3963 | 2.55 | GRANULOCYTE-MACROPHAGE COLONY- |
| | | | | STIMULATING FACTOR RECEPTOR ALPHA CHAIN |
| | | | | PRECURSOR |
| GTGAAACCCC | 1352 | 514-3963 | 2.55 | Thymopoietin |
| GTGAAACCCC | 1352 | 514-3963 | 2.55 | Basic fibroblast growth factor (bFGF) receptor (shorter |
| | | | | form) |
| GTGAAACCCC | 1352 | 514-3963 | 2.55 | Homo sapiens mRNA for KIAA0794 protein, partial cds |
| GTGAAACCCC | 1352 | 514-3963 | 2.55 | Homo sapiens RNA polymerase I subunit hRPA39 mRNA, |
| | | | | complete cds |
| GTGAAACCCC | 1352 | 514-3963 | 2.55 | Homo sapiens mRNA for KIAA0701 protein, partial cds |
| GTGAAACCCC | 1352 | 514-3963 | 2.55 | Homo sapiens mRNA for MAX.3 cell surface antigen |
| GTGAAACCCC | 1352 | 514-3963 | 2.55 | Homo sapiens mRNA for KIAA0706 protein, complete cds |
| GTGAAACCCC | 1352 | 514-3963 | 2.55 | Homo sapiens deoxyribonuclease II mRNA, complete cds |
| GTGAAACCCC | 1352 | 514-3963 | 2.55 | Homo sapiens clone 24758 mRNA sequence |
| GTGAAACCCC | 1352 | 514-3963 | 2.55 | Kangai 1 (suppression of tumorigenicity 6, prostate; CD82 |
| | | | | antigen (R2 leukocyte antigen, antigen detected by |
| | | | | monoclonal and antibody IA4)) |
| GTGAAACCCC | 1352 | 514-3963 | 2.55 | Leptin (murine obesity homolog) |
| GACACCTCCT | 45 | 7-122 | 2.55 | ESTs, Weakly similar to TIP49 [R. norvegicus] |
| GACGTGTGGG | 94 | 6-247 | 2.56 | H2AZ histone |
| GCAAAACCCC | 162 | 46-461 | 2.56 | Homo sapiens tumor necrosis factor superfamily member |
| | | | | LIGHT mRNA, complete cds |
| TACCAGTGTA | 46 | 6-124 | 2.56 | Heat shock 60 kD protein 1 (chaperon in) |
| CCCCTCCCCA | 30 | 11-90 | 2.58 | Chromosome 22q13 BAC Clone CIT987SK-384D8 |
| | | | | complete sequence |
| GGTGATGAGG | 35 | 8-98 | 2.58 | Homo sapiens BC-2 protein mRNA, complete cds |
| GTGTGTAAAA | 27 | 6-76 | 2.59 | H. sapiens CDM mRNA |
| GGCTCCTCGA | 41 | 11-117 | 2.59 | Homo sapiens tapasin (NGS-17) mRNA, complete cds |
| AAAAGAAACT | 62 | 12-174 | 2.60 | POLYADENYLATE-BINDING PROTEIN |
| CAGCGCACAG | 22 | 5-64 | 2.60 | ESTs |
| CTGGGAGAGG | 35 | 11-102 | 2.60 | ESTs |
| GAAAAATGGT | 340 | 58-943 | 2.60 | Laminin receptor (2H5 epitope) |
| ATCACGCCCT | 192 | 26-527 | 2.61 | Tag matches mitochondrial sequence |
| TAGCTCTATG | 107 | 43-323 | 2.61 | ATPase, Na+/K+ transporting, alpha 1 polypeptide |
| GTATTGGCCT | 21 | 7-61 | 2.61 | Human p76 mRNA, complete cds |
| CCCGACGTGC | 58 | 20-171 | 2.62 | ESTs, Highly similar to NADH-UBIQUINONE |
| | | | | OXIDOREDUCTASE B9 SUBUNIT [Bos taurus] |
| GAAGTTATGA | 32 | 7-89 | 2.62 | T-COMPLEX PROTEIN 1, ALPHA SUBUNIT |
| TAAAAAAAAA | 108 | 7-290 | 2.63 | ESTs |
| TAAAAAAAAA | 108 | 7-290 | 2.63 | Ubiquitin-conjugating enzyme E2A (RAD6 homolog) |
| TAAAAAAAAA | 108 | 7-290 | 2.63 | Homo sapiens protein kinase (BUB1) mRNA, complete |
| | | | | cds |
| GCCGCCCTGC | 71 | 13-199 | 2.63 | Acyl-Coenzyme A dehydrogenase, very long chain |
| TTTGGGGCTG | 78 | 30-234 | 2.63 | Human mRNA for proton-ATPase-like protein, complete |
| | | | | cds |
| GTGGCAGGCA | 86 | 18-245 | 2.63 | No match |
| GGCTGTACCC | 79 | 18-225 | 2.63 | CYSTEINE-RICH PROTEIN |
| AGCAGGGCTC | 128 | 17-353 | 2.63 | ESTs, Highly similar to PNG gene [H. sapiens] |
| AAGAAGATAG | 152 | 10-412 | 2.64 | 60S RIBOSOMAL PROTEIN L23A |
| TCTGGGGACG | 27 | 7-78 | 2.64 | Human translational initiation factor 2 beta subunit (eIF-2- |
| | | | | beta) mRNA, complete cds |
| GCTAGGTTTA | 80 | 9-220 | 2.65 | Tag matches mitochondrial sequence |
| TGGTGACAGT | 32 | 6-91 | 2.65 | Homo sapiens histone H2A.F/Z variant (H2AV) mRNA, |
| | | | | complete cds |
| TTACCATATC | 196 | 46-566 | 2.65 | Human mRNA for ribosomal protein L39, complete cds |
| GTGGCGGGTG | 59 | 9-165 | 2.65 | No match |
| TGGATCCTAG | 28 | 7-81 | 2.66 | Homo sapiens NADH:ubiquinone oxidoreductase NDUFS3 |
| | | | | subunit mRNA, nuclear gene encoding mitochondrial |
| | | | | protein, complete cds |
| GGGTTTGAAC | 22 | 7-64 | 2.66 | Homo sapiens SKB1Hs mRNA, complete cds |
| AATGCAGGCA | 83 | 9-231 | 2.67 | S-adenosylhomocysteine hydrolase |
| ACATCGTAGG | 30 | 10-90 | 2.67 | ESTs |
| AACGCTGCCT | 59 | 10-167 | 2.67 | Human APRT gene for adenine phosphoribosyltransferase |
| TGGAGGTGGG | 20 | 6-58 | 2.68 | ESTs |
| TGCCTGCTCC | 21 | 8-64 | 2.68 | ESTs |
| CTTCCAGCTA | 358 | 87-1050 | 2.69 | Annexin II (lipocortin II) |
| GTAAGTGTAC | 80 | 8-223 | 2.69 | ESTs |
| GTAAGTGTAC | 80 | 8-223 | 2.69 | Tag matches mitochondrial sequence |
| GTGTCTCGCA | 40 | 6-112 | 2.70 | Annexin XI (56 kD autoantigen) |
| ATCCGGCGCC | 114 | 14-321 | 2.70 | Homo sapiens RNA polymerase II transcription factor SIII |
| | | | | p18 subunit mRNA, complete cds |
| TGCCTGCACC | 232 | 61-688 | 2.70 | Cystatin C (amyloid angiopathy and cerebral hemorrhage) |
| TTCCTATTAA | 42 | 7-121 | 2.72 | ESTs |
| CAGGAGTTCA | 91 | 23-270 | 2.72 | Homo sapiens Arp2/3 protein complex subunit p34-Arc |
| | | | | (ARC34) mRNA, complete cds |
| GTCTGCGTGC | 51 | 5-143 | 2.72 | Proteasome component C2 |
| GAAATACAGT | 264 | 50-769 | 2.72 | ESTs |
| GAAATACAGT | 264 | 50-769 | 2.72 | Cathepsin D (lysosomal aspartyl protease) |
| TGAGCCCGGC | 36 | 8-106 | 2.74 | ESTs, Highly similar to LATENT TRANSFORMING |
| | | | | GROWTH FACTOR BETA BINDING PROTEIN 1 |
| | | | | PRECURSOR [Rattus norvegicus] |
| GTGGTGTGTG | 46 | 6-134 | 2.74 | Homo sapiens NF-AT4c mRNA, complete cds |
| GTGGTGTGTG | 46 | 6-134 | 2.74 | Acid phosphatase, prostate |
| TCACCCACAC | 383 | 111-1167 | 2.76 | Ribosomal protein L17 |
| TCACCCACAC | 383 | 111-1167 | 2.76 | ESTs, Weakly similar to !!!! ALU SUBFAMILY J WARNING |
| | | | | ENTRY !!!! [H. sapiens] |
| CTGGATCTGG | 65 | 12-190 | 2.76 | Glycogen phosphorylase B (brain form) |
| GAAGATGTGT | 95 | 24-287 | 2.77 | ESTs, Highly similar to HYPOTHETICAL 6.3 KD |
| | | | | PROTEIN ZK652.2 IN CHROMOSOME III [Caenorhabditis |
| | | | | elegans] |
| CGGATAACCA | 53 | 6-153 | 2.78 | Human cell cycle protein p38-2G4 homolog (hG4-1) |
| | | | | mRNA, complete cds |
| TCAGAAGGTG | 38 | 5-111 | 2.78 | ESTs, Weakly similar to RNA-binding protein [H. sapiens] |
| GAGAAACCCC | 95 | 22-288 | 2.78 | Human mRNA for KIAA0134 gene, complete cds |
| GAGAAACCCC | 95 | 22-288 | 2.78 | H. sapiens F11 mRNA |
| GAGAAACCCC | 95 | 22-288 | 2.78 | Human mRNA for KIAA0159 gene, complete cds |
| CTCGTTAAGA | 32 | 6-95 | 2.80 | Human calmodulin mRNA, complete cds |
| TTGGAGATCT | 93 | 20-279 | 2.80 | Human NADH:ubiquinone oxidoreductase MLRQ subunit |
| | | | | mRNA, complete cds |
| GAGGTCCCTG | 65 | 12-193 | 2.81 | PROTEASOME IOTA CHAIN |
| TTCCGCGTGC | 50 | 5-146 | 2.81 | Homo sapiens lysyl hydroxylase isoform 3 (PLOD3) |
| | | | | mRNA, complete cds |
| CAGCCCAACC | 64 | 8-187 | 2.81 | Homo sapiens eukaryotic translation initiation factor 3 |
| | | | | subunit (p42) mRNA, complete cds |
| GTGGCTCACA | 104 | 9-303 | 2.81 | Adenosine A2b receptor |
| TAGAAAGGCA | 31 | 6-92 | 2.82 | H. sapiens ERF-2 mRNA |
| TAAGTAGCAA | 33 | 7-102 | 2.83 | ESTs, Weakly similar to putative [M. musculus] |
| GGTGAGACAC | 128 | 25-389 | 2.83 | Adenine nucleotide translocator 3 (liver) |
| CCCATCGTCT | 39 | 5-116 | 2.83 | No match |
| CCGATCACCG | 59 | 14-182 | 2.83 | Human translational initiation factor 2 beta subunit (eIF-2- |
| | | | | beta) mRNA, complete cds |
| GAATCGGTTA | 43 | 10-133 | 2.83 | Homo sapiens NADH-ubiquinone oxidoreductase 15 kDa |
| | | | | subunit mRNA, complete cds |
| AACCCAGGAG | 110 | 11-323 | 2.84 | No match |
| TTTTGAAGCA | 33 | 15-108 | 2.85 | Homo sapiens hepatitis B virus X interacting protein (XIP) |
| | | | | mRNA, complete cds |
| CACAGGCAAA | 40 | 8-122 | 2.85 | Human mRNA for KIAA0005 gene, complete cds |
| TCAGCTTCAC | 30 | 7-93 | 2.85 | Human mRNA for KIAA0359 gene, complete cds |
| TCAGCTTCAC | 30 | 7-93 | 2.85 | Human putative G-protein (GP-1) mRNA, complete cds |
| GAGGGCCGGT | 61 | 10-185 | 2.85 | ESTs, Highly similar to HISTONE H2A [Cairina moschata] |
| CCCCAGCCAG | 320 | 74-988 | 2.86 | Ribosomal protein S3 |
| GTGGTGGGTG | 59 | 5-176 | 2.86 | Human RACH1 (RACH1) mRNA, complete cds |
| CTGCCAAGTT | 100 | 27-314 | 2.87 | Homo sapiens mRNA for zyxin |
| GAGAAACCCT | 46 | 12-144 | 2.87 | Homo sapiens mRNA, chromosome 1 specific transcript |
| | | | | KIAA0506 |
| GAGAAACCCT | 46 | 12-144 | 2.87 | Vitamin D (1,25-dihydroxyvitamin D3) receptor |
| ACTAACACCC | 544 | 132-1694 | 2.87 | Tag matches mitochondrial sequence |
| TTTTGGGGGC | 37 | 7-112 | 2.88 | ESTs |
| TTTTGGGGGC | 37 | 7-112 | 2.88 | Human mRNA for proton-ATPase-like protein, complete |
| | | | | cds |
| GTGAAACCCA | 43 | 15-140 | 2.88 | No match |
| GCTTTCATTG | 27 | 12-89 | 2.89 | Homo sapiens clone 23967 unknown mRNA, partial cds |
| GTGGCACGCA | 33 | 6-101 | 2.89 | No match |
| GGGTCAAAAG | 52 | 14-165 | 2.89 | HISTONE H3.3 |
| GGGGGTCACC | 61 | 9-186 | 2.90 | ATP SYNTHASE LIPID-BINDING PROTEIN P1 |
| | | | | PRECURSOR |
| GTGAAACCCT | 664 | 198-2130 | 2.91 | Carboxypeptidase M |
| GTGAAACCCT | 664 | 198-2130 | 2.91 | H. sapiens mRNA for laminin |
| GTGAAACCCT | 664 | 198-2130 | 2.91 | GC-RICH SEQUENCE DNA-BINDING FACTOR |
| GTGAAACCCT | 664 | 198-2130 | 2.91 | Homo sapiens mRNA for KIAA0596 protein, partial cds |
| GTGAAACCCT | 664 | 198-2130 | 2.91 | Homo sapiens clone 23605 mRNA sequence |
| GTGAAACCCT | 664 | 198-2130 | 2.91 | Formyl peptide receptor 1 |
| AGTTGAAATT | 20 | 6-64 | 2.91 | ESTs |
| AGAATCGCTT | 74 | 11-228 | 2.92 | Homo sapiens coatomer protein (COPA) mRNA, complete |
| | | | | cds |
| AGGTCAAGAG | 20 | 7-65 | 2.92 | No match |
| CTAACCAGAC | 43 | 11-136 | 2.93 | ANGIOTENSIN-CONVERTING ENZYME PRECURSOR, |
| | | | | SOMATIC |
| GGGATGGCAG | 38 | 5-115 | 2.93 | VALYL-TRNA SYNTHETASE |
| AGACCCACAA | 162 | 39-512 | 2.93 | Tag matches mitochondrial sequence |
| TCGAAGAACC | 50 | 7-155 | 2.94 | CD63 antigen (melanoma 1 antigen) |
| TGAAATAAAA | 71 | 6-214 | 2.95 | Nucleophosmin (nucleolar phosphoprotein B23, numatrin) |
| ACTGAGGTGC | 34 | 9-109 | 2.95 | Homo sapiens FGF-1 intracellular binding protein (FIBP) |
| | | | | mRNA, complete cds |
| ACTCAGAAGA | 50 | 12-160 | 2.95 | ESTs, Highly similar to NADH-UBIQUINONE |
| | | | | OXIDOREDUCTASE AGGG SUBUNIT PRECURSOR |
| | | | | [Bos taurus] |
| GAACACATCC | 440 | 113-1414 | 2.96 | Ribosomal protein L19 |
| AACTAATACT | 67 | 6-203 | 2.96 | ESTs, Weakly similar to !!!! ALU SUBFAMILY J WARNING |
| | | | | ENTRY !!!! [H. sapiens] |
| AGATGTGTGG | 30 | 8-98 | 2.96 | Hydroxyacyl-Coenzyme A dehydrogenase/3-ketoacyl- |
| | | | | Coenzyme A thiolase/enoyl-Coenzyme A hydratase |
| | | | | (trifunctional protein), beta subunit |
| GTGGTGTGCA | 27 | 8-89 | 2.97 | Homo sapiens RNA transcript from U17 small nucleolar |
| | | | | RNA host gene, variant U17HG-AB |
| GGCGTCCTGG | 55 | 9-172 | 2.98 | ESTs, Weakly similar to No definition line found |
| | | | | [C. elegans] |
| CCTGCAATCC | 47 | 11-152 | 2.98 | No match |
| GCCTGGCCAT | 57 | 14-184 | 2.99 | GUANINE NUCLEOTIDE-BINDING PROTEIN BETA |
| | | | | SUBUNIT-LIKE PROTEIN 12.3 |
| GCCTGGCCAT | 57 | 14-184 | 2.99 | ESTs, Moderately similar to SULFATED SURFACE |
| | | | | GLYCOPROTEIN 185 [Volvox carteri] |
| GCTGCCCTTG | 134 | 14-415 | 2.99 | Human alpha-tubulin mRNA, 3′ end |
| GCTGCCCTTG | 134 | 14-415 | 2.99 | Human alpha-tubulin mRNA, complete cds |
| GCCAGCCCAG | 90 | 12-281 | 3.00 | Human transcriptional corepressor hKAP1/TIF1B mRNA, |
| | | | | complete cds |
| TCCTATTAAG | 160 | 34-515 | 3.00 | ESTs |
| ATTGTGCCAC | 34 | 8-110 | 3.00 | No match |
| CCATTGCACT | 237 | 58-773 | 3.02 | Ataxia telangiectasia mutated (includes complementation |
| | | | | groups A, C and D) |
| GCACCTCAGC | 38 | 8-122 | 3.02 | ESTs |
| TTGGTCAGGC | 129 | 24-419 | 3.05 | Calcium modulating ligand |
| TTGGTCAGGC | 129 | 24-419 | 3.05 | Human melanoma antigen recognized by T-cells (MART- |
| | | | | 1) mRNA |
| GGGCCCCGCA | 30 | 6-98 | 3.05 | Human mRNA for KIAA0123 gene, partial cds |
| GTGGCACACA | 70 | 15-228 | 3.06 | Homo sapiens AIBC1 (AIBC1) mRNA, complete cds |
| GTGGCACACA | 70 | 15-228 | 3.06 | Homo sapiens mRNA for MEGF8, partial cds |
| TTGGCCAGGC | 346 | 87-1149 | 3.07 | Human cytochrome P450-IIB (hIIB3) mRNA, complete cds |
| TTGGCCAGGC | 346 | 87-1149 | 3.07 | Homo sapiens X-ray repair cross-complementing protein 2 |
| | | | | (XRCC2) mRNA, complete cds |
| TTGGCCAGGC | 346 | 87-1149 | 3.07 | Homo sapiens oligodendrocyte-specific protein (OSP) |
| | | | | mRNA, complete cds |
| TTGGCCAGGC | 346 | 87-1149 | 3.07 | MHC class II transactivator |
| TTGGCCAGGC | 346 | 87-1149 | 3.07 | Fc fragment of IgA, receptor for |
| TTGGCCAGGC | 346 | 87-1149 | 3.07 | Protein kinase, interferon-inducible double stranded RNA |
| | | | | dependent |
| TTGGCCAGGC | 346 | 87-1149 | 3.07 | Zinc finger protein 157 (HZF22) |
| GTCACTGCCT | 20 | 5-68 | 3.08 | Homo sapiens mRNA for Ribosomal protein kinase B |
| | | | | (RSK-B) |
| GCCACCCCGT | 61 | 8-197 | 3.09 | Glucose-6-phosphate dehydrogenase |
| TCCCTATAAG | 107 | 17-347 | 3.09 | No match |
| CCTGTAATCC | 1302 | 453-4484 | 3.10 | Breast cancer 2, early onset |
| CCTGTAATCC | 1302 | 453-4484 | 3.10 | Integrin, beta 3 (platelet glycoprotein IIIa, antigen CD61) |
| CCTGTAATCC | 1302 | 453-4484 | 3.10 | Transcription factor 1, hepatic; LF-B1, hepatic nuclear |
| | | | | factor (HNF1), albumin proximal factor |
| CCTGTAATCC | 1302 | 453-4484 | 3.10 | Homo sapiens interferon induced tetratricopeptide protein |
| | | | | IFI60 (IFIT4) mRNA, complete cds |
| CCTGTAATCC | 1302 | 453-4484 | 3.10 | H. sapiens RBQ-3 mRNA |
| CCTGTAATCC | 1302 | 453-4484 | 3.10 | Human hVps41p (HVPS41) mRNA, complete cds |
| CCTGTAATCC | 1302 | 453-4484 | 3.10 | Human TNF-alpha converting enzyme precursor, mRNA, |
| | | | | alternatively spliced, complete cds |
| CCTGTAATCC | 1302 | 453-4484 | 3.10 | Homo sapiens mRNA for KIAA0526 protein, complete cds |
| CCTGTAATCC | 1302 | 453-4484 | 3.10 | Homo sapiens melastatin 1 (MLSN1) mRNA, complete cds |
| CCTGTAATCC | 1302 | 453-4484 | 3.10 | Homo sapiens clone 23716 mRNA sequence |
| CCTGTAATCC | 1302 | 453-4484 | 3.10 | Homo sapiens mRNA for KIAA0538 protein, partial cds |
| CCTGTAATCC | 1302 | 453-4484 | 3.10 | HLA CLASS I HISTOCOMPATIBILITY ANTIGEN, E |
| | | | | E*0101/E*0102 ALPHA CHAIN PRECURSOR |
| CCTGTAATCC | 1302 | 453-4484 | 3.10 | Homo sapiens decoy receptor 2 mRNA, complete cds |
| CCTGTAATCC | 1302 | 453-4484 | 3.10 | CATHEPSIN S PRECURSOR |
| CCTGTAATCC | 1302 | 453-4484 | 3.10 | Homo sapiens type 6 nucleoside diphosphate kinase |
| | | | | NM23-H6 (NM23-H6) mRNA, complete cds |
| CCTGTAATCC | 1302 | 453-4484 | 3.10 | 5′ nucleotidase (CD73) |
| CCTGTAATCC | 1302 | 453-4484 | 3.10 | Homo sapiens mRNA, chromosome 1 specific transcript |
| | | | | KIAA0508 |
| CCTGTAATCC | 1302 | 453-4484 | 3.10 | H. sapiens mRNA for p85 beta subunit of phosphatidyl- |
| | | | | inositol-3-kinase |
| CCTGTAATCC | 1302 | 453-4484 | 3.10 | Interleukin 12 receptor, beta-2 |
| TCCCCGTACA | 3918 | 290-12438 | 3.10 | No match |
| GTCACACCAC | 30 | 9-104 | 3.11 | ESTs |
| GTCACACCAC | 30 | 9-104 | 3.11 | Prothymosin alpha |
| ATGGCAAGGG | 56 | 9-182 | 3.11 | ESTs, Weakly similar to !!!! ALU SUBFAMILY J WARNING |
| | | | | ENTRY !!!! [H. sapiens] |
| CTGTTGGCAT | 111 | 27-372 | 3.11 | Ribosomal protein L21 |
| CTAGCCTCAC | 623 | 161-2105 | 3.12 | Actin, gamma 1 |
| AGTGCAAGAC | 57 | 10-187 | 3.12 | Tag matches mitochondrial sequence |
| CCTGTAGTCC | 231 | 67-791 | 3.13 | No match |
| TTTTCTGAAA | 66 | 12-218 | 3.13 | Thioredoxin |
| CTCCCCTGCC | 62 | 9-203 | 3.14 | Capping protein (actin filament), gelsolin-like |
| TCTCTTTTTC | 32 | 6-108 | 3.14 | H. sapiens tissue specific mRNA |
| GCGGACGAGG | 35 | 8-118 | 3.14 | Homo sapiens TFAR19 mRNA, complete cds |
| GCGGACGAGG | 35 | 8-118 | 3.14 | Human tip associating protein (TAP) mRNA, complete cds |
| GGAGTCATTG | 56 | 12-190 | 3.16 | Human mRNA for proteasome subunit HsC10-II, complete |
| | | | | cds |
| GTAGCAGGTG | 67 | 21-233 | 3.17 | Homo sapiens cargo selection protein TIP47 (TIP47) |
| | | | | mRNA, complete cds |
| CGCAAGCTGG | 65 | 13-221 | 3.17 | LAMIN A |
| GTGAAACCCG | 36 | 11-126 | 3.18 | No match |
| AGGTCAGGAG | 359 | 133-1274 | 3.18 | Major histocompatibility complex, class II, DR beta 5 |
| AGGTCAGGAG | 359 | 133-1274 | 3.18 | Human mRNA for KIAA0331 gene, complete cds |
| AGGTCAGGAG | 359 | 133-1274 | 3.18 | Human mRNA for KIAA0226 gene, complete cds |
| GAATGCAGTT | 13 | 5-45 | 3.18 | ESTs |
| GAATGCAGTT | 13 | 5-45 | 3.18 | ESTs |
| GAATGCAGTT | 13 | 5-45 | 3.18 | ESTs |
| GTGAGCCCAT | 77 | 21-269 | 3.21 | HEAT SHOCK PROTEIN HSP 90-BETA |
| GTAATCCTGC | 109 | 23-375 | 3.22 | Tag matches ribosomal RNA sequence |
| TGAAGTAACA | 31 | 7-108 | 3.22 | PROTEIN TRANSLATION FACTOR SUI1 HOMOLOG |
| TGCCTGTAAT | 59 | 15-206 | 3.22 | ISLET AMYLOID POLYPEPTIDE PRECURSOR |
| GTAGCATAAA | 28 | 6-95 | 3.23 | Human ubiquitin gene, complete cds |
| CCGTGGTCGT | 67 | 9-224 | 3.23 | Fibrillarin |
| ATGAAACCCC | 67 | 24-240 | 3.23 | Homo sapiens mRNA expressed in osteoblast, complete |
| | | | | cds |
| AAGATTGGTG | 81 | 13-275 | 3.25 | CD9 antigen |
| ATCCGTGCCC | 35 | 11-124 | 3.25 | Human calmodulin mRNA, complete cds |
| CCCTTCACTG | 16 | 5-58 | 3.26 | ESTs, Moderately similar to !!!! ALU SUBFAMILY J |
| | | | | WARNING ENTRY !!!! [H. sapiens] |
| CCCTTCACTG | 16 | 5-58 | 3.26 | ESTs |
| CAGCTGGGGC | 54 | 6-183 | 3.26 | Polypyrimidine tract binding protein (hnRNP I) {alternative |
| | | | | products} |
| CAGGCCCCAC | 109 | 17-370 | 3.26 | Human mRNA for calgizzarin, complete cds |
| TGTTTATCCT | 25 | 7-89 | 3.26 | — |
| TAACCAATCA | 52 | 14-184 | 3.26 | Human Rab5c-like protein mRNA, complete cds |
| CACCTGTAGT | 32 | 5-110 | 3.27 | Ribosomal protein L5 |
| TACCCTAAAA | 103 | 16-351 | 3.27 | Human kpni repeat mrna (cdna clone pcd-kpni-4), 3′ end |
| TACCCTAAAA | 103 | 16-351 | 3.27 | Homo sapiens mRNA for KIAA0675 protein, complete cds |
| TACCCTAAAA | 103 | 16-351 | 3.27 | Human Line-1 repeat mRNA with 2 open reading frames |
| TGCCTCTGCG | 175 | 83-655 | 3.28 | Human platelet-endothelial tetraspan antigen 3 mRNA, |
| | | | | complete cds |
| GCAAAACCCT | 81 | 19-284 | 3.28 | No match |
| AAGGACCTTT | 115 | 18-396 | 3.28 | ESTs |
| CTGGCGCCGA | 39 | 9-138 | 3.30 | ESTs, Weakly similar to F35G12.9 [C. elegans] |
| GAAGCTTTGC | 133 | 15-454 | 3.30 | HEAT SHOCK PROTEIN HSP 90-ALPHA |
| GCTCCGAGCG | 57 | 6-195 | 3.30 | Ribosomal protein S16 |
| TTGCCCAGGC | 69 | 21-251 | 3.30 | Cell division cycle 42 (GTP-binding protein, 25 kD) |
| TTGCCCAGGC | 69 | 21-251 | 3.30 | Human brain mRNA homologous to 3′UTR of human |
| | | | | CD24 gene, partial sequence |
| ACCCACGTCA | 55 | 9-189 | 3.31 | Jun B proto-oncogene |
| GCTCCACTGG | 29 | 8-103 | 3.31 | Mannose-6-phosphate receptor (cation dependent) |
| TTTAACGGCC | 142 | 18-489 | 3.31 | Tag matches mitochondrial sequence |
| CTTGTAATCC | 71 | 11-248 | 3.32 | ESTs, Moderately similar to !!!! ALU SUBFAMILY J |
| | | | | WARNING ENTRY !!!! [H. sapiens] |
| CACTTTTGGG | 47 | 8-165 | 3.33 | ESTs |
| CCGGGTGATG | 92 | 20-325 | 3.33 | Human copper transport protein HAH1 (HAH1) mRNA, |
| | | | | complete cds |
| GGGGTAAGAA | 62 | 6-213 | 3.33 | Prostatic binding protein |
| TGACTGGCAG | 49 | 7-172 | 3.34 | CD59 antigen p18-20 (antigen identified by monoclonal |
| | | | | antibodies 16.3A5, EJ16, EJ30, EL32 and G344) |
| CAATGTGTTA | 47 | 17-176 | 3.39 | H. sapiens mRNA for NADH dehydrogenase |
| GGCTCGGGAT | 74 | 6-257 | 3.40 | CALPAIN 1, LARGE |
| TGCCTGTAGT | 71 | 15-258 | 3.40 | Hum ORF (CEI5) mRNA, 3′ flank |
| CGCCGCCGGC | 807 | 148-2906 | 3.42 | Human ribosomal protein L35 mRNA, complete cds |
| GGTGGGGAGA | 68 | 6-239 | 3.44 | Human chromosome 17q21 mRNA clone LF113 |
| GTAAAACCCT | 24 | 8-90 | 3.44 | No match |
| GGCTCCTGGC | 100 | 9-354 | 3.44 | Homo sapiens b(2)gcn homolog mRNA, complete cds |
| AGTAGGTGGC | 53 | 5-188 | 3.46 | Tag matches mitochondrial sequence |
| GGAGGTGGGG | 126 | 19-456 | 3.48 | Granulin |
| CCTTTGGCTA | 27 | 5-100 | 3.49 | ESTs, Highly similar to 40S RIBOSOMAL PROTEIN S27 |
| | | | | [Rattus norvegicus] |
| AGAAAGATGT | 74 | 11-268 | 3.50 | Annexin I (lipocortin I) |
| AGAACAAAAC | 75 | 6-271 | 3.52 | Proliferation-associated gene A (natural killer-enhancing |
| | | | | factor A) |
| AACTAAAAAA | 110 | 9-396 | 3.53 | Ubiquitin A-52 residue ribosomal protein fusion product 1 |
| ATTGCACCAC | 38 | 5-138 | 3.53 | Human transglutaminase mRNA, 3′ untranslated region |
| GATCCCAACT | 389 | 27-1402 | 3.54 | H. sapiens mRNA for metallothionein isoform 2 |
| GATCCCAACT | 389 | 27-1402 | 3.54 | Human mRNA for metallothionein from cadmium-treated |
| | | | | cells |
| CACTACTCAC | 356 | 99-1361 | 3.54 | Tag matches mitochondrial sequence |
| CTGTACAGAC | 132 | 20-487 | 3.55 | Homo sapiens beta 2 gene |
| TACCCTAGAA | 43 | 5-159 | 3.58 | Estrogen receptor |
| GTAAAACCCC | 57 | 8-213 | 3.58 | Tumor necrosis factor receptor 2 (75 kD) |
| GTAAAACCCC | 57 | 8-213 | 3.58 | Homo sapiens mRNA for KIAA0632 protein, partial cds |
| GTAAAACCCC | 57 | 8-213 | 3.58 | Homo sapiens protease-activated receptor 4 mRNA, |
| | | | | complete cds |
| CTGAGAGCTG | 32 | 9-125 | 3.61 | Homo sapiens growth-arrest-specific protein (gas) mRNA, |
| | | | | complete cds |
| GGCTGGTCTG | 57 | 6-211 | 3.62 | ESTs |
| ACGCAGGGAG | 360 | 29-1334 | 3.63 | HEAT SHOCK PROTEIN HSP 90-ALPHA |
| GCCCTCGGCC | 44 | 5-165 | 3.63 | Homo sapiens mRNA for protein phosphatase 2C gamma |
| CTCCCTTGCC | 20 | 5-78 | 3.64 | ESTs, Highly similar to COATOMER ZETA SUBUNIT |
| | | | | [Bos taurus] |
| CCTGTAATCT | 81 | 27-323 | 3.65 | V-erb-b2 avian erythroblastic leukemia viral oncogene |
| | | | | homolog 3 {alternative products} |
| AGGTCCTAGC | 391 | 16-1448 | 3.66 | Glutathione-S-transferase pi-1 |
| ACTGAAGGCG | 68 | 15-266 | 3.68 | Human metargidin precursor mRNA, complete cds |
| AAGGAAGATG | 24 | 6-94 | 3.68 | PROTEASOME COMPONENT C13 PRECURSOR |
| CCGACGGGCG | 60 | 14-237 | 3.71 | Tag matches ribosomal RNA sequence |
| GCCCCCAATA | 428 | 6-1601 | 3.73 | Lectin, galactoside-binding, soluble, 1 (galectin 1) |
| AGGATGTGGG | 49 | 9-193 | 3.74 | Homo sapiens mRNA for KIAA0706 protein, complete cds |
| GGAGGCCGAG | 26 | 5-103 | 3.75 | ESTs, Weakly similar to allograft inflammatory factor-1 |
| | | | | [H. sapiens] |
| ACCCCCCCGC | 65 | 6-251 | 3.76 | Jun D proto-oncogene |
| CTGGCCTGTG | 30 | 6-120 | 3.80 | Homo sapiens mRNA for CIRP, complete cds |
| CTGGCCTGTG | 30 | 6-120 | 3.80 | Villin 2 (ezrin) |
| CTGGCCTGTG | 30 | 6-120 | 3.80 | Homo sapiens clone 23565 unknown mRNA, partial cds |
| CACCCCCAGG | 29 | 7-118 | 3.80 | ESTs |
| CACCCCCAGG | 29 | 7-118 | 3.80 | Human Gps2 (GPS2) mRNA, complete cds |
| GTGAAACTCC | 66 | 16-269 | 3.81 | Human 53K isoform of Type II phosphatidylinositol-4- |
| | | | | phosphate 5-kinase (PIPK) mRNA, complete cds |
| GTGAAACTCC | 66 | 16-269 | 3.81 | Human mRNA for KIAA0328 gene, partial cds |
| AGAATTGCTT | 50 | 12-201 | 3.81 | Homo sapiens nephrin (NPHS1) mRNA, complete cds |
| AGAATTGCTT | 50 | 12-201 | 3.81 | H. sapiens mRNA for phosphorylase-kinase, beta subunit |
| ATGGCCTCCT | 19 | 5-76 | 3.84 | Human syntaxin mRNA, complete cds |
| AACTGTCCTT | 34 | 5-138 | 3.84 | H. sapiens mRNA for major astrocytic phosphoprotein |
| | | | | PEA-15 |
| AAGGAATCGG | 34 | 5-136 | 3.85 | PROTEASOME BETA CHAIN PRECURSOR |
| TCTGTTTATC | 29 | 8-119 | 3.86 | Signal recognition particle 14 kD protein |
| ACTTTTTCAA | 704 | 20-2741 | 3.87 | Tag matches mitochondrial sequence |
| TCTGTAATCC | 46 | 8-185 | 3.87 | Tag matches mitochondrial sequence |
| TCTGTAATCC | 46 | 8-185 | 3.87 | Human aryl sulfotransferase mRNA, complete cds |
| GTGAAAACCC | 27 | 5-110 | 3.90 | No match |
| GGCAGGCACA | 24 | 5-97 | 3.91 | H. sapiens mRNA for phenylalkylamine binding protein |
| GGGGCAGGGC | 281 | 33-1138 | 3.93 | ESTs, Weakly similar to EPIDERMAL GROWTH FACTOR |
| | | | | PRECURSOR, KIDNEY |
| GGGGCAGGGC | 281 | 33-1138 | 3.93 | Eukaryotic translation initiation factor 5A |
| GTGAAACTCT | 32 | 8-134 | 3.94 | No match |
| TGGACCAGGC | 28 | 7-118 | 3.95 | ESTs, Weakly similar to No definition line found |
| | | | | [C. elegans] |
| CCTATAATCC | 109 | 16-452 | 4.01 | Retinoblastoma-like 1 (p107) |
| CCTATAATCC | 109 | 16-452 | 4.01 | Cyclic nucleotide gated channel (photoreceptor), cGMP |
| | | | | gated 2 (beta) |
| CCTATAATCC | 109 | 16-452 | 4.01 | Homo sapiens mRNA for KIAA0694 protein, complete cds |
| AACTGCTTCA | 77 | 12-323 | 4.05 | Homo sapiens Arp2/3 protein complex subunit p41-Arc |
| | | | | (ARC41) mRNA, complete cds |
| GGATTGTCTG | 55 | 11-233 | 4.07 | Small nuclear ribonucleoprotein polypeptides B and B1 |
| CCTGTAATTC | 48 | 8-201 | 4.07 | Homo sapiens mRNA for KIAA0591 protein, partial cds |
| CTGGGCCTGG | 84 | 7-351 | 4.07 | Human HU-K4 mRNA, complete cds |
| ACCCTTGGCC | 551 | 83-2334 | 4.08 | Tag matches mitochondrial sequence |
| ATGGCGATCT | 27 | 7-117 | 4.09 | Ribosomal protein S24 |
| TTGTCTGCCT | 39 | 8-166 | 4.10 | ESTs |
| TGAATCTGGG | 35 | 6-150 | 4.11 | SET translocation (myeloid leukemia-associated) |
| AGCCTTTGTT | 57 | 6-240 | 4.13 | Human mRNA for collagen binding protein 2, complete cds |
| CTTTTCAGCA | 29 | 9-129 | 4.17 | Human 14-3-3 epsilon mRNA, complete cds |
| CCTGGAGTGG | 28 | 5-123 | 4.17 | ESTs |
| CGGAGACCCT | 87 | 14-380 | 4.20 | Homo sapiens dbpB-like protein mRNA, complete cds |
| CCCTGGGTTC | 1027 | 93-4414 | 4.21 | Ferritin, light polypeptide |
| ATTTGAGAAG | 643 | 93-2814 | 4.23 | Tag matches mitochondrial sequence |
| ACAACTCAAT | 61 | 6-265 | 4.24 | ESTs, Highly similar to BRAIN PROTEIN I3 [Mus |
| | | | | musculus] |
| CTTGATTCCC | 45 | 8-202 | 4.30 | Homo sapiens quiescin (Q6) mRNA, complete cds |
| GGCTGGTCTC | 48 | 9-216 | 4.32 | ESTs |
| AGGTGGCAAG | 194 | 45-891 | 4.36 | Tag matches mitochondrial sequence |
| CTAGCTTTTA | 46 | 10-210 | 4.36 | Tag matches mitochondrial sequence |
| TCACCGGTCA | 143 | 23-648 | 4.38 | GELSOLIN PRECURSOR, PLASMA |
| GGCCGCGTTC | 110 | 5-487 | 4.38 | Ribosomal protein S17 |
| GAGAGCTCCC | 64 | 6-290 | 4.41 | Tag matches mitochondrial sequence |
| GAGAGCTCCC | 64 | 6-290 | 4.41 | EST |
| GAGAGCTCCC | 64 | 6-290 | 4.41 | ESTs |
| GAGAGCTCCC | 64 | 6-290 | 4.41 | Homo sapiens clone 24751 unknown mRNA |
| CCCCGTACAT | 122 | 7-549 | 4.43 | No match |
| TGGCGTACGG | 67 | 11-314 | 4.50 | Tag matches ribosomal RNA sequence |
| TCCCCGACAT | 97 | 5-444 | 4.53 | No match |
| CCTGGCTAAT | 32 | 11-155 | 4.53 | No match |
| TCACAGCTGT | 50 | 10-238 | 4.61 | B-cell translocation gene 1, anti-proliferative |
| TCCCATTAAG | 119 | 12-560 | 4.61 | No match |
| GTGCACTGAG | 259 | 21-1228 | 4.65 | Major histocompatibility complex, class I, C |
| GTGCACTGAG | 259 | 21-1228 | 4.65 | MHC class I protein HLA-A (HLA-A28, -B40, -Cw3) |
| GCTTACCTTT | 35 | 6-170 | 4.68 | Homo sapiens calumein (Calu) mRNA, complete cds |
| CTGGCCCGGA | 54 | 7-264 | 4.71 | Vasodilator-stimulated phosphoprotein |
| CTGGCCCGGA | 54 | 7-264 | 4.71 | Homo sapiens Sox-like transcriptional factor mRNA, |
| | | | | complete cds |
| GGGCCTGTGC | 133 | 11-647 | 4.79 | Homo sapiens monocarboxylate transporter (MCT3) |
| | | | | mRNA, complete cds |
| GGGCCTGTGC | 133 | 11-647 | 4.79 | ESTs |
| GCCCCTCCGG | 121 | 18-598 | 4.79 | ESTs, Weakly similar to TRANS-ACTING |
| | | | | TRANSCRIPTIONAL PROTEIN ICP0 |
| TTGTGATGTA | 21 | 5-109 | 4.87 | Neurotrophic tyrosine kinase, receptor, type 1 |
| TTGTGATGTA | 21 | 5-109 | 4.87 | Fibroblast growth factor receptor 4 |
| CATCTTCACC | 62 | 5-311 | 4.97 | Ribosomal protein S25 |
| TTGGCCAGGA | 100 | 35-539 | 5.06 | No match |
| AGAATCACTT | 37 | 5-194 | 5.09 | No match |
| TTAGCCAGGA | 23 | 8-129 | 5.22 | Human LLGL mRNA, complete cds |
| GTTGTGGTTA | 496 | 43-2646 | 5.25 | BETA-2-MICROGLOBULIN PRECURSOR |
| CAAGCATCCC | 547 | 36-2910 | 5.26 | Tag matches mitochondrial sequence |
| GACATATGTA | 39 | 8-217 | 5.29 | Cytochrome c oxidase subunit VIIb |
| AGTATCTGGG | 63 | 6-337 | 5.29 | Homo sapiens Arp2/3 protein complex subunit p41-Arc |
| | | | | (ARC41) mRNA, complete cds |
| ACCGCCTGTG | 120 | 19-659 | 5.35 | Human transcriptional activator mRNA, complete cds |
| CTCTTCGAGA | 177 | 15-963 | 5.35 | Glutathione peroxidase 1 |
| ATGAGCTGAC | 104 | 11-571 | 5.42 | CYSTATIN B |
| GCCTCTGTCT | 36 | 5-202 | 5.43 | Ribosomal protein, large, P1 |
| AAGGAAGATC | 38 | 6-214 | 5.43 | Human glutathione-S-transferase homolog mRNA, |
| | | | | complete cds |
| AAAACATTCT | 306 | 30-1698 | 5.45 | Tag matches mitochondrial sequence |
| CTCAGACAGT | 64 | 5-385 | 5.95 | ESTs, Highly similar to 40S RIBOSOMAL PROTEIN S27 |
| | | | | [Rattus norvegicus] |
| CCCAAGCTAG | 435 | 54-2698 | 6.08 | Heat shock 27 kD protein 1 |
| CCCAAGCTAG | 435 | 54-2698 | 6.08 | Tag matches ribosomal RNA sequence |
| TCAATCAAGA | 34 | 8-236 | 6.67 | Tyrosine 3-monooxygenase/tryptophan 5-monooxygenase |
| | | | | activation protein, eta polypeptide |
| TGCAGCGCCT | 111 | 9-762 | 6.80 | H. sapiens mRNA for uridine phosphorylase |
| TTCACTGTGA | 223 | 7-1557 | 6.94 | Lectin, galactoside-binding, soluble, 3 (galectin 3) (NOTE: |
| | | | | redefinition of symbol) |
| CTGACCTGTG | 226 | 16-1683 | 7.38 | HLA CLASS I HISTOCOMPATIBILITY ANTIGEN, B-27 |
| | | | | ALPHA CHAIN PRECURSOR |
| GGGGTCAGGG | 118 | 9-882 | 7.43 | Glycogen phosphorylase B (brain form) |
| GGCTTTAGGG | 125 | 10-1019 | 8.05 | Tag matches mitochondrial sequence |
| TGGGTGAGCC | 304 | 45-2538 | 8.21 | Cathepsin B |
| AGGGTGTTTT | 78 | 8-668 | 8.43 | Dual-specificity tyrosine-(Y)-phosphorylation regulated |
| | | | | kinase |
| AGGGTGTTTT | 78 | 8-668 | 8.43 | Tag matches mitochondrial sequence |
| TGGTGTATGC | 93 | 6-810 | 8.62 | Tag matches mitochondrial sequence |
| GAGTAGAGAA | 50 | 8-465 | 9.15 | SET translocation (myeloid leukemia-associated) |
| TGCAGGCCTG | 115 | 11-1165 | 10.02 | TRYPTOPHANYL-TRNA SYNTHETASE |
| GCGAAACCCT | 210 | 34-2242 | 10.51 | V-erb-b2 avian erythroblastic leukemia viral oncogene |
| | | | | homolog 3 {alternative products} |
| GTGACCACGG | 4374 | 29-47260 | 10.80 | Human N-methyl-D-aspartate receptor 2C subunit |
| | | | | precursor (NMDAR2C) mRNA, complete cds |
| GTGACCACGG | 4374 | 29-47260 | 10.80 | Tag matches ribosomal RNA sequence |

TABLE 7
Transcripts uniformly elevated in cancer tissues

| | Cancer | | | | | Normal | | | | | | |
|---|---|---|---|---|---|---|---|---|---|---|---|---|
| Tag | tissues | | | | | Tissues | | | | | Avg | |
| Sequence | CC | BC | BrC | LC | M | NC | NB | NBr | NL | NM | T/N | UniGene Description |
| ATGTGTAACG | 93 | 72 | 13 | 5 | 48 | 0 | 0 | 3 | 0 | 0 | 30 | S100 calcium-binding protein A4 |
| | | | | | | | | | | | | (calcium protein, calvasculin, |
| | | | | | | | | | | | | metastasin) |
| CCCTGCCTTG | 53 | 66 | 120 | 56 | 20 | 21 | 27 | 0 | 8 | 0 | 21 | Midkine (neurite growth-promoting |
| | | | | | | | | | | | | factor 2) |
| GTGCGCTGAG | 85 | 103 | 380 | 23 | 58 | 0 | 30 | 56 | 0 | 8 | 18 | Major histocompatibility complex, |
| | | | | | | | | | | | | class I, C |
| CTGGCCGCTC | 26 | 19 | 53 | 16 | 25 | 3 | 1 | 0 | 0 | 5 | 14 | Apoptosis inhibitor 4 (survivin) |
| GCCCCCCCGT | 38 | 40 | 54 | 31 | 29 | 9 | 7 | 3 | 3 | 0 | 12 | ESTs |
| TGGCCCCAGG | 13 | 201 | 8 | 24 | 336 | 0 | 30 | 3 | 3 | 19 | 9 | Apolipoprotein CI |
| CCCTGGTGGG | 16 | 14 | 17 | 16 | 6 | 0 | 0 | 0 | 0 | 3 | 9 | ESTs |
| AGTGACCGAA | 5 | 8 | 37 | 8 | 7 | 0 | 1 | 0 | 3 | 0 | 8 | ESTs |
| CTGCACTTAC | 52 | 34 | 81 | 64 | 78 | 3 | 12 | 22 | 5 | 30 | 8 | DNA REPLICATION LICENSING FACTOR |
| | | | | | | | | | | | | CDC47 HOMOLOG |
| CTGGCGAGCG | 168 | 137 | 290 | 73 | 178 | 9 | 21 | 64 | 13 | 60 | 8 | Human ubiquitin carrier protein |
| | | | | | | | | | | | | (E2-EPF) mRNA, complete cds |
| TTGCCGCTGC | 4 | 10 | 12 | 19 | 7 | 0 | 1 | 0 | 0 | 0 | 7 | ESTs |
| TGCGCTGGCC | 22 | 63 | 74 | 28 | 14 | 6 | 18 | 6 | 8 | 0 | 7 | No match |
| CTCCTGGAAC | 20 | 10 | 26 | 18 | 18 | 3 | 4 | 0 | 8 | 5 | 6 | ESTs, Highly similar to MYO- |
| | | | | | | | | | | | | INOSITOL-1-PHOSPHATE SYNTHASE |
| | | | | | | | | | | | | [Arabidopsis thaliana] |
| CGCCCGTCGT | 4 | 151 | 30 | 9 | 30 | 0 | 13 | 6 | 0 | 5 | 6 | No match |
| TTGCCCCCGT | 10 | 61 | 15 | 19 | 23 | 0 | 22 | 6 | 5 | 0 | 6 | AXL receptor tyrosine kinase |
| TTGCTAAAGG | 8 | 8 | 16 | 16 | 22 | 3 | 0 | 3 | 8 | 0 | 6 | ESTs, Weakly similar to KIAA0005 |
| | | | | | | | | | | | | [H.sapiens] |
| AGCCACGTTG | 13 | 8 | 11 | 11 | 6 | 0 | 0 | 0 | 0 | 3 | 6 | Acid phosphatase 1, soluble |
| CCTGGGCACT | 14 | 6 | 23 | 22 | 8 | 3 | 1 | 3 | 3 | 0 | 6 | ESTs, Highly similar to |
| | | | | | | | | | | | | transcription factor ARF6 chain B |
| | | | | | | | | | | | | [M.musculus] |
| GGGCTCACCT | 23 | 13 | 52 | 16 | 17 | 3 | 4 | 6 | 3 | 5 | 6 | Homo sapiens clone 24767 mRNA |
| | | | | | | | | | | | | sequence/ESTs, Weakly similar to |
| | | | | | | | | | | | | colt [D.melanogaster] |
| CTTACAGCCA | 11 | 6 | 19 | 12 | 6 | 0 | 0 | 3 | 0 | 3 | 6 | ESTs |
| AGGGCCCTCA | 14 | 6 | 15 | 5 | 4 | 0 | 3 | 0 | 0 | 0 | 6 | Homo sapiens mRNA, complete cds |
| GGGTAATGTG | 7 | 13 | 5 | 11 | 12 | 0 | 1 | 0 | 0 | 5 | 5 | ESTs, Moderately similar to |
| | | | | | | | | | | | | unknown [M.musculus] |
| CTGACAGCCC | 4 | 5 | 17 | 7 | 9 | 0 | 1 | 0 | 0 | 3 | 5 | Human mRNA for HsMcm6, complete cds |
| TGACCTCCAG | 7 | 14 | 15 | 12 | 11 | 0 | 6 | 3 | 3 | 0 | 5 | ESTs, Weakly similar to No |
| | | | | | | | | | | | | definition line found [C.elegans]/ |
| | | | | | | | | | | | | ESTs |
| AAACCTCTTC | 10 | 5 | 12 | 11 | 8 | 0 | 1 | 3 | 0 | 3 | 5 | ESTs, Highly similar to G2/MITOTIC- |
| | | | | | | | | | | | | SPECIFIC CYCLIN B2 [Mesocricetus |
| | | | | | | | | | | | | auratus] |
| TCATTGCACT | 7 | 13 | 5 | 4 | 9 | 3 | 1 | 0 | 0 | 0 | 5 | ESTs, Highly similar to HYPOTHETICAL |
| | | | | | | | | | | | | 16.3 KD PROTEIN [Saccharomyces |
| | | | | | | | | | | | | cerevisiae] |
| CCCCCTCCGG | 31 | 14 | 73 | 38 | 58 | 15 | 3 | 8 | 19 | 11 | 5 | Small nuclear ribonucleoprotein |
| | | | | | | | | | | | | polypeptide N/B and B1 |
| GTAGGGGCCT | 11 | 14 | 11 | 19 | 18 | 3 | 6 | 0 | 3 | 8 | 4 | ESTs |
| GAACCCAAAG | 7 | 8 | 12 | 8 | 10 | 0 | 0 | 3 | 3 | 3 | 4 | Plasminogen/PEPTIDYL-PROLYL CIS- |
| | | | | | | | | | | | | TRANS ISOMERASE A |
| TGTGAGCCTC | 5 | 11 | 11 | 7 | 7 | 0 | 3 | 0 | 0 | 3 | 4 | Cyclin F |
| ATCTCTGGAG | 7 | 3 | 9 | 8 | 7 | 0 | 0 | 0 | 0 | 3 | 4 | ESTs |
| AAAGTGCATC | 10 | 19 | 11 | 4 | 7 | 0 | 9 | 0 | 0 | 3 | 4 | No match |
| GCCTTGGGTG | 7 | 8 | 4 | 9 | 10 | 3 | 3 | 0 | 0 | 0 | 4 | Leukemia inhibitory factor |
| | | | | | | | | | | | | (cholinergic differentiation factor) |
| ACCTCACTCT | 9 | 3 | 12 | 16 | 9 | 0 | 0 | 6 | 3 | 3 | 4 | ESTs |
| TAAAGACTTG | 9 | 13 | 24 | 12 | 38 | 3 | 1 | 11 | 5 | 11 | 4 | Adenylate kinase 2 (adk2) |
| TCGGCGCCGG | 15 | 16 | 21 | 14 | 6 | 6 | 3 | 8 | 3 | 0 | 4 | SET translocation (myeloid leukemia- |
| | | | | | | | | | | | | associated) |
| AACCTCGAGT | 6 | 10 | 7 | 8 | 11 | 0 | 4 | 0 | 3 | 3 | 4 | ESTs, Moderately similar to |
| | | | | | | | | | | | | putative [M.musculus] |
| GTTTACCCGC | 6 | 3 | 4 | 7 | 4 | 0 | 0 | 0 | 0 | 0 | 3 | No match |
| GCCTCTGCCT | 4 | 5 | 5 | 5 | 6 | 0 | 0 | 0 | 0 | 3 | 3 | ESTs |
| CCTGGGTCCT | 4 | 10 | 8 | 5 | 7 | 0 | 4 | 3 | 0 | 3 | 3 | ESTs |

1. A method of identifying a cell as either a colon epithelial cell, a brain cell, a keratinocyte, a breast epithelial cell, a lung epithelial cell, a melanocyte, a prostate cell, or a kidney epithelial cell, comprising the step of:
determining expression in a test cell of a gene product of at least one gene comprising a sequence selected from at least one of the following groups:
(a) the sequences shown in SEQ ID NOS:2, 5-18, 20-84, and 85;
(b) the sequences shown in SEQ ID NOS:87-96, 98, 100-103, 105, 107-110, 112-129, and 131-150, and 151;
(c) the sequences shown in SEQ ID NOS:152-154, and 155;
(d) the sequences shown in SEQ ID NOS:156-159, and 160;
(e) the sequences shown in SEQ ID NOS:161-166, and 167;
(f) the sequences shown in SEQ ID NOS:168, 170, 172-177, 179-188, 190-207, and 208;
(g) the sequences shown in SEQ ID NOS:209 and 210; and
(h) the sequences shown in SEQ ID NOS:211-224 and 225,
wherein expression of a gene product of at least one gene comprising a sequence shown in (a) identifies the test cell as a colon epithelial cell;
wherein expression of a gene product of at least one gene comprising a sequence shown in (b) identifies the test cell as a brain cell;
wherein expression of a gene product of at least one gene comprising a sequence shown in (c) identifies the test cell as a keratinocyte;
wherein expression of a gene product of at least one gene comprising a sequence shown in (d) identifies the test cell as a breast epithelial cell;
wherein expression of a gene product of at least one gene comprising a sequence shown in (e) identifies the test cell as a lung epithelial cell;
wherein expression of a gene product of at least one gene comprising a sequence shown in (f) identifies the test cell as a melanocyte;
wherein expression of a gene product of at least one gene comprising a sequence shown in (g) identifies the test cell as a prostate cell; and
wherein expression of a gene product of at least one gene comprising a sequence shown in (h) identifies the test cell as a kidney epithelial cell.

2. An isolated polynucleotide comprising a sequence selected from the group consisting of SEQ ID NOS:2, 5, 6, 8, 10, 12, 13, 15, 17, 18, 21, 24-26, 28, 30, 31, 34-36, 38, 40, 47-51, 53-57, 59-62, 65-69, 71-76, 78, 80-84, 98, 103, 113, 115, 122, 129, 132, 134, 135, 140, 144, 149, 150, 153-168, 174-176, 182, 185, 186, 188, 190, 200, 201, 205-213, 216-224, 237, 239, 257, 263, 485, 487, 495, 499, 514, 586, 686, 751, 835, 844, 878, 910, 925, 932, 951, 1000, 1005, 1070, 1122, 1130, 1170, 1173, 1187, 1189, 1200, 1213, 1220, 1237, 1257, 1264, 1273, 1293, 1300, 1320, 1367, 1371, 1401, 1403, 1404, 1406, 1418, and 1419.

3. A solid support comprising at least one polynucleotide of claim 2.

4. A method of identifying a test cell as a cancer cell, comprising the step of:
determining expression in a test cell of a gene product of at least one gene comprising a sequence selected from the group consisting of SEQ ID NOS:228, 230-257, 259-260, and 262-265, wherein an increase in said expression of at least two-fold relative to expression of the at least one gene in a normal cell identifies the test cell as a cancer cell.

5. A method of reducing expression of a cancer-specific gene in a human cell, comprising the step of:
administering to the cell a reagent which specifically binds to an expression product of a cancer-specific gene comprising a sequence selected from the group consisting of SEQ ID NOS:228, 230-257, 259-260, and 262-265, whereby expression of the cancer-specific gene is reduced relative to expression of the cancer-specific gene in the absence of the reagent.

6. A method for comparing expression of a gene in a test sample to expression of a gene in a standard sample, comprising the steps of:
determining a first ratio and a second ratio, wherein the first ratio is an amount of an expression product of a test gene in a test sample to an amount of an expression product of at least one gene comprising a sequence selected from the group consisting of SEQ ID NOS:266-375, 377-652, 654-796, and 798-1448 in the test sample, and wherein the second ratio is an amount of an expression product of the test gene in a standard sample to an amount of an expression product of the at least one gene in the standard sample; and
comparing the first and second ratios, wherein a difference between the first and second ratios indicates a difference in the amount of the expression product of the test gene in the test sample.

7. A method of screening candidate anti-cancer drugs, comprising the steps of:
contacting a cancer cell with a test compound; and
measuring expression in the cancer cell of a gene product of at least one gene comprising a sequence selected from the group consisting of SEQ ID NOS: 228, 230-257, 259, 260, 262-263, and 265, wherein a decrease in expression of the gene product in the presence of a test compound relative to expression of the gene product in the absence of the test compound identifies the test compound as a potential anti-cancer drug.

8. A method of screening test compounds for the ability to increase an organ or cell function, comprising the step of:
contacting a cell selected from the group consisting of a colon epithelial cell, a brain cell, a keratinocyte, a breast epithelial cell, a lung epithelial cell, a melanocyte, a prostate cell, and a kidney cell with a test compound; and
measuring expression in the cell of a gene product of at least one gene comprising a sequence selected from at least one of the following groups:
(a) the sequences shown in SEQ ID NOS:2, 5-18, 20-84, and 85;
(b) the sequences shown in SEQ ID NOS:87-96, 98, 100-103, 105, 107-110, 112-129, 131-150, and 151;
(c) the sequences shown in SEQ ID NOS:152-154, and 155;
(d) the sequences shown in SEQ ID NOS:156-159 and 160;
(e) the sequences shown in SEQ ID NOS:161-166 and 167;
(f) the sequences shown in SEQ ID NOS:168, 170, 172-177, 179-188, 190-207, and 208;
(g) the sequences shown in SEQ ID NOS:209 and 210; and
(h) the sequences shown in SEQ ID NOS:211-224 and 225,
wherein an increase in expression of a gene product of at least one gene comprising a sequence selected from (a) identifies the test compound as a potential drug for increasing a function of a colon cell;
wherein an increase in expression of a gene product of at least one gene comprising a sequence selected from (b) identifies the test compound as a potential drug for increasing a function of a brain cell;
wherein an increase in expression of a gene product of at least one gene comprising a sequence selected from (c) identifies the test compound as a potential drug for increasing a function of a skin cell;
wherein an increase in expression of a gene product of at least one gene comprising a sequence selected from (d) identifies the test compound as a potential drug for increasing a function of a breast cell;
wherein an increase in expression of a gene product of at least one gene comprising a sequence selected from (e) identifies the test compound as a potential drug for increasing a function of a lung cell;
wherein an increase in expression of a gene product of at least one gene comprising a sequence selected from (f) identifies the test compound as a potential drug for increasing a function of a melanocyte;
wherein an increase in expression of a gene product of at least one gene comprising a sequence selected from (g) identifies the test compound as a potential drug for increasing a function of a prostate cell; and
wherein an increase in expression of a gene product of at least one gene comprising a sequence selected from (h) identifies the test compound as a potential drug for increasing a function of a kidney cell.

9. A method to restore function to a diseased tissue or cell comprising the step of:
delivering a gene to a diseased cell selected from the group consisting of a colon epithelial cell, a brain cell, a keratinocyte, a breast epithelial cell, a lung epithelial cell, a melanocyte, a prostate cell, and a kidney cell, wherein the gene comprises a nucleotide sequence selected from at least one of the following groups:
(a) the sequences shown in SEQ ID NOS:2, 5-18, 20-84, and 85;
(b) the sequences shown in SEQ ID NOS:87-96, 98, 100-103, 105, 107-110, 112-129, 131-150, and 151;
(c) the sequences shown in SEQ ID NOS:152-154, and 155;
(d) the sequences shown in SEQ ID NOS:156-159 and 160;
(e) the sequences shown in SEQ ID NOS:161-166 and 167;
(f) the sequences shown in SEQ ID NOS:168, 170, 172-177, 179-188, 190-207, and 208;
(g) the sequences shown in SEQ ID NOS:209 and 210; and
(h) the sequences shown in SEQ ID NOS:211-224 and 225,
wherein expression of the gene in the diseased cell is less than expression of the gene in a corresponding cell which is normal,
wherein if the diseased cell is a colon epithelial cell, then the nucleotide sequence is selected from (a);
wherein if the diseased cell is a brain cell, then the nucleotide sequence is selected from (b);
wherein if the diseased cell is a keratinocyte, then the nucleotide sequence is selected from (c);
wherein if the diseased cell is a breast epithelial cell, then the nucleotide sequence is selected from (d);
wherein if the diseased cell is a lung epithelial cell, then the nucleotide sequence is selected from (e);
wherein if the diseased cell is a melanocyte, then the nucleotide sequence is selected from (f);
wherein if the diseased cell is a prostate cell, then the nucleotide sequence is selected from (g); and
wherein if the diseased cell is a kidney cell, then the nucleotide sequence is selected from (h).
